(19) United States
(12) Patent Application Publication
(10) Pub. No.: US 2026/0216072 A1
(43) Pub. Date: Jul. 30, 2026

(54) COMPOSITION

(71) Applicants: BioNTech Delivery Technologies GmbH, Halle (DE); BioNTech SE, Mainz (DE)

(72) Inventors: Steffen Panzner, Halle (DE); Mario Saucedo-Espinosa, Halle (DE); Roy Pattipeiluhu, Mainz (DE); Martin Kirchberg, Halle (DE); Kaushik Thanki, Wiesbaden (DE)

(21) Appl. No.: 19/141,394

(22) PCT Filed: Dec. 21, 2023
(86) PCT No.: PCT/EP2023/087201

§ 371 Date: Jun. 20, 2025

(30) Foreign Application Priority Data
Dec. 23, 2022 (EP) 22216463.4
Oct. 20, 2023 (GB) 2316113.6

(51) Int. Cl. A61K 9/1272; A61K 9/1277; A61K 9/51; A61K 31/7105; A61K 31/711; A61K 39/00; C12N 15/88

(52) U.S. Cl. CPC A61K 9/1272; A61K 9/1277; A61K 9/5123; A61K 9/5192; A61K 31/7105; A61K 31/711; A61K 39/0011; C12N 15/88; A61K 2039/53; A61K 2039/55555

(57) ABSTRACT

A composition comprising: (a) an active ingredient; and (b) a lipid mixture comprising: (i) a cationically ionisable lipid capable of forming a lipid nanoparticle; (ii) a steroid; and (iii) a negatively charged amphiphile having a hydrophilic portion and a lipophilic portion; wherein the composition is a lipid nanoparticle composition and is substantially free of a polyethylene glycol-conjugated lipid, wherein the polyethylene glycol (PEG) moiety of the PEG-conjugated lipid has at least 5 consecutive ethylene glycol repeating units, is provided.

### TECHNICAL FIELD

The present disclosure relates generally to the field of nucleic acid (such as DNA or RNA, in particular mRNA) compositions comprising an anionic amphiphile (as alternative to PEG-conjugated lipids or other stealth polymers conjugated to lipids), and to the use of such compositions, in particular for delivering nucleic acids to cells of a subject or in therapy.

### BACKGROUND TO THE INVENTION

The use of a recombinant nucleic acid (such as DNA or RNA) for delivery of foreign genetic information into target cells is well known. A recombinant nucleic acid may be administered in naked form to a subject in need thereof; however, usually a recombinant nucleic acid is administered using a composition. For example, nucleic acid, such as RNA, may be delivered to a subject using different delivery vehicles, based mostly on cationic polymers or lipids which together with the nucleic acid form nanoparticles. The nanoparticles are intended to protect the nucleic acid, such as RNA, from degradation, enable delivery of the nucleic acid, such as RNA, to the target site and facilitate cellular uptake and processing by the target cells. The efficiency of the nucleic acid delivery depends, in part, on the molecular composition of the nanoparticle and can be influenced by numerous parameters, including particle size, formulation, and charge.

Lipid nanoparticles (LNP) are well established delivery vehicles for RNA. Typically, LNPs comprise a cationically ionisable lipid, a steroid (typically cholesterol), a neutral phospholipid (typically phosphatidylcholine or phosphatidylethanolamine), and a conjugated lipid which is often a PEG-lipid. The cationically ionizable lipid is a main component and serves the purpose of (i) binding and encapsulating the RNA cargo and (ii) binding to and eventually mixing with lipids of the endosomal membrane thereby facilitating an endosome escape. The cationically ionizable lipid together with the steroid and the neutral phospholipid form the body of the LNP which is a polycationic particle. Typically, the LNP bodies alone cannot be isolated or stored due to colloidal instability. This is understood as aggregation between polycationic particles in the presence of even small amounts of non-encapsulated polynucleotides such as RNA or DNA. A similar process of aggregate formation occurs when contacting cationic particles with bodily fluids such as blood or lymph as biomaterials such as proteins (albumin, immunoglobulins etc), lipoproteins or cells are polyanions.

PEG lipids are one example of a wider class of lipids generally termed “stealth lipids”. As described in Le, T. C. et al. Sci. Rep. 2019, 9, 265, according to “Whitesides' rules”, stealth polymers or protein resistant surfaces should have the following characteristics: (a) polar (hydrophilic) functional groups; (b) hydrogen bond acceptor groups, (c) no hydrogen bond donor groups; and (d) no net charge. Examples of stealth polymers previously incorporated into nanomedicine include poly(sarcosine) (pSar), poly(2-oxazoline) (pOx); poly(oxazine) (pOz), poly(vinyl pyrrolidone) (PVP); poly(N-(2-hydroxypropyl) methacrylamide) (pHPMA); poly(dehydroalanine) (pDha); poly(aminoethoxy ethoxy acetic acid) (pAEEA) and poly(2-methylaminoethoxy ethoxy acetic acid) (pmAEEA). All of the above may be conjugated to lipids, the term “stealth lipid” being used generally to describe stealth polymers when conjugated to lipids.

The problem of colloidal instability and bioincompatibility of LNPs is addressed by PEG-conjugated lipids or other stealth polymers providing a steric barrier between the polycationic particle and the polyanionic surface of cells or proteins. PEG steric hindrance prevents inter-particle fusion and promotes the formation of a homogeneous population of LNPs where diameters <100 nm can be achieved.

However, a number of problems are associated with the inclusion of PEG-conjugated lipids or other stealth polymers in LNPs as vehicles for nucleic acid delivery. PEG-conjugated lipids inhibit an interaction between the particles and cells which inhibits cellular uptake and reduces overall transfection efficiency. PEGylation has been associated with accelerated blood clearance (ABC) phenomenon induced by anti-PEG antibodies and/or complement activation as well as storage diseases (Bendele A et al., Toxicological Sciences 1998, 42, 152-157; Young M A et al., 2007, Translational Research 149 (6), 333-342; S. M. Moghimi, J. Szebeni, Progress in Lipid Research 2003 42:463-478). The presence of anti-PEG antibodies in the plasma induces a higher clearance of the particles by the Monophagocyte System (MPS) which at the end reduces the efficacy of the drug. Furthermore, due to broad use of PEGs as ingredients of food, cosmetic, hygienic products, and medicines, a certain percentage of the general population have “pre-existing” anti-PEG antibodies. Such anti-PEG antibodies are associated with decreased efficacy of PEGylated drugs and hypersensitivity reactions that can lead to severe allergic symptoms.

Thus, the use of PEG-conjugated lipids is associated with hypersensitivity and generation of anti-PEG antibodies, which are limiting or even preventing the therapeutic efficacy of PEGylated LNP upon repeated administrations. Without wishing to be bound by theory, similar immune recognition and generation of antibodies may also occur in situations of recurrent treatment with nucleic acid therapeutics using LNPs containing other stealth polymers as a delivery vehicle.

The combined challenges therefore create a need for alternative designs of carrier for nucleic acids. It would therefore be desirable to provide carriers capable of acting as vehicles for delivery of active ingredients, in particular having colloidal stability and being biocompatible upon administration, while avoiding the use of PEG-conjugated lipids and other stealth polymers.

WO02/66012 and Siepi et al. Biophys J. 2011, 100, 2412-2421 describe the use of anionic lipids for carriers of nucleic acids. However, the carrier vehicles described in these documents are liposomes. Liposomes are typically self-closed unilamellar or multilamellar vesicular particles wherein the lamellae comprise lipid bilayers and the encapsulated lumen comprises an aqueous phase. Such liposomes differ from the lipid nanoparticle carriers of the present invention, which do not have an aqueous core. Instead, lipid nanoparticles (LNPs) typically comprise a central complex of nucleic acid and lipid embedded in a disordered, non-lamellar phase made of lipid. In some instances, such an assembly may be surrounded or partially surrounded by a lamellar lipid phase (Trollmann M. F. W. & Böckmann R. A., Biophysical Journal 2022 121 (20): 3927-3939). Typically, LNPs do not comprise or encapsulate an aqueous core or compartment(s).

Semple et al. Nature Biotech 2010, 28, 172-176, describes a mechanism of fusion between LNP and endosomal membranes, wherein a lipid salt is formed between the cationically ionizable lipid of the LNP and anionic lipid of the endosomal membrane. The anionic lipids in Semple are those of the endosomal membrane and the lipid salt is only formed upon fusion. Thus, any use of anionic lipids in the LNP is therefore discouraged, as anionic lipids in the LNP cannot form a lipid salt complex with anionic lipid of the endosomal membrane as required for fusion and endosomal escape. It would be understood from the teachings of Semple et al., that anionic lipids of the LNP could compete with anionic lipids from endosomal membranes for pairing with the cationically ionizable lipid, and thereby even hinder LNP: membrane fusion. Furthermore, typical understanding in the art has been that anionic lipids would compete with the negatively-charged RNA for complex formation with the cationically ionizable lipid in LNPs. Accordingly, general teaching in the art, based on the structure and function of LNP, has been away from using anionic lipids in LNP.

Despite such conceptual drawbacks, use of anionic lipids in LNP has been investigated by other authors. For example, Cheng et al. Nature Nanotechnol., 2020, 15 (4) 313-320 describes incorporation of anionic and/or cationic lipids to PEGylated LNP, and Liu et al. Nat. Mater., 2021, 20, 701-710 describes the introduction of ionizable phospholipids being net-anionic at neutral pH to LNP.

WO2023/036960, unpublished at the earliest priority date of the present application, describes LNP compositions for nucleic acid delivery which include phosphatidylserine and polysorbate 20 (i.e., Tween20), which is a form of PEG-lipid having 20 repeating units of polyethylene glycol. Notably, the carriers described in all of these documents all also comprise PEG-lipid and therefore do not solve the above-described problems addressed by the present invention.

LNPs including other alternatives to PEG-lipids are also known in the art. WO2020/069718 describes RNA particles comprising polysarcosine. WO2023/193892, unpublished at the earliest priority date of the present application, describes LNP compositions for nucleic acid delivery which include an inorganic polyphosphate. U.S. 63/370,046, unpublished at the filing date of the present application, describes LNP compositions for nucleic acid delivery which include an amphiphilic oligo ethylene glycol (OEG)-conjugated compound.

### SUMMARY OF THE INVENTION

In a first aspect, the present disclosure provides a composition comprising:
(a) an active ingredient; and
(b) a lipid mixture comprising:
(i) a cationically ionisable lipid capable of forming a lipid nanoparticle;
(ii) a steroid; and
(iii) a negatively charged amphiphile having a hydrophilic portion and a lipophilic portion;
wherein the composition is a lipid nanoparticle composition and is substantially free of a polyethylene glycol-conjugated lipid, wherein the polyethylene glycol (PEG) moiety of the PEG-conjugated lipid has at least 5 consecutive ethylene glycol repeating units. In the composition of the invention, the lipid mixture may further comprise a neutral or zwitterionic lipid, optionally a neutral or zwitterionic phospholipid.

In a second aspect, the present disclosure relates to a method for delivering an active ingredient (particularly although not exclusively a nucleic acid) to cells of a subject, the method comprising administering to a subject a composition of the first aspect. It is understood that any embodiment described herein in the context of the first aspect may also apply to any embodiment of the second aspect.

In a third aspect, the present disclosure provides the composition of the first aspect for use in medicine, particularly although not exclusively for use in a prophylactic and/or therapeutic treatment of a disease involving an antigen, and/or for use in treating cancer, and/or for use in inducing an immune response.

In a fourth aspect, the present disclosure provides processes for making the composition of the first aspect. In one embodiment of the fourth aspect, provided is a method of preparing the composition of the invention, wherein the composition comprises:
(a) an active ingredient; and
(b) a lipid mixture comprising:
(i) a cationically ionisable lipid capable of forming a lipid nanoparticle;
(ii) a steroid; and
(iii) a negatively charged amphiphile having a hydrophilic portion and a lipophilic portion; wherein the composition is substantially free of a polyethylene glycol-conjugated lipid, wherein the polyethylene glycol (PEG) moiety of the PEG-conjugated lipid has at least 5 consecutive ethylene glycol repeating units;
wherein the method comprises:
(a) providing the active ingredient in an aqueous phase;
(b) providing an organic phase comprising the lipid mixture;
(c) mixing the aqueous phase provided under (a) with the organic phase provided under (b), to form the composition. In such methods, the lipid mixture may further comprise a neutral or zwitterionic lipid, optionally a neutral or zwitterionic phospholipid.

### Advantages and Surprising Findings

It has surprisingly been found by the present inventors that inclusion of anionic amphiphiles, as described herein, into the lipid nanoparticles of the present invention resulted in the lipid nanoparticles having excellent colloidal stability even in the absence of PEG-lipids or other stealth polymers. The lipid nanoparticles comprising anionic amphiphiles have a negative surface charge. Without being bound by theory, the colloidal stability of lipid nanoparticles comprising anionic amphiphiles is thought to be based on electrostatic repulsion, which differs from the colloidal stability by steric hindrance conferred by PEGylated lipid nanoparticles (or those containing other stealth lipids). The lipid nanoparticles containing anionic amphiphiles are colloidally stable and can be frozen and thawed without loss of colloidal stability, and are biocompatible upon administration despite even in the absence of PEGylated lipids or other stealth polymers. It has also surprisingly been found that these effects can be achieved even if only low amounts of anionic amphiphiles are present in the lipid nanoparticle.

It has further been found that in specific embodiments the LNP comprising anionic amphiphiles, but being devoid of any stealth lipid, have a higher transfection efficiency when compared to matching controls comprising such stealth lipid. Without wishing to be bound to a certain theory, the absence of any stealth moiety might eliminate a blockade in cellular or membrane interactions.

### BRIEF DESCRIPTION OF THE FIGURES

FIG. 1 illustrates the particle size of the lipid nanoparticle upon partial replacement of the neutral lipids DSPC by anionic amphiphiles in the presence of PEG-lipid as described in Example 1.

FIG. 2 illustrates the particle size of the lipid nanoparticle upon partial replacement of the neutral lipids DSPC by anionic amphiphiles in the absence of PEG-lipid as described in Example 1.

FIG. 3 shows the in vitro expression across different cell lines for PEGylated LNPs upon partial replacement of the neutral lipid DSPC by anionic amphiphiles in the presence or absence of serum as described in Example 1.

FIG. 4 shows the in vitro expression across different cell lines for LNPs not comprising PEG-lipid upon partial replacement of the neutral lipid DSPC by anionic amphiphiles in the presence or absence of serum as described in Example 1.

FIG. 5 shows in vitro expression for LNP not comprising PEG-lipid upon replacement of neutral lipid by anionic amphiphiles in a full combinatorial design as described in Example 2.

FIG. 6 shows in vitro expression for LNP not comprising PEG-lipid upon replacement of neutral lipid by anionic amphiphiles in a full combinatorial design as described in Example 2.

FIG. 7 shows in vitro expression for LNP not comprising PEG-lipid upon replacement of neutral lipid by anionic amphiphiles in a full combinatorial design as described in Example 2.

FIG. 8 shows the distribution of luciferase expression in mice upon intramuscular injection of LNP comprising anionic amphiphiles as described in Example 4.

FIG. 9 shows the expression kinetics distribution of luciferase expression in mice upon intramuscular injection of LNP comprising anionic amphiphiles as described in Example 4.

FIG. 10 shows the number of spot forming units in an ELISPOT assay for LNP comprising anionic amphiphiles as described in Example 4.

FIG. 11 shows in vitro expression for anionic LNP not comprising a stealth lipid containing different cationically ionizable lipid in a full combinatorial design as described in Example 6.

FIGS. 12 and 13 show in vitro expression for anionic LNP not comprising a stealth lipid containing different anionic amphiphiles for four different cationically ionizable lipids in a full combinatorial design as described in Example 7.

FIGS. 14 and 15 show in vitro expression for anionic LNP not comprising a stealth lipid containing low amounts of three different anionic amphiphiles for two different cationically ionizable lipids in a full combinatorial design as described in Example 8.

FIG. 16 shows a process flow diagram illustrating an improved exemplary method of manufacturing for the anionic LNP as described in Example 9.

FIG. 17 shows a cryogenic transmission electron microscopy (cryo-TEM) image of LNP comprising anionic amphiphile but no stealth lipid.

FIG. 18 shows monitoring the antibody concentration in plasma (cpd Cₛₑᵣᵤₘ) of rats administered a dose of mRNA for five repeated injections (‘X’.Appl).

### DETAILED DESCRIPTION

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995). The practice of the present disclosure will employ, unless otherwise indicated, conventional methods of chemistry, biochemistry, cell biology, immunology, and recombinant DNA techniques which are 25 explained in the literature in the field (cf., e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; Römpp Chemie Lexikon, Falbe/Regitz (Hrsg.), Georg Thieme Verlag Stuttgart, New York, 1989; Molecular Cloning: A 30 Laboratory Manual, 2nd Edition, J. Sambrook et al. eds., Cold Spring Harbor Laboratory Press, Cold Spring Harbor 1989.

All methods described herein can be performed in any suitable order unless otherwise indicated herein or otherwise clearly contradicted by the context. The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±5%, such as ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

“Physiological pH” as used herein refers to a pH of about 7.5 or about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5.

“Physiological conditions” as used herein refer to the conditions (in particular pH and temperature) in a living subject, in particular a human. Preferably, physiological conditions mean a physiological pH and/or a temperature of about 37° C.

As used in the present disclosure, “mol %” is defined as the ratio of the number of moles of one component to the total number of moles of all components, multiplied by 100.

As used in the present disclosure, “mol % of the total lipid” is defined as the ratio of the number of 10 moles of one lipid component to the total number of moles of all lipids, multiplied by 100. In this context, in some embodiments, the term “total lipid” includes lipids and lipid-like material.

The expression “substantially free of X”, as used herein, means that a mixture (such as a composition described herein or an aqueous phase thereof) is free of X in such manner as it is practically and realistically feasible. For example, if the mixture is substantially free of X, the amount of X in the mixture may be less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, or less than 0.001% by weight), based on the total weight of the mixture.

For example, “substantially free of a polyethylene glycol-conjugated lipid, wherein the polyethylene glycol (PEG) moiety of the PEG-conjugated lipid has at least 5 consecutive ethylene glycol repeating units” as used herein, means the composition is free of the stated polyethylene glycol-conjugated lipid in such manner as it is practically and realistically feasible. For example, if the composition is substantially free of a lipid comprising the stated polyethylene glycol-conjugated lipid, the amount of a lipid comprising the stated polyethylene glycol-conjugated lipid in the composition may be less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, 35 less than 0.01% by weight, less than 0.005% by weight, or less than 0.001% by weight), based on the total weight of the mixture. Similar considerations apply to expressions containing the phrase “substantially free of” (such as but not limited to, “substantially free of a polysarcosine-lipid conjugate or a conjugate of polysarcosine and a lipid-like material”, “substantially free of a stealth polymer”, “substantially free of phosphatidylserine”, “substantially free of an inorganic polyphosphate”, “substantially free of a polyoxazoline (POX)-conjugated and/or a polyoxazine (POZ)-conjugated lipid”, “substantially free of a poly(vinyl pyrrolidone) (PVP)”, “substantially free of poly(N-(2-hydroxypropyl) methacrylamide) (pHPMA)”, “substantially free of poly(dehydroalanine) (pDha)”, “substantially free of amphiphilic oligoethylene glycol (OEG)-conjugated lipids” “substantially free of poly(aminoethoxy ethoxy acetic acid) (pAEEA) and/or poly(m N-methyl aminoethoxy ethoxy acetic acid) (pmAEEA)”.

The term “hydrocarbyl” as used herein relates to a monovalent organic group obtained by removing one H atom from a hydrocarbon molecule. In some embodiments, hydrocarbyl groups are non-cyclic, e.g., linear (straight) or branched. Typical examples of hydrocarbyl groups include alkyl, alkenyl, alkynyl, cycloalkyl, aryl groups, and combinations thereof (such as arylalkyl (aralkyl), etc.). Particular examples of hydrocarbyl groups are C₁₋₄₀ alkyl (such as C₆₋₄₀ alkyl, C₆₋₃₀ alkyl, C₆₋₂₀ alkyl, or C₁₀₋₂₀ alkyl), C₂₋₄₀ alkenyl (such as C₆₋₄₀ alkenyl, C₆₋₃₀ alkenyl, or C₆₋₂₀ alkenyl) having 1, 2, or 3 double bonds, aryl, and aryl(C₁₋₆ alkyl). In some embodiments, the hydrocarbyl group is optionally substituted with one or more, such as 1, 2 or 3, such as 1 or 2, such as 1 substituents selected from List A.

The term “heterohydrocarbyl” means a hydrocarbyl group as defined above in which from 1, 2, 3, or 4 carbon atoms in the hydrocarbyl group are replaced by heteroatoms of oxygen, nitrogen, silicon, selenium, phosphorus, or sulfur, preferably O, S, or N. In one embodiment, the heterohydrocarbyl is substituted with one or more, such as 1, 2 or 3, such as 1 or 2, such as 1 substituents selected from List A.

The term “alkyl” refers to a monoradical of a saturated straight or branched hydrocarbon. Preferably, the alkyl group comprises from 1 to 40, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, carbon atoms, such as 1 to 30, such as 1 to 20 carbon atoms, such as 1 to 12 carbon atoms, such as 1 to 10 carbon atoms, such as 1 to 8 carbon atoms, such as 1 to 6 or 1 to 4 carbon atoms. Exemplary alkyl groups include methyl, ethyl, propyl, iso-propyl (also called 2-propyl or 1 methylethyl), butyl, iso-butyl, tert-butyl, n-pentyl, iso-pentyl, sec-pentyl, neo-pentyl, 1,2-dimethylpropyl, iso-amyl, n-hexyl, iso-hexyl, sec-hexyl, n-heptyl, iso-heptyl, n-octyl, 2-ethyl-hexyl, n-nonyl, ndecyl, n-undecyl, n-dodecyl, n-undecyl, n-dodecyl, n-tridecyl, n-tetradecyl, n-pentadecyl, n-hexadecyl, n-heptadecyl, n-octadecyl, n-nonadecyl, n-icosyl, n-triacontyl, n-tetracontyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkyl group are replaced with a substituent other than hydrogen (when more than one hydrogen atom is replaced the substituents may be the same or different). In one embodiment, the alkyl is substituted with one or more, such as 1, 2 or 3, such as 1 or 2, such as 1 substituents selected from List A. Examples of a substituted alkyl include chloromethyl, dichloromethyl, fluoromethyl, and difluoromethyl.

The term “alkylene” refers to a diradical of a saturated straight or branched hydrocarbon. Preferably, the alkylene group comprises from 1 to 40, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, carbon atoms, such as 1 to 30, such as 1 to 20 carbon atoms, such as 1 to 12 carbon atoms, such as 1 to 10 carbon atoms, such as 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH₃) CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene), and the like. The straight alkylene moieties having at least 3 carbon atoms and a free valence at each end can also be designated as a multiple of methylene (e.g., 1,4-butylene can also be called tetramethylene). Generally, instead of using the ending “ylene” for alkylene moieties as specified above, one can also use the ending “diyl” (e.g., 1,2-butylene can also be called butan-1,2-diyl). A “substituted alkylene” means that one or more (such as 1 to the maximum number of hydrogen atoms bound to an alkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituent may be the same or different). In one embodiment, the alkylene is substituted with one or more, such as 1, 2 or 3, such as 1 or 2, such as 1 substituents selected from List A.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds. Preferably, the alkenyl group comprises from 2 to 40 carbon atoms, such as 2 to 30 carbon atoms, such as 2 to 20 carbon atoms, such as 2 to 12 carbon atoms, such as 2 to 10 carbon atoms, such as 2 to 8 carbon atoms, such as 2 to 6 carbon atoms or 2 to 4 carbon atoms. Thus, in a preferred embodiment, the alkenyl group comprises from 2 to 40, such as 2 to 30, such as 2 to 20, such as 2 to 12, such as 2 to 10 carbon atoms and 1, 2, 3, 4, 5, or 6 (e.g., 1, 2, 3, 4, or 5) carbon-carbon double bonds, such as comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, and the like. A “substituted alkenyl” means that one or more (such as 1 to the maximum number of hydrogen atoms bound to an alkenyl group, e.g., 1, 2, 3, 4, 5, 6, 7, 8, 9, or up to 10, such as between 1 to 5, 1 to 4, or 1 to 3, or 1 or 2) hydrogen atoms of the alkenyl group are replaced with a substituent other than hydrogen (when more than one hydrogen atom is replaced the substituents may be the same or different). In one embodiment, the alkenyl is substituted with one or more, such as 1, 2 or 3, such as 1 or 2, such as 1 substituents selected from List A.

The term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds. Preferably, the alkenylene group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenylene group comprises from 2 to 12 (such as 2 to 10 carbon) atoms and 1, 2, 3, 4, 5, or 6 (such as 1, 2, 3, 4, or 5) carbon-carbon double bonds, more preferably 5 it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinylidene (also called ethenylidene), 1-propen-1,2-diyl, 1-propen-1,3-diyl, 1-propen-2,3-diyl, allylidene, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, and the like. A “substituted alkenylene” means that one or more (such as 1 to the maximum number of hydrogen atoms bound to an alkenylene group, e.g., 1, 2, 3, 4, 5, 6, 7, 8, 9, or 15 up to 10, such as between 1 to 5, 1 to 4, or 1 to 3, or 1 or 2) hydrogen atoms of the alkenylene group are replaced with a substituent other than hydrogen (when more than one hydrogen atom is replaced, the substituents may be the same or different). In one embodiment, the alkenylene is substituted with one or more, such as 1, 2 or 3, such as 1 or 2, such as 1 substituents selected from List A.

The term “alkynyl” refers to a linear or branched monovalent hydrocarbon moiety having at least one carbon-carbon triple bond in which the total carbon atoms may be six to forty, such as six to thirty, typically six to twenty, such as six to eighteen. Alkynyl groups can optionally have one or more carbon-carbon triple bonds. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds. A “substituted alkynyl” means that one or more (such as 1 to the maximum number of hydrogen atoms bound to an alkynyl group, e.g., 1, 2, 3, 4, 5, 6, 7, 8, 9, or up to 10, such as between 1 to 5, 1 to 4, or 1 to 3, or 1 or 2) hydrogen atoms of the alkynyl group are replaced with a substituent other than hydrogen (when more than one hydrogen atom is replaced the substituents may be the same or different). In one embodiment, the alkynyl is substituted with one or more, such as 1, 2 or 3, such as 1 or 2, such as 1 substituents selected from List A.

The terms “cycloalkyl” and “cycloalkenyl” represents cyclic non-aromatic versions of “alkyl” and “alkenyl” with preferably 3 to 40, such as 3 to 30, such as 3 to 20, such as 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclobutyl, cyclopentyl, cyclohexyl, cycloheptyl, cyclooctyl, cyclononyl, cyclodecyl, and adamantyl. Exemplary cycloalkenyl groups include cyclopropenyl, cyclobutenyl, cyclopentenyl, cyclohexenyl, cycloheptenyl, cyclooctenyl, cyclononenyl, and cyclodecenyl. The cycloalkyl or cycloalkenyl group may consist of one ring (monocyclic), two rings (bicyclic), or more than two rings (polycyclic). A “substituted cycloalkyl” means that one or more (such as 1 to the maximum number of hydrogen atoms bound to a cycloalkyl group, e.g., 1, 2, 3, 4, 5, 6, 7, 8, 9, or up to 10, such as between 1 to 5, 1 to 4, or 1 to 3, or 1 or 2) hydrogen atoms of the cycloalkyl group are replaced with a substituent other than hydrogen (when more than one hydrogen atom is replaced the substituents may be the same or different). In one embodiment, the cycloalkyl or cycloalkenyl is substituted with one or more, such as 1, 2 or 3, such as 1 or 2, such as 1 substituents selected from List A.

The terms “cycloalkylene” and “cycloalkenylene” represents cyclic non-aromatic versions of “alkylene” and “alkenylene” with preferably 3 to 40, such as 3 to 30, such as 3 to 20, such as 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkylene groups include cyclopropylene, cyclobutylene, cyclopentylene, cyclohexylene, and cycloheptylene. Exemplary cycloalkylenene groups include cyclopentenylene and cyclohexenylene.

The term “aryl” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes. A “substituted aryl” means that one or more (such as 1 to the maximum number of hydrogen atoms bound to an aryl group, e.g., 1, 2, 3, 4, 5, 6, 7, 8, 9, 5 or up to 10, such as between 1 to 5, 1 to 4, or 1 to 3, or 1 or 2) hydrogen atoms of the aryl group are replaced with a substituent other than hydrogen (when more than one hydrogen atom is replaced the substituents may be the same or different). In one embodiment, the aryl is substituted with one or more, such as 1, 2 or 3, such as 1 or 2, such as 1 substituents selected from List A. Examples of a substituted aryl include biphenyl, 2-fluorophenyl, 2-chloro-6-methylphenyl, anilinyl, 4-hydroxyphenyl, and methoxyphenyl (i.e., 2-, 3-, or 4-methoxyphenyl).

The term “heteroaryl” or “heteroaromatic ring” means an aryl group as defined above in which one or more carbon atoms in the aryl group are replaced by heteroatoms of O, S, or N. Preferably, heteroaryl refers to a five or six-membered aromatic monocyclic ring wherein 1, 2, or 3 carbon atoms are replaced by the same or different heteroatoms of O, N, or S. Alternatively, it means an aromatic bicyclic or tricyclic ring system wherein 1, 2, 3, 4, or 5 carbon atoms are replaced with the same or different heteroatoms of O, N, or S. Preferably, in each ring of the heteroaryl group the maximum number of O atoms is 1, the maximum number of S atoms is 1, and the maximum total number of O and S atoms is 2. Exemplary heteroaryl groups include furanyl, thienyl, oxazolyl, isoxazolyl, oxadiazolyl, pyrrolyl, imidazolyl, pyrazolyl, triazolyl, tetrazolyl, thiazolyl, isothiazolyl, thiadiazolyl, pyridyl, pyrimidinyl, pyrazinyl, triazinyl, benzofuranyl, indolyl, isoindolyl, benzothienyl, 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl, pyridazinyl, phenoxazinyl, thiazolopyridinyl, pyrrolothiazolyl, phenothiazinyl, isobenzofuranyl, chromenyl, xanthenyl, pyrrolizinyl, indolizinyl, indazolyl, purinyl, quinolizinyl, phthalazinyl, naphthyridinyl, cinnolinyl, pteridinyl, carbazolyl, phenanthridinyl, acridinyl, perimidinyl, phenanthrolinyl, and phenazinyl. Exemplary 5- or 6-memered heteroaryl groups include furanyl, thienyl, oxazolyl, isoxazolyl, oxadiazolyl, pyrrolyl, imidazolyl (e.g., 2-imidazolyl), pyrazolyl, triazolyl, tetrazolyl, thiazolyl, isothiazolyl, thiadiazolyl, pyridyl (e.g., 4-pyridyl), pyrimidinyl, pyrazinyl, triazinyl, and pyridazinyl. A “substituted heteroaryl” means that one or more (such as 1 to the maximum number of hydrogen atoms bound to a heteroaryl group, e.g., 1, 2, 3, 4, 5, 6, 7, 8, 9, or up to 10, such as between 1 to 5, 1 to 4, or 1 to 3, or 1 or 2) hydrogen atoms of the heteroaryl group are replaced with a substituent other than hydrogen (when more than one hydrogen atom is replaced the substituents may be the same or different). In one embodiment, the heteroaryl is substituted with one or more, such as 1, 2 or 3, such as 1 or 2, such as 1 substituents selected from List A.

The term “heterocyclyl” or “heterocyclic ring” means a cycloalkyl group as defined above in which from 1, 2, 3, or 4 carbon atoms in the cycloalkyl group are replaced by heteroatoms of oxygen, nitrogen, silicon, selenium, phosphorus, or sulfur, preferably O, S, or N. A heterocyclyl group has preferably 1 or 2 rings containing from 3 to 10, such as 3, 4, 5, 6, or 7, ring atoms. Preferably, in each ring of the heterocyclyl group the maximum number of O atoms is 1, the 5 maximum number of S atoms is 1, and the maximum total number of O and S atoms is 2. The term “heterocyclyl” is also meant to encompass partially or completely hydrogenated forms (such as dihydro, tetrahydro or perhydro forms) of the above-mentioned heteroaryl groups. Exemplary heterocyclyl groups include morpholinyl, pyrrolidinyl, imidazolidinyl, pyrazolidinyl, piperidinyl (also called piperidyl), piperazinyl, di- and tetrahydrofuranyl, di- and tetrahydrothienyl, di- and tetrahydropyranyl, urotropinyl, lactones, lactams, cyclic imides, and cyclic anhydrides. A “substituted heterocyclyl” means that one or more (such as 1 to the maximum number of hydrogen atoms bound to a heterocyclyl group, e.g., 1, 2, 3, 4, 5, 6, 7, 8, 9, or up to 10, such as between 1 to 5, 1 to 4, or 1 to 3, or 1 or 2) hydrogen atoms of the heterocyclyl group are replaced with a substituent other than hydrogen (when more than one hydrogen atom is replaced the substituents may be the same or different). In one embodiment, the heterocyclyl is substituted with one or more, such as 1, 2 or 3, such as 1 or 2, such as 1 substituents selected from List A.

The term “alkylcycloalkyl” means a cycloalkyl group, as defined above, which is substituted with an alkyl group, as defined above, the cycloalkyl portion being connected to the rest of the molecule. Each of the cycloalkyl and alkyl portions of the group may take any of the broadest or preferred meanings recited above. A “substituted alkylcycloalkyl” means that one or more (such as 1 to the maximum number of hydrogen atoms bound to a alkylcycloalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or cycloalkyl portions of the group are replaced with a substituent other than hydrogen (when more than one hydrogen atom is replaced the substituents may be the same or different). In one embodiment, the alkylcycloalkyl is substituted with one or more, such as 1, 2 or 3, such as 1 or 2, such as 1 substituents selected from List A.

The term “cycloalkylalkyl” means an alkyl group, as defined above, which is substituted with a cycloalkyl group, as defined above, the alkyl portion being connected to the rest of the molecule. Each of the cycloalkyl and alkyl portions of the group may take any of the broadest or preferred meanings recited above. A “substituted cycloalkylalkyl” means that one or more (such as 1 to the maximum number of hydrogen atoms bound to a cycloalkylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or cycloalkyl portions of the group are replaced with a substituent other than hydrogen (when more than one hydrogen atom is replaced the substituents may be the same or different). In one embodiment, the cycloalkylalkyl is substituted with one or more, such as 1, 2 or 3, such as 1 or 2, such as 1 substituents selected from List A.

The term “alkylcycloalkylalkyl” means an alkyl group, as defined above, which is substituted with a cycloalkyl group, as defined above, the alkyl portion being connected to the rest of the molecule and the cycloalkyl portion in turn being substituted with a further alkyl group. Each of the cycloalkyl and alkyl portions of the group may take any of the broadest or preferred meanings recited above. A “substituted alkylcycloalkylalkyl” means that one or more (such as 1 to the maximum number of hydrogen atoms bound to a alkylcycloalkylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or cycloalkyl portions of the group are replaced with a substituent other than hydrogen (when more than one hydrogen atom is replaced the substituents may be the same or different). In one embodiment, the alkylcycloalkylalkyl is substituted with one or more, such as 1, 2 or 3, such as 1 or 2, such as 1 substituents selected from List A.

The term “alkylaryl” means an aryl group, as defined above, which is substituted with an alkyl group, as defined above, the aryl portion being connected to the rest of the molecule. Each of the aryl and alkyl portions of the group may take any of the broadest or preferred meanings recited above. A “substituted alkylaryl” means that one or more (such as 1 to the maximum number of hydrogen atoms bound to an alkylaryl group, e.g., 1, 2, 3, 4, 5, 6, 7, 8, 9, or up to 10, such as between 1 to 5, 1 to 4, or 1 to 3, or 1 or 2) hydrogen atoms of either the alkyl or aryl portions of the group are replaced with a substituent other than hydrogen (when more than one hydrogen atom is replaced the substituents may be the same or different). In one embodiment, the alkylaryl is substituted with one or more, such as 1, 2 or 3, such as 1 or 2, such as 1 substituents selected from List A.

The term “arylalkyl” means an alkyl group, as defined above, which is substituted with an aryl group, as defined above, the alkyl portion being connected to the rest of the molecule. Each of the aryl and alkyl portions of the group may take any of the broadest or preferred meanings recited above. A “substituted arylalkyl” means that one or more (such as 1 to the maximum number of hydrogen atoms bound to a arylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or aryl portions of the group are replaced with a substituent other than hydrogen (when more than one hydrogen atom is replaced the substituents may be the same or different). In one embodiment, the arylalkyl is substituted with one or more, such as 1, 2 or 3, such as 1 or 2, such as 1 substituents selected from List A.

The term “alkylheteroaryl” means a heteroaryl group, as defined above, which is substituted with an alkyl group, as defined above, the heteroaryl portion being connected to the rest of the molecule. Each of the heteroaryl and alkyl portions of the group may take any of the broadest or preferred meanings recited above. A “substituted alkylheteroaryl” means that one or more (such as 1 to the maximum number of hydrogen atoms bound to an alkylheteroaryl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heteroaryl portions of the group are replaced with a substituent other than hydrogen (when more than one hydrogen atom is replaced the substituents may be the same or different). In one embodiment, the alkylheteroaryl is substituted with one or more, such as 1, 2 or 3, such as 1 or 2, such as 1 substituents selected from List A.

The term “heteroarylalkyl” means an alkyl group, as defined above, which is substituted with a heteroaryl group, as defined above, the alkyl portion being connected to the rest of the molecule. Each of the aryl and alkyl portions of the group may take any of the broadest or preferred meanings recited above. A “substituted heteroarylalkyl” means that one or more (such as 1 to the maximum number of hydrogen atoms bound to a heteroarylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heteroaryl portions of the group are replaced with a substituent other than hydrogen (when more than one hydrogen atom is replaced the substituents may be the same or different). In one embodiment, the heteroarylalkyl is substituted with one or more, such as 1, 2 or 3, such as 1 or 2, such as 1 substituents selected from List A.

The term “alkylheterocyclyl” means a heterocyclyl group, as defined above, which is substituted with an alkyl group, as defined above, the heteroaryl portion being connected to the rest of the molecule. Each of the heterocyclyl and alkyl portions of the group may take any of the broadest or preferred meanings recited above. A “substituted alkylheterocyclyl” means that one or more (such as 1 to the maximum number of hydrogen atoms bound to an alkylheterocyclyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heteroaryl portions of the group are replaced with a substituent other than hydrogen (when more than one hydrogen atom is replaced the substituents may be the same or different). In one embodiment, the alkylheterocyclyl is substituted with one or more, such as 1, 2 or 3, such as 1 or 2, such as 1 substituents selected from List A.

The term “heterocyclylalkyl” means an alkyl group, as defined above, which is substituted with a heterocyclyl group, as defined above, the alkyl portion being connected to the rest of the molecule. Each of the heterocyclyl and alkyl portions of the group may take any of the broadest or preferred meanings recited above. A “substituted heterocyclylalkyl” means that one or more (such as 1 to the maximum number of hydrogen atoms bound to a heterocyclylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heterocyclyl portions of the group are replaced with a substituent other than hydrogen (when more than one hydrogen atom is replaced the substituents may be the same or different). In one embodiment, the heterocyclylalkyl is substituted with one or more, such as 1, 2 or 3, such as 1 or 2, such as 1 substituents selected from List A.

The term “organosulfuric acid” or “sulfate” means a compound of formula R—OSO₂—OH, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). The term “sulfate” is used when the group is deprotonated. Depending on the pH, the sulfate group may be protonated or deprotonated (in the anionic amphiphiles as defined below, the sulfonic acid group is typically deprotonated at physiological pH).

The term “sulfonic acid” or “sulfonate” means a compound of formula R—SO₂—OH, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). The term “sulfonate” is used when the group is deprotonated. Depending on the pH, the sulfonate group may be protonated or deprotonated (in the anionic amphiphiles as defined below, the sulfonate group is typically deprotonated at physiological pH).

The term “carboxylic acid” or “carboxylate” means a compound of formula R—CO₂H, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). The term “carboxylate” is used when the group is deprotonated. Depending on the pH, the carboxylic acid may be protonated or deprotonated (in the anionic amphiphiles as defined below, the carboxylic acid group is typically protonated at acidic pH and deprotonated at neutral or alkaline pH).

The term “dicarboxylic acid” or “dicarboxylate” means a compound of formula HO₂C—R′—CO₂H, wherein R′ is alkylene or alkenylene group (all as defined above, either in a broadest aspect or a preferred aspect). The term “dicarboxylate” is used when the group is deprotonated. Depending on the pH, the dicarboxylic acid may be protonated or deprotonated (in the anionic amphiphiles as defined below, the dicarboxylic acid group is typically protonated at acidic or neutral pH and deprotonated at alkaline pH).

The term “ester” as used herein means a compound having the structure R—C(O)O—R′ (including its isomerically arranged structure R—OC(O)—R′, unless it is specified to the contrary), wherein R and R′ are each independently hydrocarbyl or heterohydrocarbyl groups, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). When the term denotes a substituent connected to the rest of a molecule, the ester moiety may have the structure R—C(O)O— or R—OC(O)—, where R is as defined above. In one embodiment, each of both ends of the ester structure is covalently linked to a C atom of the same organic group or of two separate organic groups (e.g., an alkylene group as further component of the linker).

The term “hemiester” as used herein with respect to a functional moiety relates to an ester of a dicarboxylic acid, as defined above, where one of the carboxylic acid groups forms an ester bond with the rest of the molecule, and the other carboxylic acid group is free. Depending on the pH, the free carboxylic acid group may be protonated or deprotonated (in the anionic amphiphiles as defined below, the free carboxylic acid group is typically protonated at acidic pH and deprotonated at neutral or alkaline pH).

The term “phosphate” means a compound of formula RO—P(═O)(OH)₂, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). Depending on the pH, the phosphate group may be protonated or deprotonated (in the anionic amphiphiles as defined below, the phosphate group is typically deprotonated at physiological pH).

The term “phosphonate” means a compound of formula R—P(═O)(OH)₂, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). Depending on the pH, the phosphonate group may be protonated or deprotonated (in the anionic amphiphiles as defined below, the phosphonate group is typically deprotonated at physiological pH).

“Halo” means fluoro (—F), chloro (—CI), bromo (—Br) or iodo (—I).

“Amine” means the group —NR₂, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group. When both groups R are hydrogen, the amine group is a primary amine group. When one R is hydrogen and the other R is other than hydrogen, the amine group is a secondary amine group. When both groups R are other than hydrogen, the amine group is a tertiary amine group. Examples of preferred tertiary amine groups are defined and exemplified below in relation to cationically ionizable lipids.

“Hydroxyl”—means the group —OH. “Sulfhydryl”—means the group —SH. “Nitro” means the group —NO₂.

“Ether” means an oxygen atom to which two hydrocarbyl or heterohydrocarbyl groups, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl groups (all as defined above, either in a broadest aspect or a preferred aspect) are attached. The ether may be a cyclic ether, wherein the two hydrocarbyl groups together form a ring, and may include dioxolane groups.

“Thioether” means a sulfur atom to which two a hydrocarbyl or heterohydrocarbyl groups, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl groups (all as defined above, either in a broadest aspect or a preferred aspect) are attached. The ether may be a cyclic thioether, wherein the two hydrocarbyl groups together form a ring, and may include dithiane groups.

“Amide” means the group-C(═O)NR(R′), wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect) and is preferably an alkyl group, such as a C₁₋₆ alkyl group.

“Hydroxylamide” means the group —C(═O)O—NR(R′), wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect).

“Sulfonamide” means the group —S(═O)₂NRR′, wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group.

“Carbamate” means the group —O—C(═O)NRR′ wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group.

“Carbohydrate” means a compound having the empirical formula Cₘ(H₂O)ₙ where m may or may not be different from n. The term “carbohydrate residue” or “carbohydrate moiety” defines a residue attached to another atom, where one hydrogen atom of the carbohydrate is replaced by a bond attached to the rest of the molecule. The carbohydrate moiety may be a monosaccharide moiety. The monosaccharide moiety may have the D- or L-configuration. Furthermore, the monosaccharide moiety may be an aldose or ketose moiety. Suitably, the monosaccharide moiety may have 3 to 8, preferably 4 to 6, more preferably 5 or 6, carbon atoms. In one embodiment, the monosaccharide moiety is a hexose moiety (ie it has 6 carbon atoms), examples of which include aldohexoses such as glucose, galactose, allose, altrose, mannose, gulose, idose and talose and ketohexoses such as fructose and sorbose. Preferably, the hexose moiety is a glucose moiety.

In another embodiment, the monosaccharide moiety is a pentose moiety (ie it has 5 carbon atoms), such as ribose, arabinose, xylose or lyxose. Preferably, the pentose moiety is an arabinose or xylose moiety.

In another embodiment, the carbohydrate may be a higher saccharide (ie a di-, or oligosaccharide) comprising more than one monosaccharide moiety joined together by glycoside bonds. When the monosaccharide moieties are hexose moieties, the glycoside bonds may be 1-α, 1′-α glycoside bonds, 1,2′-glycoside bonds (which maybe 1-α2′ or 1′-β-2′ glycoside bonds), 1,3′-glycoside bonds (which may be 1-α-3′ or 1-β-3′-glycoside bonds), 1,4′-glycoside bonds (which may be 1-α-4′ or 1-β-4′-glycoside bonds), 1,6′-glycoside bonds (which may be 1-α-6′ or 1-β-6′-glycoside bonds), or any combination thereof. In one embodiment, the higher saccharide comprises 2 monosaccharide units (i.e. is a disaccharide). Examples of suitable disaccharides include maltose, isomaltose, isomaltulose, lactose, sucrose, cellobiose, nigerose, kojibiose, trehalose and trehalulose. In another embodiment, the higher saccharide comprises 3 to 10 monosaccharide units (ie is an oligosaccharide) in a chain, which may be branched or unbranched. Preferably, the oligosaccharide comprises 3 to 8, more preferably 3 to 6, monosaccharide units. Examples of suitable oligosaccharides include maltodextrin, maltotriose, maltotetraose, maltopentaose, maltohexaose, maltoheptaose, melezitose, cellotriose, cellotetraose, cellopentaose, cellohexaose and celloheptaose.

A “phosphatidic acid” is a compound of formula R—C(═O)—O—CH₂—CH—[O—(C—O)—R′]—CH₂—O—P(═O)(OH)₂, i.e. a compound having a glycerol backbone with an acyl group R—C(═O)—O— bonded to the carbon at the 1-position, another acyl group R′C(═O)—O—bonded to the carbon at the 2-position, and a phosphate group bonded to the carbon at the 3-position. Typically, the phosphate group is deprotonated such that the group is anionic at physiological pH. The groups R and R′ may be the same or different and each is independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). Preferably the group R is an alkyl group, such as a C₆₋₃₀ alkyl group. Preferably the group R′ is an alkenyl group, such as a C₆₋₃₀ alkenyl group.

A “phosphatidylserine” is a compound of formula R—C(═O)—O—CH₂—CH—[O—(C═O)—R′]—CH₂—O—P(═O)(OH)O—CH₂—CH(NH₂) COOH, i.e. a compound having a glycerol backbone with an acyl group R—C(═O)—O— bonded to the carbon at the 1-position, another acyl group R′C(═O)—O—bonded to the carbon at the 2-position, and a phosphate group bonded to the carbon at the 3-position, the phosphate also being esterified with the hydroxyl moiety of a serine residue. Typically, the phosphate group is deprotonated such that the group is anionic at physiological pH. The serine amino acid moiety may be in a zwitterionic form. The groups R and R′ may be the same or different and each is independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). Preferably the group R is an alkyl group, such as a C₆₋₃₀ alkyl group. Preferably the group R′ is an alkenyl group, such as a C₆₋₃₀ alkenyl group.

A “phosphatidylglycerol” is a compound of formula R—C(═O)—O—CH₂—CH—[—O—(C═O)—R′]—CH₂—O—P(═O)(OH)O—CH₂—CH(OH) CH₂OH, i.e. a compound having a first glycerol backbone with an acyl group R—C(═O)—O—bonded to the carbon at the 1-position, another acyl group R′C(═O)—O— bonded to the carbon at the 2-position, and a phosphate group bonded to the carbon at the 3-position, this phosphate group being esterified with a second glycerol moiety. Typically, the phosphate group is deprotonated such that the group is anionic at physiological pH. The groups R and R′ may be the same or different and each is independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). Preferably the group R is an alkyl group, such as a C₆₋₃₀ alkyl group. Preferably the group R′ is an alkenyl group, such as a C₆₋₃₀ alkenyl group.

“List A” substituents are selected from the group consisting of C₁₋₆ alkyl, C₂₋₆ alkenyl, C₂₋₆ alkynyl, 6- to 14-membered (such as 6- to 10-membered) aryl, 3- to 14-membered (such as 5- or 6-membered) heteroaryl, 3- to 14-membered (such as 3- to 7-membered) cycloalkyl, 3- to 14-membered (such as 3- to 7-membered) heterocyclyl, halogen, —CN, azido, —NO₂, —OR′, —N(R′)₂, —S(O)₀₋₂R′, —S(O)₁₋₂OR′, —OS(O)₁₋₂R′, —OS(O)₁₋₂OR′, —S(O)₁₋₂N(R′)₂, —OS(O)₁₋₂N(R′)₂, —N(R′) S(O)₁₋₂R′, —N(R′)S(O)₁₋₂OR′, —C(═X¹)R′, —C(═X¹)X¹R′, —X¹C(═X¹)R′, and —X¹C(═X¹)X¹R′, wherein X¹ is independently selected from O, S, NH and N(CH₃); and each R′ is independently selected from the group consisting of H, C₁₋₄ alkyl, C₂₋₄ alkenyl, C₂₋₄ alkynyl, 5- or 6-membered cycloalkyl, 5- or 6-membered aryl, 5- or 6-membered heteroaryl, and 5- or 6-membered heterocyclyl, wherein each of the alkyl, alkenyl, alkynyl, cycloalkyl, aryl, heteroaryl, and heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —S(C₁₋₃ alkyl), —NH₂, —NH(C₁₋₃ alkyl), —N(C₁₋₃ alkyl)₂, —NHS(O)₂ (C₁₋₃ alkyl), —S(O)₂NH_{2-z}(C₁₋₃ alkyl)_{z}, —C(═O)OH, —C(═O)O(C1-3 alkyl), —C(═O)NH_{2-z}(C₁₋₃ alkyl)_{z}, —NHC(═O)(C₁₋₃ alkyl), —NHC(═NH) NH_{z-2}(C1-3 alkyl)_{z}, and —N(C₁₋₃ alkyl) C(═NH) NH_{2-z} (C₁₋₃ alkyl)_{z}, wherein each z is independently 0, 1, or 2 and each C₁₋₃ alkyl is independently methyl, ethyl, propyl or isopropyl. In some embodiments, List A substituents are selected from List A1, consisting of C₁₋₃ alkyl, phenyl, halogen, —CF₃, —OH, —OCH₃, —SCH₃, —NH_{2-z}(CH₃)_{z}, —C(═O)OH, and —C(═O)OCH₃, wherein z is 0, 1, or 2 and C₁₋₃ alkyl is methyl, ethyl, propyl or isopropyl. In some embodiments, List A substituents are selected from List A2, consisting of methyl, ethyl, propyl, isopropyl, halogen (such as F, Cl, or Br), and —CF₃.

### Lipid Nanoparticle

In the present disclosure, the structural unit of the composition is a lipid nanoparticle (LNP). The function of the LNP is to stabilise and encapsulate the active ingredient (particularly although not exclusively a nucleic acid) to enable it to be delivered into a cell while facilitating its uptake into the cell and release into the cytosol. The LNPs and/or their lipid components may have adjuvant activity.

LNPs are traditionally understood to typically comprise four components: cationically ionizable; neutral lipids such as phospholipids; a steroid such as cholesterol; and a polymer-conjugated lipid (also referred to below as a “stealth lipid”), such as a PEG-conjugated lipid. However, due to the disadvantages of PEG-conjugated lipids (which are the most common polymer-conjugated lipids), LNPs of the present disclosure have a different composition: a cationically ionizable lipid; optionally a neutral lipid such as a phospholipid; a steroid such as cholesterol; and a negatively charged amphiphile having a hydrophilic portion and a lipophilic portion.

Therefore, in one embodiment, the present disclosure provides a composition comprising:
(a) an active ingredient; and
(b) a lipid mixture comprising:
(i) a cationically ionisable lipid capable of forming a lipid nanoparticle;
(ii) a steroid; and
(iii) a negatively charged amphiphile having a hydrophilic portion and a lipophilic portion;
wherein the composition is a lipid nanoparticle composition and is substantially free of a polyethylene glycol-conjugated lipid, wherein the polyethylene glycol (PEG) moiety of the PEG-conjugated lipid has at least 5 consecutive ethylene glycol repeating units.

In another embodiment, the present disclosure provides a composition comprising:
(a) an active ingredient; and
(b) a lipid mixture comprising:
(i) a cationically ionisable lipid capable of forming a lipid nanoparticle;
(ii) a steroid; and
(iii) a negatively charged amphiphile having a hydrophilic portion and a lipophilic portion;
wherein the composition is a lipid nanoparticle composition and is substantially free of a stealth lipid.

LNPs may be understood as oil-in-water emulsions having an oil phase comprising a cationically ionizable lipid and cholesterol. Within the LNP core, typically no charged lipid head groups are present at neutral pH, and the structure is disordered and essentially free of water. LNP core materials are preferably in liquid state and hence have a melting point below body temperature. LNPs thus typically comprise a central complex of nucleic acid and lipid embedded in a disordered, non-lamellar phase made of lipid. This is in contrast to the structure of a liposome which comprises unilamellar or multilamellar vesicular particles wherein the lamellae comprise lipid bilayers surrounding an encapsulated aqueous lumen.

Lipid nanoparticles (LNP) are obtainable from combining active ingredient (particularly although not exclusively a nucleic acid, such as RNA) with lipids. The lipids used for LNP formation do not form lamellar (bilayer) phases in water under physiological conditions. For forming the LNPs of the present invention, the lipids comprise a cationically ionizable lipid, an anionic amphiphile as disclosed herein, a steroid as disclosed herein (such as cholesterol), and optionally an additional lipid, e.g. a neutral or structural lipid as disclosed herein (such as a phospholipid). The LNPs typically do not comprise or encapsulate an aqueous core. The LNPs typically comprise a lipidic (or oily) core.

### Method of Forming Lipid Nanoparticle

In a further aspect, the present disclosure provides methods for producing the lipid nanoparticle composition of the invention.

A number of methods of making LNPs are known in the art and the skilled person would be readily capable of selecting a suitable method and applying this to make the LNP compositions of the present invention. For example, LNP preparations can be produced by rapid mixing of an aqueous solution comprising an active ingredient (typically a nucleic acid such as RNA) and an organic phase comprising a lipid mixture, under conditions such that a sudden change in solubility of lipids is triggered, which drives the lipids towards self-assembly in the form of LNPs. Described herein are exemplary methods of preparing the LNP compositions of the invention.

Thus, provided herein is a method of preparing the composition of the invention, wherein the composition comprises:
(a) an active ingredient; and
(b) a lipid mixture comprising:
(i) a cationically ionisable lipid capable of forming a lipid nanoparticle;
(ii) a steroid;
(iii) a negatively charged amphiphile having a hydrophilic portion and a lipophilic portion; wherein the composition is substantially free of a polyethylene glycol-conjugated lipid, wherein the polyethylene glycol (PEG) moiety of the PEG-conjugated lipid has at least 5 consecutive ethylene glycol repeating units; and
(iv) optionally, a neutral or zwitterionic lipid, optionally a neutral or zwitterionic phospholipid;
wherein the method comprises:
(a) providing the active ingredient in an aqueous phase;
(b) providing an organic phase comprising the lipid mixture;
(c) mixing the aqueous phase provided under (a) with the organic phase provided under (b), to form the composition.

In one embodiment, provided herein is a method of preparing the composition of the invention, wherein the composition comprises:
(a) an active ingredient; and
(b) a lipid mixture comprising:
(i) a cationically ionisable lipid capable of forming a lipid nanoparticle;
(ii) a steroid;
(iii) a negatively charged amphiphile having a hydrophilic portion and a lipophilic portion;
wherein the composition is substantially free of a stealth lipid; and
(iv) optionally, a neutral or zwitterionic lipid, optionally a neutral or zwitterionic phospholipid;
wherein the method comprises:
(a) providing the active ingredient in an aqueous phase;
(b) providing an organic phase comprising the lipid mixture;
(c) mixing the aqueous phase provided under (a) with the organic phase provided under (b), to form the composition.

In one embodiment, provided herein is a method of preparing the composition of the invention, wherein the composition comprises:
(a) an active ingredient; and
(b) a lipid mixture comprising:
(i) a cationically ionisable lipid capable of forming a lipid nanoparticle;
(ii) a steroid;
(iii) a negatively charged amphiphile having a hydrophilic portion and a lipophilic portion; wherein the composition is substantially free of a polyethylene glycol-conjugated lipid, wherein the polyethylene glycol (PEG) moiety of the PEG-conjugated lipid has at least 5 consecutive ethylene glycol repeating units; and
(iv) optionally, a neutral or zwitterionic lipid, optionally a neutral or zwitterionic phospholipid;
wherein the method comprises:
(a) providing the active ingredient in an aqueous phase;
(b) providing an organic phase comprising the lipid mixture;
(c) mixing the aqueous phase provided under (a) with the organic phase provided under (b), to form an intermediate composition; and
(d) immediately after step (c), adjusting the pH of the intermediate composition; to form the composition.

In one embodiment, provided herein is a method of preparing the composition of the invention, wherein the composition comprises:
(a) an active ingredient; and
(b) a lipid mixture comprising:
(i) a cationically ionisable lipid capable of forming a lipid nanoparticle;
(ii) a steroid;
(iii) a negatively charged amphiphile having a hydrophilic portion and a lipophilic portion;
wherein the composition is substantially free of a stealth lipid; and
(iv) optionally, a neutral or zwitterionic lipid, optionally a neutral or zwitterionic phospholipid;
wherein the method comprises:
(a) providing the active ingredient in an aqueous phase;
(b) providing an organic phase comprising the lipid mixture;
(c) mixing the aqueous phase provided under (a) with the organic phase provided under (b), to form an intermediate composition; and
(d) immediately after step (c), adjusting the pH of the intermediate composition; to form the composition.

In the above-defined embodiments, step (d) is typically carried out without delay after step (c). In some embodiments, step (d) is carried out less than 1 minute after step (c). In some embodiments, step (d) is carried out less than 30 seconds after step (c). In some embodiments, step (d) is carried out less than 20 seconds after step (c). In some embodiments, step (d) is carried out less than 10 seconds after step (c). In some embodiments, step (d) is carried out less than 5 seconds after step (c). In some embodiments, step (d) is carried out less than 2 seconds after step (c). In some embodiments, step (d) is carried out less than 1 second after step (c).

The LNPs typically comprise or encapsulate the active ingredient. The active ingredient may be a nucleic acid (e.g., DNA or RNA), preferably RNA (such as mRNA). Additional and preferred features of the composition comprising LNPs, the active ingredient, the lipid mixture, and each of components (i), (ii), (iii) and optionally (iv), are further described herein and that disclosure applies equally to the methods of the invention.

Step (a) of providing the active ingredient in an aqueous phase may comprise mixing the active ingredient with an aqueous phase to prepare a solution of the active ingredient in a first aqueous phase. The aqueous phase may comprise or consist essentially of water. The aqueous phase may comprise or consist essentially of water and a salt (e.g., sodium chloride). The aqueous phase may be acidified (for example, using the acids defined below). The aqueous phase may have a pH below 7.0, such as a pH between about 4.0 and about 6.5, preferably between about 4.0 and about 6.0; in one embodiment, between about 5.2 and about 5.8; in one embodiment, between about 3.7 and about 4.3, in specific embodiments about 4.0 and in other specific embodiments about pH 5.5. The aqueous phase may comprise acetic acid or citric acid. The aqueous phase may comprise water and a buffer system (as defined and exemplified below). The aqueous phase may comprise water and no buffer or essentially no buffer. For example, the aqueous phase may comprise 10 mM or less of a buffer substance.

The aqueous phase may comprise a buffer system, e.g., a buffer system which has a pH below 7.0, such as a pH between about 4.0 and about 6.0. As is known to the person skilled in the art, a buffer system is typically an aqueous solution consisting of a mixture of a weak acid and its conjugate base, or vice versa. Its pH changes very little when a small amount of strong acid or base is added to it. The buffer may be any suitable buffer known in the art. Suitable buffering agents include citrate buffers, acetate buffers, succinate buffers, glutarate buffers, adipate buffer, maleate buffers, malate buffers, tartrate buffers, lactate buffers, PIPES (piperazine-N,N′-bis(2-ethanesulfonic acid)) and MES (2-(N-morpholino)-ethanesulfonic acid). In one embodiment, the buffering agent is a citrate buffer or an acetate buffer.

The organic phase may comprise an organic solvent selected from a lower alcohol, such as alcohols (in particular aliphatic alcohols) having up to 6 carbon atoms, and mixtures of two or more of these alcohols. The organic solvent is preferably completely miscible with water. The organic phase may be an alcohol. The organic phase may be selected from the group consisting of methanol, ethanol, propanol, isopropanol, 1,2-propanediol, butanol, isobutanol, tert-butanol, acetone, dimethyl sulfoxide (DMSO), tetrahydrofuran (THF), and mixtures thereof. Preferred organic phases are ethanol, propanol, isopropanol, acetone, or mixtures thereof. The organic phase may be acidified, e.g. by providing between 0.5 and 1.5 equivalent of acid relative to the cationically ionizable lipid, said acid being selected from hydrochloric acid, acetic acid, succinic acid, glutaric acid, malonic acid, adipic acid, malic acid, malonic acid, tartaric acid, citric acid, lactic acid and the like.

The aqueous phase and/or the organic phase may be acidified. Preferably, at least one of the aqueous phase or the organic phase is acidified. The aqueous phase or the organic phase may comprise an acid. In one embodiment the acid is an inorganic acid, in particular a monobasic inorganic acid, e.g. hydrochloric acid, hydrobromic acid, or nitric acid. In one embodiment the acid is an organic acid, which may be as a mono-, di- or polybasic organic acid. Examples of the organic acid include a monocarboxylic acid such as acetic acid, propionic acid, or lactic acid, a dicarboxylic acid such as oxalic acid, malonic acid, succinic acid, glutaric acid, adipic acid, maleic acid, fumaric acid, tartaric acid, or malic acid, and a polycarboxylic acid such as citric acid, isocitric acid, or trimesic acid. Preferably, the acid is citric acid or acetic acid. The aqueous phase may have a pH of below 7.0, optionally at most about 6.5, such as at most about 6.0, at most about 5.9, at most about 5.8, at most about 5.7. For example, the aqueous phase may have a pH of between about 4.5 and about 6.5, such as between about 5.0 and about 6.0. In one embodiment, the aqueous phase may have a pH of between about 5.2 and about 5.8. In one embodiment, the aqueous phase may have a pH of between about 3.7 and about 4.3.

In one embodiment, the composition is subjected to one or more further steps following the mixing of the organic and aqueous phases in step (c). In one embodiment the composition is subjected to pH adjustment. In one embodiment the composition is subjected to dialysis. In one embodiment the composition is subjected to dilution. In one embodiment the composition is subjected to filtration.

In one embodiment, a pH adjustment step is used to adjust the pH of the composition. The target pH for the pH adjustment step may be between about 6.5 and about 8.0, between about 7.0 and about 8.0, preferably between about 7.2 and about 7.6, most preferably about 7.4. Typically, this is carried out by treating the composition with the required amount of an aqueous alkali so as to reach the target pH. Examples of suitable alkalis are well known to those skilled in the art, and include alkali metal hydroxides (such as lithium, sodium or potassium hydroxides), aqueous ammonia, buffers comprising one or more amine moieties such as tris(hydroxymethyl)-aminomethane (Tris), triethanolamine, 4-(2-hydroxyethyl)-1-piperazine-ethanesulfonic acid (HEPES), histidine, or phosphate buffer. Preferably, the aqueous alkali is a buffer comprising an amine moiety.

After mixing the aqueous phase comprising the active ingredient with the organic phase comprising the lipid mixture, the pH may be adjusted to the target pH to form the composition. Such a pH adjustment step may be performed by addition of an aqueous buffer system, such as those described and exemplified above, and which is preferably selected from tris(hydroxymethyl)aminomethane (Tris), triethanolamine, 4-(2-hydroxyethyl)-1-piperazineethanesulfonic acid (HEPES), histidine, or phosphate buffer. Typically the aqueous buffer system has a pH of above 6.5, optionally above 7.0, preferably above 7.5 (e.g., a pH of between 6.5-8.5, preferably between 7.4 and 8.0). Suitable examples and preferred concentrations include 10-1000 mM HEPES, pH 7.4-8.0 or 10-1000 mM Tris pH7.8-8.2 or 10-1000 mM triethanolamine pH 7.5-8.0 or 10-200 mM sodium diphosphate); these are added to the mixture obtained in step (c).

The pH adjustment step may be performed rapidly so that, for example, the buffer addition and mixing takes less than 5 minutes, preferably less than 1 minute, more preferred less than 30 seconds, more preferred less than 20 seconds, optionally less than 10 seconds, such as less than 5 seconds, such as less than 2 seconds, such as less than 1 second. In a preferred embodiment the buffer is added in a continuous flow mode.

In one embodiment, the composition is subjected to dialysis. As is known to the person skilled in the art, dialysis is the process of separating molecules in solution by the difference in their rates of diffusion through a semipermeable membrane, such as dialysis tubing. The dialysis method may be diffusion dialysis, tangential flow dialysis, electrodialysis, Donnan dialysis, reverse electrodialysis or electro-electrodialysis.

Typically, the dialysis is carried out using one or more buffer systems. Examples of buffer systems are known to those skilled in the art, and are defined and exemplified above. In one embodiment, the buffer system is selected from Tris, triethanolamine, HEPES, histidine, or phosphate buffer.

In one embodiment, the composition is subjected to a diluting step. A diluting step may comprise adding a dilution solution (e.g., water) to an intermediate composition. Such dilution solution may comprise one or more additional compounds (e.g., a cryoprotectant) and optionally a buffer system (as defined and exemplified above). The diluting step may comprise adding the final aqueous phase to an intermediate composition. A diluting step may be carried out to change the pH and/or to change the buffer system and/or to add one or more additional compounds (e.g., a cryoprotectant).

In one embodiment, the composition is subjected to filtration. As is known to the person skilled in the art, filtration typically comprises the removal of solid particles by passing the composition through a filter or membrane, such that particles (especially those of a set particle size exceeding that of the membrane or filter) are removed. Typical filtration methods include sterile filtration and tangential flow filtration.

In one embodiment, provided herein is a method of preparing a composition comprising lipid nanoparticles (LNPs) dispersed in a final aqueous phase, wherein the composition comprises:
(a) an active ingredient; and
(b) a lipid mixture comprising:
(i) a cationically ionisable lipid capable of forming a lipid nanoparticle;
(ii) a steroid;
(iii) a negatively charged amphiphile having a hydrophilic portion and a lipophilic portion; wherein the composition is substantially free of a polyethylene glycol-conjugated lipid, wherein the polyethylene glycol (PEG) moiety of the PEG-conjugated lipid has at least 5 consecutive ethylene glycol repeating units; and
(iv) optionally, a neutral or zwitterionic lipid, optionally a neutral or zwitterionic phospholipid;
wherein the method comprises:
(a) providing the active ingredient in a first aqueous phase;
(b) providing an organic phase comprising the lipid mixture;
(c) mixing the first aqueous phase provided under (a) with the organic phase provided under (b), thereby preparing a first intermediate composition comprising a lipid colloid dispersed in a second aqueous phase;
(d) optionally diluting and/or adjusting the pH of the second aqueous phase; and optionally
(e) dialysing and/or diluting (preferably dialysing) the first intermediate composition prepared under (c) or (d) using the first, second or a final aqueous phase, thereby preparing the composition comprising the LNPs dispersed in the final aqueous phase.

In one embodiment, provided herein is a method of preparing a composition comprising lipid nanoparticles (LNPs) dispersed in a final aqueous phase, wherein the composition comprises:
(a) an active ingredient; and
(b) a lipid mixture comprising:
(i) a cationically ionisable lipid capable of forming a lipid nanoparticle;
(ii) a steroid;
(iii) a negatively charged amphiphile having a hydrophilic portion and a lipophilic portion; wherein the composition is substantially free of a stealth lipid; and
(iv) optionally, a neutral or zwitterionic lipid, optionally a neutral or zwitterionic phospholipid;
wherein the method comprises:
(a) providing the active ingredient in a first aqueous phase;
(b) providing an organic phase comprising the lipid mixture;
(c) mixing the first aqueous phase provided under (a) with the organic phase provided under (b), thereby preparing a first intermediate composition comprising a lipid colloid dispersed in a second aqueous phase;
(d) optionally diluting and/or adjusting the pH of the second aqueous phase; and optionally
(e) dialysing and/or diluting (preferably dialysing) the first intermediate composition prepared under (c) or (d) using the first, second or a final aqueous phase, thereby preparing the composition comprising the LNPs dispersed in the final aqueous phase.

This method is referred to generally herein as “Method A”.

In another embodiment, provided herein is a method of preparing a composition comprising lipid nanoparticles (LNPs) dispersed in a final aqueous phase, wherein the composition comprises
(a) an active ingredient; and
(b) a lipid mixture comprising:
(i) a cationically ionisable lipid capable of forming a lipid nanoparticle;
(ii) a steroid;
(iii) a negatively charged amphiphile having a hydrophilic portion and a lipophilic portion; wherein the composition is substantially free of a polyethylene glycol-conjugated lipid, wherein the polyethylene glycol (PEG) moiety of the PEG-conjugated lipid has at least 5 consecutive ethylene glycol repeating units; and
(iv) optionally, a neutral or zwitterionic lipid, optionally a neutral or zwitterionic phospholipid;
wherein the method comprises:
(a) providing the active ingredient in a first aqueous phase;
(b) providing an organic phase comprising (i) the cationically ionisable lipid capable of forming a lipid nanoparticle; (ii) the steroid, and optionally (iii) the neutral or zwitterionic lipid;
(c) mixing the first aqueous phase provided under (a) with the organic phase provided under (b), thereby preparing a first intermediate composition comprising a lipid colloid dispersed in a second aqueous phase;
(d) optionally diluting and/or adjusting the pH of the second aqueous phase;
(e) providing a solvent comprising (iii) the negatively charged amphiphile having a hydrophilic portion and a lipophilic portion;
(f) mixing the first intermediate composition prepared under (c) or (d) with the negatively charged amphiphile having a hydrophilic portion and a lipophilic portion provided under (e), thereby preparing a second intermediate composition comprising a lipid colloid dispersed in a third aqueous phase; and optionally
(g) dialysing and/or diluting (preferably dialysing) the second intermediate composition prepared under (f) using the final aqueous phase,
thereby preparing the composition comprising the LNPs dispersed in the final aqueous phase.

In another embodiment, provided herein is a method of preparing a composition comprising lipid nanoparticles (LNPs) dispersed in a final aqueous phase, wherein the composition comprises
(a) an active ingredient; and
(b) a lipid mixture comprising:
(i) a cationically ionisable lipid capable of forming a lipid nanoparticle;
(ii) a steroid;
(iii) a negatively charged amphiphile having a hydrophilic portion and a lipophilic portion; wherein the composition is substantially free of a stealth lipid; and
(iv) optionally, a neutral or zwitterionic lipid, optionally a neutral or zwitterionic phospholipid;
wherein the method comprises:
(a) providing the active ingredient in a first aqueous phase;
(b) providing an organic phase comprising (i) the cationically ionisable lipid capable of forming a lipid nanoparticle; (ii) the steroid, and optionally (iii) the neutral or zwitterionic lipid;
(c) mixing the first aqueous phase provided under (a) with the organic phase provided under (b), thereby preparing a first intermediate composition comprising a lipid colloid dispersed in a second aqueous phase;
(d) optionally diluting and/or adjusting the pH of the second aqueous phase;
(e) providing a solvent comprising (iii) the negatively charged amphiphile having a hydrophilic portion and a lipophilic portion;
(f) mixing the first intermediate composition prepared under (c) or (d) with the negatively charged amphiphile having a hydrophilic portion and a lipophilic portion provided under (e), thereby preparing a second intermediate composition comprising a lipid colloid dispersed in a third aqueous phase; and optionally
(g) dialysing and/or diluting (preferably dialysing) the second intermediate composition prepared under (f) using the final aqueous phase,
thereby preparing the composition comprising the LNPs dispersed in the final aqueous phase. This method is referred to generally herein as “Method B”.

This method is referred to generally herein as “Method B”.

Step (a) of providing the active ingredient in a first aqueous phase may comprise mixing an aqueous solution containing the active ingredient with a first aqueous buffer solution to prepare a nucleic acid solution in the first aqueous phase. The first aqueous phase may comprise or consist essentially of water. The first aqueous phase may comprise or consist essentially of water and salt (e.g., sodium chloride). The first aqueous phase may be acidified. The first aqueous phase may have a pH below 6.0, such as a pH between about 3.5 and about 5.9, preferably between about 4.5 and about 5.0. The first aqueous phase may comprise acetic acid or citric acid.

The first aqueous phase may comprise water and a buffer system. The first aqueous phase may comprise water and no buffer or essentially no buffer. The first aqueous phase may comprise a buffer system, e.g., a buffer system which has a pH below 6.0, such as a pH between about 3.5 and about 5.9, preferably between about 4.5 and about 5.0. Examples of buffer systems are known to those skilled in the art, and are defined and exemplified above. Preferably, the buffer system is a citrate buffer or an acetate buffer.

The organic phase provided in step (b) is as defined and exemplified above. Preferred organic phases are ethanol, propanol, isopropanol, acetone, or mixtures thereof. The organic phase may be acidified so as to protonate the cationically ionizable lipid. Typically, between 0.5 and 1.5 equivalents of acid (relative to the cationically ionisable lipid) is added.

The first aqueous phase and/or the organic phase may be acidified, preferably at least one of the first aqueous phase or the organic phase is acidified. The acid may be selected from those defined and exemplified above. Preferably the first aqueous phase or the organic phase comprises hydrochloric acid or acetic acid. The first aqueous phase may have a pH of below 6.0, optionally at most about 5.5, such as at most about 5.0, at most about 4.9, at most about 4.8, at most about 4.7, at most about 4.6, or preferably at most about 4.5. For example, the first aqueous phase may have a pH of between about 3.5 and about 5.9, such as between about 4.0 and about 5.5, or preferably between about 4.5 and about 5.0. Typically, in step (c) the first aqueous phase provided under (a) with the organic phase provided under (b) are mixed in a ratio (a:b) from 6:1 to 1:1 (v/v), preferably 4:1 to 2:1 (v/v), more preferably 3:1 (v/v).

Typically, in step (d) the second aqueous phase is diluted with water in a ratio (second aqueous phase:water) of from 1:1 to 1:5 (v/v), preferably 1:2 (v/v). Without wishing to be bound by theory, it is believed that the water addition is thought to “harden” the very soft and fragile particles obtained in the step (a) which still contain up to 25% of the organic solvent used in that step.

The term “colloid” as used herein relates to a type of homogeneous mixture in which dispersed particles do not settle out.

The dilution or adjustment of pH in step (d) can be done with water in case of a dilution or can be done with buffers. Typically, the buffers are selected from Tris, triethanolamine, HEPES, histidine, or phosphate buffer. Typically, the aqueous buffer system has a pH of above 6.5, optionally above 7.0, preferably above 7.5 (e.g., a pH of between 6.5-8.5, preferably between 7.4 and 8.0). Suitable examples and preferred concentrations include 10-1000 mM HEPES, pH 7.4-8.0 or 10-1000 mM Tris pH7.8-8.2 or 10-1000 mM triethanolamine pH 7.5-8.0 or 10-200 mM sodium diphosphate.

In one embodiment, in step (d) of either Method A or Method B, the pH is adjusted to neutral conditions. In view of the absence of stealth lipids from the composition, pH adjustment preferably needs to be rapid so as to avoid the particles fusing at an intermediate pH between that of the step (a) and neutral pH of about 7 to 8. In one embodiment, pH adjustment step (d) may be performed within 5 seconds, within 10 seconds, within 30 seconds, within 1 minute, within 2 minutes, within 5 minutes, within 10 minutes of mixing step (c). Typically, the pH adjustment is carried out using a buffer: preferably, the buffers are selected from Tris, triethanolamine, HEPES, histidine, or phosphate buffer.

Following the pH adjustment step (d), in method B, the anionic amphiphile prepared under step (e) is added under step (f). Following this step, the product may be subjected to standard purification steps such as dialysis, increasing the concentration, filtration, compounding, final filtration, and filling and finishing, methods of which are well known to those skilled in the art.

In Method A, dialysis or dilution step (e) may be performed essentially immediately after dilution and/pH adjustment in step (d) In Method A, filtrating step (d) may be performed at least 5 seconds, at least 10 seconds, at least 30 seconds, at least 1 minute, at least 2 minutes, at least 5 minutes, at least 10 minutes, at least 20 minutes, at least 30 minutes, at least an hour, at least 6 hours, at least 12 hours, at least 24 hours, at least 48 hours or more than 72 hours, after dilution/pH adjustment step (d).

A difference between Method A and Method B is that in Method A, the active ingredient is mixed with all of the components of the lipid mixture in a single step (c) to form a first intermediate composition comprising a lipid colloid dispersed in the second aqueous phase, wherein the second aqueous phase is typically a mixture of the first aqueous phase and the organic phase resulting from the first mixing step (c), and is typically acidified. The dilution/pH adjustment step (d) of Method A is typically performed to provide a fast transition from an acidic pH to the target pH, which is typically neutral. As discussed above, the target pH for the final LNP composition in the final aqueous phase is between about 7.0 and about 8.0, preferably between about 7.2 and about 7.8, most preferably between about 7.3 and about 7.5.

In contrast, Method B describes an alternative two-step process, where a lipid colloid encapsulating the active ingredient is formed with the cationically ionisable lipid, the steroid, and, optionally, the neutral or zwitterionic lipid in a first step, and then the negatively charged amphiphile is added in a subsequent step to form a second intermediate composition comprising a lipid colloid. The negatively charged amphiphile is incorporated into the lipid colloid encapsulating the active ingredient. One possible advantage of this two-step process is that the negatively charged amphiphile does not contact the active ingredient, which may be negatively charged nucleic acid (such as RNA), before it is incorporated into a lipid colloid. This may help to reduce formation of unstable by-products, such as so called ‘late migrating species’ (LMS) contaminants.

In Method B, the solvent comprising (iii) the negatively charged amphiphile having a hydrophilic portion and a lipophilic portion may be an organic solvent, as described herein, such as an alcohol, preferably a C1-4 alcohol, such as ethanol or isopropanol. The solvent may be water or the solvent may be a mixture of an alcohol (preferably a C1-4 alcohol) in water such as 30% ethanol or isopropanol in water or 50% ethanol or isopropanol in water or 75% ethanol or isopropanol in water. The solvent may be an aqueous phase comprising water and a buffer system (as defined and exemplified above). The pH of the solvent may be basic, for example the pH may be between 7 and 9, preferably between pH 7.5 and 8.5, to achieve both deprotonation of the anionic amphiphile and, upon combination with the second aqueous phase, adjustment to the target pH. Such a pH adjustment step may be performed by addition of alkaline, such as sodium hydroxide or potassium hydroxide or ammonium hydroxide, or by adding an aqueous buffer system, such as those described and exemplified above and which are preferably selected from Tris, triethanolamine, HEPES, histidine, or phosphate buffer). It is of course possible to use the anionic amphiphile in its salt form, for example as sodium salt or ammonium salt or tris(hydroxymethyl)-aminomethanium salt or triethylammonium salt. This avoids the risk of using excess alkali.

The second intermediate composition of Method B typically comprises a lipid colloid comprising the active ingredient and all of components (i), (ii), (iii) and optionally (iv) of the lipid mixture, dispersed in a third aqueous phase. The third aqueous phase is typically a mixture of the first aqueous phase, the organic phase and the solvent, which is formed by mixing steps (c) and (f).

Mixing step (f) of Method B may be performed after mixing step (c) and before dialysing and/or diluting step (g). Alternatively, mixing step (f) of Method B may be performed after mixing step (c) and concurrently with dialysing and/or diluting step (g). Alternatively, Method B may further comprise a pH adjustment step (d), after step (c) and before mixing step (e). Such a pH adjustment step (d) may be performed to change the pH of the first intermediate composition to the target pH, or to a pH between about 4.0 and about 8.0, such as between about 4.5 and about 8.0, such as between about 5.0 and about 8.0, between about 5.5 and about 8.0, between about 6.0 and about 8.0, between about 6.5 and about 8.0, between about 7.0 and about 8.0, preferably between about 7.2 and about 7.8, most preferably between about 7.3 and about 7.5. Thus, in some instances of Method B, the first intermediate composition may be brought to a neutral pH prior to addition of the negatively charged amphiphile.

In Method B, mixing step (f) may be performed essentially immediately after mixing step (c). In Method B, mixing step (e) may be performed within at least 5 seconds, at least 10 seconds, at least 30 seconds, at least 1 minute, at least 2 minutes, at least 5 minutes, at least 10 minutes, at least 20 minutes, at least 30 minutes, at least an hour, at least 6 hours, at least 12 hours, at least 24 hours, within at least 48 hours and no more than 72 hours, after mixing step (c). In Method B, dialysing and/or diluting step (g) may be performed concurrently with mixing step (e). In Method B, dialysing and/or diluting step (g) may be performed essentially immediately after mixing step (f). In Method B, filtering step (f) may be performed within at least 5 seconds, at least 10 seconds, at least 30 seconds, at least 1 minute, at least 2 minutes, at least 5 minutes, at least 10 minutes, at least 20 minutes, at least 30 minutes, at least an hour, at least 6 hours, at least 12 hours, at least 24 hours, within at least 48 hours and no more than 72 hours, after mixing step (e).

Where the active ingredient is a nucleic acid (such as RNA), the concentration of the nucleic acid in the composition comprising the LNPs dispersed in the final aqueous phase may be about 1 mg/l to about 2000 mg/l, such as about 100 mg/l to about 800 mg/l. The concentration of the nucleic acid in the composition may be about 5 mg/l to about 500 mg/l, such as about 10 mg/l to about 400 mg/l, about 10 mg/l to about 300 mg/l, about 10 mg/l to about 200 mg/l, about 10 mg/l to about 150 mg/l, or about 10 mg/l to about 100 mg/l, preferably about 10 mg/l to about 140 mg/l, more preferably about 20 mg/l to about 130 mg/l, more preferably about 30 mg/l to about 120 mg/l. In some embodiments, the concentration of the nucleic acid (in particular RNA) in the composition is 1 mg/l to about 50 mg/l or about 10 mg/l to about 100 mg/l.

The methods may further comprise a step of freezing the composition comprising the LNPs dispersed in the final aqueous phase. The composition may be frozen to a temperature of −10° C. or below (e.g., to −15° C. or below, preferably to −20° C. or below, in preferred embodiments to about −70° C.). Thus, provided herein are also compositions of the invention in frozen form. The methods may further comprise a step of lyophilizing the composition comprising the LNPs dispersed in the final aqueous phase. Thus, provided herein are also compositions of the invention in lyophilized form.

In some embodiments, the lipid nanoparticles described herein have an average diameter that in some embodiments ranges from about 50 nm to about 1000 nm, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nm to about 800 nm, from about 150 nm to about 700 nm, from about 150 nm to about 600 nm, from about 150 nm to about 500 nm, from about 150 nm to about 450 nm, from about 150 nm to about 400 nm, from about 150 nm to about 350 nm, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, or from about 200 nm to about 250 nm. The LNPs described herein may have an average diameter of from about 40 nm to about 300 nm, preferably from about 40 nm to about 250 nm; most preferably from about 50 nm to about 200 nm and even more preferred from about 60 nm to 120 nm.

### Active Ingredient

The compositions of the present application contain an active ingredient. The active ingredient may be any substance capable of exerting a therapeutic effect, particularly although not exclusively when transfected into a cell. The active ingredient may be a nucleic acid. The active ingredient is preferably RNA, such as mRNA.

### Nucleic Acid

In one embodiment, the active ingredient is a nucleic acid. The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. In one embodiment, the nucleic acid is RNA. In one embodiment, the nucleic acid is mRNA.

A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis.

The term “nucleoside” relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine. Nucleic acids may include one or more modified nucleosides or nucleotides. Examples of modified nucleosides or nucleotides which may be incorporated into nucleic acids include N7-alkylguanine, N6-alkyl-adenine, 5-alkyl-cytosine, 5-alkyl-uracil, and N (1)-alkyl-uracil, such as N7-C1-4 alkylguanine, N6-C1-4 alkyl-adenine, 5-C1-4 alkyl-cytosine, 5-C1-4 alkyl-uracil, and N (1)-C1-4 alkyl-uracil, preferably N7-methyl-guanine, N6-methyl-adenine, 5-methyl-cytosine, 5-methyl-uridine (m5U), pseudouridine (ψ), and N1-methyl-pseudouridine (m1Ψ).

### DNA

In some embodiments of all aspects of the disclosure, the nucleic acid is DNA. Herein, the term “DNA” relates to a nucleic acid molecule which includes deoxyribonucleotide residues. DNA typically comprises the naturally occurring nucleic acids adenosine (dA), thymidine (dT), cytidine (dC) and guanosine (dG) (“d” represents “deoxy”). In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxy-ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof). DNA may be recombinant DNA and may be obtained by cloning of a nucleic acid, in particular cDNA. The cDNA may be obtained by reverse transcription of RNA.

### RNA

In some embodiments of all aspects of the disclosure, the nucleic acid is RNA. According to the present disclosure, the term “RNA” means a nucleic acid molecule which includes ribonucleotide residues. RNA typically comprises the naturally occurring nucleic acids adenosine (A), uridine (U), cytidine (C) and guanosine (G). In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides (or modified nucleosides) can be referred to as analogs of naturally occurring nucleotides (nucleosides), and the corresponding RNAs containing such altered/modified nucleotides or nucleosides (i.e., altered/modified RNAs) can be referred to as analogs of naturally occurring RNAs. A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof). “RNA” includes mRNA, tRNA, ribosomal RNA (rRNA), small nuclear RNA (snRNA), self-amplifying RNA (saRNA), trans-amplifying RNA (taRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA). In some embodiments, “RNA” refers to mRNA. The active ingredient may be mRNA, saRNA, taRNA, or mixtures thereof. The active ingredient is preferably mRNA. In some instances, the active ingredient is not siRNA.

In a preferred embodiment, the RNA comprises an open reading frame (ORF) encoding a peptide, polypeptide or protein. Said RNA may capable of or configured to express the encoded peptide, polypeptide, or protein. For example, said RNA may be RNA encoding and capable of or configured for expressing a pharmaceutically active peptide or protein. In some embodiments, RNA is able to interact with the cellular translation machinery allowing translation of the peptide or protein. A cell may produce the encoded peptide or protein intracellularly (e.g. in the cytoplasm), may secrete the encoded peptide or protein, or may produce it on the surface. Alternatively, the RNA can be non-coding RNA such as antisense-RNA, micro RNA (miRNA) or siRNA.

mRNA

In preferred embodiments of all aspects of the disclosure, the nucleic acid is mRNA. According to the present disclosure, the term “mRNA” means “messenger-RNA” and includes a “transcript” which may be generated by using a DNA template. Generally, mRNA encodes a peptide, polypeptide or protein. As established in the art, the RNA (such as mRNA) generally contains a 5′ untranslated region (5′-UTR), a peptide/polypeptide/protein coding region and a 3′ untranslated region (3′-UTR).

mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In preferred embodiments of the present disclosure, the mRNA relates to an RNA transcript which encodes a peptide, polypeptide or protein.

In some embodiments, the RNA which preferably encodes a peptide, polypeptide or protein has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

In some embodiments, the RNA (such as mRNA) is produced by in vitro transcription or chemical synthesis. Preferably, the RNA (such as mRNA) is produced by in vitro transcription using a DNA template. The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)). The in vitro transcription methodology is known to the skilled person; cf., e.g., Molecular Cloning: A Laboratory Manual, 2nd Edition, J. Sambrook et al. eds., Cold Spring Harbor Laboratory Press, Cold Spring Harbor 1989. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™).

For providing modified RNA (such as mRNA), correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription. The RNA (such as mRNA) may be modified. The RNA (such as mRNA) may comprise modified nucleotides or nucleosides, such as 5-methyl-cytosine, 5-methyl-uridine (m5U), pseudouridine (ψ) or N(1)-methyl-pseudouridine (m1ψ). One or more uridine in the RNA described herein may be replaced by a modified nucleoside. The modified nucleoside may be a modified uridine. The RNA may comprise a modified nucleoside in place of at least one uridine. Preferably, the RNA may comprise a modified nucleoside in place of each uridine (e.g., all of the uridines in the RNA are replaced with a modified nucleoside). The modified nucleoside may be independently selected from pseudouridine (ψ), N1-methyl-pseudouridine (m1ψ), and 5-methyl-uridine (m5U). The modified nucleoside is preferably pseudouridine (ψ) or N1-methyl-pseudouridine (m1ψ).

In some embodiments, RNA (such as mRNA) is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments of the present disclosure, the RNA (such as mRNA) is “replicon RNA” (such as “replicon mRNA”) or simply a “replicon”, in particular “self-replicating RNA” (such as “self-replicating mRNA”) or “self-amplifying RNA” (or “self-amplifying mRNA”).

### Inhibitory RNA

In some embodiments of all aspects of the disclosure, the nucleic acid is an inhibitory RNA.

The term “inhibitory RNA” as used herein means RNA which selectively hybridizes to and/or is specific for a target mRNA, thereby inhibiting (e.g., reducing) transcription and/or translation thereof. Inhibitory RNA includes RNA molecules having sequences in the antisense orientation relative to the target mRNA. Suitable inhibitory oligonucleotides typically vary in length from five to several hundred nucleotides, more typically about 20 to 70 nucleotides in length or shorter, even more typically about 10 to 30 nucleotides in length. Examples of inhibitory RNA include antisense RNA, ribozyme, iRNA, siRNA and miRNA. In some embodiments of all aspects of the disclosure, the inhibitory RNA is siRNA.

The term “antisense RNA” as used herein refers to an RNA which hybridizes under physiological conditions to DNA comprising a particular gene or to mRNA of said gene, thereby inhibiting transcription of said gene and/or translation of said mRNA. The size of the antisense RNA may vary from 15 nucleotides to 15,000, preferably 20 to 12,000, in particular 100 to 10,000, 150 to 8,000, 200 to 7,000, 250 to 6,000, 300 to 5,000 nucleotides, such as 15 to 2,000, 20 to 1,000, 25 to 800, 30 to 600, 35 to 500, 40 to 400, 45 to 300, 50 to 250, 55 to 200, 60 to 150, or 65 to 100 nucleotides.

By “small interfering RNA” or “siRNA” as used herein is meant an RNA molecule, preferably greater than 10 nucleotides in length, more preferably greater than 15 nucleotides in length, and most preferably 18, 19, 20, 21, 22, 23, 24, 25, 26, 27, 28, 29, or 30 nucleotides in length that is capable of binding specifically to a portion of a target mRNA. This binding induces a process, in which said portion of the target mRNA is cut or degraded and thereby the gene expression of said target mRNA inhibited. A range of 19 to 25 nucleotides is the most preferred size for siRNAs. Typically siRNAs comprise a single molecule in which two complementary portions are base-paired and are covalently linked by a single-stranded “hairpin” area. Without wishing to be bound by any theory, it is believed that the hairpin area of the siRNA molecule is cleaved intracellularly by the “Dicer” protein (or its equivalent) to form an siRNA of two individual base-paired RNA molecules.

As used herein, “target mRNA” refers to an RNA molecule that is a target for downregulation. In some embodiments, the target mRNA comprises an ORF encoding a pharmaceutically active peptide or polypeptide as specified herein. In some embodiments, the pharmaceutically active peptide or polypeptide is one whose expression (in particular increased expression, e.g., compared to the expression in a healthy subject) is associated with a disease. In some embodiments, the target mRNA comprises an ORF encoding a pharmaceutically active peptide or polypeptide whose expression (in particular increased expression, e.g., compared to the expression in a healthy subject) is associated with cancer.

According to the present disclosure, siRNA can be targeted to any stretch of approximately 19 to 25 contiguous nucleotides in any of the target mRNA sequences (the “target sequence”). Techniques for selecting target sequences for siRNA are given, for example, in Tuschl T. et al., “The siRNA User Guide”, revised Oct. 11, 2002, the entire disclosure of which is herein incorporated by reference. Further guidance with respect to the selection of target sequences and/or the design of siRNA can be found on the webpages of Protocol Online (www.protocol-online.com) using the keyword “siRNA”. Thus, in some embodiments, the sense strand of the siRNA used in the present disclosure comprises a nucleotide sequence substantially identical to any contiguous stretch of about 19 to about 25 nucleotides in the target mRNA.

siRNA can be obtained using a number of techniques known to those of skill in the art. For example, siRNA can be chemically synthesized or recombinantly produced. Preferably, siRNA is transcribed from recombinant circular or linear DNA plasmids using any suitable promoter. Selection of other suitable promoters is within the skill in the art. Selection of plasmids suitable for transcribing siRNA, methods for inserting nucleic acid sequences for expressing the siRNA into the plasmid, and IVT methods of in vitro transcription of said siRNA are within the skill in the art.

The term “miRNA” (microRNA) as used herein relates to non-coding RNAs which have a length of 21 to 25 (such as 21 to 23, preferably 22) nucleotides and which induce degradation and/or prevent translation of target mRNAs. miRNAs are typically found in plants, animals and some viruses, wherein they are encoded by eukaryotic nuclear DNA in plants and animals and by viral DNA (in viruses whose genome is based on DNA), respectively. miRNAs are post-transcriptional regulators that bind to complementary sequences on target messenger RNA transcripts (mRNAs), usually resulting in translational repression or target degradation and gene silencing.

miRNA can be obtained using a number of techniques known to those of skill in the art. For example, miRNA can be chemically synthesized or recombinantly produced using methods known in the art (e.g., by using commercially available kits such as the miRNA cDNA Synthesis Kit sold by Applied Biological Materials Inc.). Preferably, miRNA is transcribed from recombinant circular or linear DNA plasmids using any suitable promoter.

### Pharmaceutically Active Peptides or Polypeptides

“Encoding” refers to the inherent property of specific sequences of nucleotides in a polynucleotide, such as a gene, a cDNA, or an RNA (preferably mRNA), to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of RNA (preferably mRNA) corresponding to that gene produces the protein in a cell or other biological system. Similarly, an RNA (such as mRNA) encodes a protein if translation of that RNA (e.g., in a cell) produces that protein.

In some embodiments, the active ingredient is an RNA (preferably mRNA), as described in the present disclosure, which comprises a nucleic acid sequence (e.g., an ORF) encoding one or more polypeptides, e.g., a peptide or protein, preferably a pharmaceutically active peptide or protein. In some embodiments, the RNA (preferably mRNA) described in the present disclosure is capable of expressing said peptide or protein, in particular if transferred into a cell or subject. Thus, in some embodiments, the RNA (preferably mRNA) described in the present disclosure contains a coding region (ORF) encoding a peptide or protein, preferably encoding a pharmaceutically active peptide or protein. In this respect, an “open reading frame” or “ORF” is a continuous stretch of codons beginning with a start codon and ending with a stop codon. Such RNA (preferably mRNA) encoding a pharmaceutically active peptide or protein is also referred to herein as “pharmaceutically active RNA” (or “pharmaceutically active mRNA”). In some embodiments, RNA (preferably mRNA) described in the present disclosure comprises a nucleic acid sequence encoding more than one peptide or polypeptide, e.g., two, three, four or more peptides or polypeptides.

In one embodiment, the active ingredient is a pharmaceutically active peptide or protein. According to the present disclosure, the term “pharmaceutically active peptide or protein” means a peptide or protein that can be used in the treatment of an individual where the expression of the peptide or protein would be of benefit, e.g., in ameliorating the symptoms of a disease or disorder. Preferably, a pharmaceutically active peptide or protein has curative or palliative properties and may be administered to ameliorate, relieve, alleviate, reverse, delay onset of or lessen the severity of one or more symptoms of a disease or disorder. In some embodiments, a pharmaceutically active peptide or protein has a positive or advantageous effect on the condition or disease state of an individual when administered to the individual in a therapeutically effective amount. A pharmaceutically active peptide or protein may have prophylactic properties and may be used to delay the onset of a disease or disorder or to lessen the severity of such disease or disorder. The term “pharmaceutically active peptide or protein” includes entire proteins or polypeptides, and can also refer to pharmaceutically active fragments thereof. It can also include pharmaceutically active analogs of a peptide or protein. The terms “pharmaceutically active peptide or protein” and “therapeutic protein” are used interchangeable herein.

Specific examples of pharmaceutically active peptides and proteins include, but are not limited to, immunostimulants, e.g., cytokines, hormones, adhesion molecules, immunoglobulins, immunologically active compounds, growth factors, protease inhibitors, enzymes, receptors, apoptosis regulators, transcription factors, tumor suppressor proteins, structural proteins, reprogramming factors, genomic engineering proteins, and blood proteins. In some embodiments, the pharmaceutically active peptide and polypeptide includes a replacement protein.

An “immunostimulant” is any substance that stimulates the immune system by inducing activation or increasing activity of any of the immune system's components, in particular immune effector cells. The immunostimulant may be pro-inflammatory (e.g., when treating infections or cancer), or anti-inflammatory (e.g., when treating autoimmune diseases).

In some embodiments, the immunostimulant is a cytokine or a variant thereof. Examples of cytokines include interferons, such as interferon-alpha (IFN-α), interferon beta (IFNβ) or interferon-gamma (IFN-γ), interleukins, such as interleukin 2 (IL2), IL-4, IL7, IL-10, IL-11, IL12, IL15, IL-21 and IL23, colony stimulating factors, such as colony stimulating factor (CSF), granulocyte colony stimulating factor (G-CSF), macrophage colony stimulating factor (M-CSF) and granulocyte-macrophage colony stimulating factor (GM-CSF), tumor necrosis factor (TNF), erythropoietin (EPO), and bone morphogenetic protein (BMP). According to another aspect, the immunostimulant includes an adjuvant-type immunostimulatory agent such as APC Toll-like Receptor agonists or costimulatory/cell adhesion membrane proteins. Examples of Toll-like Receptor agonists include costimulatory/adhesion proteins such as CD80, CD86, and ICAM-1.

The term “cytokines” relates to proteins which have a molecular weight of about 5 to 60 kDa (such as about 5 to 20 kDa) and which participate in cell signaling (e.g., paracrine, endocrine, and/or autocrine signaling). In particular, when released, cytokines exert an effect on the behavior of cells around the place of their release. Examples of cytokines include lymphokines, interleukins, chemokines, interferons, and tumor necrosis factors (TNFs). According to the disclosure, a cytokine may be a naturally occurring cytokine or a functional fragment or variant thereof. A cytokine may be human cytokine and may be derived from any vertebrate, especially any mammal.

Immunostimulant polypeptides described herein can be prepared as fusion or chimeric polypeptides that include an immunostimulant portion and a heterologous polypeptide (i.e., a polypeptide that is not an immunostimulant). The immunostimulant may be fused to an extended-PK group, which increases circulation half-life. Non-limiting examples of extended-PK groups are described infra. It should be understood that other PK groups that increase the circulation half-life of immunostimulants such as cytokines, or variants thereof, are also applicable to the present disclosure.

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in US2005/0287153 and US2007/0003549). Other exemplary extended-PK groups are disclosed in Kontermann, Expert Opin. Biol Ther, 2016, 16 (7): 903-15 which is herein incorporated by reference in its entirety. As used herein, an “extended-PK” immunostimulant refers to an immunostimulant moiety in combination with an extended-PK group. In some embodiments, the extended-PK immunostimulant is a fusion protein in which an immunostimulant moiety is linked or fused to an extended-PK group.

In certain embodiments, the serum half-life of an extended-PK immunostimulant is increased relative to the immunostimulant alone (i.e., the immunostimulant not fused to an extended-PK group). As used herein, “half-life” refers to the time taken for the serum or plasma concentration of a compound such as a peptide or polypeptide to reduce by 50%, in vivo, for example due to degradation and/or clearance or sequestration by natural mechanisms. An extended-PK immunostimulant suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

In some embodiments, a pharmaceutically active peptide or protein comprises a replacement protein. In these embodiments, the present disclosure provides a method for treatment of a subject having a disorder requiring protein replacement (e.g., protein deficiency disorders) comprising administering to the subject RNA as described herein encoding a replacement protein. The term “protein replacement” refers to the introduction of a protein (including functional variants thereof) into a subject having a deficiency in such protein. The term also refers to the introduction of a protein into a subject otherwise requiring or benefiting from providing a protein, e.g., suffering from protein insufficiency. The term “disorder characterized by a protein deficiency” refers to any disorder that presents with a pathology caused by absent or insufficient amounts of a protein. This term encompasses protein folding disorders, i.e., conformational disorders, that result in a biologically inactive protein product. Protein insufficiency can be involved in infectious diseases, immunosuppression, organ failure, glandular problems, radiation illness, nutritional deficiency, poisoning, or other environmental or external insults.

The term “hormones” relates to a class of signaling molecules produced by glands, wherein signaling usually includes the following steps: (i) synthesis of a hormone in a particular tissue; (ii) storage and secretion; (iii) transport of the hormone to its target; (iv) binding of the hormone by a receptor; (v) relay and amplification of the signal; and (vi) breakdown of the hormone. Hormones differ from cytokines in that (1) hormones usually act in less variable concentrations and (2) generally are made by specific kinds of cells. In some embodiments, a “hormone” is a peptide or protein hormone, such as insulin, vasopressin, prolactin, adrenocorticotropic hormone (ACTH), thyroid hormone, growth hormones (such as human grown hormone or bovine somatotropin), oxytocin, atrial-natriuretic peptide (ANP), glucagon, somatostatin, cholecystokinin, gastrin, and leptins.

The term “adhesion molecules” relates to proteins which are located on the surface of a cell and which are involved in binding of the cell with other cells or with the extracellular matrix (ECM). Adhesion molecules are typically transmembrane receptors and can be classified as calcium-independent (e.g., integrins, immunoglobulin superfamily, lymphocyte homing receptors) and calcium-dependent (cadherins and selectins). Particular examples of adhesion molecules are integrins, lymphocyte homing receptors, selectins (e.g., P-selectin), and addressins.

The term “immunoglobulins” or “immunoglobulin superfamily” refers to molecules which are involved in the recognition, binding, and/or adhesion processes of cells. Molecules belonging to this superfamily share the feature that they contain a region known as immunoglobulin domain or fold. Members of the immunoglobulin superfamily include antibodies (e.g., IgG), T cell receptors (TCRs), major histocompatibility complex (MHC) molecules, co-receptors (e.g., CD4, CD8, CD19), antigen receptor accessory molecules (e.g., CD-3γ, CD3-8, CD-38, CD79a, CD79b), co-stimulatory or inhibitory molecules (e.g., CD28, CD80, CD86), and other.

The term “immunologically active compound” relates to any compound altering an immune response, preferably by inducing and/or suppressing maturation of immune cells, inducing and/or suppressing cytokine biosynthesis, and/or altering humoral immunity by stimulating antibody production by B cells. Immunologically active compounds possess potent immunostimulating activity including, but not limited to, antiviral and antitumor activity, and can also down-regulate other aspects of the immune response, for example shifting the immune response away from a TH2 immune response, which is useful for treating a wide range of TH2 mediated diseases. Immunologically active compounds can be useful as vaccine adjuvants. Particular examples of immunologically active compounds include interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, selectins, homing receptors, and antigens, in particular tumor-associated antigens, pathogen-associated antigens (such as bacterial, parasitic, or viral antigens), allergens, and autoantigens. A preferred immunologically active compound is a vaccine antigen, i.e., an antigen whose inoculation into a subject induces an immune response.

In some embodiments, RNA (in particular, mRNA) described in the present disclosure comprises a nucleic acid sequence encoding a peptide or polypeptide comprising an epitope for inducing an immune response against an antigen in a subject. The “peptide or polypeptide comprising an epitope for inducing an immune response against an antigen in a subject” is also designated herein as “vaccine antigen”, “peptide and protein antigen” or simply “antigen”.

In some embodiments, the RNA (in particular, mRNA) encoding vaccine antigen is a single-stranded, 5′ capped mRNA that is translated into the respective protein upon entering cells of a subject being administered the RNA, e.g., antigen-presenting cells (APCs). Preferably, the RNA (i) contains structural elements optimized for maximal efficacy of the RNA with respect to stability and translational efficiency (5′ cap, 5′ UTR, 3′ UTR, poly(A) sequence); (ii) is modified for optimized efficacy of the RNA (e.g., increased translation efficacy, decreased immunogenicity, and/or decreased cytotoxicity) (e.g., by replacing (partially or completely, preferably completely) naturally occurring nucleosides (in particular cytidine) with synthetic nucleosides (e.g., modified nucleosides selected from the group consisting of pseudouridine (w), N1-methyl-pseudouridine (m1ψ), and 5-methyl-uridine); and/or codon-optimization), or (iii) both (i) and (ii).

The vaccine antigen comprises an epitope for inducing an immune response against an antigen in a subject. Accordingly, the vaccine antigen comprises an antigenic sequence for inducing an immune response against an antigen in a subject. Such antigenic sequence may correspond to a target antigen or disease-associated antigen, e.g., a protein of an infectious agent (e.g., viral or bacterial antigen) or tumor antigen, or may correspond to an immunogenic variant thereof, or an immunogenic fragment of the target antigen or disease-associated antigen or the immunogenic variant thereof. Thus, the antigenic sequence may comprise at least an epitope of a target antigen or disease-associated antigen or an immunogenic variant thereof.

The antigenic sequences, e.g., epitopes, suitable for use according to the disclosure typically may be derived from a target antigen, i.e. the antigen against which an immune response is to be elicited. For example, the antigenic sequences contained within the vaccine antigen may be a target antigen or a fragment or variant of a target antigen.

The antigenic sequence or a procession product thereof, e.g., a fragment thereof, may bind to the antigen receptor such as TCR or CAR carried by immune effector cells. In some embodiments, the antigenic sequence is selected from the group consisting of the antigen expressed by a target cell to which the immune effector cells are targeted or a fragment thereof, or a variant of the antigenic sequence or the fragment.

A vaccine antigen which may be provided to a subject according to the present disclosure by administering RNA encoding the vaccine antigen, preferably results in the induction of an immune response, e.g., in the stimulation, priming and/or expansion of immune effector cells, in the subject being provided the vaccine antigen. Said immune response, e.g., stimulated, primed and/or expanded immune effector cells, is preferably directed against a target antigen, in particular a target antigen expressed by diseased cells, tissues and/or organs, i.e., a disease-associated antigen. Thus, a vaccine antigen may comprise the disease-associated antigen, or a fragment or variant thereof. In some embodiments, such fragment or variant is immunologically equivalent to the disease-associated antigen.

In the context of the present disclosure, the term “fragment of an antigen” or “variant of an antigen” means an agent which results in the induction of an immune response, e.g., in the stimulation, priming and/or expansion of immune effector cells, which immune response, e.g., stimulated, primed and/or expanded immune effector cells, targets the antigen, i.e. a disease-associated antigen, in particular when presented by diseased cells, tissues and/or organs. Thus, the vaccine antigen may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen comprises a fragment of the disease-associated antigen or an amino acid sequence which is homologous to a fragment of the disease-associated antigen said fragment or amino acid sequence may comprise an epitope of the disease-associated antigen to which the antigen receptor of the immune effector cells is targeted or a sequence which is homologous to an epitope of the disease-associated antigen. Thus, according to the disclosure, a vaccine antigen may comprise an immunogenic fragment of a disease-associated antigen or an amino acid sequence being homologous to an immunogenic fragment of a disease-associated antigen. An “immunogenic fragment of an antigen” according to the disclosure preferably relates to a fragment of an antigen which is capable of inducing an immune response against, e.g., stimulating, priming and/or expanding immune effector cells carrying an antigen receptor binding to, the antigen or cells expressing the antigen. It is preferred that the vaccine antigen (similar to the disease-associated antigen) provides the relevant epitope for binding by the antigen receptor present on the immune effector cells. In some embodiments, the vaccine antigen or a fragment thereof (similar to the disease-associated antigen) is expressed on the surface of a cell such as an antigen-presenting cell (optionally in the context of MHC) so as to provide the relevant epitope for binding by immune effector cells. The vaccine antigen may be a recombinant antigen.

In some embodiments of all aspects of the invention, the RNA encoding the vaccine antigen is expressed in cells of a subject to provide the antigen or a procession product thereof for binding by the antigen receptor expressed by immune effector cells, said binding resulting in stimulation, priming and/or expansion of the immune effector cells. An “antigen” according to the present disclosure covers any substance that will elicit an immune response and/or any substance against which an immune response or an immune mechanism such as a cellular response and/or humoral response is directed. This also includes situations wherein the antigen is processed into antigen peptides and an immune response or an immune mechanism is directed against one or more antigen peptides, in particular if presented in the context of MHC molecules. In particular, an “antigen” relates to any substance, such as a peptide or polypeptide, that reacts specifically with antibodies or T-lymphocytes (T-cells). The term “antigen” may comprise a molecule that comprises at least one epitope, such as a T cell epitope. In some embodiments, an antigen is a molecule which, optionally after processing, induces an immune reaction, which may be specific for the antigen (including cells expressing the antigen). In some embodiments, an antigen is a disease-associated antigen, such as a tumor antigen, a viral antigen, or a bacterial antigen, or an epitope derived from such antigen.

The term “autoantigen” or “self-antigen” refers to an antigen which originates from within the body of a subject (i.e., the autoantigen can also be called “autologous antigen”) and which produces an abnormally vigorous immune response against this normal part of the body. Such vigorous immune reactions against autoantigens may be the cause of “autoimmune diseases”.

In some embodiments, antigen for vaccination which may be administered in the form of RNA coding therefor comprises a naturally occurring antigen or a fragment such as an epitope thereof.

### Lipids

The compositions of the invention also contain a mixture of lipids. The terms “lipid” and “lipid-like material” are broadly defined herein as molecules which comprise one or more hydrophobic moieties or groups and also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. Lipids may comprise a polar portion and an apolar (or non-polar) portion.

Hydrophobicity can be conferred by the inclusion of apolar groups that include, but are not limited to, long-chain saturated and unsaturated hydrocarbyl groups (as defined and exemplified above), such as alkyl, alkenyl and/or alkynyl groups and such groups substituted by one or more aryl, heteroaryl, or cycloalkyl groups (as defined and exemplified above). The hydrophilic groups may comprise polar and/or charged groups and include at least one amine and optionally hydrophilic non-charged groups such as hydroxyl, carbohydrate, sulfhydryl, nitro or like groups and may further include anionic groups such as phosphate, phosphonate, carboxylic acid, sulfate, sulfonate (all as defined and exemplified above) and other like groups.

The term “hydrophobic” as used herein with respect to a compound, group or moiety means that said compound, group, or moiety is not attracted to water molecules and, when present in an aqueous solution, excludes water molecules. In some embodiments, the term “hydrophobic” refers to any compound, group or moiety which is substantially immiscible or insoluble in aqueous solution. In some embodiments, a hydrophobic compound, group or moiety is substantially nonpolar.

Examples of hydrophobic groups are hydrocarbyl groups (as defined and exemplified above), such as alkyl, alkenyl and/or alkynyl groups and such groups substituted by one or more aryl, heteroaryl, or cycloalkyl groups (as defined and exemplified above). In some embodiments, a hydrophobic compound, group or moiety is lipophilic. The hydrophobic group can have functional groups (e.g., ether, thioether, ester, dioxolane, halide, amide, sulfonamide, carbamate, etc.) and atoms other than carbon and hydrogen as long as the group satisfies the condition of being substantially immiscible or insoluble in aqueous solution.

The term “lipophilic” as used herein with respect to a compound, group or moiety means that said compound, group or moiety is soluble in nonpolar solvents (such as hexane, tetrahydrofuran (THF), and/or chloroform). In some embodiments, the term “lipophilic” refers to any compound, group or moiety which is soluble in nonpolar solvents (such as hexane, tetrahydrofuran (THF), and/or chloroform) and which is substantially immiscible or insoluble in aqueous solution. Examples of lipophilic groups are hydrocarbyl groups, such as non-cyclic, preferably straight, hydrocarbyl groups (such as hydrocarbyl groups having at least 10 carbon atoms), e.g., the lipophilic chain of a natural lipid. Other examples are branched hydrocarbyls having at least 10 carbon atoms, e.g. the lipophilic segment in lipids such as SM-102 or ALC-315.

The hydrophobic moieties of a lipid may have between 24 and 60 carbon atoms and can be hydrocarbyls (as described and exemplified above, typically comprising alkyl, alkenyl or alkynyl groups as described and exemplified above). The 24 to 60 carbon atoms can be segmented into two or more hydrophobic moieties, with each such moiety typically having at least 6 carbon atoms. An example for segmented hydrophobic moieties wherein each segment is hydrocarbyl are lipids comprising the diacylglycerol or dialkylglycerol moiety wherein each of the acyl or alkyl groups comprise between 12 and 20 carbon atoms.

The hydrophobic moieties of a lipid preferably have between 24 and 60 carbon atoms and can also be heterohydrocarbyls wherein the heteroatoms are selected from N, O or S forming one, two, three or four non-charged groups of ether, thioether, ester, amide, carbamate, sulfonamide and the like. The 24 to 60 carbon atoms can be segmented into two or more hydrophobic moieties, provided that each such moiety has at least 6 carbon atoms. An example for segmented hydrophobic moieties wherein each segment is hydrocarbyl are lipids comprising the diacylglycerol or dialkylglycerol moiety wherein each of the acyl or alkyl comprise between 12 and 20 carbon atoms. An example for hydrophobic moieties wherein each segment is heterohydrocarbyl are the ester-branched moieties in lipids such as SM-102 or ALC-315, as defined and exemplified below.

### Cationically Ionizable Lipid

The lipid nanoparticle compositions described herein comprise at least one cationically ionizable lipid as particle forming agent. The cationically ionizable lipids are typically able to electrostatically bind the active ingredient (particularly although not exclusively nucleic acid). Cationically ionizable lipids can be associated with nucleic acid, e.g. by forming complexes with the nucleic acid or forming organized structures in which the nucleic acid is enclosed or encapsulated.

As used herein, a “cationically ionizable lipid” refers to a lipid or lipid-like material which, depending on whether it is protonated or deprotonated, has a net positive charge or is neutral, i.e., a lipid which is not permanently cationic. Thus, depending on the pH of the composition in which the cationically ionizable lipid is solved, the cationically ionizable lipid is either positively charged or neutral.

In some embodiments, the cationically ionizable lipid comprises a head group which includes at least one nitrogen atom (N) which is capable of being protonated, preferably under physiological or slightly acidic conditions.

In one embodiment, the cationically ionizable lipid has polar and apolar portions, wherein the polar portion is cationically ionizable with a pKₐ between 8 and 10.5 and the ratio of the molecular volume between the polar and apolar portion is 0.15 or less. In this specification, all references to the pKₐ of a base (such as but not limited to a tertiary amine) take its normal meaning in chemistry as that of the conjugate acid.

It is known that the apparent pKₐ of the cationically ionizable lipid in an LNP is between 5.5 and 7, more preferred between 5.8 and 6.7 and most preferred between 6.0 and 6.5. The difference between the pKₐ of the polar portion in solution and the ionizable lipid is described, inter alia, by Carraso et al Nature Communications Biology 2021, 4, 956.

### Molecular Volume and Lipid Shapes

Methods for calculating molecular volumes are described, for example, in WO2008/043575 and WO2009/047006. Lipid shape theory is built on a shape balance between the hydrophobic or apolar portion and the polar head-group portion of a given amphiphile rather than on absolute values for the two molecular portions. Kappa (κ) may be used to describe the volume ratio between the polar and apolar section of a lipid.

$$κ = molecular volume ( head , polar ) / molecular volume ( tail , apolar )$$

Various different ways are available to those skilled in the art to calculate molecular volumes and alternative methods and sources are discussed for example in Connolly, M. J. Am. Chem. Soc. 1985, 107, 1118-1124 and the references therein or are given at: http://www.ccl.net/cca/documents/molecular-modeling/node5.html.

Molecular volume is commonly calculated by assigning a value called a van der Waals radius, rⁱ_{vdW}, to each atom type in such a way that the sum of these quantities for a given atom pair, i and j, is equal to their closest possible distance (dij):

$${t}_{vdW}^{i} + {r}_{vdW}^{j} \leq dij$$

Many different tables of “best” van der Waals radii exist, even though the values for corresponding atoms coming from different authors are similar. In geometric terms, the van der Waals radius may be imagined as a spherical “shield” surrounding the atom, and the closest distance between two non-bonded atoms is when their respective shields touch. However, the shields of covalently bonded atoms intersect since bond lengths are shorter than the sum of the van der Waals radii partaking atoms. A molecular van der Waals surface, also called a van der Waals envelope, is composed of the spheres for individual atoms with their intersecting sections removed.

For a single molecule (i.e., molecule for which there is a path between any two atoms along covalent bonds), the van der Waals envelope is a closed surface, and hence, it contains volume. This volume is called the molecular volume, or van der Waals volume, and is usually given in Å³. The straightforward way of calculating molecular volume on a computer is by numerical integration.

In some embodiments, molecular volumes for lipid molecules and the respective head and tail fragments may be calculated using DS Viewer Pro 5.0 (Accelrys Inc., San Diego, CA) and volumes within the respective van der Waals radii may be calculated. Another software for such calculations is RDkit.

In terms of defining polar and apolar portions of particular lipids, typical rules may be applied, which are customary in the field. For the cholesterol derivatives, the entire sterol, but not the 3′ oxygen, may be defined as the hydrophobic section and the head-group being complementary to that. For cationic or anionic alkyl derivatives the polar head-group may be defined as the polar fragment involving the C1 carbon of the alkyl chain. Consequently, the residual chain with n−1 carbon atoms typically represents the hydrophobic apolar part. For phospholipids (i.e., neutral lipids), typical membrane fragments are 1,2-diacyl-ethyleneglycols that represent the hydrophobic section, leaving the 3′ carbon atom of the original glycerol with the phosphocholine head-group. Molecular volumes typically depend on the constants used for the calculations and may be affected by the conformation of the molecule.

Exemplary values for representative polar and apolar lipid fragments for cationically ionizable lipids are provided in Table 1 below, the compounds are disclosed further below. Compounds X-3, X-2, X-22, and X-20 are those of formula (X) listed in Table 2 below, compound D in Table 3, and the remaining compounds in Table 4. The polar and apolar fragments have been defined for the non-protonated form of the ionizable lipid as it would prevail at pH of about 7.4.

TABLE 1

| | partial | | |
|---|---|---|---|
| | molecular | | lipid molecular |
| Compound | volume | κ | volume |
| MC3-Tail | 531 | 0.11 | 589 |
| MC3-Head | 58 | | |
| X-3 Tail | 598 | 0.07 | 641 |
| X-3 Head | 43 | | |
| X2 Tail | 587 | 0.07 | 629 |
| X2 Head | 43 | | |
| X22 Tail | 651 | 0.07 | 694 |
| X22 Head | 43 | | |
| Compound 45 Tail | 573 | 0.07 | 616 |
| Compound 45 Head | 43 | | |
| X20 Tail | 650 | 0.07 | 692 |
| X20 Head | 43 | | |
| D Tail | 469 | 0.09 | 512 |
| D Head | 43 | | |
| Compound 18 Tail | 519 | 0.08 | 562 |
| Compound 18 Head | 43 | | |
| Compound 28 Tail | 522 | 0.08 | 564 |
| Compound 28 Head | 43 | | |
| Compound 47 Tail | 511 | 0.08 | 553 |
| Compound 47 Head | 43 | | |
| Compound 54 Tail | 459 | 0.09 | 502 |
| Compound 54 Head | 43 | | |
| Compound 68 Tail | 521 | 0.08 | 564 |
| Compound 68 Head | 43 | | |
| Compound 122 Tail | 494 | 0.09 | 536 |
| Compound 122 Head | 43 | | |
| Compound 540 Tail | 607 | 0.13 | 684 |
| Compound 540 Head | 77 | | |
| Compound 325 Tail | 567 | 0.10 | 625 |
| Compound 325 Head | 58 | | |

It is possible to use other methods to determine molecular volumes for the lipids. Also, some parameters such as the exact split-point between membrane tail and polar head; number of water molecules in the hydration cage or the van der Waals radii can be varied without affecting the general applicability of the model. In some embodiments the molecular volumes recited above may be used.

In one embodiment, the cationically ionisable lipids capable of forming LNP have a cone-shaped lipid tail region. The need for a cone-shaped lipid tail region follows the molecular shape theory as described in Witzigmann et al Adv. Drug Delivery Rev. 2020, 159, 344-363. The term “cone-shaped” in this context means a lipid containing tail group(s) with larger cross-sectional areas than the lipid head group. In the context of the present invention, k of the cationically ionizable lipid when unprotonated is preferably 0.15 or less, in order to be capable of forming LNPs. In one embodiment, the cationically ionisable lipids capable of forming LNP have a ratio between head and tail volumes (i.e., a θ value), when unprotonated, which is below 0.15, preferably below 0.12 and more preferably below 0.1. This ratio may be calculated using the method of Siepi et al. Biophys J. 2011, 100, 2412-2421 which defines the apolar portion as 2 carbons away from the linker region, or, by analogy from the tertiary amine of the head group, in ionizable lipids. The cationically ionizable lipid may have a κ of from about 0.05 to about 0.15, optionally from about 0.06 to about 0.13, such as from about 0.07 to about 0.1. κ is low when the polar head-groups are small and the hydrophobic tail portions are large. The polar head-group volume may be smaller than about 100 Å³, preferably smaller than 60 A³ and more preferred smaller than 50 A³. The polar head-group volume may be from about 30 Å³ to about 100 Å³, optionally from about 40 Å³ to about 80 Å³, such as from about 40 Å³ to about 60 Å³. The apolar tail group volume may be from about 400 Å³ to about 800 Å³, optionally from about 500 Å³ to about 700 Å³, preferably from about 550 Å³ to about 650 Å³. The cationically ionizable lipids typically do not form lamellar (bilayer) phases in water under physiological conditions.

In one embodiment, the cationically ionizable lipid has an apparent pKₐ of the conjugate acid between 5.5 and 6.8 when formulated as a lipid nanoparticle.

In one embodiment, the cationically ionizable lipid has a molecular volume of from about 400 Å³ to about 900 Å³, preferably from about 500 Å³ to about 700 Å³. The molecular volume may be calculated according to the methods described herein.

In one embodiment, the cationically ionizable lipid has a molecular mass of between 500 and 2000 Dalton.

The cationically ionizable lipid has a polar portion (also referred to herein as a hydrophilic group or a “head” group). Such a polar group typically comprises polar and/or charged groups. The cationically ionizable lipid may have one or more (such as 2, 3, 4 or 5) polar portions, but typically only one or two polar portions. Non-limiting examples of the polar groups which may be present on the polar portion include amine (preferably primary, secondary or tertiary amine), hydroxyl, sulfhydryl, and nitro groups (all as defined and exemplified above), a carbohydrate moiety (as defined and exemplified above, preferably a monosaccharide moiety) and may further include anionic groups such as phosphate, carboxylic acid, sulfate (all as defined and exemplified above) and other like groups.

The cationically ionizable lipid also has an apolar portion, which is a hydrophobic portion, as defined and exemplified above in relation to the general definitions of lipids.

In some embodiments, the cationically ionizable lipid comprises a head group (or polar group) which includes at least one tertiary amine moiety. The term “tertiary amine moiety” as used herein takes its usual meaning in organic chemistry and relates to an amine group, i.e. a moiety containing a nitrogen atom which is substituted with three organic substituents (wherein the substituents may be the same or different from each other). The organic substituents may be hydrocarbyl groups or heterohydrocarbyl groups, as defined above. Where the organic moieties are hydrocarbyl groups, they may be selected from alkyl groups, alkenyl groups, alkynyl groups, cycloalkyl groups, cycloalkenyl groups, cycloalkylalkyl groups, alkylcycloalkyl groups, alkylcycloalkylalkyl groups, aryl groups, alkylaryl groups, arylalkyl groups, and alkylarylalkyl groups (all as defined above, either in a broadest aspect or a preferred aspect). Preferably, the organic moieties on the tertiary amine are alkyl groups or alkenyl groups having 6 to 40 carbon atoms, each being optionally substituted by a polar group as described and exemplified above, such as ester, amine (preferably primary or secondary amine), amide, hydroxylamide, hydroxyl, sulfhydryl, and nitro groups (all as defined and exemplified above), or a carbohydrate moiety (as defined and exemplified above, preferably a monosaccharide moiety).

In one embodiment, at least one of the organic moieties of the tertiary amine moiety is an alkyl group (as defined and exemplified above) having 1 to 6 carbon atoms, the alkyl group being optionally substituted by a polar group, preferably a hydroxyl or amino group (preferably a primary amino group). In one embodiment, one or two of the organic moieties of the tertiary amine group comprise alkyl groups having 1 to 4 carbon atoms (as defined and exemplified above) the alkyl group being optionally substituted by a hydroxyl group. In one embodiment, one or two of the organic moieties of the tertiary amine group comprise methyl, ethyl, or hydroxyethyl groups.

In one embodiment, at least one of the organic moieties of the tertiary amine moiety is an alkyl group (as defined and exemplified above) having 1 to 6 carbon atoms, the alkyl group being optionally substituted by a polar group, preferably a hydroxyl or amino group (preferably a primary amino group). In one embodiment, one or two of the organic moieties of the tertiary amine group comprise alkyl groups having 1 to 4 carbon atoms (as defined and exemplified above) the alkyl group being optionally substituted by a hydroxyl group. In one embodiment, one or two of the organic moieties of the tertiary amine group comprise methyl, ethyl, or hydroxyethyl groups.

In one embodiment, two of the organic moieties of the tertiary amine moiety are linked to form a nitrogen-containing heterocyclic ring (as defined above). The heterocyclyl group may preferably contain from 3 to 10, such as 3, 4, 5, 6, or 7, ring atoms, of which at least one ring atom is a nitrogen atom and the other heteroatoms may be selected from N, O and S. At least one of the nitrogen atoms in the ring must be connected to the rest of the molecule to form a tertiary amine group. Exemplary heterocyclic tertiary amine groups include piperazinyl, morpholinyl, pyrrolidinyl, imidazolidinyl, pyrazolidinyl, and piperidinyl groups of which piperazinyl groups are preferred. The nitrogen-containing heterocyclic ring may be further linked to another tertiary amine group, as defined and exemplified above.

The polar group (e.g. tertiary amine) of the cationically ionisable lipid typically has a pKₐ of between about 8.0 and 10.5. The pKₐ of the tertiary amine when formulated in an LNP usually differs by between about 2 units and about 4 units from the pKₐ of the same tertiary amine in solution. For example, the pKₐ of the tertiary amine in the LNP may differ by between about 2.5 units and about 3.5 units, optionally by about 3 units, from the pKₐ of the same tertiary amine in solution. Thus, in one embodiment, the pKₐ of the tertiary amine moiety is between 5.5 and 6.8 when the cationically ionizable lipid is present in an LNP. Preferably, the pKₐ of the tertiary amine moiety may be between 6.2 and 6.5 when present in the LNP.

Examples of cationically ionizable lipids are disclosed, for example, in WO 2016/176330 and WO 2018/078053. In some embodiments, the cationically ionizable lipid has the structure of Formula (X):

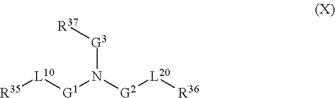

or a pharmaceutically acceptable salt, tautomer, prodrug or stereoisomer thereof,
wherein:
one of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₂₋₁₂ alkenylene;
G³ is C₁₋₂₄ alkylene, C₂₋₂₄ alkenylene, C₃₋₈ cycloalkylene, or C₃₋₈ cycloalkenylene;
R^{a} is H or C₁₋₁₂ alkyl;
R³⁵ and R³⁶ are each independently C₆₋₂₄ alkyl or C₆₋₂₄ alkenyl;
R³⁷ is H, OR⁵⁰, CN, —C(═O)OR⁴⁰, —OC(═O)R⁴⁰ or —NR⁵⁰C(═O)R⁴⁰;
R⁴⁰ is C₁₋₁₂ alkyl;
R⁵⁰ is H or C₁₋₆ alkyl; and
x is 0, 1 or 2.

In some of the foregoing embodiments of Formula (X), the lipid has one of the following structures (XA) or (XB):

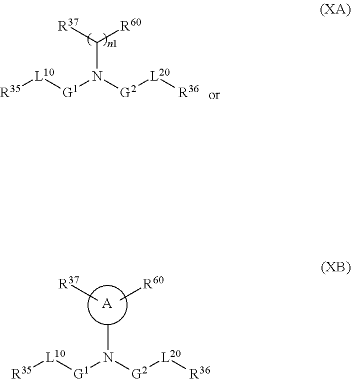

wherein:
A is a 3 to 8-membered cycloalkyl or cycloalkylene group;
R⁶⁰ is, at each occurrence, independently H, OH or C₁-C₂₄ alkyl;
n1 is an integer ranging from 1 to 15.

In some of the foregoing embodiments of Formula (X), the lipid has structure (XA), and in other embodiments, the lipid has structure (XB).

In other embodiments of Formula (X), the lipid has one of the following structures (XC) or (XD):

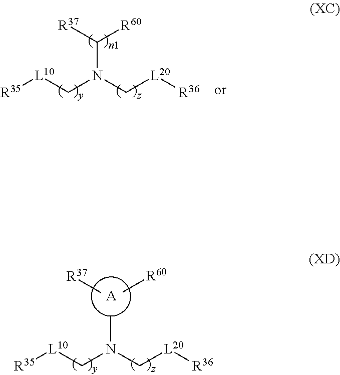

wherein y and z are each independently integers ranging from 1 to 12.

In any of the foregoing embodiments of Formula (X), one of L¹⁰ and L²⁰ is —O(C═O)—. For example, in some embodiments each of L¹⁰ and L²⁰ are —O(C═O)—. In some different embodiments of any of the foregoing, L¹⁰ and L²⁰ are each independently —(C═O)O— or —O(C═O)—. For example, in some embodiments each of L¹⁰ and L²⁰ is —(C═O)O—.

In some different embodiments of Formula (X), the lipid has one of the following structures (XE) or (XF):

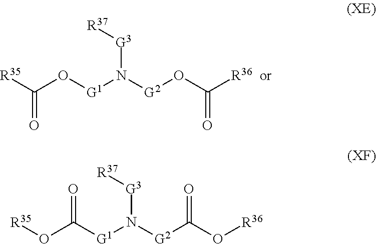

In some of the foregoing embodiments of Formula (X), the lipid has one of the following structures (XG), (XH), (XJ), or (XK):

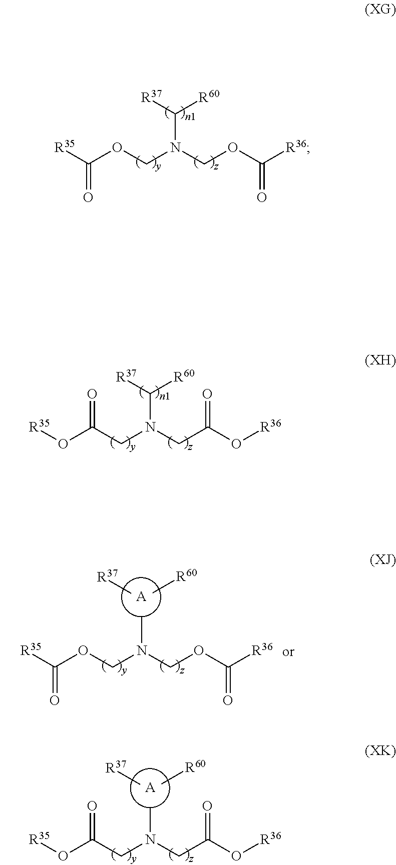

In some of the foregoing embodiments of Formula (X), n1 is an integer ranging from 2 to 12, for example from 2 to 8 or from 2 to 4. For example, in some embodiments, n1 is 3, 4, 5 or 6. In some embodiments, n1 is 3. In some embodiments, n1 is 4. In some embodiments, n1 is 5. In some embodiments, n1 is 6.

In some other of the foregoing embodiments of Formula (X), y and z are each independently an integer ranging from 2 to 10. For example, in some embodiments, y and z are each independently an integer ranging from 4 to 9 or from 4 to 6.

In some of the foregoing embodiments of Formula (X), R⁶⁰ is H. In other of the foregoing embodiments, R⁶⁰ is C₁-C₂₄ alkyl. In other embodiments, R⁶⁰ is OH.

In some embodiments of Formula (X), G³ is unsubstituted. In other embodiments, G³ is substituted. In various different embodiments, G³ is linear C₁-C₂₄ alkylene or linear C₂-C₂₄ alkenylene.

In some other foregoing embodiments of Formula (X), R³⁵ or R³⁶, or both, is C₆-C₂₄ alkenyl. For example, in some embodiments, R³⁵ and R³⁶ each, independently have the following structure:

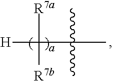

wherein:
R^{7a} and R^{7b} are, at each occurrence, independently H or C₁-C₁₂ alkyl; and
a is an integer from 2 to 12,
wherein R^{7a}, R^{7b} and a are each selected such that R³⁵ and R³⁶ each independently comprise from 6 to 20 carbon atoms. For example, in some embodiments a is an integer ranging from 5 to 9 or from 8 to 12.

In some of the foregoing embodiments of Formula (X), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈alkyl is methyl, ethyl, n-propyl, iso-propyl, n-butyl, iso-butyl, tert-butyl, n-hexyl or n-octyl.

In different embodiments of Formula (X), R³⁵ or R³⁶, or both, has one of the following structures:

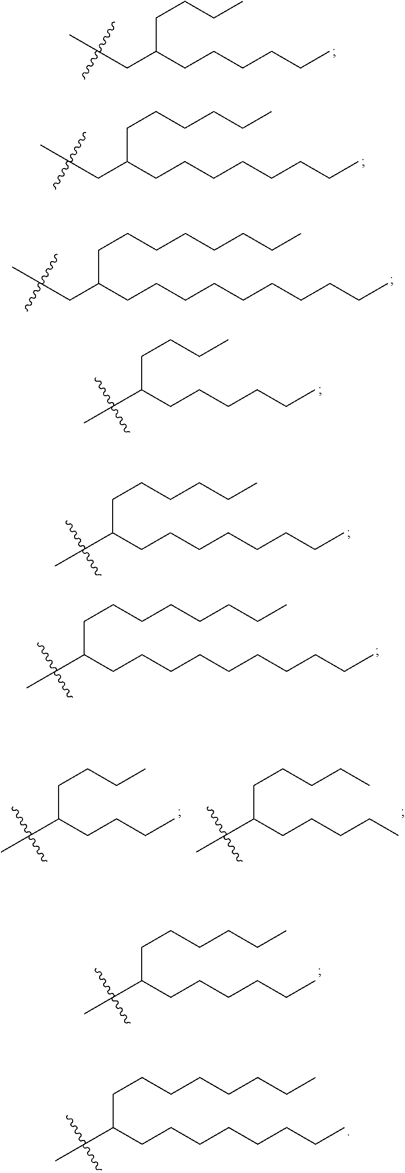

In some of the foregoing embodiments of Formula (X), R³⁷ is OH, CN, —C(═O)OR⁴⁰, —OC(═O)R⁴⁰ or —NHC(═O)R⁴⁰. In some embodiments, R⁴⁰ is methyl or ethyl.

In various different embodiments, the cationically ionizable lipid of Formula (X) has one of the structures set forth in Table 2 below.

TABLE 2
Representative Compounds of Formula (X)

| | |
|---|---|
| 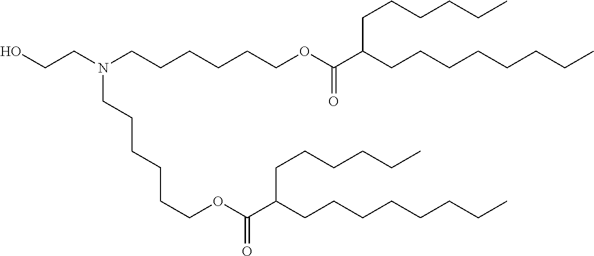 [SMILES: CCCCCCCCC(CCCCCC)C(=O)OCCCCCCN(CCO)CCCCCCOC(=O)C(CCCCCC)CCCCCCCC] | X-1 |
| 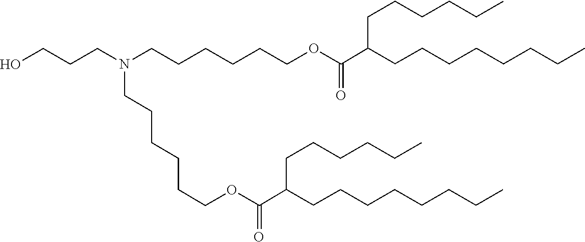 [SMILES: CCCCCCCCC(CCCCCC)C(=O)OCCCCCCN(CCCO)CCCCCCOC(=O)C(CCCCCC)CCCCCCCC] | X-2 |
| 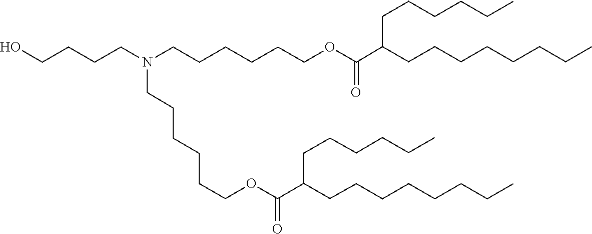 [SMILES: CCCCCCCCC(CCCCCC)C(=O)OCCCCCCN(CCCCO)CCCCCCOC(=O)C(CCCCCC)CCCCCCCC] | X-3 |
| 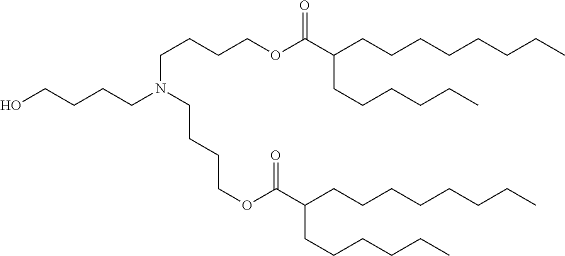 [SMILES: CCCCCCCCC(CCCCCC)C(=O)OCCCCN(CCCCO)CCCCOC(=O)C(CCCCCC)CCCCCCCC] | X-4 |
| 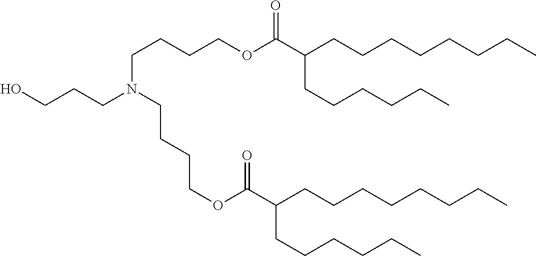 [SMILES: CCCCCCCCC(CCCCCC)C(=O)OCCCCN(CCCO)CCCCOC(=O)C(CCCCCC)CCCCCCCC] | X-5 |
| 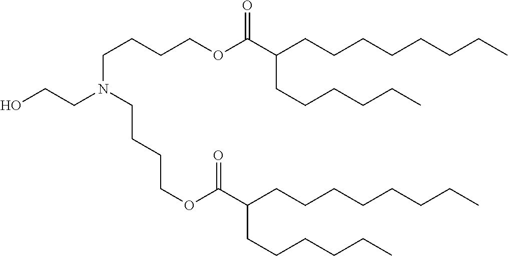 [SMILES: CCCCCCCCC(CCCCCC)C(=O)OCCCCN(CCO)CCCCOC(=O)C(CCCCCC)CCCCCCCC] | X-6 |
| 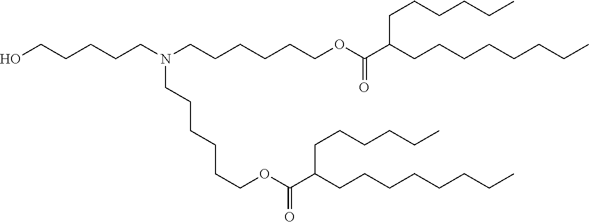 [SMILES: CCCCCCCCC(CCCCCC)C(=O)OCCCCCCN(CCCCCO)CCCCCCOC(=O)C(CCCCCC)CCCCCCCC] | X-7 |
| 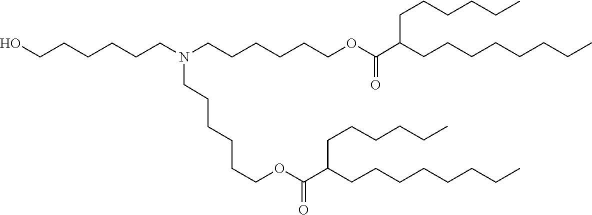 [SMILES: CCCCCCCCC(CCCCCC)C(=O)OCCCCCCN(CCCCCCO)CCCCCCOC(=O)C(CCCCCC)CCCCCCCC] | X-8 |
| 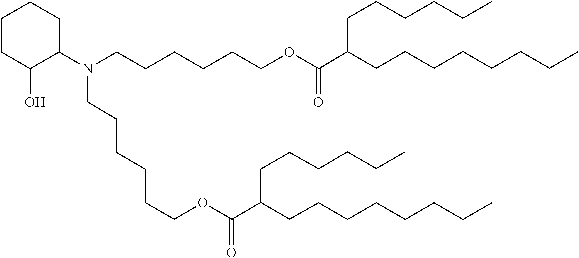 [SMILES: CCCCCCCCC(CCCCCC)C(=O)OCCCCCCN(CCCCCCOC(=O)C(CCCCCC)CCCCCCCC)C1CCCCC1O] | X-9 |
| 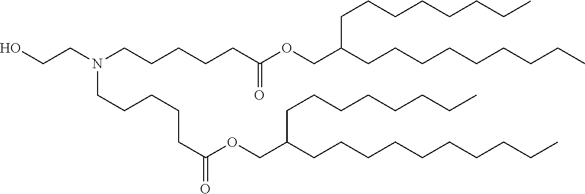 [SMILES: CCCCCCCCCCC(CCCCCCCC)COC(=O)CCCCCN(CCO)CCCCCC(=O)OCC(CCCCCCCC)CCCCCCCCC] | X-10 |
| 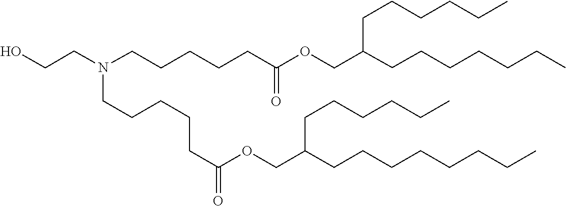 [SMILES: CCCCCCCCC(CCCCCC)COC(=O)CCCCCN(CCO)CCCCCC(=O)OCC(CCCCCC)CCCCCCC] | X-11 |
| 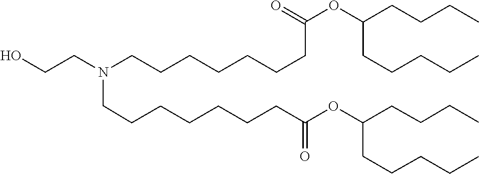 [SMILES: CCCCCC(CCCC)OC(=O)CCCCCCCN(CCO)CCCCCCCC(=O)OC(CCCC)CCCCC] | X-12 |
| 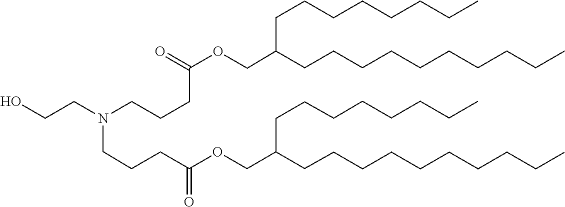 [SMILES: CCCCCCCCCCC(CCCCCCCC)COC(=O)CCCN(CCO)CCCC(=O)OCC(CCCCCCCC)CCCCCCCCCC] | X-13 |
| 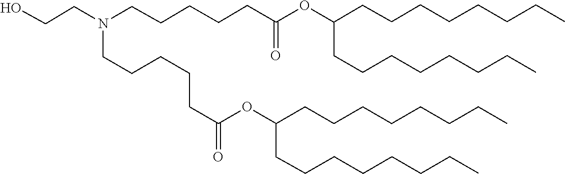 [SMILES: CCCCCCCCC(CCCCCCCC)OC(=O)CCCCCN(CCO)CCCCCC(=O)OC(CCCCCCCC)CCCCCCCC] | X-14 |
| 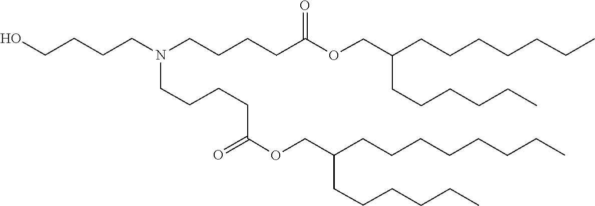 [SMILES: CCCCCCCCC(CCCCCC)COC(=O)CCCCN(CCCCO)CCCCC(=O)OCC(CCCCCC)CCCCCCC] | X-15 |
| 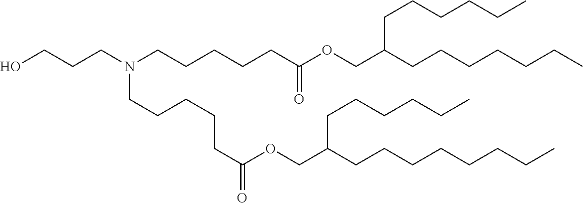 [SMILES: CCCCCCCCC(CCCCCC)COC(=O)CCCCCN(CCCO)CCCCCC(=O)OCC(CCCCCC)CCCCCCC] | X-16 |
| 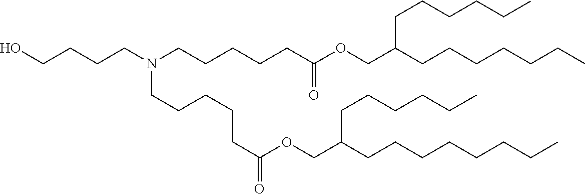 [SMILES: CCCCCCCCC(CCCCCC)COC(=O)CCCCCN(CCCCO)CCCCCC(=O)OCC(CCCCCC)CCCCCCC] | X-17 |
| 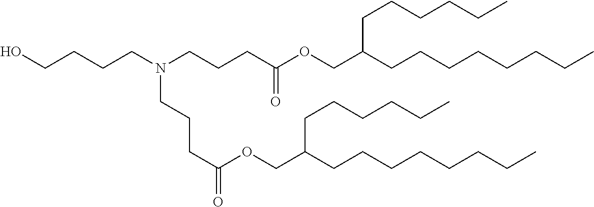 [SMILES: CCCCCCCCC(CCCCCC)COC(=O)CCCN(CCCCO)CCCC(=O)OCC(CCCCCC)CCCCCCCC] | X-18 |
| 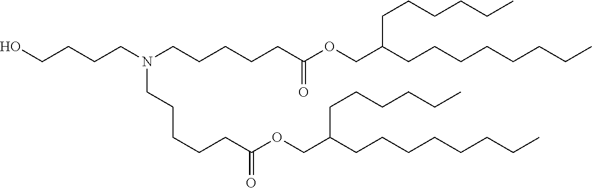 [SMILES: CCCCCCCCC(CCCCCC)COC(=O)CCCCCN(CCCCO)CCCCCC(=O)OCC(CCCCCC)CCCCCCCC] | X-19 |
| 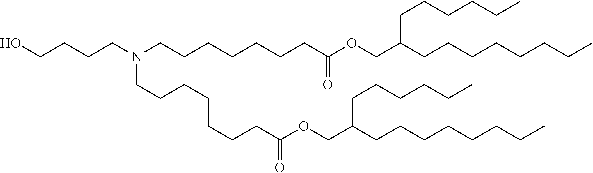 [SMILES: CCCCCCCCC(CCCCCC)COC(=O)CCCCCCCN(CCCCO)CCCCCCCC(=O)OCC(CCCCCC)CCCCCCCC] | X-20 |
| 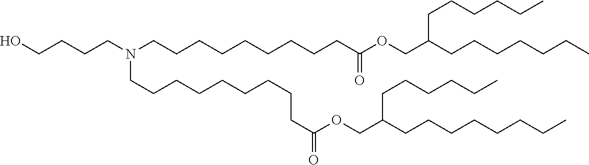 [SMILES: CCCCCCCCC(CCCCCC)COC(=O)CCCCCCCCCN(CCCCO)CCCCCCCCCC(=O)OCC(CCCCCC)CCCCCCC] | X-21 |
| 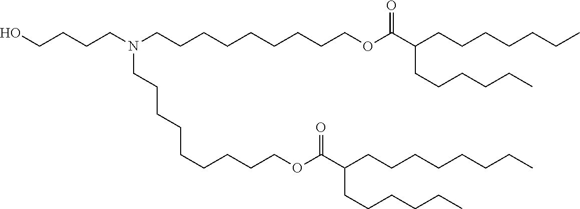 [SMILES: CCCCCCCCC(CCCCCC)C(=O)OCCCCCCCCCN(CCCCO)CCCCCCCCCOC(=O)C(CCCCCC)CCCCCCC] | X-22 |
| 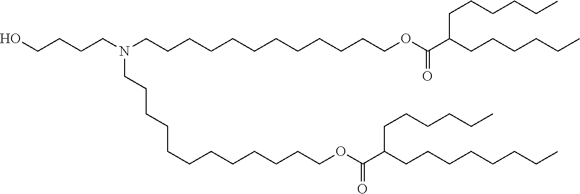 [SMILES: CCCCCCCCC(CCCCCC)C(=O)OCCCCCCCCCCCCN(CCCCO)CCCCCCCCCCCCOC(=O)C(CCCCCC)CCCCCC] | X-23 |
| 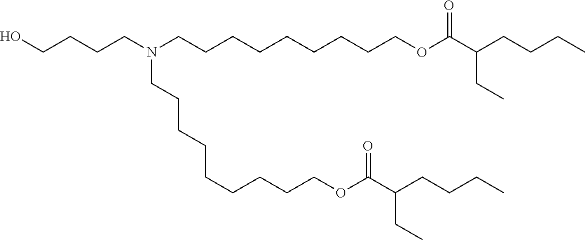 [SMILES: CCCCC(CC)C(=O)OCCCCCCCCCN(CCCCO)CCCCCCCCCOC(=O)C(CC)CCCC] | X-24 |
| 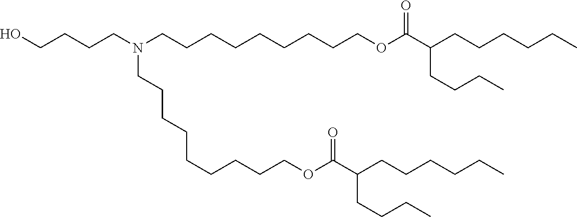 [SMILES: CCCCCCC(CCCC)C(=O)OCCCCCCCCCN(CCCCO)CCCCCCCCCOC(=O)C(CCCC)CCCCCC] | X-25 |
| 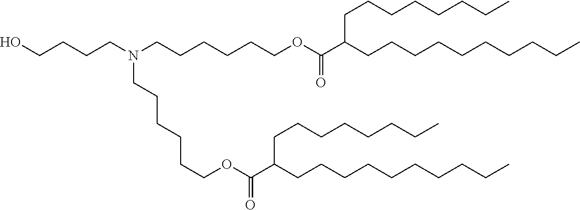 [SMILES: CCCCCCCCCCC(CCCCCCCC)C(=O)OCCCCCCN(CCCCO)CCCCCCOC(=O)C(CCCCCCCC)CCCCCCCCCC] | X-26 |
| 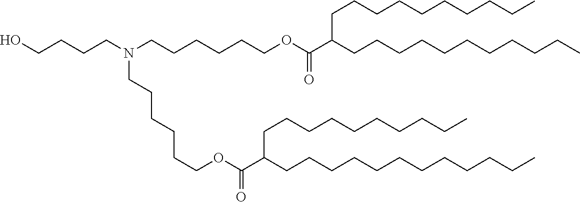 [SMILES: CCCCCCCCCCCCC(CCCCCCCCCC)C(=O)OCCCCCCN(CCCCO)CCCCCCOC(=O)C(CCCCCCCCCC)CCCCCCCCCCC] | X-27 |
| 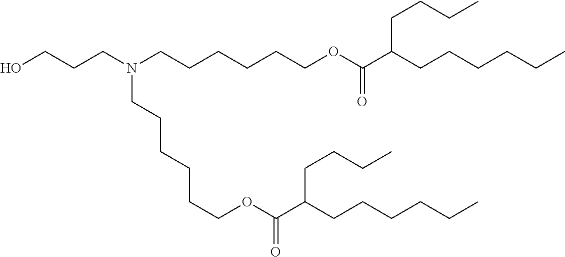 [SMILES: CCCCCCC(CCCC)C(=O)OCCCCCCN(CCCO)CCCCCCOC(=O)C(CCCC)CCCCCC] | X-28 |
| 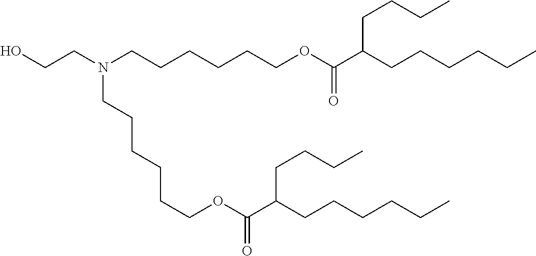 [SMILES: CCCCCCC(CCCC)C(=O)OCCCCCCN(CCO)CCCCCCOC(=O)C(CCCC)CCCCCC] | X-29 |
| 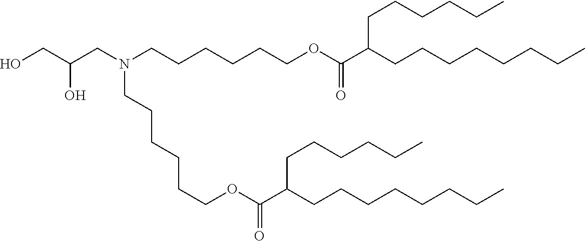 [SMILES: CCCCCCCCC(CCCCCC)C(=O)OCCCCCCN(CCCCCCOC(=O)C(CCCCCC)CCCCCCCC)CC(O)CO] | X-30 |
| 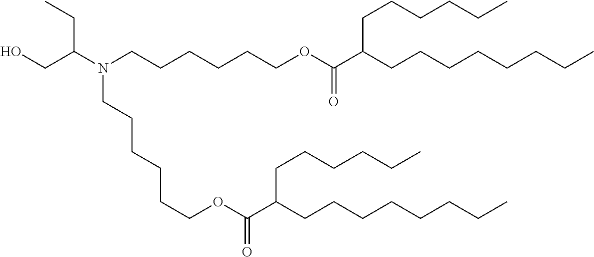 [SMILES: CCCCCCCCC(CCCCCC)C(=O)OCCCCCCN(CCCCCCOC(=O)C(CCCCCC)CCCCCCCC)C(CC)CO] | X-31 |
| 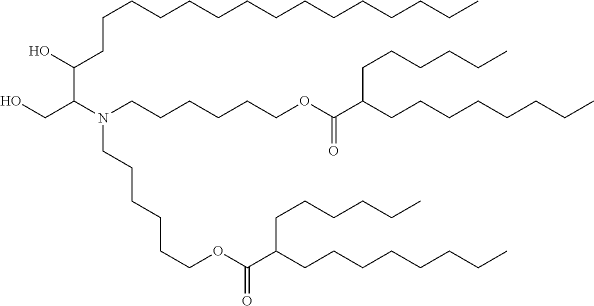 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] | X-32 |
| 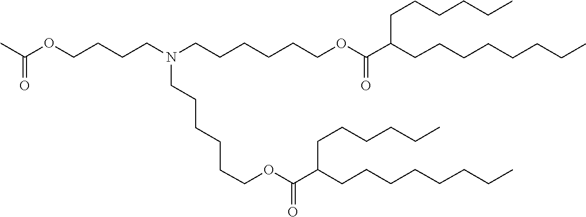 [SMILES: CCCCCCCCC(CCCCCC)C(=O)OCCCCCCN(CCCCCCOC(=O)C(CCCCCC)CCCCCCCC)CCCCOC(C)=O] | X-33 |
| 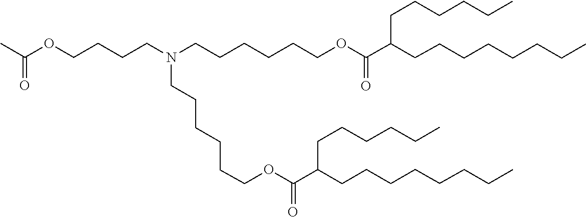 [SMILES: CCCCCCCCC(CCCCCC)C(=O)OCCCCCCN(CCCCCCOC(=O)C(CCCCCC)CCCCCCCC)CCCCOC(C)=O] | X-34 |
| 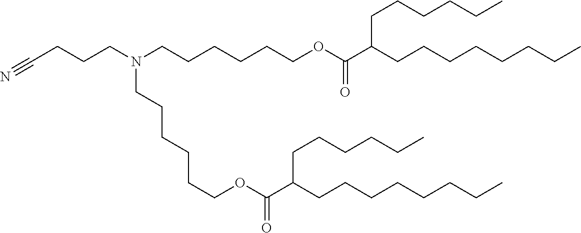 [SMILES: CCCCCCCCC(CCCCCC)C(=O)OCCCCCCN(CCCC#N)CCCCCCOC(=O)C(CCCCCC)CCCCCCCC] | X-35 |
| 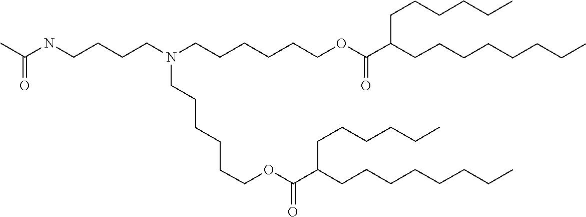 [SMILES: CCCCCCCCC(CCCCCC)C(=O)OCCCCCCN(CCCCCCOC(=O)C(CCCCCC)CCCCCCCC)CCCCNC(C)=O] | X-36 |

In various different embodiments, the cationically ionizable lipid has one of the structures set forth in Table 3 below.

TABLE 3

| No. | Structure |
|---|---|
| A | 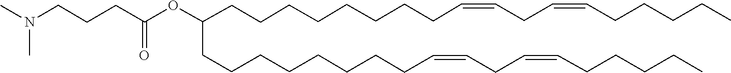 [SMILES: CCCCC/C=C\C/C=C\CCCCCCCCC(CCCCCCCC/C=C\C/C=C\CCCCC)OC(=O)CCCN(C)C] |
| B | 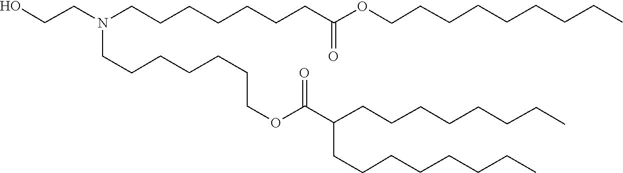 [SMILES: CCCCCCCCCOC(=O)CCCCCCCN(CCO)CCCCCCCOC(=O)C(CCCCCCCC)CCCCCCCC] |
| C | 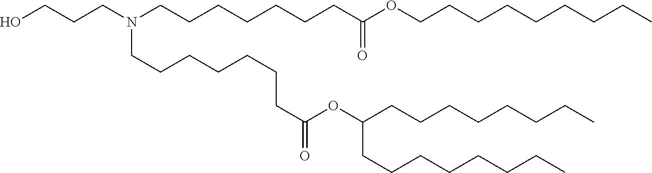 [SMILES: CCCCCCCCCOC(=O)CCCCCCCN(CCCO)CCCCCCCC(=O)OC(CCCCCCCC)CCCCCCCC] |
| D | 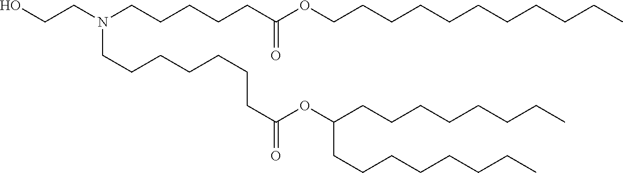 [SMILES: CCCCCCCCCCCOC(=O)CCCCCN(CCO)CCCCCCCC(=O)OC(CCCCCCCC)CCCCCCCC] |
| E | 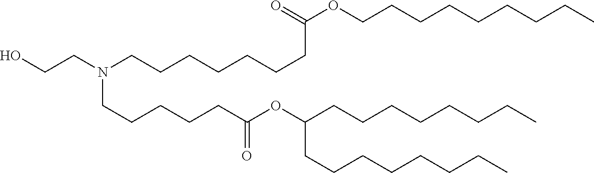 [SMILES: CCCCCCCCCOC(=O)CCCCCCCN(CCO)CCCCCC(=O)OC(CCCCCCCC)CCCCCCCC] |
| F | 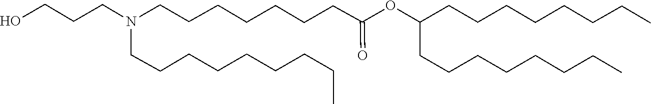 [SMILES: CCCCCCCCCN(CCCO)CCCCCCCC(=O)OC(CCCCCCCC)CCCCCCCC] |
| G | 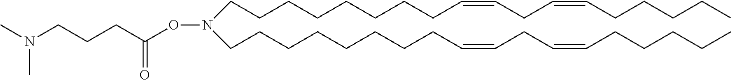 [SMILES: CCCCC/C=C\C/C=C\CCCCCCCCN(CCCCCCCC/C=C\C/C=C\CCCCC)OC(=O)CCCN(C)C] |

In some embodiments, the cationically ionizable lipid has the structure of Formula (XI):

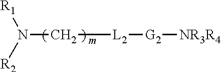

wherein
each of R₁ and R₂ is independently R₅ or -G₁-L₁-R₆, wherein at least one of R₁ and R² is -G₁-L₁-R₆;
each of R₃ and R₄ is independently selected from the group consisting of C₁₋₆ alkyl, C₂₋₆ alkenyl, aryl, and C₃₋₁₀ cycloalkyl;
each of R₅ and R₆ is independently a non-cyclic hydrocarbyl group having at least 10 carbon atoms;
each of G₁ and G₂ is independently unsubstituted C₁₋₁₂ alkylene or C₂₋₁₂ alkenylene;
each of L₁ and L₂ is independently selected from the group consisting of —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}— and —NR^{a}C(═O)O—;
Ra is H or C₁₋₁₂ alkyl;
m is 0, 1, 2, 3, or 4; and
x is 0, 1 or 2.

In some of the foregoing embodiments of Formula (XI), G₁ is independently unsubstituted C₁-C₁₂ alkylene or unsubstituted C₂₋₁₂ alkenylene, e.g., unsubstituted, straight C₁₋₁₂ alkylene or unsubstituted, straight C₂₋₁₂ alkenylene. In some embodiments, each G₁ is independently unsubstituted C₆₋₁₂ alkylene or unsubstituted C₆₋₁₂ alkenylene, e.g., unsubstituted, straight C₆₋₁₂ alkylene or unsubstituted, straight C₆₋₁₂ alkenylene. In some embodiments, each G₁ is independently unsubstituted C₈₋₁₂ alkylene or unsubstituted C₈₋₁₂ alkenylene, e.g., unsubstituted, straight C₈₋₁₂ alkylene or unsubstituted, straight C₈₋₁₂ alkenylene. In some embodiments, each G₁ is independently unsubstituted C₆₋₁₀ alkylene or unsubstituted C₆₋₁₀ alkenylene, e.g., unsubstituted, straight C₆₋₁₀ alkylene or unsubstituted, straight C₆₋₁₀ alkenylene. In some embodiments, each G₁ is independently unsubstituted alkylene having 8, 9 or 10 carbon atoms, e.g., unsubstituted, straight alkylene having 8, 9 or 10 carbon atoms. In some embodiments, where R₁ and R₂ are both independently -G₁-L₁-R₆, G₁ for R₁ may be different from G₁ for R₂. In some of these embodiments, for example, G₁ for R₁ is unsubstituted, straight C₁₋₁₂ alkylene and G₁ for R₂ is unsubstituted, straight C₂₋₁₂ alkenylene; or G₁ for R₁ is an unsubstituted, straight C₁₋₁₂ alkylene group and G₁ for R₂ is a different unsubstituted, straight C₁₋₁₂ alkylene group. In some embodiments, where R₁ and R₂ are both independently -G₁-L₁-R₆, G₁ for R₁ may be identical to G₁ for R₂. In some of these embodiments, for example, each G₁ is the same unsubstituted, straight C₈₋₁₂ alkylene, such as unsubstituted, straight C₈₋₁₀ alkylene, or each G₁ is the same unsubstituted, straight C₆₋₁₂ alkenylene.

In some of the foregoing embodiments of Formula (XI), each L₁ is independently selected from the group consisting of —O(C═O)—, —(C═O)O—, —C(═O)S—, —SC(═O)—, —NR^{a}C(═O)—, and —C(═O)NR^{a}—. In some embodiments, Rₐ of L₁ is H or C₁₋₁₂ alkyl. In some embodiments, Rₐ of L₁ is H or C₁₋₆ alkyl, e.g., H or C₁₋₃ alkyl. In some embodiments, Rₐ of L₁ is H, methyl, or ethyl. In some embodiments, each L₁ is independently selected from the group consisting of —O(C═O)—, —(C═O)O—, —C(═O)S—, and —SC(═O)—. In some embodiments, each L₁ is independently —O(C═O)— or —(C═O)O—. In some embodiments, where R₁ and R₂ are both independently -G₁-L₁-R₆, L₁ for R₁ may be different from L₁ for R₂. In some of these embodiments, for example, L₁ for R₁ is one moiety selected from the group consisting of —O(C═O)—, —(C═O)O—, —C(═O)S—, —SC(═O)—, —NR^{a}C(═O)—, and —C(═O)NR^{a}— (e.g., L₁ for R₁ is —O(C═O)—), and L₁ for R₂ is a different moiety selected from the group consisting of —O(C═O)—, —(C═O)O—, —C(═O)S—, —SC(═O)—, —NR^{a}C(═O)—, and —C(═O)NR^{a}— (e.g., L₁ for R₂ is —(C═O)O—). In some embodiments, where R₁ and R₂ are both independently -G₁-L₁-R₆, L₁ for R₁ may be identical to L₁ for R₂. In some of these embodiments, for example, each L₁ is the same moiety selected from the group consisting of —O(C═O)—, —(C═O)O—, —C(═O)S—, —SC(═O)—, —NR^{a}C(═O)—, and —C(═O)NR^{a}—, e.g., each L₁ is —O(C═O)— or each L₁ is —(C═O)O—.

In some of the foregoing embodiments of Formula (XI), each R₆ is independently a non-cyclic hydrocarbyl group having at least 10 carbon atoms, e.g., a straight hydrocarbyl group having at least 10 carbon atoms. In some embodiments, each R₆ has independently at most 30 carbon atoms, such as at most 28, at most 26, at most 24, at most 22, or at most 20 carbon atoms. In some embodiments, each R₆ is independently a non-cyclic hydrocarbyl group having 10 to 30 carbon atoms (such as 10 to 28, 10 to 26, 10 to 24, 10 to 22, or 10 to 20 carbon atoms), e.g., a straight hydrocarbyl group having 10 to 30 carbon atoms (such as 10 to 28, 10 to 26, 10 to 24, 10 to 22, or 10 to 20 carbon atoms). In some embodiments, each R₆ is attached to L₁ via an internal carbon atom of R₆. In some embodiments, each R₆ has independently at most 30 carbon atoms (such as at most 28, at most 26, at most 24, at most 22, or at most 20 carbon atoms), and each R₆ is attached to L₁ via an internal carbon atom of R₆. In some embodiments, each R₆ is independently a non-cyclic hydrocarbyl group having at least 10 carbon atoms, e.g., a straight hydrocarbyl group having at least 10 carbon atoms, and each R₆ is attached to L₁ via an internal carbon atom of R₆. In some embodiments, each R₆ is independently a non-cyclic hydrocarbyl group having 10 to 30 carbon atoms (such as 10 to 28, 10 to 26, 10 to 24, 10 to 22, or 10 to 20 carbon atoms), e.g., a straight hydrocarbyl group having 10 to 30 carbon atoms (such as 10 to 28, 10 to 26, 10 to 24, 10 to 22, or 10 to 20 carbon atoms), and each R₆ is attached to L₁ via an internal carbon atom of R₆. In some embodiments, the hydrocarbyl group of R₆ is an alkyl or alkenyl group, e.g., a C₁₀₋₃₀ alkyl or alkenyl group. Thus, in some embodiments, each R₆ is independently a non-cyclic alkyl group having at least 10 carbon atoms or a non-cyclic alkenyl group having at least 10 carbon atoms, e.g., a straight alkyl group having at least 10 carbon atoms or a straight alkenyl group having at least 10 carbon atoms. In some embodiments, each R₆ is independently a non-cyclic alkyl group having 10 to 30 carbon atoms (such as 10 to 28, 10 to 26, 10 to 24, 10 to 22, or 10 to 20 carbon atoms) or a non-cyclic alkenyl group having 10 to 30 carbon atoms (such as 10 to 28, 10 to 26, 10 to 24, 10 to 22, or 10 to 20 carbon atoms), e.g., a straight alkyl group having 10 to 30 carbon atoms (such as 10 to 28, 10 to 26, 10 to 24, 10 to 22, or 10 to 20 carbon atoms) or a straight alkenyl group having 10 to 30 carbon atoms (such as 10 to 28, 10 to 26, 10 to 24, 10 to 22, or 10 to 20 carbon atoms). In some embodiments, each R₆ is independently a non-cyclic alkyl group having 11 to 19 carbon atoms (such as 11, 13, 15, 17, or 17 carbon atoms), e.g., a straight alkyl group having 11 to 19 carbon atoms (such as 11, 13, 15, 17, or 17 carbon atoms). In some embodiments, each R₆ is independently a non-cyclic alkyl group having 10 to 30 carbon atoms (such as 10 to 28, 10 to 26, 10 to 24, 10 to 22, or 10 to 20 carbon atoms) or a non-cyclic alkenyl group having 10 to 30 carbon atoms (such as 10 to 28, 10 to 26, 10 to 24, 10 to 22, or 10 to 20 carbon atoms), e.g., a straight alkyl group having 10 to 30 carbon atoms (such as 10 to 28, 10 to 26, 10 to 24, 10 to 22, or 10 to 20 carbon atoms) or a straight alkenyl group having 10 to 30 carbon atoms (such as 10 to 28, 10 to 26, 10 to 24, 10 to 22, or 10 to 20 carbon atoms), and each R₆ is attached to L₁ via an internal carbon atom of R₆. In some embodiments, each R₆ is independently a non-cyclic alkyl group having 11 to 19 carbon atoms (such as 11, 13, 15, 17, or 17 carbon atoms), e.g., a straight alkyl group having 11 to 19 carbon atoms (such as 11, 13, 15, 17, or 17 carbon atoms), and each R₆ is attached to L₁ via an internal carbon atom of R₆. The expression “internal carbon atom” means that the carbon atom of R₆ by which R₆ is attached to L₁ is directly bonded to at least 2 other carbon atoms of R₆. For example, for the following C₁₁ alkyl group, each carbon atom at any one of positions 2, 3, 4, 5, and 7 qualifies as “internal carbon atom” according to the present disclosure, whereas the carbon atoms at positions 1, 6, 8, 9, 10, and 11 do not.

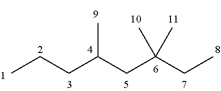

Consequently, R₆ being a C₁₁ alkyl group attached to L₁ via an internal carbon of R₆ includes the following groups:

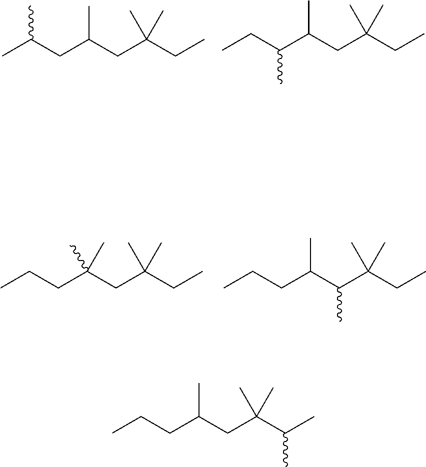

wherein [symbol] represents the bond by which R₆ is bound to L₁. Furthermore, for a straight alkyl group, e.g., a straight C₁₁ alkyl group, each carbon atom except for the first and last carbon atoms of the straight alkyl group (i.e., except the carbon atoms at positions 1 and 11 of the straight C₁₁ alkyl group) qualifies as “internal carbon atom”. Thus, in some embodiments, R₆ being a straight alkyl group having p carbon atoms and being attached to L₁ via an internal carbon atom of R₆ means that R₆ is attached to L₁ via a carbon atom of R₆ at any one of positions 2 to (p−1) (thereby excluding the terminal C atoms at positions 1 and p). In some embodiments, where R₆ is a straight alkyl group having p′ carbon atoms (wherein p′ is an even number) and being attached to L₁ via an internal carbon atom of R₆, R₆ is attached to L₁ via a carbon at any one of positions (p′/2−1), (p′/2), and (p′/2+1) of R₆ (e.g., if p′ is 10, R₆ is attached to L₁ via a carbon atom at any one of positions 4, 5, and 6 of R₆). In some embodiments, where R₆ is a straight alkyl group having p″ carbon atoms (wherein p″ is an uneven number) and being attached to L₁ via an internal carbon atom of R₆, R₆ is attached to L₁ via a carbon atom at any one of positions (p″−1)/2 and (p″+1)/2 of R₆ (e.g., if p″ is 11, R₆ is attached to L₁ via a carbon at any one of positions 5 and 6 of R₆). Generally, it is to be understood that if both R₁ and R₂ are -G₁-L₁-R₆ and each R₆ is attached to L₁ via an internal carbon atom of R₆, R₆ of R₁ is attached to L₁ of R₁ (and not to L₁ of R₂) via an internal carbon atom of R₆ of R₁ and R₆ of R₂ is attached to L₁ of R₂ (and not to L₁ of R₁) via an internal carbon atom of R₆ of R₂. In some embodiments, each R₆ is independently selected from the group consisting of:

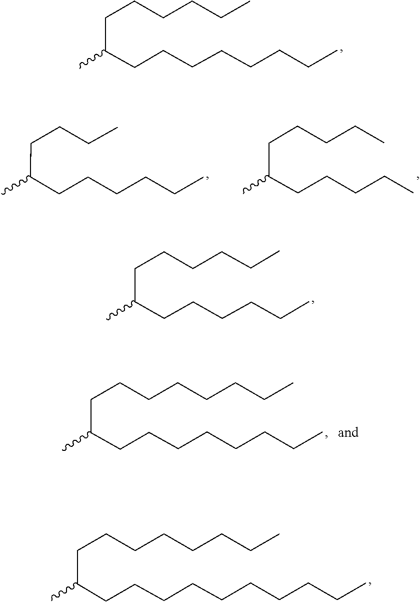

wherein [symbol] represents the bond by which R₆ is bound to L₁. In some embodiments, where R₁ and R₂ are both independently-G₁-L₁-R₆, R₆ for R₁ is different from R₆ for R₂. In some of these embodiments, for example, R₆ for R₁ may be a non-cyclic, preferably straight, hydrocarbyl group having at least 10 carbon atoms (e.g., R₆ for R₁ is

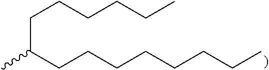 [SMILES: CCCCCCCCC(C)CCCCCC]

and R₆ for R₂ may be a different non-cyclic, preferably straight, hydrocarbyl group having at least 10 carbon atoms (e.g., R₆ for R₂ is

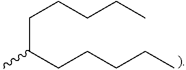

In some embodiments, where R₁ and R₂ are both independently -G₁-L₁-R₆, R₆ for R₁ is identical to R₆ for R₂. In some of these embodiments, for example, each R₆ is the same non-cyclic, preferably straight, hydrocarbyl group having at least 10 carbon atoms (e.g., each R₆ is

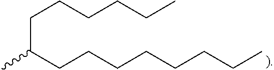 [SMILES: CCCCCCCCC(C)CCCCCC]

In some of the foregoing embodiments of Formula (XI), R₅ is a non-cyclic hydrocarbyl group having at least 10 carbon atoms, e.g., a straight hydrocarbyl group having at least 10 carbon atoms. In some embodiments, R₅ is a non-cyclic hydrocarbyl group having at least 12 carbon atoms, such as at least 14, at least 16, or at least 18 carbon atoms, e.g., a straight hydrocarbyl group having at least 12, at least 14, at least 16, or at least 18 carbon atoms. In some embodiments, R₅ has at most 30 carbon atoms, such as at most 28, at most 26, at most 24, at most 22, or at most 20 carbon atoms. In some embodiments, R₅ is a non-cyclic hydrocarbyl group, e.g., a straight hydrocarbyl group, wherein each hydrocarbyl group has 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments, the hydrocarbyl group of R₅ is an alkyl or alkenyl group, e.g., a C₁₀₋₃₀ alkyl or alkenyl group. Thus, in some embodiments, R₅ is a non-cyclic alkyl group having at least 10 carbon atoms (such as at least 12, at least 14, at least 16, or at least 18 carbon atoms) or a non-cyclic alkenyl group having at least 10 carbon atoms (such as at least 12, at least 14, at least 16, or at least 18 carbon atoms), e.g., a straight alkyl group having at least 10 carbon atoms (such as at least 12, at least 14, at least 16, or at least 18 carbon atoms) or a straight alkenyl group having at least 10 carbon atoms (such as at least 12, at least 14, at least 16, or at least 18 carbon atoms). In some embodiments, R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments, the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments, R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments, R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments, R₅ has the following structure:

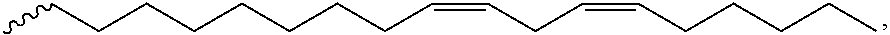

wherein [symbol] represents the bond by which R₅ is bound to the remainder of the compound.

In some of the foregoing embodiments of Formula (XI), L₂ is selected from the group consisting of —O(C═O)—, —(C═O)O—, —C(═O)—, —S—S—, —C(═O)S—, —SC(═O)—, —NR^{a}C(═O)—, —C(═O)NR^{a}—, —NR^{a}C(═O)NR^{a}—, —OC(═O)NR^{a}— and —NR^{a}C(═O)O—. In some embodiments, L₂ is selected from the group consisting of —O(C═O)—, —(C═O)O—, —C(═O)—, —C(═O)S—, —SC(═O)—, —NR^{a}C(═O)—, and —C(═O)NR^{a}—. In some embodiments, Rₐ of L₂ is H or C₁₋₁₂ alkyl. In some embodiments, Rₐ of L₂ is H or C₁₋₆ alkyl, e.g., H or C₁₋₃ alkyl. In some embodiments, Rₐ of L₂ is H, methyl, or ethyl. In some embodiments, L₂ is selected from the group consisting of —O(C═O)—, —(C═O)O—, —C(═O)S—, and —SC(═O)—. In some embodiments, L₂ is —O(C═O)— or —(C═O)O—.

In some of the foregoing embodiments of Formula (XI), G₂ is unsubstituted C₁₋₁₂ alkylene or unsubstituted C₂₋₁₂ alkenylene, e.g., unsubstituted, straight C₁₋₁₂ alkylene or unsubstituted, straight C₂₋₁₂ alkenylene. In some embodiments, G₂ is unsubstituted C₂₋₁₀ alkylene or unsubstituted C₂₋₁₀ alkenylene, e.g., unsubstituted, straight C₂₋₁₀ alkylene or unsubstituted, straight C₂₋₁₀ alkenylene. In some embodiments, G₂ is unsubstituted C₂₋₆ alkylene or unsubstituted C₂₋₆ alkenylene, e.g., unsubstituted, straight C₂₋₆ alkylene or unsubstituted, straight C₂₋₆ alkenylene. In some embodiments, G₂ is unsubstituted C₂₋₄ alkylene or unsubstituted C₂₋₄ alkenylene, e.g., unsubstituted, straight C₂₋₄ alkylene or unsubstituted, straight C₂₋₄ alkenylene. In some embodiments, G₂ is ethylene or trimethylene.

In some of the foregoing embodiments of Formula (XI), each of R₃ and R₄ is independently C₁₋₆ alkyl or C₂₋₆ alkenyl. In some embodiments, each of R₃ and R₄ is independently C₁₋₄ alkyl or C₂₋₄ alkenyl. In some embodiments, each of R₃ and R₄ is independently C₁₋₃ alkyl. In some embodiments, each of R₃ and R₄ is independently methyl or ethyl. In some embodiments, each of R₃ and R₄ is methyl.

In some of the foregoing embodiments of Formula (XI), m is 0, 1, 2 or 3. In some embodiments, m is 0 or 2. In some embodiments, m is 0. In some embodiments, m is 2.

In some of the foregoing embodiments of Formula (XI), the cationically ionizable lipid has the structure of Formula (XIIa) or (XIIb):

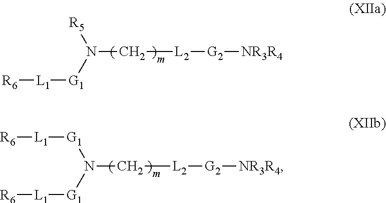

wherein
each of R₃ and R₄ is independently C₁-C₆ alkyl or C₂₋₆ alkenyl;
R₅ is a straight hydrocarbyl group having at least 14 carbon atoms (such as at least 16 carbon atoms), wherein the hydrocarbyl group preferably has at least 2 carbon-carbon double bonds;
each R₆ is independently a straight hydrocarbyl group (e.g., a straight alkyl group) having at least 10 carbon atoms and/or each R₆ is attached to L₁ via an internal carbon atom of R₆, preferably each R₆ is independently a straight hydrocarbyl group (e.g., a straight alkyl group) having at least 10 carbon atoms and each R₆ is attached to L₁ via an internal carbon atom of R₆;
each G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene, such as unsubstituted, straight C₈₋₁₂ alkylene or unsubstituted, straight C₈₋₁₂ alkenylene;
G₂ is unsubstituted C₂-C₁₀ alkylene or C₂₋₁₀ alkenylene, preferably unsubstituted C₂-C₆ alkylene or C₂₋₆ alkenylene;
each of L₁ and L₂ is independently —O(C═O)— or —(C═O)O—; and
m is 0, 1, 2 or 3, preferably 0 or 2.

In some of the foregoing embodiments of Formula (XIIa), R₅ has at most 30 carbon atoms, such as at most 28, at most 26, at most 24, at most 22, or at most 20 carbon atoms. In some embodiments of formulas (XIIa), R₅ is a straight hydrocarbyl group having 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIa), R₅ is a straight alkyl or alkenyl group having 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIa), the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has 2 or 3 carbon-carbon double bonds, wherein at least 1 carbon-carbon double bond, such as 1, 2, or 3 carbon-carbon double bonds, is in cis configuration. In some embodiments of formula (XIIa), R₅ has the following structure:

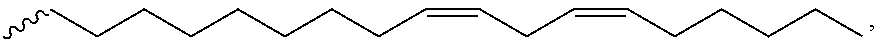

wherein [symbol] represents the bond by which R₅ is bound to the remainder of the compound. In some embodiments of formula (XIIa), R₆ has at most 30 carbon atoms, such as at most 28, at most 26, at most 24, at most 22, or at most 20 carbon atoms. In some embodiments of formula (XIIa), R₆ is a non-cyclic hydrocarbyl group (e.g., a non-cyclic alkyl group) having 10 to 30 carbon atoms (such as 10 to 28, 10 to 26, 10 to 24, 10 to 22, or 10 to 20 carbon atoms), e.g., a straight hydrocarbyl group (e.g., a straight alkyl group) having 10 to 30 carbon atoms (such as 10 to 28, 10 to 26, 10 to 24, 10 to 22, or 10 to 20 carbon atoms). In some embodiments of formula (XIIa), R₆ is a straight hydrocarbyl group (e.g., a straight alkyl group) having at least 10 carbon atoms and R₆ is attached to L₁ via an internal carbon atom of R₆. In some embodiments of formula (XIIa), R₆ is a non-cyclic hydrocarbyl group (e.g., a non-cyclic alkyl group) having 10 to 30 carbon atoms (such as 10 to 28, 10 to 26, 10 to 24, 10 to 22, or 10 to 20 carbon atoms), e.g., a straight hydrocarbyl group (e.g., a straight alkyl group) having 10 to 30 carbon atoms (such as 10 to 28, 10 to 26, 10 to 24, 10 to 22, or 10 to 20 carbon atoms), and R₆ is attached to L₁ via an internal carbon atom of R₆. In some embodiments of formula (XIIa), G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene. In some embodiments of formula (XIIa), R₅ is a straight hydrocarbyl group, e.g., a straight alkenyl group, having at least 14 carbon atoms (such as 14 to 30 carbon atoms) and 2 or 3 carbon-carbon double bonds; R₆ is a straight hydrocarbyl group (e.g., a straight alkyl group) having at least 10 carbon atoms (e.g., having 10 to 30 carbon atoms) and R₆ is attached to L₁ via an internal carbon atom of R₆; and G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene.

In some of the foregoing embodiments of Formula (XIIb), each R₆ has independently at most 30 carbon atoms, such as at most 28, at most 26, at most 24, at most 22, or at most 20 carbon atoms. In some embodiments of formula (XIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms). In some embodiments of formula (XIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and each R₆ is attached to L₁ via an internal carbon atom of R₆. In some embodiments of formula (XIIb), each R₆ is independently selected from the group consisting of:

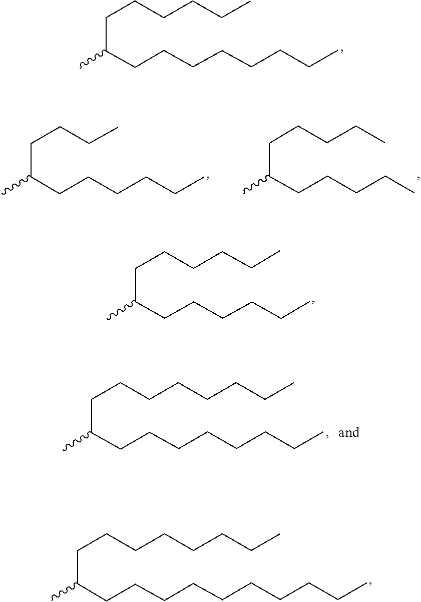

wherein [symbol] represents the bond by which R₆ is bound to L₁. In some embodiments of formula (XIIb), each G₁ is independently unsubstituted, straight C₆₋₁₂ alkylene or C₆₋₁₂ alkenylene. In some embodiments of formula (XIIb), each G₁ is independently unsubstituted, straight C₈₋₁₂ alkylene or C₈₋₁₂ alkenylene. In some embodiments of formula (XIIb), each R₆ is independently a straight hydrocarbyl group (e.g., a straight alkyl group) having at least 10 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and is attached to L₁ via an internal carbon atom of R₆; and each G₁ is independently unsubstituted, straight C₈₋₁₂ alkylene or C₈₋₁₂ alkenylene.

In some of the foregoing embodiments of Formula (XI), the cationically ionizable lipid has the structure of Formula (XIIIa) or (XIIIb):

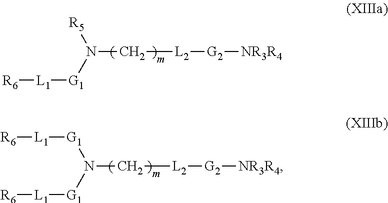

wherein
each of R₃ and R₄ is independently C₁₋₄ alkyl or C₂₋₄ alkenyl, more preferably C₁₋₃ alkyl, such as methyl or ethyl;
R₅ is a straight alkyl or alkenyl group having at least 16 carbon atoms, wherein the alkenyl group preferably has at least 2 carbon-carbon double bonds;
each R₆ is independently a straight hydrocarbyl group having at least 10 carbon atoms, wherein R₆ is attached to L₁ via an internal carbon atom of R₆;
each G₁ is independently unsubstituted, straight C₆₋₁₂ alkylene or unsubstituted, straight C₆₋₁₂ alkenylene, e.g., unsubstituted, straight C₈₋₁₂ alkylene or unsubstituted, straight C₈₋₁₂ alkenylene, such as unsubstituted, straight C₈₋₁₀ alkylene or unsubstituted, straight C₈₋₁₀ alkenylene, such as unsubstituted, straight C₈ alkylene;
G₂ is unsubstituted C₂₋₆ alkylene or C₂₋₆ alkenylene, preferably unsubstituted C₂₋₄ alkylene or C₂₋₄ alkenylene, such as ethylene or trimethylene;
each of L₁ and L₂ is independently —O(C═O)— or —(C═O)O—; and
m is 0, 1, 2 or 3, preferably 0 or 2.

In some of the foregoing embodiments of Formula (XIIIa), R₅ has at most 30 carbon atoms, such as at most 28, at most 26, at most 24, at most 22, or at most 20 carbon atoms. In some embodiments of formulas (XIIIa), R₅ is a straight alkyl or alkenyl group having 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIIa), the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has 2 or 3 carbon-carbon double bonds, wherein at least 1 carbon-carbon double bond, such as 1, 2, or 3 carbon-carbon double bonds, is in cis configuration. In some embodiments of formula (XIIIa), R₅ has the following structure:

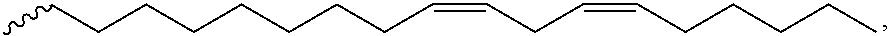

wherein [symbol] represents the bond by which R₅ is bound to the remainder of the compound. In some embodiments of formula (XIIIa), R₆ has at most 30 carbon atoms, such as at most 28, at most 26, at most 24, at most 22, or at most 20 carbon atoms. In some embodiments of formula (XIIIa), R₆ is a straight hydrocarbyl group (e.g., a straight alkyl group) having 10 to 30 carbon atoms (such as 10 to 28, 10 to 26, 10 to 24, 10 to 22, or 10 to 20 carbon atoms) and R₆ is attached to L₁ via an internal carbon atom of R₆. In some embodiments of formula (XIIIa), R₆ is a straight alkyl group having 10 to 30 carbon atoms (such as 10 to 28, 10 to 26, 10 to 24, 10 to 22, or 10 to 20 carbon atoms) and R₆ is attached to L₁ via an internal carbon atom of R₆. In some embodiments of formula (XIIIa), G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene. In some embodiments of formula (XIIIa), R₅ is a straight hydrocarbyl group, e.g., a straight alkenyl group, having at least 16 carbon atoms (such as 16 to 30 carbon atoms) and 2 or 3 carbon-carbon double bonds; R₆ is a straight hydrocarbyl group (e.g., a straight alkyl group) having at least 10 carbon atoms (e.g., having 10 to 30 carbon atoms) and R₆ is attached to L₁ via an internal carbon atom of R₆; and G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene.

In some of the foregoing embodiments of Formula (XIIIb), each R₆ has independently at most 30 carbon atoms, such as at most 28, at most 26, at most 24, at most 22, or at most 20 carbon atoms. In some embodiments of formula (XIIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and each R₆ is attached to L₁ via an internal carbon atom of R₆. In some embodiments of formula (XIIIb), each R₆ is attached to L₁ via an internal carbon atom of R₆ and is independently selected from the group consisting of:

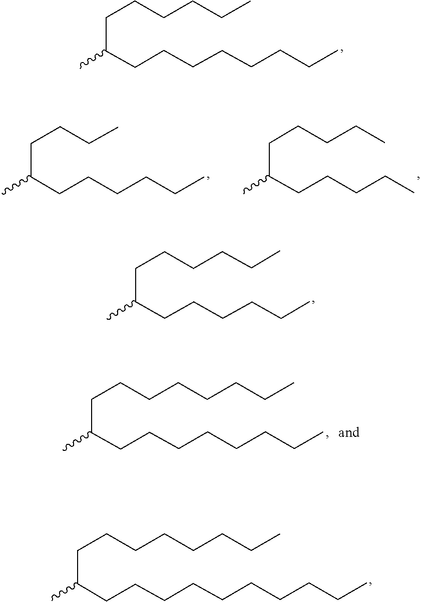

wherein [symbol] represents the bond by which R₆ is bound to L₁. In some embodiments of formula (XIIIb), each G₁ is independently unsubstituted, straight C₈₋₁₂ alkylene or C₈₋₁₂ alkenylene, e.g., unsubstituted, straight C₈₋₁₀ alkylene or C₈₋₁₀ alkenylene. In some embodiments of formula (XIIIb), each R₆ is independently a straight hydrocarbyl group (e.g., a straight alkyl group) having at least 10 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and is attached to L₁ via an internal carbon atom of R₆; and each G₁ is independently unsubstituted, straight C₈₋₁₂ alkylene or C₈₋₁₂ alkenylene, e.g., unsubstituted, straight C₈₋₁₀ alkylene or C₈₋₁₀ alkenylene.

In some of the foregoing embodiments of Formula (XI), the cationically ionizable lipid has one of the following formulas (XIV-1), (XIV-2), and (XIV-3):

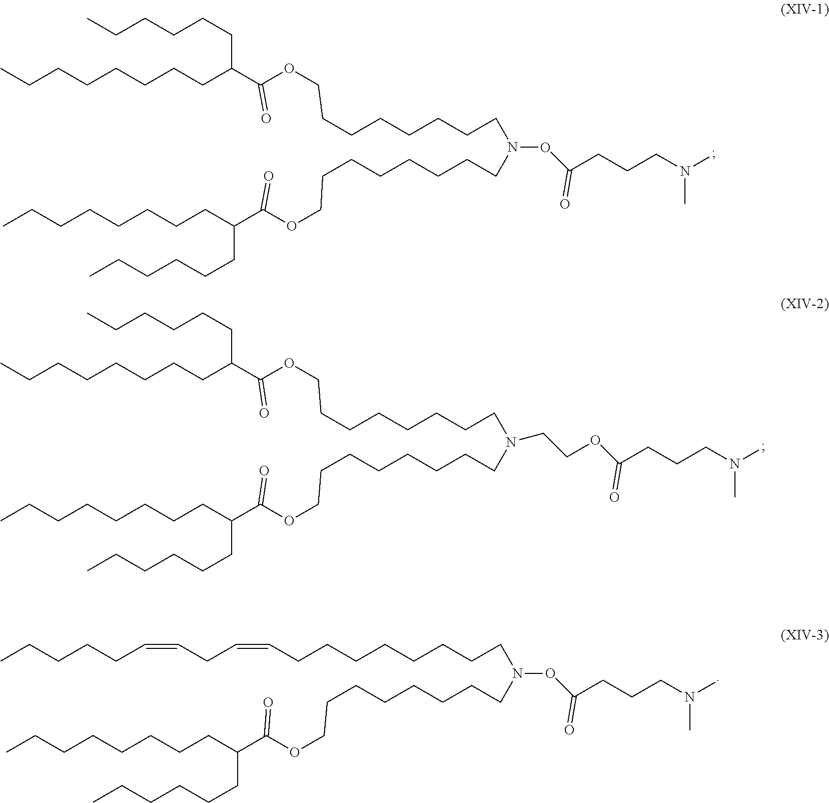

In some embodiments, the cationically ionizable lipid is (6Z,16Z)-12-((Z)-dec-4-en-1-yl) docosa-6,16-dien-11-yl 5-(dimethylamino) pentanoate (3D-P-DMA). The structure of 3D-P-DMA may be represented as follows:

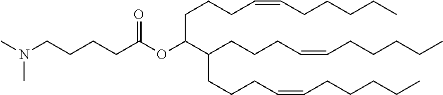 [SMILES: CCCCC/C=C\CCCC(CCC/C=C\CCCCC)C(CCC/C=C\CCCCC)OC(=O)CCCCN(C)C]

In some embodiments, the cationically ionizable lipid is selected from those described generally and specifically in WO 2018/087753.

In some embodiments, the cationically ionizable lipid is selected from the group consisting of:

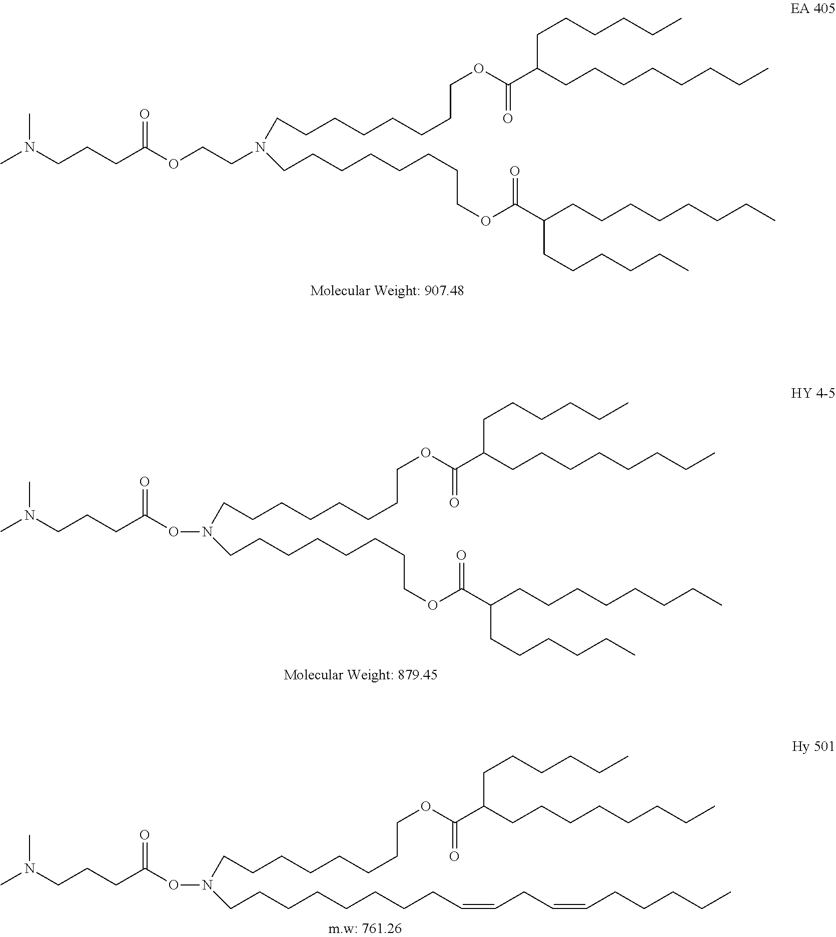

In some embodiments, the cationically ionizable lipid is selected from those described generally and specifically in U.S. Pat. No. 10,221,127 B2. In some embodiments, the cationically ionizable lipid is selected from those described generally and specifically in WO 2017/049245A2. In some embodiments, the cationically ionizable lipid is selected from those described generally and specifically in US2022/0218622A1. In some embodiments, the cationically ionizable lipid is selected from those described generally and specifically in WO 2021/000041A1. In some embodiments, the cationically ionizable lipid is selected from those described generally and specifically in WO 2020/252589A1. In some embodiments, the cationically ionizable lipid is selected from those described generally and specifically in Cornebise et al. Adv. Funct. Mater. 2022, 32, 2106727a.

In some embodiments, the cationically ionizable lipid is selected from the following as listed in Table 4 below.

TABLE 4

| | |
|---|---|
| U.S. Pat. No. 10,221,127 B2 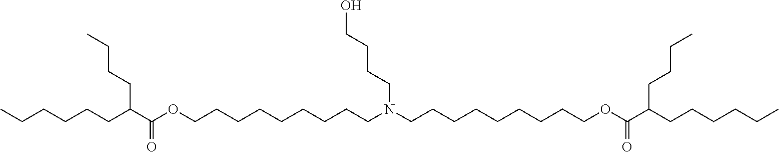 | Compound 25 |
| U.S. Pat. No. 10,221,127 B2 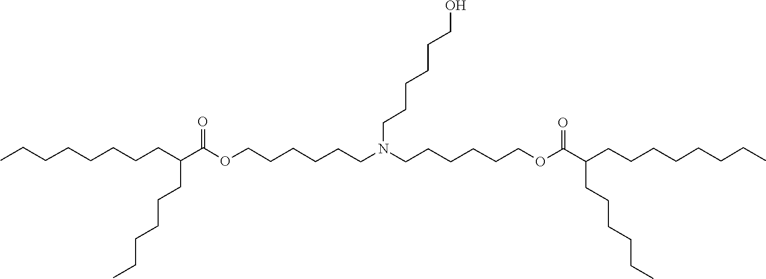 | Compound 8 |
| U.S. Pat. No. 10,221,127 B2 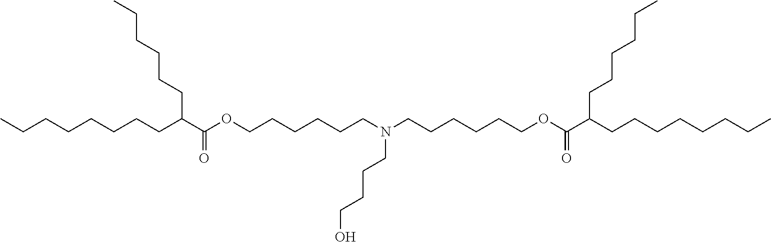 | Compound 3 |
| U.S. Pat. No. 10,221,127 B2 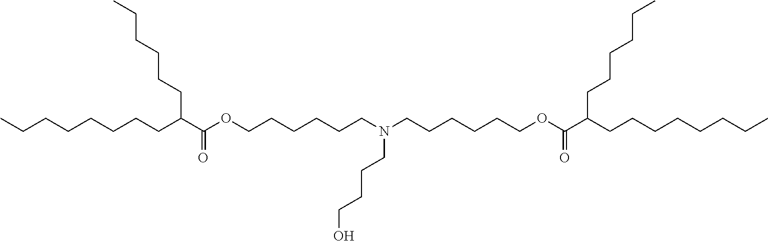 | Compound 2 |
| U.S. Pat. No. 10,221,127 B2 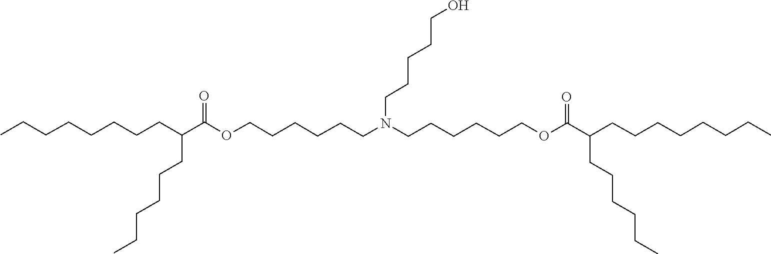 | Compound 7 |
| U.S. Pat. No. 10,221,127 B2 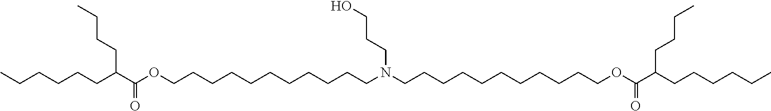 | Compound 45 |
| U.S. Pat. No. 10,221,127 B2 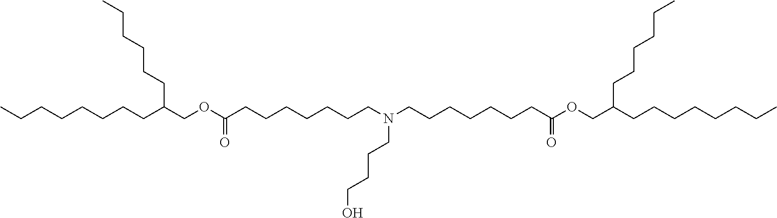 | Compound 20 |
| U.S. Pat. No. 10,221,127 B2 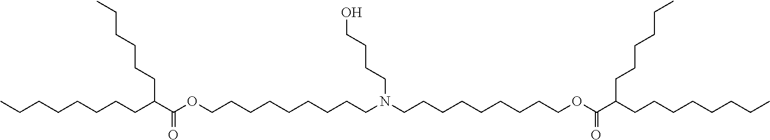 | Compound 22 |
| U.S. Pat. No. 10,221,127 B2 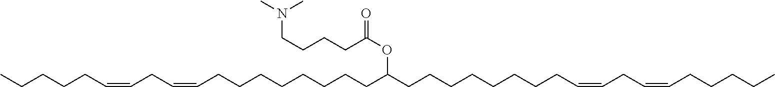 | Compound MC4 |
| U.S. Pat. No. 10,221,127 B2 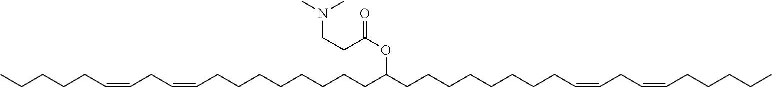 | Compound MC2 |
| U.S. Pat. No. 10,221,127 B2 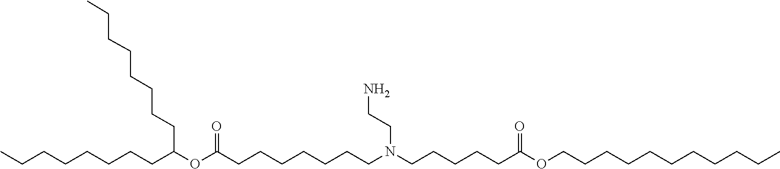 | Compound 25 |
| WO 2017/049245A2 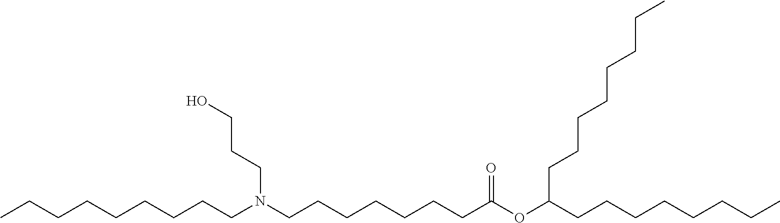 | Compound 7 |
| WO 2017/049245A2 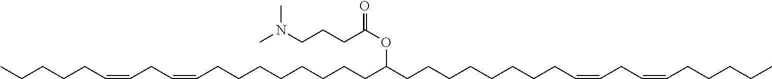 | Compound MC3 |
| WO 2017/049245A2 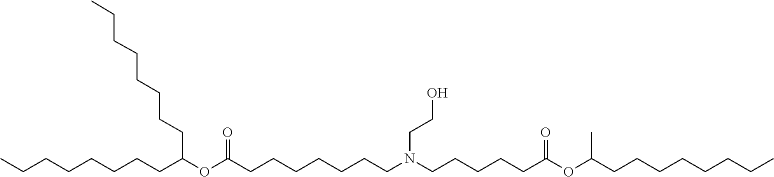 | Compound 122 |
| WO 2017/049245A2 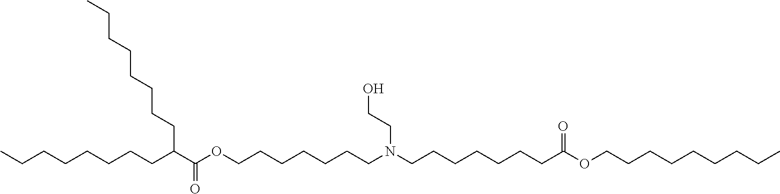 | Compound 30 |
| WO 2017/049245A2 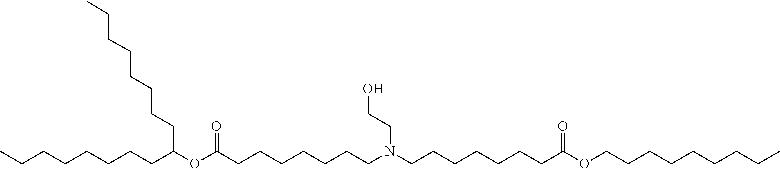 | Compound 18 |
| WO 2017/049245A2 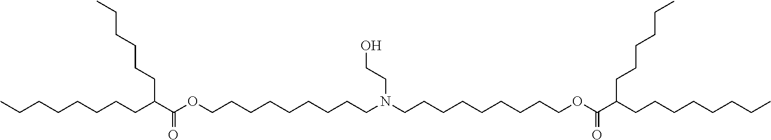 | Compound 133 |
| WO 2017/049245A2 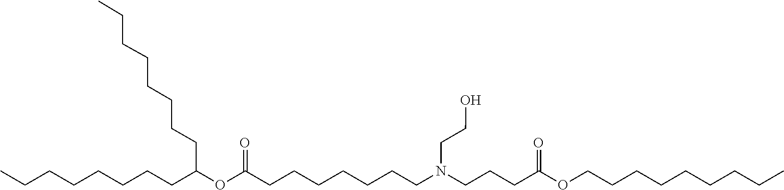 | Compound 26 |
| WO 2017/049245A2 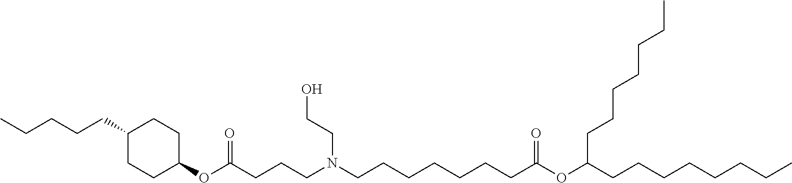 | Compound 67 |
| WO 2017/049245A2 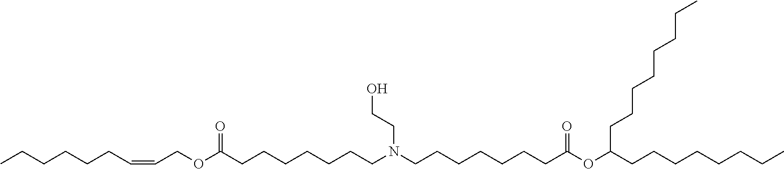 | Compound 24 |
| WO 2017/049245A2 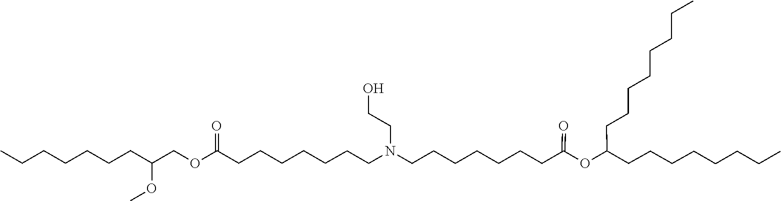 | Compound 148 |
| WO 2017/049245A2 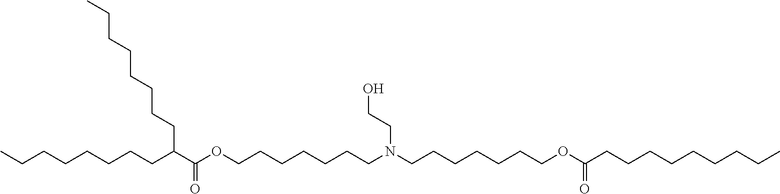 | Compound 151 |
| WO 2017/049245A2 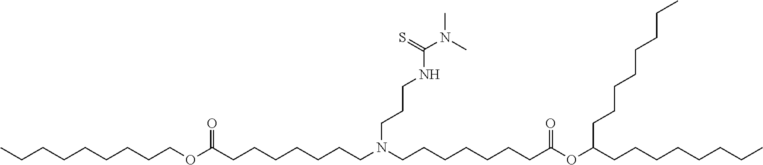 | Compound 111 |
| WO 2017/049245A2 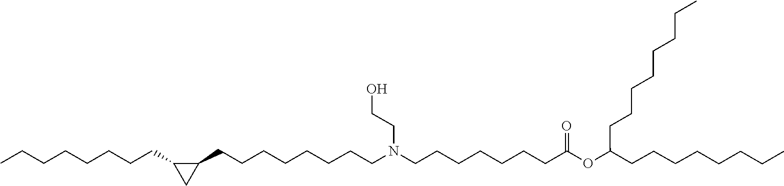 | Compound 47 |
| WO 2017/049245A2 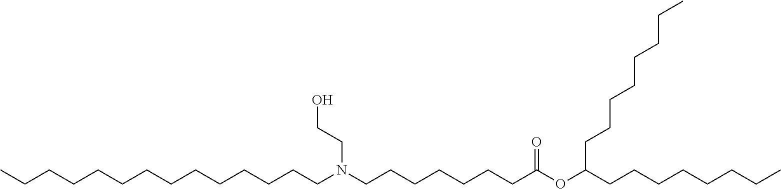 | Compound 2 |
| WO 2017/049245A2 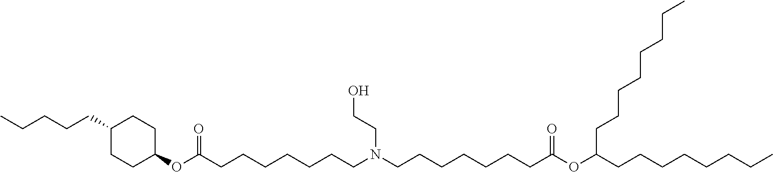 | Compound 66 |
| WO 2017/049245A2 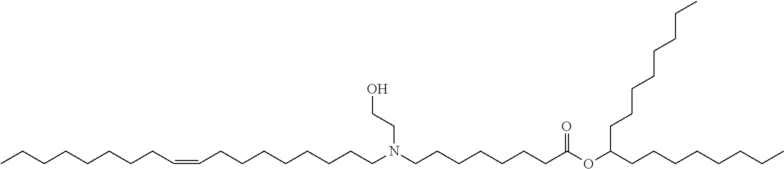 | Compound 31 |
| WO 2017/049245A2 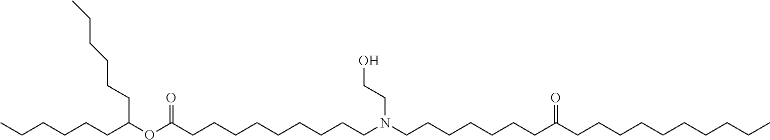 | Compound 147 |
| WO 2017/049245A2 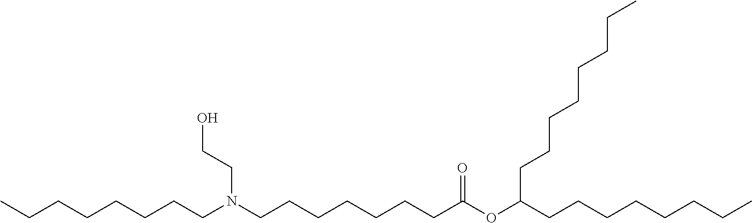 | Compound 4 |
| WO 2017/049245A2 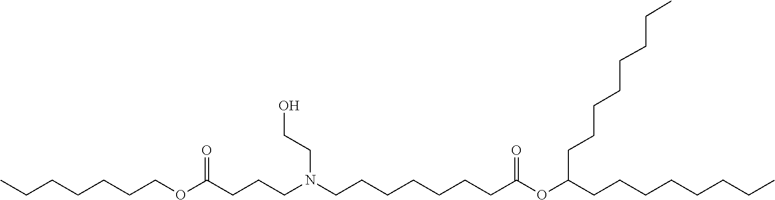 | Compound 54 |
| WO 2017/049245A2 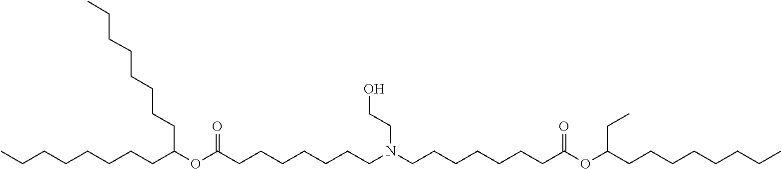 | Compound 49 |
| WO 2017/049245A2 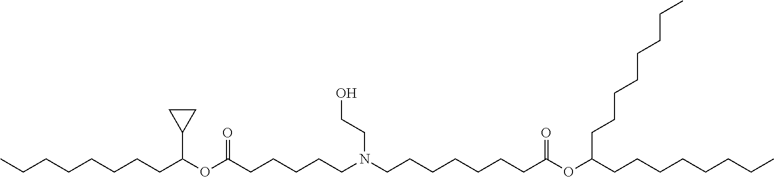 | Compound 128 |
| WO 2017/049245A2 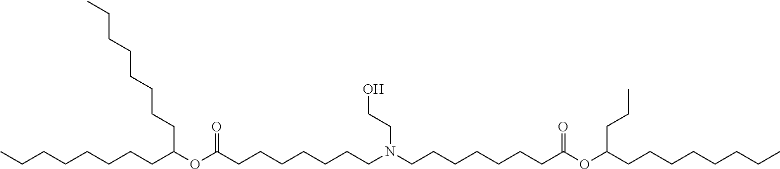 | Compoud 50 |
| WO 2017/049245A2 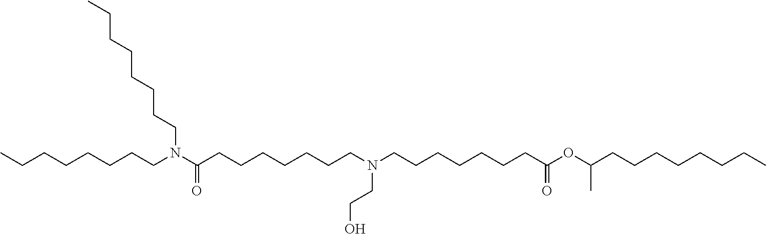 | Compound 130 |
| WO 2017/049245A2 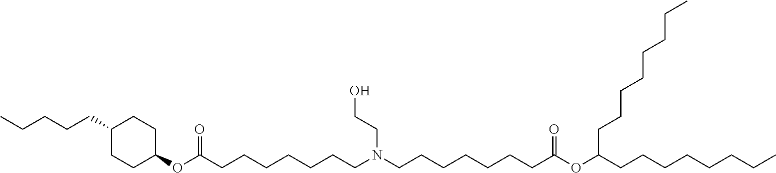 | Compound 68 |
| WO 2017/049245A2 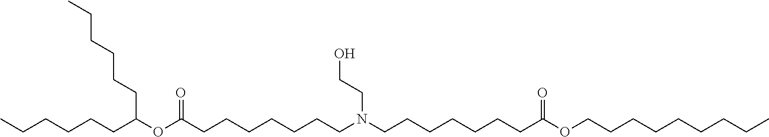 | Compound 32 |
| WO 2017/049245A2 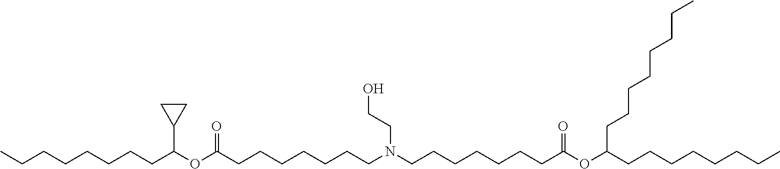 | Compound 65 |
| WO 2017/049245A2 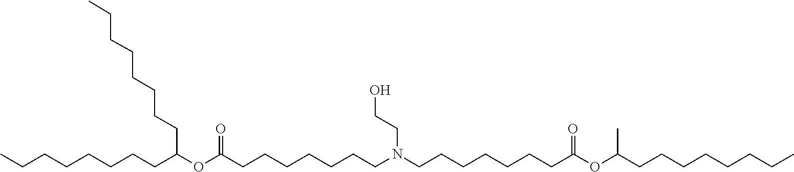 | Compound 48 |
| WO 2017/049245A2 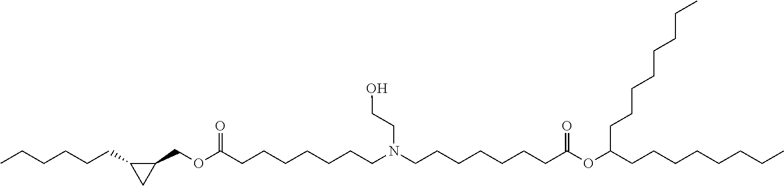 | Compound 28 |
| WO 2017/049245A2 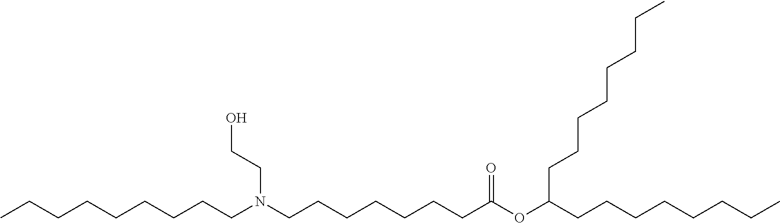 | Compound 3 |
| WO 2017/049245A2 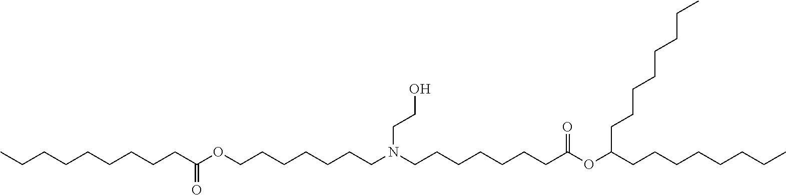 | Compound 96 |
| WO 2017/049245A2 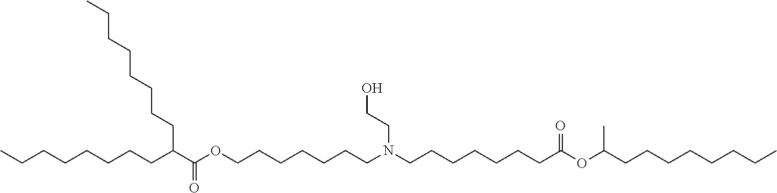 | Compound 135 |
| WO 2017/049245A2 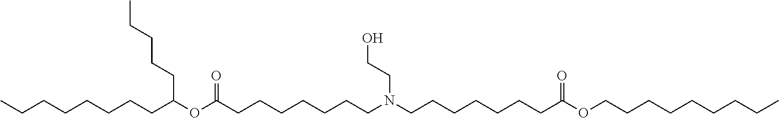 | Compound 33 |
| US2022/0218622A1-C2C2-1Me-Pyr 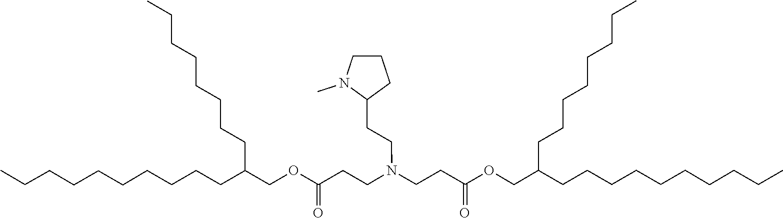 | Compound BODD |
| US2022/0218622A1-C2C2-DMA 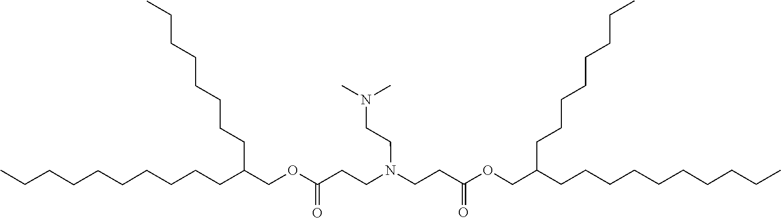 | Compound BODD |
| US2022/0218622A1-C2C4-PipZ 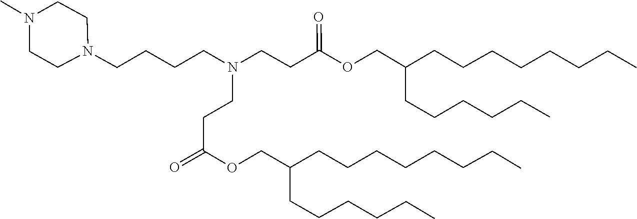 | Compound BHD |
| US2022/0218622A1-C2C4-Pyr 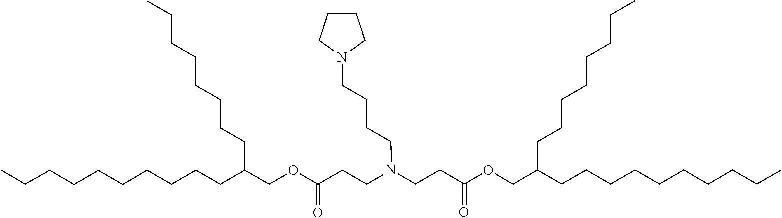 | Compound BODD |
| US2022/0218622A1-C2C2-PipZ 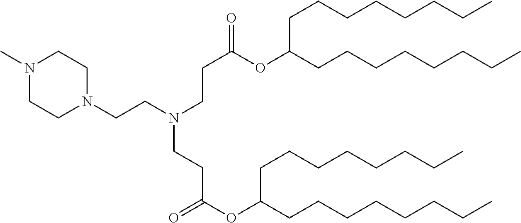 | Compound BHD |
| US2022/0218622A1-C2C4-PipZ 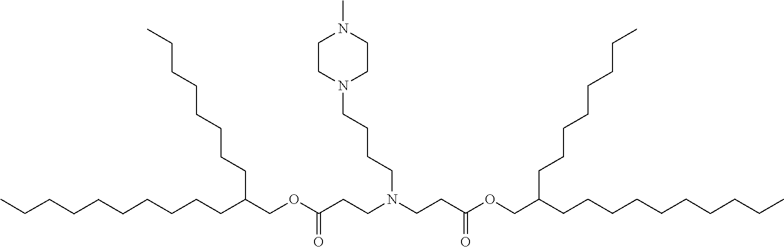 | Compound BODD |
| US2022/0218622A1-C2C2-PipZ 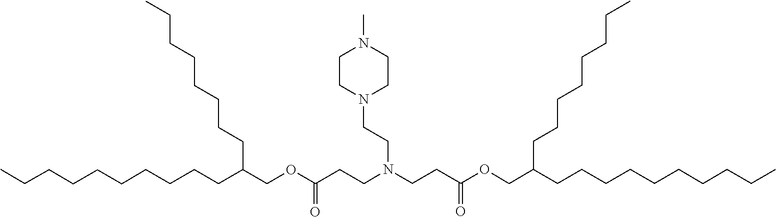 | Compound BODD |
| WO 2021/000041A1 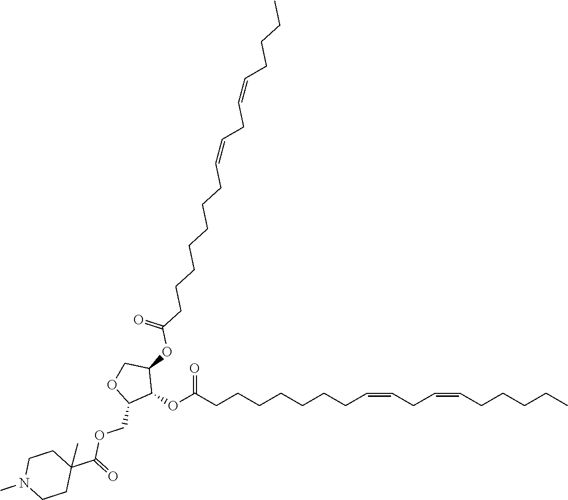 | Compound 127 |
| WO 2021/000041A1 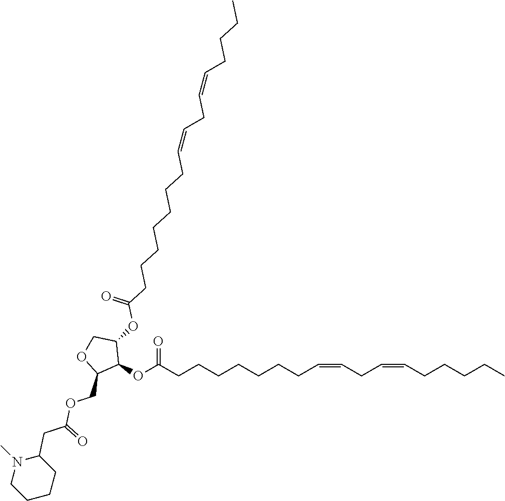 | Compound 540 |
| WO 2021/000041A1 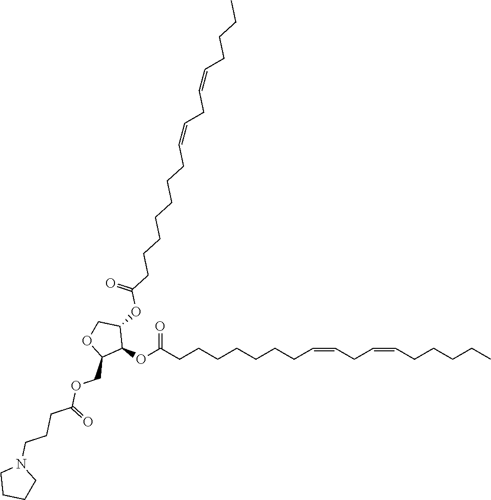 | Compound 539 |
| WO 2021/000041A1 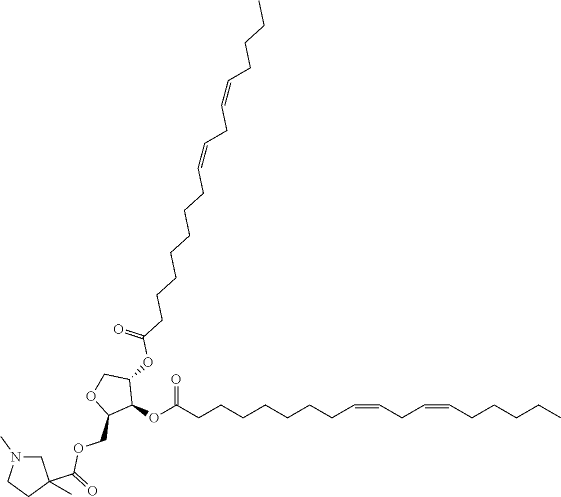 | Compound 329 |
| WO 2021/000041A1 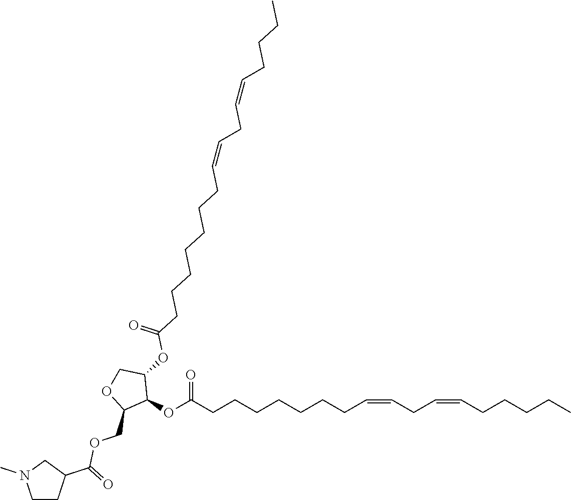 | Compound 328 |
| WO 2021/000041A1 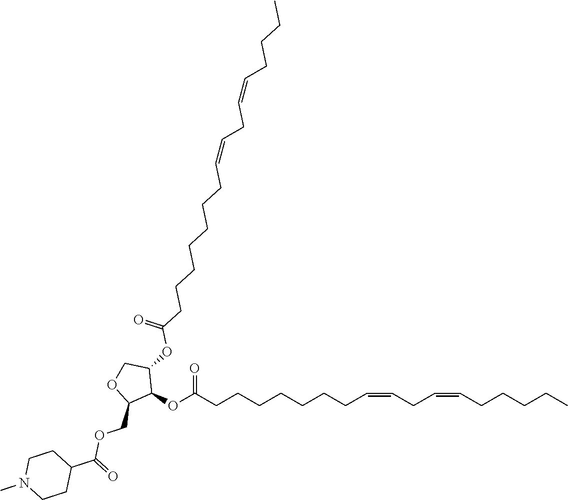 | Compound 325 |
| WO 2020/252589A1 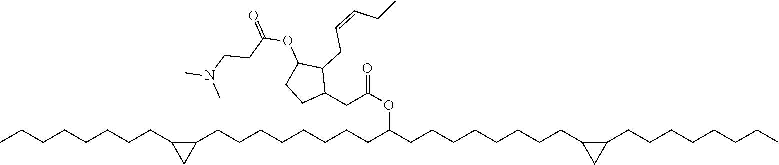 | Compound 516 |
| WO 2020/252589A1 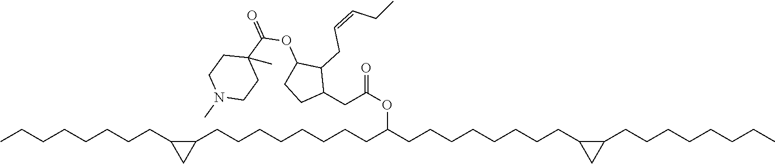 | Compound 560 |
| WO 2020/252589A1 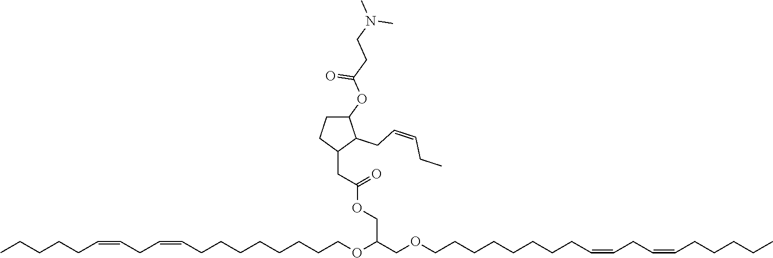 | Compound 128 |
| WO 2020/252589A1 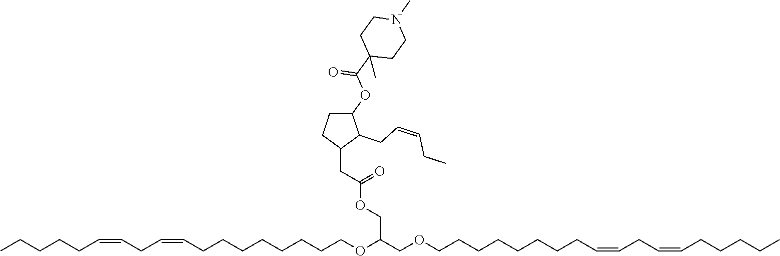 | Compound 132 |
| Cornebise et al. 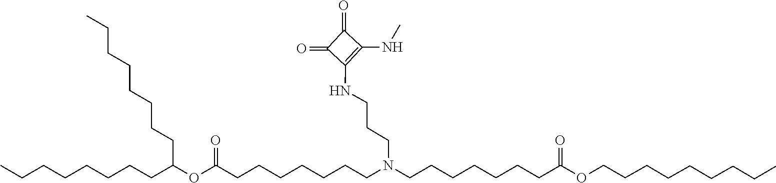 | Compound 23 |
| Cornebise et al. 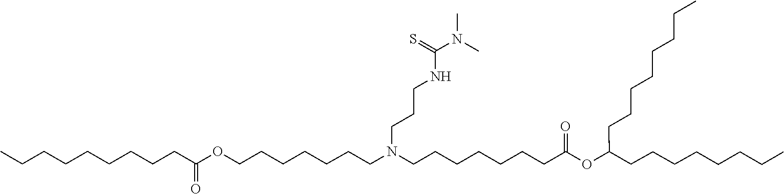 | Compound 24 |
| Cornebise et al. 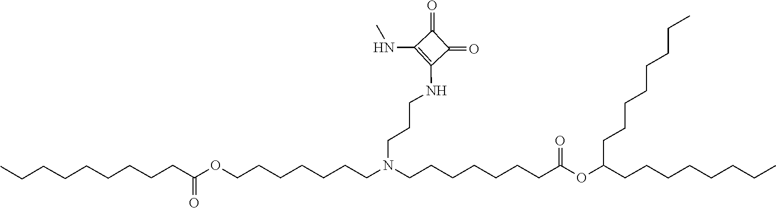 | Compound 25 |
| Cornebise et al. 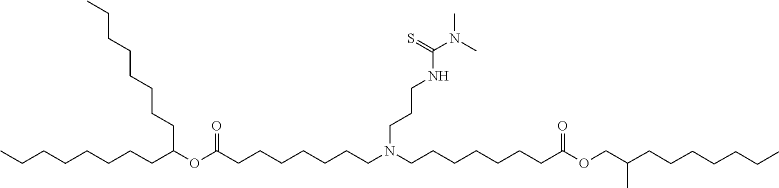 | Compound 26 |
| Cornebise et al. 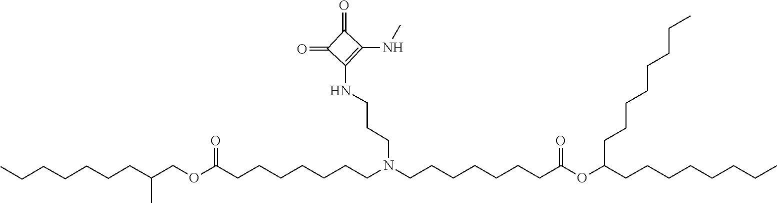 | Compound 27 |
| Cornebise et al. 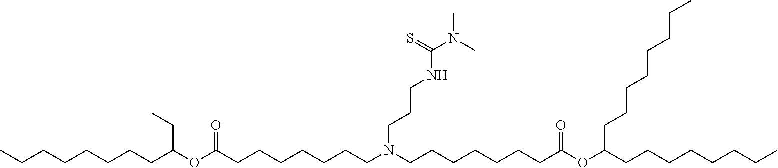 | Compound 28 |
| Cornebise et al. 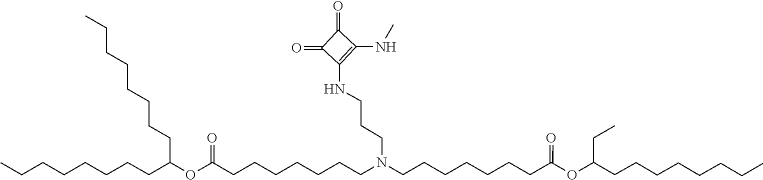 | Compound 29 |
| Cornebise et al. 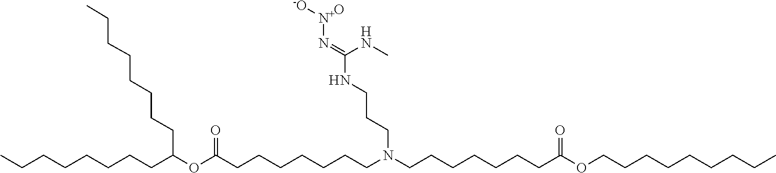 | Compound 15 |
| Cornebise et al. 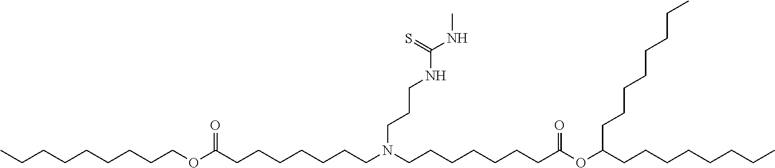 | Compound 9 |
| Cornebise et al. 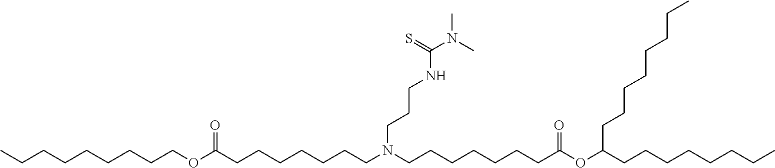 | Compound 7 |
| Cornebise et al. 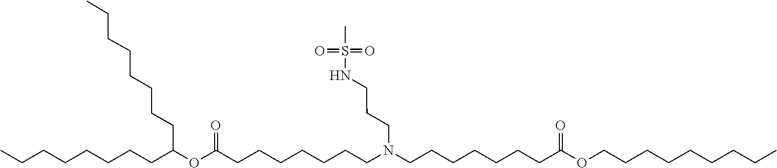 | Compound 5 |
| US2022/0218622A1-BODD-C2C2-Py 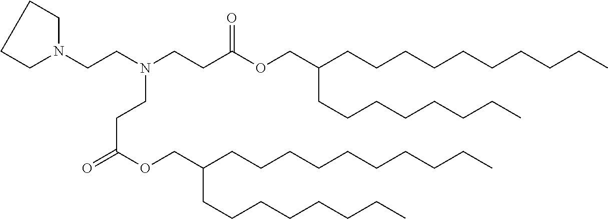 | |
| US2022/0218622A1-BODD-C2C2-1Me-3PipD 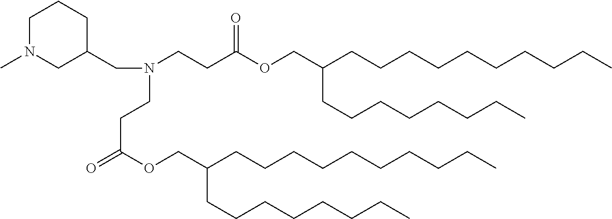 | |
| US2022/0218622A1-DND-C2-C4-PipZ 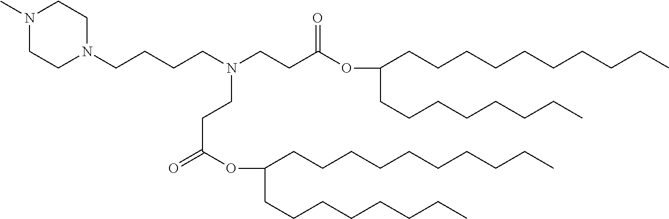 | |

Examples of preferred cationically ionizable lipids include, but are not limited to:
N,N-dimethyl-2,3-dioleyloxypropylamine (DODMA);
1,2-distearyloxy-N,N-dimethyl-3-aminopropane (DSDMA),
1,2-dilinoleyloxy-N,N-dimethylaminopropane (DLinDMA),
3-dimethylamino-2-(cholest-5-en-3-beta-oxybutan-4-oxy)-1-(cis,cis-9,12-octadecadienoxy) propane (CLinDMA),
2-[5′-(cholest-5-en-3-beta-oxy)-3′-oxapentoxy)-3-dimethyl-1-(cis,cis-9′,12′-octadecadienoxy) propane (CpLinDMA),
N,N-dimethyl-3,4-dioleyloxybenzylamine (DMOBA),
1,2-N,N′-dioleylcarbamyl-3-dimethylaminopropane (DOcarbDAP),
2,3-dilinoleoyloxy-N,N-dimethylpropylamine (DLinDAP),
1,2-N,N′-Dilinoleylcarbamyl-3-dimethylaminopropane (DLincarbDAP),
1,2-dilinoleoylcarbamyl-3-dimethylaminopropane (DLinCDAP),
2,2-dilinoleyl-4-dimethylaminomethyl-[1,3]-dioxolane (DLin-K-DMA),
2,2-dilinoleyl-4-dimethylaminoethyl-[1,3]-dioxolane (DLin-K5-XTC2-DMA),
2,2-dilinoleyl-4-(2-dimethylaminoethyl)-[1,3]-dioxolane (DLin-KC2-DMA),
heptatriaconta-6,9,28,31-tetraen-19-yl-4-(dimethylamino)butanoate (DLin-MC3-DMA),
2-({8-[(3B)-cholest-5-en-3-yloxy]octyl}oxy)-N,N-dimethyl-3-[(9Z,12Z)-octadeca-9,12-dien-1-yloxy]propan-1-amine (octyl-CLinDMA),
di((Z)-non-2-en-1-yl) 8,8′-((((2 (dimethylamino)ethyl)thio) carbonyl) azanediyl)-dioctanoate (ATX),
N,N-dimethyl-2,3-bis(dodecyloxy) propan-1-amine (DLDMA),
N,N-dimethyl-2,3-bis(tetradecyloxy) propan-1-amine (DMDMA),
Di((Z)-non-2-en-1-yl)-9-((4-(dimethylaminobutanoyl)oxy) heptadecanedioate (L319),
N-dodecyl-3-((2-dodecylcarbamoyl-ethyl)-{2-[(2-dodecylcarbamoylethyl)-2-{(2-dodecylcarbamoyl-ethyl)-[2-(2-dodecylcarbamoyl-ethylamino)-ethyl]-amino}-ethylamino) propionamide (lipidoid 98N12-5),
1-[2-[bis(2-hydroxydodecyl)amino]ethyl-[2-[4-[2-[bis(2-hydroxydodecyl)-amino]ethyl]piperazin-1-yl]ethyl]amino]dodecan-2-ol (lipidoid C12-200),
(6Z,9Z,28Z,31Z)-heptatriaconta-6,9,28,31-tetraen-19-yl 4-(dimethylamino)butanoate;
bis-(2-butyloctyl) 10-(N-(3-(dimethylamino) propyl) nonanamido) nonadecanedioate (A9);
[(4-hydroxybutyl) azanediyl]di(hexane-6,1-diyl)bis(2-hexyldecanoate)) (ALC-315);
(heptadecan-9-yl 8-{(2-hydroxyethyl) [6-oxo-6-(undecyloxy) octyl]amino}octanoate) (L5);
heptadecan-9-yl 8-{(2-hydroxyethyl) [6-oxo-6-(undecyloxy) hexyl]amino}octanoate) (SM-102);
((2-((4-(dimethylamino)butanoyl)oxy)ethyl) azanediyl)bis(octane-8,1-diyl)bis(2-hexyldecanoate) (EA 405) (as described further herein);
(((4-(dimethylamino)butanoyl)oxy) azanediyl)bis(octane-8,1-diyl)bis(2-hexyldecanoate) (HY405) (as described further herein);
8-(((4-(dimethylamino)butanoyl)oxy)((9Z,12Z)-octadeca-9,12-dien-1-yl)amino) octyl 2-hexyldecanoate (HY501) (as described further herein);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
or mixtures of any thereof.

In one embodiment, the cationically ionisable lipid is selected from the group consisting of:
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
N,N-dimethyl-2,3-dioleyloxypropylamine (DODMA);
di((Z)-non-2-en-1-yl)-9-((4-(dimethylaminobutanoyl)oxy) heptadecanedioate (L319);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
HY501 (as described further herein);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
di(heptadecan-9-yl) 3,3′-((2-(4-methylpiperazin-1-yl)ethyl) azanediyl)dipropionate (BHD-C2C2-PipZ);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   or a mixture of any thereof.

In one embodiment, the cationically ionisable lipid is selected from the group consisting of:
N,N-dimethyl-2,3-dioleyloxypropylamine (DODMA);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   or a mixture of any thereof.

In one embodiment, the cationically ionisable lipid is selected from the group consisting of:
N,N-dimethyl-2,3-dioleyloxypropylamine (DODMA);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ); and
bis(2-octyldodecyl) 3,3′-((4-(pyrrolidin-1-yl)butyl) azanediyl)dipropionate (BODD-C2C4-Pyr);
   or a mixture of any thereof.

In some embodiments, the cationically ionisable lipid is not BHD-C2C4-PipZ. In some embodiments, the cationically ionisable lipid is not DND-C2-C4-PipZ.

In one embodiment, the cationically ionisable lipid is present in the composition in an amount of 20 to 80 mol % of the total lipid present in the composition. In one embodiment, the cationically ionizable lipid may comprise from about 20 mol % to about 75 mol %, from about 20 mol % to about 70 mol %, from about 20 mol % to about 60 mol %, from about 25 mol % to about 55 mol %, from about 30 mol % to about 50 mol %, from about 35 mol % to about 45 mol %, or from about 40 mol % to about 55 mol % of the total lipid present in the composition. Typically in the LNP compositions of the present invention, the molar ratio of cationically ionizable lipid to negatively charged amphiphile is not 1:1. In some cases, the molar ratio of cationically ionizable lipid to negatively charged amphiphile is not between 0.9 and 1.1. In preferred embodiments the molar ratio between the cationically ionizable lipid to the negatively charged amphiphile is between 2 and 10, more preferred between 3 and 7.

### Negatively Charged Amphiphile

The composition of the present disclosure also includes a negatively charged amphiphiles (also referred to herein as an “anionic amphiphile”). In this specification the term “amphiphile” is defined generally as a molecule having both hydrophilic and lipophilic moieties (as defined above). The amphiphiles useful in the compositions of the present invention are anisotropic and have a hydrophilic portion and a lipophilic portion. The negative charge is situated in the hydrophilic portion of the amphiphile. The negatively charged amphiphile may have one negatively charged group or multiple (e.g. 2, 3, 4, or 5) negatively charged groups.

As the skilled person will readily understand, depending on the pKₐ of the amphiphile and the pH, the amphiphile may be present in a protonated form (described in standard chemical nomenclature as the acid) or in a negatively charged, deprotonated form (described in standard chemical nomenclature by the name of the acid with the suffix “-ate” substituting for “acid”). The present invention encompasses anionic amphiphiles in both protonated and deprotonated forms, regardless of the form in which they are described in this specification.

The lipophilic moiety of the negatively charged amphiphile may be hydrocarbyl groups or heterohydrocarbyl groups, as defined above. Where the lipophilic moieties are hydrocarbyl groups, they may be selected from alkyl groups, alkenyl groups, alkynyl groups, cycloalkyl groups, cycloalkenyl groups, cycloalkylalkyl groups, alkylcycloalkyl groups, alkylcycloalkylalkyl groups, aryl groups, alkylaryl groups, arylalkyl groups, and alkylarylalkyl groups (all as defined above, either in a broadest aspect or a preferred aspect). Where the lipophilic moieties are heterohydrocarbyl groups, they may be alkylheteroaryl, heteroarylalkyl, alkylheterocyclyl, or heterocyclylalkyl groups (all as defined above, either in a broadest aspect or a preferred aspect). The lipophilic moiety may comprise two or more groups from the aforementioned list.

In one embodiment, the lipophilic portion of the negatively charged amphiphile has from 6 to 40 carbon atoms. In one embodiment, the lipophilic portion of the negatively charged amphiphile has from 6 to 30 carbon atoms. In one embodiment, the lipophilic portion of the negatively charged amphiphile has from 6 to 20 carbon atoms. In one embodiment, the lipophilic portion of the negatively charged amphiphile has from 6 to 20 carbon atoms.

In one embodiment, the lipophilic portion of the negatively charged amphiphile is an alkyl group having from 6 to 40 carbon atoms. In one embodiment, the lipophilic portion of the negatively charged amphiphile is an alkyl group having from 6 to 30 carbon atoms. In one embodiment, the lipophilic portion of the negatively charged amphiphile is an alkyl group having from 6 to 20 carbon atoms.

In one embodiment, the lipophilic portion of the negatively charged amphiphile is an alkenyl group having from 6 to 40 carbon atoms. In one embodiment, the lipophilic portion of the negatively charged amphiphile is an alkenyl group having from 6 to 30 carbon atoms. In one embodiment, the lipophilic portion of the negatively charged amphiphile is an alkenyl group having from 6 to 20 carbon atoms.

In one embodiment, the lipophilic portion of the negatively charged amphiphile is an alkynyl group having from 6 to 40 carbon atoms. In one embodiment, the lipophilic portion of the negatively charged amphiphile is an alkynyl group having from 6 to 30 carbon atoms. In one embodiment, the lipophilic portion of the negatively charged amphiphile is an alkynyl group having from 6 to 20 carbon atoms.

In one embodiment, the lipophilic portion of the negatively charged amphiphile is a cycloalkyl group having from 6 to 40 carbon atoms. In one embodiment, the lipophilic portion of the negatively charged amphiphile is a cycloalkyl group having from 6 to 30 carbon atoms. In one embodiment, the lipophilic portion of the negatively charged amphiphile is a cycloalkyl group having from 6 to 20 carbon atoms.

In one embodiment, the lipophilic portion of the negatively charged amphiphile is a cycloalkenyl group having from 6 to 40 carbon atoms. In one embodiment, the lipophilic portion of the negatively charged amphiphile is a cycloalkenyl group having from 6 to 30 carbon atoms. In one embodiment, the lipophilic portion of the negatively charged amphiphile is a cycloalkenyl group having from 6 to 20 carbon atoms.

In one embodiment, the lipophilic portion of the negatively charged amphiphile is an alkylcycloalkyl group having a total of from 6 to 40 carbon atoms. In one embodiment, the lipophilic portion of the negatively charged amphiphile is an alkylcycloalkyl group having a total of from 6 to 30 carbon atoms. In one embodiment, the lipophilic portion of the negatively charged amphiphile is an alkylcycloalkyl group having a total of from 6 to 20 carbon atoms.

In one embodiment, the lipophilic portion of the negatively charged amphiphile is an alkylcycloalkylalkyl group having a total of from 6 to 40 carbon atoms. In one embodiment, the lipophilic portion of the negatively charged amphiphile is an alkylcycloalkylalkyl group having a total of from 6 to 30 carbon atoms. In one embodiment, the lipophilic portion of the negatively charged amphiphile is an alkylcycloalkylalkyl group having a total of from 6 to 20 carbon atoms.

In one embodiment, the lipophilic portion of the negatively charged amphiphile is an aryl group having from 6 to 40 carbon atoms. In one embodiment, the lipophilic portion of the negatively charged amphiphile is an aryl group having from 6 to 30 carbon atoms. In one embodiment, the lipophilic portion of the negatively charged amphiphile is an aryl group having from 6 to 20 carbon atoms.

In one embodiment, the lipophilic portion of the negatively charged amphiphile is an alkylaryl group having a total of from 6 to 40 carbon atoms. In one embodiment, the lipophilic portion of the negatively charged amphiphile is an alkylaryl group having a total of from 6 to 30 carbon atoms. In one embodiment, the lipophilic portion of the negatively charged amphiphile is an alkylaryl group having a total of from 6 to 20 carbon atoms.

In one embodiment, the lipophilic portion of the negatively charged amphiphile is an arylalkyl group having a total of from 6 to 40 carbon atoms. In one embodiment, the lipophilic portion of the negatively charged amphiphile is an arylalkyl group having a total of from 6 to 30 carbon atoms. In one embodiment, the lipophilic portion of the negatively charged amphiphile is an arylalkyl group having a total of from 6 to 20 carbon atoms.

In one embodiment, the lipophilic portion of the negatively charged amphiphile is an alkylarylalkyl group having a total of from 6 to 40 carbon atoms. In one embodiment, the lipophilic portion of the negatively charged amphiphile is an alkylarylalkyl group having a total of from 6 to 30 carbon atoms. In one embodiment, the lipophilic portion of the negatively charged amphiphile is an alkylarylalkyl group having a total of from 6 to 20 carbon atoms.

In one embodiment, the negatively charged amphiphile has a constitutive negative charge. In this context a “constitutive negative charge” means that the amphiphile carries the negative charge at all physiological pH. Amphiphiles carrying constitutive charged anionic moieties are typically salts of organic strong acids (i.e. organic acids of formula HA which dissociates when dissolved in a solvent S that the proton is transferred completely to the solvent molecule, such that the concentration of the undissociated species HA is too low to be measured).

Typical classes of amphiphiles having a constitutive negative charge include sulfates, sulfonates, phosphates and phosphonates.

In one embodiment, the negatively charged amphiphile is a sulfate (as defined above, either in its broadest aspect or a preferred aspect). In one embodiment, the sulfate is an alkyl sulfate (i.e. in which the group R in the general definition above is an alkyl group) having 6 to 30, preferably 8 to 24, more preferably 12 to 18 carbon atoms. Typical examples of sulfates include sodium lauryl sulfate.

In one embodiment, the negatively charged amphiphile is a sulfonate (as defined above, either in its broadest aspect or a preferred aspect). In one embodiment, the sulfonate is an alkyl sulfonate (i.e. in which the group R in the general definition above is an alkyl group) having 6 to 30, preferably 8 to 24, more preferably 12 to 18 carbon atoms. In one embodiment, the sulfonate is an alkylaryl sulfonate (i.e. in which the group R in the general definition above is an aryl group substituted with an alkyl group) having a total of 10 to 40, preferably 12 to 30, more preferably 16 to 24 carbon atoms. Typical examples of sulfonates include sodium hexadecane sulfonate (sodium cetyl sulfonate) and sodium dodecylbenzene sulfonate.

In one embodiment, the negatively charged amphiphile is a phosphate (as defined above, either in its broadest aspect or a preferred aspect). In one embodiment, the phosphate is an alkyl phosphate (i.e. in which the group R in the general definition above is an alkyl group) having 6 to 30, preferably 8 to 24, more preferably 12 to 18 carbon atoms. Typical examples of phosphonates include octadecylphosphoric acid and dodecylphosphoric acid.

In one embodiment, the negatively charged amphiphile is a phosphonate (as defined above, either in its broadest aspect or a preferred aspect). In one embodiment, the phosphonate is an alkyl phosphonate (i.e. in which the group R in the general definition above is an alkyl group) having 6 to 30, preferably 8 to 24, more preferably 12 to 18 carbon atoms. Typical examples of phosphonates include octadecylphosphonic acid and dodecylphosphonic acid.

In one embodiment, the negatively charged amphiphile has a pH-sensitive charge. In this context a “pH-sensitive charge” means that the amphiphile carries the negative charge at alkaline pH, but may be neutral at neutral or acidic pH. Amphiphiles carrying constitutive charged anionic moieties are typically salts of organic weak acids (i.e. organic acids of formula HA which remains largely undissociated when dissolved in a solvent S so that the proton is only partially transferred completely to the solvent molecule).

In one embodiment, the negatively charged amphiphile is a carboxylic acid or carboxylate (as defined above, either in its broadest aspect or a preferred aspect). Typical examples of carboxylic acids include hexanoic acid, octanoic acid, decanoic acid, lauric acid, myristic acid, palmitic acid, stearic acid, icosanoic acid, tricosanic acid, 2-hydroxytetradecanoic acid, 2-methyloctadecanoic acid, 2-bromohexadecanoic acid, 2-propylpentanoic acid, 2-butyloctanoic acid, 2-hexyldecanoic acid, 9-hydroxy-stearic-acid, trans-2-decenoic acid, (9Z)-9-hexadecenoic acid, linolic acid, linolenic acid, oleic acid, elaidic acid, arachidonic acid, cyclododecanoic acid, adamantylacetic acid, dicyclohexylacetic acid, trans-4-pentylcyclohexane-carboxylic acid, 4-(decyloxy)benzoic acid, 4-octylbenzoic acid, cholic acid, lithocholic acid, or a mixture of any thereof.

In one embodiment, the carboxylic acid is selected from the group consisting of:
alkylcarboxylic acids (i.e. where the lipophilic portion of the carboxylic acid is an alkyl group) having a total of 6 to 40 carbon atoms, optionally substituted by a hydroxyl group;
alkenylcarboxylic acids (i.e. where the lipophilic portion of the carboxylic acid is an alkenyl group) having a total of 6 to 40 carbon atoms;
cycloalkylcarboxylic acids (i.e. where the lipophilic portion of the carboxylic acid is a cycloalkyl group) having a total of 6 to 40 carbon atoms;
alkylcycloalkylcarboxylic acids (i.e. where the lipophilic portion of the carboxylic acid is an alkylcycloalkyl group) having a total of 6 to 40 carbon atoms;
alkylarylcarboxylic acids (i.e. where the lipophilic portion of the carboxylic acid is an alkylaryl group) having a total of 6 to 40 carbon atoms;
dicarboxylic acids having 4 to 10 carbon atoms in the dicarboxyl moiety, optionally esterified with an alkyl group having 6 to 40 carbon atoms or an alkenyl group having 6 to 40 carbon atoms;
or a mixture of any thereof.

In one embodiment, the carboxylic acid is selected from the group consisting of, octanoic acid, decanoic acid, lauric acid, myristic acid, palmitic acid, stearic acid, arachidic acid, 2-butyloctanoic acid, 2-hexyldecanoic acid, 2-hydroxypalmitic acid, 2-methyloctadecanoic acid, hexadecenylsuccinic acid, neodecanoic acid, cyclohexanepentanoic acid, 1-adamantaneacetic acid, 4-pentylcyclohexanecarboxylic acid, cyclododecanecarboxylic acid, p-nonylbenzoic acid, 2-decenoic acid, 3-decenoic acid, palmitoleic acid, linolenic acid, linoleic acid, oleic acid, elaidic acid, arachidonic acid, lithocholic acid, chenodeoxycholic acid, deoxycholic acid, ursodeoxycholic acid, and cholic acid, or a mixture of any thereof.

In one embodiment, the negatively charged amphiphile has both constitutively and pH-sensitive negatively charged groups. Examples of negatively charged amphiphiles having both such groups include phosphatidylserines.

In one embodiment, the negatively charged amphiphile has a pH sensitive charge and pH sensitive anionic moiety is a carboxylic acid. One or more charged groups can be present in the amphiphile and in preferred embodiments a single charged moiety is present in an amphiphile.

The polar region of the negatively charged amphiphile may comprise additional uncharged polar moieties. Preferred uncharged polar moieties are hydroxyl or amide groups and one or more uncharged polar moieties can be present in the negatively charged amphiphile.

In one embodiment, the negatively charged amphiphile is a hemiester of a dicarboxylic acid with diacylglycerol. The hydrocarbyl portion of the acyl moieties of the diacylglycerol portion is as defined above, but is preferably an alkyl group (as defined above) having 6 to 40, preferably 8 to 18, carbon atoms or an alkenyl group (as defined above) having 6 to 40, preferably 14 to 18, carbon atoms. Typically, the acyl moieties are present on the 1- and 2-positions of the glycerol moiety. The acyl parts of the diacylglycerol moiety may be the same or different. In one embodiment, the acyl moieties are saturated fatty acid moieties, preferably selected from the group consisting of stearoyl, palmitoyl, myristoyl, lauroyl, decanoyl and octanoyl moieties. In one embodiment, the acyl moieties are unsaturated fatty acid moieties, preferably selected from the group consisting of oleoyl, linoyl, and lineoyl moieties. The dicarboxylic acid moiety is as defined above, and preferably has 2 to 20 carbon atoms, more preferably 2 to 10, even more preferably 2 to 8 carbon atoms. Examples of the dicarboxylic acid moiety include oxalate, malonate, succinate, glutarate, adipate, pimelate and suberate. In one embodiment, the negatively charged amphiphile is a hemiester of succinic acid with diacylglycerol (i.e. the dicarboxylic acid moiety is a succinic acid moiety)—also referred to herein as a “diacylglycerol hemisuccinate”. Typical examples of such negatively charged amphiphiles include 1,2-dilauroylglyceryl hemisuccinate (DLGS), 1,2-dimyristoylglyceryl hemisuccinate (DMGS), 1,2-dipalmitoylglyceryl hemisuccinate (DPGS), 1-palmitoyl-2-stearoylglyceryl hemisuccinate (PSGS), distearoylglyceryl hemisuccinate (DSGS), 1,2-dioleoylglyceryl hemisuccinate (DOGS), 1-stearoyl, 2-myristoyl-glycerylhemisuccinate (SMGS), 1-palmitoyl-2-oleoylglyceryl hemisuccinate (POGS) and analogues of any of the above thereof wherein the dicarboxylic acid portion is oxalate, malonate, succinate, glutarate, adipate, pimelate or suberate Dimyristoylglyceryl hemisuccinate, dipalmitoylglyceryl hemisuccinate or distearoylglyceryl hemisuccinate are preferred.

In one embodiment, the negatively charged amphiphile is a hemiester of a dicarboxylic acid with a steroid. The dicarboxylic acid moiety is as defined and exemplified above, and typically contains a total (including the acyl carbons) of 2 to 10, preferably 3 to 6, carbon atoms. The ester group may esterify any free hydroxyl group on the steroid molecule.

In one embodiment, the negatively charged amphiphile is a hemiester of a dicarboxylic acid with cholesterol. The dicarboxylic acid moiety is as defined above, and preferably has 2 to 20 carbon atoms, more preferably 2 to 10, even more preferably 2 to 8 carbon atoms. Examples of the dicarboxylic acid moiety include oxalate, malonate, succinate, glutarate, adipate, pimelate and suberate. In one embodiment, the negatively charged amphiphile is a hemiester of succinic acid with cholesterol (i.e. the dicarboxylic acid moiety is a succinic acid moiety)—also referred to herein as “cholesteryl hemisuccinate”. Typical examples of such negatively charged amphiphiles include those listed in Table 5 below.

TABLE 5
Cholesteryl hemisuccinate

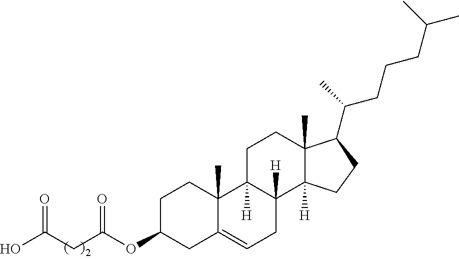

Cholesteryl hemiadipate

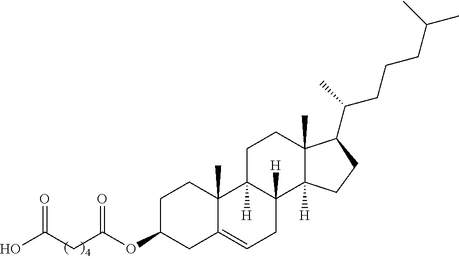

Cholesteryl hemisuberate

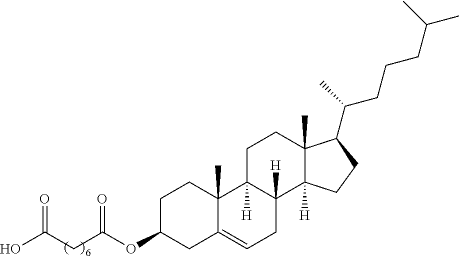

Cholesteryl hemi(decanedioate)

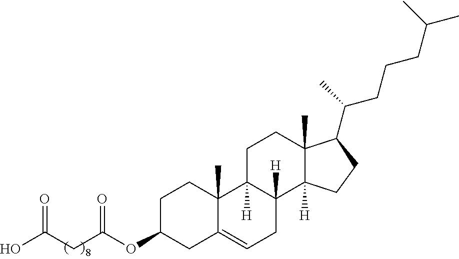

Cholesteryl hemi(dodecanedioate)

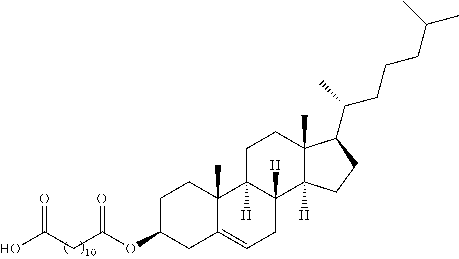

Cholesteryl hemi(tetradecanedioate)

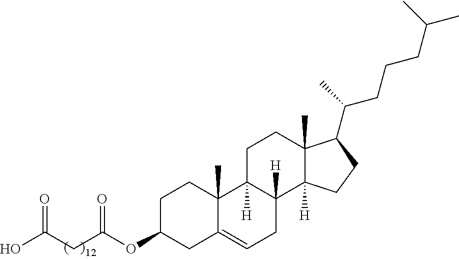

Cholesteryl hemi(hexadecanedioate)

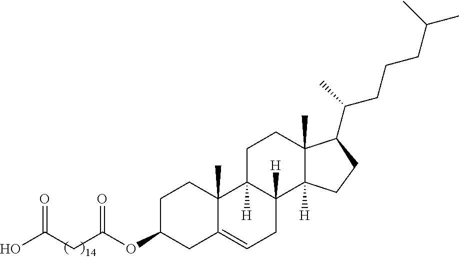

Cholesteryl hemi(octadecanedioate)

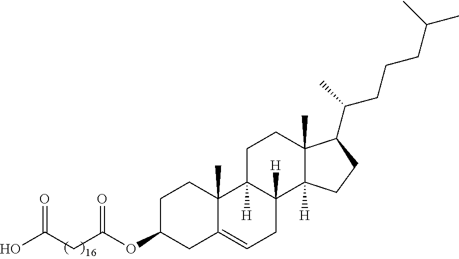

Of the above, cholesterol hemisuccinate is preferred.

In one embodiment, the negatively charged amphiphile is a monoester or diester of a phosphoric acid, wherein one of the phosphoric acid hydroxyl groups is esterified with diacylglycerol. The hydrocarbyl portion of the acyl moieties of the diacylglycerol portion is as defined above, but is preferably an alkyl group (as defined above) having 6 to 40, preferably 8 to 18, carbon atoms or an alkenyl group (as defined above) having 6 to 40, preferably 14 to 18, carbon atoms. The acyl parts of the diacylglycerol moiety may be the same or different. In one embodiment, the acyl moieties are saturated fatty acid moieties, preferably selected from the group consisting of stearoyl, palmitoyl, myristoyl, lauroyl, decanoyl and octanoyl moieties. In one embodiment, the acyl moieties are unsaturated fatty acid moieties, preferably selected from the group consisting of oleoyl, linoyl, and lineoyl moieties. When the negatively charged amphiphile is a diester of a phosphoric acid, the second hydroxyl group may be esterified with an alkyl group (as defined above) having 1 to 6 carbon atoms, a glyceryl group, or an O-serinyl group.

In one embodiment, the negatively charged amphiphile is an anionic phospholipid. Typical examples of such anionic phospholipids suitable as negatively charged amphiphiles include phosphatidylserines, phosphatidylglycerols or phosphatidic acids (all as defined above, either in its broadest aspect or a preferred aspect). The hydrocarbyl portion of the acyl moieties of such anionic phospholipids is as defined above, but is preferably an alkyl group (as defined above) having 6 to 40, preferably 8 to 24, carbon atoms or an alkenyl group (as defined above) having 6 to 40, preferably 14 to 22, carbon atoms and 1 to 6 carbon-carbon double bonds. The acyl parts of the phospholipids may be the same or different. In one embodiment, the acyl moieties are present on the 1- and 2-positions of the phospholipid. In one embodiment, the acyl moieties are present on the 1- and 3-positions of the phospholipid. In one embodiment, the acyl moieties are saturated fatty acid moieties having 8 to 24 carbon atoms (including the acyl carbon), preferably selected from the group consisting of lignoceroyl, behenoyl, arachidoyl, stearoyl, palmitoyl, myristoyl, lauroyl, decanoyl and octanoyl moieties. In a specific embodiment, neutral phospholipids have a Tₘ of 30° C. or higher and are selected from di-stearoyl or di-palmitoyl or stearoyl-palmitoyl moieties. In one embodiment, the acyl moieties are unsaturated fatty acid moieties having 14 to 22 carbon atoms (including the acyl carbon), preferably selected from the group consisting of oleoyl, linoyl, and lineoyl moieties.

In one embodiment, the negatively charged amphiphile is a phosphatidylserine, the acyl parts of which can be any of those defined and exemplified above. In one embodiment the negatively charged amphiphile is 1,2-dioleoylphosphatidylserine (DOPS).

In one embodiment, the negatively charged amphiphile is a phosphatidic acid, the acyl parts of which can be any of those defined and exemplified above. In one embodiment the negatively charged amphiphile is 1,2-dioleoylphosphatidic acid (DOPA).

In one embodiment, the negatively charged amphiphile is a phosphatidyl glycerol, the acyl parts of which can be any of those defined and exemplified above. In one embodiment the negatively charged amphiphile is 1,2-palmitoyloleoylphosphatidyl glycerol (POPG).

Where the negatively charged amphiphile is a phosphatidylserine, the composition typically does not comprise a stealth lipid, as defined and exemplified below. Where the negatively charged amphiphile is a phosphatidylserine, the composition typically does not comprise PEG. Where the negatively charged amphiphile is a phosphatidylserine, the composition typically does not comprise a neutral surfactant. Where the negatively charged amphiphile is a phosphatidylserine, the composition typically does not comprise polysorbate 20 (i.e., Tween 20), TPGS, Solutol, polysorbate 80 (i.e. Tween 80), or Myrj52.

In yet other embodiments, the negatively charged amphiphile is comprising a transfection enhancer element as described in WO2008/074487.

Suitable examples of negatively charged amphiphiles are listed in Table 6 below.

TABLE 6
2-Hydroxypalmitic acid

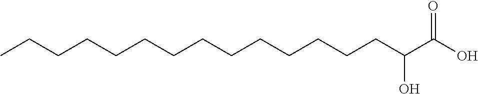 [SMILES: CCCCCCCCCCCCCCC(O)C(=O)O]

Butyloctanoic acid

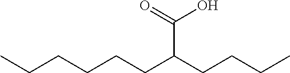

Palmitic-acid-9-hydroxystearic acid

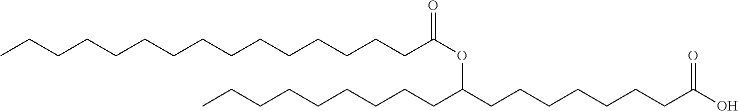 [SMILES: CCCCCCCCCCCCCCCC(=O)OC(CCCCCCCCC)CCCCCCCC(=O)O]

Cholesteryl hemisuccinate

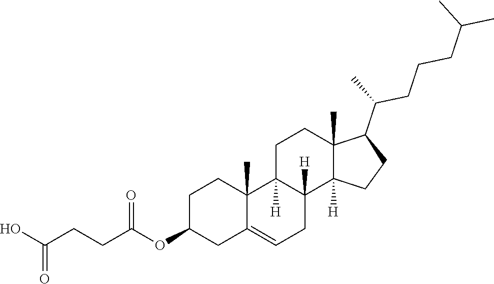

(3α,5β,12α,20R)-3,12-Dihydroxycholan-24-oic acid

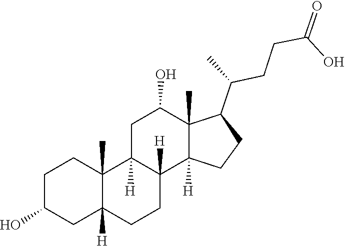

Neo-decanoic acid

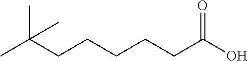

Trans-2-decenoic acid

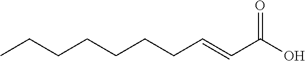

Ursodeoxycholic acid

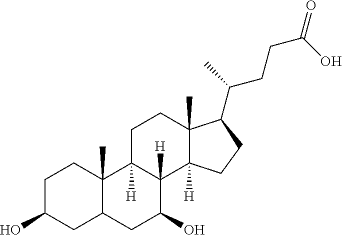

1-Hexadecanesulfonic acid

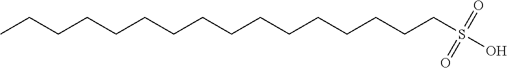 [SMILES: CCCCCCCCCCCCCCCCS(=O)(=O)O]

Cyclododecanoic acid

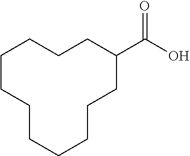 [SMILES: O=C(O)C1CCCCCCCCCCC1]

1,2-dioleoyl-sn-glycero-3-succinate

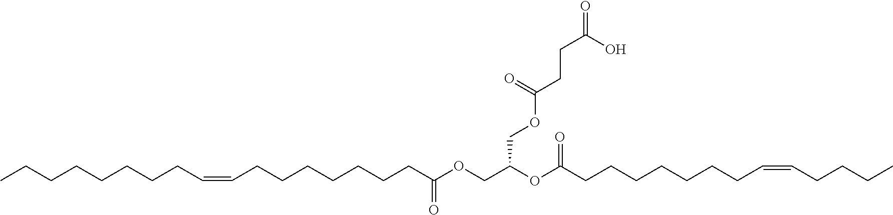 [SMILES: CCCC/C=C\CCCCCCCC(=O)O[C@H](COC(=O)CCCCCCC/C=C\CCCCCCCC)COC(=O)CCC(=O)O]

DSPG

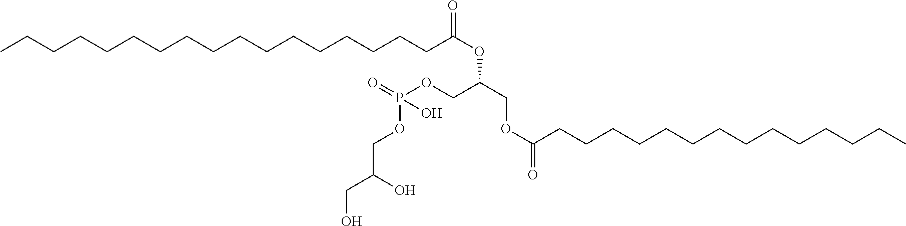 [SMILES: CCCCCCCCCCCCCCCCCC(=O)O[C@H](COC(=O)CCCCCCCCCCCCCC)COP(=O)(O)OCC(O)CO]

Myristic acid

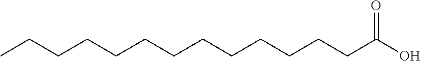 [SMILES: CCCCCCCCCCCCCC(=O)O]

Dodecyl phosphonic acid

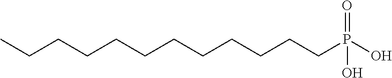 [SMILES: CCCCCCCCCCCCP(=O)(O)O]

Octadecylphosphonic acid

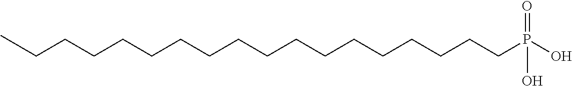 [SMILES: CCCCCCCCCCCCCCCCCCP(=O)(O)O]

POPG

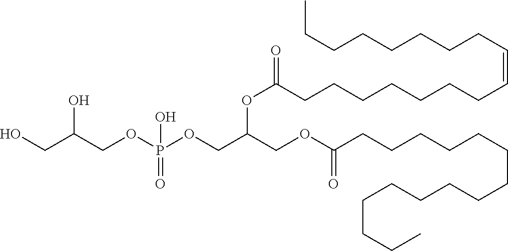 [SMILES: CCCCCCCC/C=C\CCCCCCCC(=O)OC(COC(=O)CCCCCCCCCCCCCCC)COP(=O)(O)OCC(O)CO]

1-palmitoyl-2-oleoyl-sn-glycero-3-phosphate

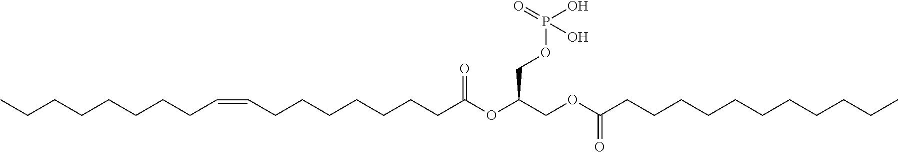 [SMILES: CCCCCCCC/C=C\CCCCCCCC(=O)O[C@H](COC(=O)CCCCCCCCCCC)COP(=O)(O)O]

1-palmitoyl-2-oleoyl-sn-glycero-3-phospho-L-serine

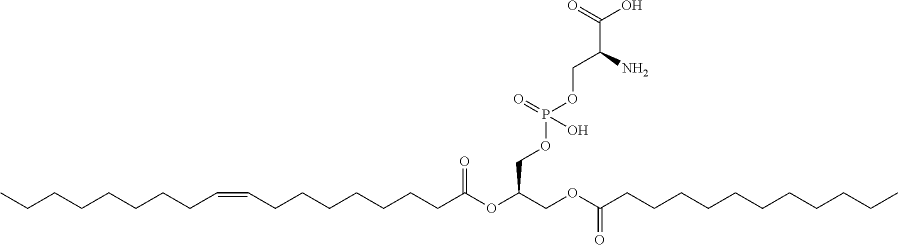 [SMILES: CCCCCCCC/C=C\CCCCCCCC(=O)O[C@H](COC(=O)CCCCCCCCCCC)COP(=O)(O)OC[C@H](N)C(=O)O]

Sodium dodecylbenzene sulfonate

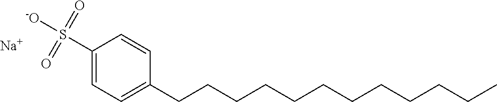

Sodium lauryl sulfate

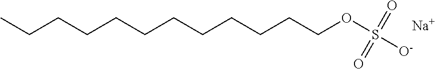 [SMILES: CCCCCCCCCCCCOS(=O)(=O)[O-] ; [Na+]]

2-hexyldecanoic acid

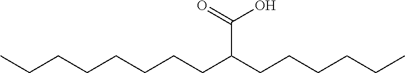 [SMILES: CCCCCCCCC(CCCCCC)C(=O)O]

Acetic acid

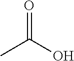

Adamantylacetic acid

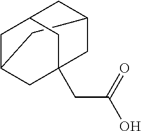 [SMILES: O=C(O)CC12CC3CC(CC(C3)C1)C2]

Cholic acid

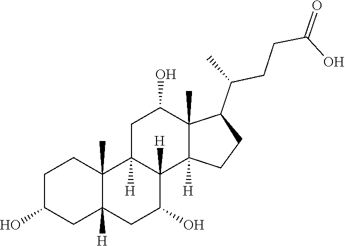

Dodecanoic acid

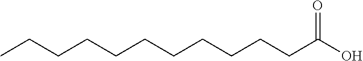

Hexadecenoic acid

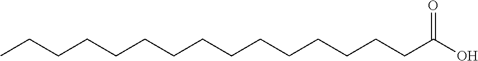 [SMILES: CCCCCCCCCCCCCCCC(=O)O]

Icosanoic acid

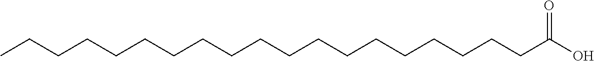 [SMILES: CCCCCCCCCCCCCCCCCCCC(=O)O]

Lithocholic acid

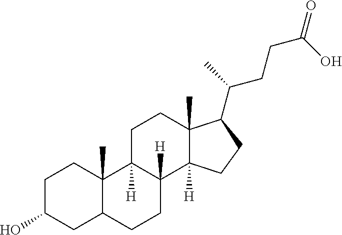

Octadecanoic acid

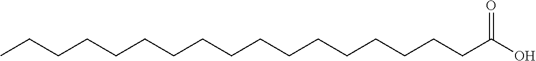 [SMILES: CCCCCCCCCCCCCCCCCC(=O)O]

Octanoic acid

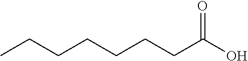

1-hexadecyl succinic acid

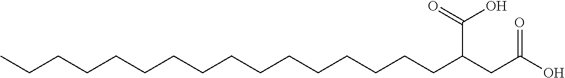 [SMILES: CCCCCCCCCCCCCCCCC(CC(=O)O)C(=O)O]

3-decanoic acid

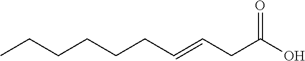

3α,7α-Dihydroxy-5β-cholan-24-oic acid

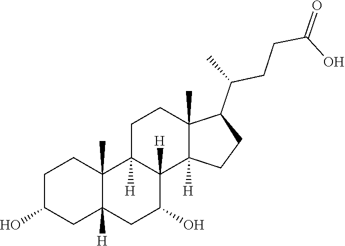

Cyclohexanepentanoic acid

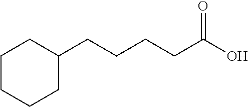 [SMILES: O=C(O)CCCCC1CCCCC1]

2-hexadecylicosanoic acid

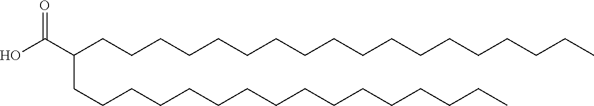 [SMILES: CCCCCCCCCCCCCCCCCCC(CCCCCCCCCCCCCCCC)C(=O)O]

Decanoic acid

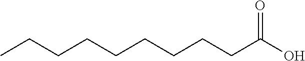

p-nonyloxybenzoic acid

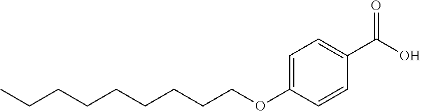 [SMILES: CCCCCCCCCOc1ccc(C(=O)O)cc1]

trans-4-pentylcyclohexanecarboxylic acid

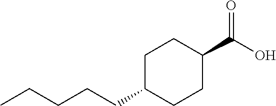

2-methyloctadecanoic acid

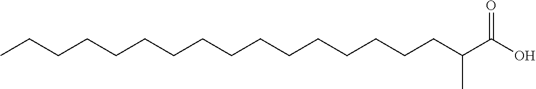 [SMILES: CCCCCCCCCCCCCCCCC(C)C(=O)O]

Arachidonic acid

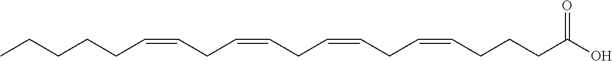 [SMILES: CCCCC/C=C\C/C=C\C/C=C\C/C=C\CCCC(=O)O]

Elaidic acid

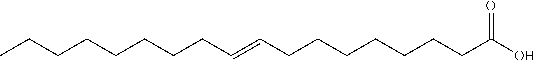 [SMILES: CCCCCCCC/C=C/CCCCCCCC(=O)O]

Linolenic acid

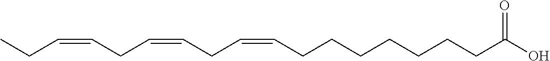 [SMILES: CC/C=C\C/C=C\C/C=C\CCCCCCCC(=O)O]

Linoleic acid

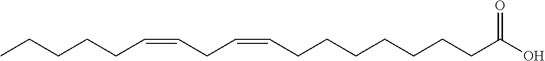 [SMILES: CCCCC/C=C\C/C=C\CCCCCCCC(=O)O]

Oleic acid

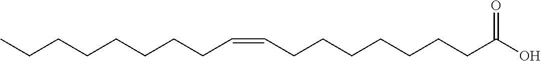 [SMILES: CCCCCCCC/C=C\CCCCCCCC(=O)O]

Palmitoleic acid

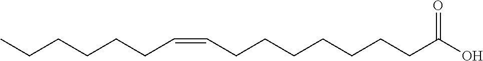 [SMILES: CCCCCC/C=C\CCCCCCCC(=O)O]

The anionic amphiphile can further be characterized by its molecular volume and the shape factor κ. The anionic amphiphile is typically non-protonated and in its charged state. The anionic amphiphile typically adsorbs a counterion from the mobile phase. For the purposes of describing the shape factor κ, the counterion is modelled as a sodium ion including its shell of hydration, having a molecular volume of 93 Å³ according to Siepi et al (2011). Thus, in one embodiment the anionic amphiphile (including a hydrated sodium ion) has a shape factor κ between 0.25 and 2, preferably between 0.4 and 1.0.

In one embodiment the partial molecular volume of the polar head group of the anionic amphiphile itself is between 40 and 120 Å³, preferably between 50 and 80 Å³. In one embodiment the partial molecular volume of the apolar tail group is between 120 and 600 Å³, preferably between 200 and 400 Å³.

Values of K and partial molecular volumes for certain anionic amphiphiles are provided in Table 7 below:

TABLE 7

| | partial | | | |
|---|---|---|---|---|
| | molecular | Na⁺ | | molecular |
| Compound/Group | volume | counterion | κ | volume |
| 2-Hydroxypalmitic acid-Tail | 174 | | 0.96 | 248 |
| 2-Hydroxypalmitic acid-Head | 74 | 93 | | |
| Butyloctanoic acid-Tail | 148 | | 0.98 | 200 |
| Butyloctanoic acid-Head | 52 | 93 | | |
| Palmitic-acid-9-hydroxystearic acid-Tail | 446 | | 0.32 | 498 |
| Palmitic-acid-9-hydroxystearic acid-Head | 52 | 93 | | |
| Chems-Tail | 325 | | 0.45 | 377 |
| Chems-Head | 52 | 93 | | |
| (3α,5β,12β,20R)-3,12-Dihydroxycholan-24- | 255 | | 0.57 | 306 |
| oic acid-Tail | | | | |
| (3α,5β,12β,20R)-3,12-Dihydroxycholan-24- | 52 | 93 | | |
| oic acid-Head | | | | |
| Neo-Decanoic acid-Tail | 100 | | 1.45 | 152 |
| Neo-Decanoic acid-Head | 52 | 93 | | |
| trans-2-decenoic acid-Tail | 122 | | 1.19 | 174 |
| trans-2-decenoic acid-Head | 52 | 93 | | |
| Ursodeoxycholic acid-Tail | 256 | | 0.56 | 308 |
| Ursodeoxycholic acid-Head | 52 | 93 | | |
| 1-Hexadecanesulfonic acid-Tail | 174 | | 1.02 | 259 |
| 1-Hexadecanesulfonic acid-Head | 85 | 93 | | |
| Cyclododecanoic acid-Tail | 149 | | 0.97 | 201 |
| Cyclododecanoic acid-Head | 52 | 93 | | |
| 1,2-dioleoyl-sn-glycero-3-succinate-Tail | 585 | | 0.25 | 637 |
| 1,2-dioleoyl-sn-glycero-3-succinate-Head | 52 | 93 | | |
| DSPG-Tail | 542 | | 0.40 | 668 |
| DSPG-Head | 126 | 93 | | |
| Myristic acid-Tail | 161 | | 0.90 | 213 |
| Myristic acid-Head | 52 | 93 | | |
| Dodecyl phosphonic acid-Tail | 72 | | 3.19 | 208 |
| Dodecyl phosphonic acid-Head | 136 | 93 | | |
| Octadecylphosphonic acid-Tail | 148 | | 1.55 | 284 |
| Octadecylphosphonic acid-Head | 136 | 93 | | |
| POPG-Tail | 405 | | 0.55 | 533 |
| POPG-Head | 128 | 93 | | |
| 1-palmitoyl-2-oleoyl-sn-glycero-3- | 507 | | 0.35 | 590 |
| phosphate-Tail | | | | |
| 1-palmitoyl-2-oleoyl-sn-glycero-3- | 84 | 93 | | |
| phosphate-Head | | | | |
| 1-palmitoyl-2-oleoyl-sn-glycero-3- | 507 | | 0.48 | 659 |
| phospho-L-serine | | | | |
| 1-palmitoyl-2-oleoyl-sn-glycero-3- | 152 | 93 | | |
| phospho-L-serine | | | | |
| Dodecylbenzene sulfonate-Tail | 174 | | 1.17 | 285 |
| Dodecylbenzene sulfonate-Head | 111 | 93 | | |
| Laurylsulfonic acid-Tail | 123 | | 1.53 | 219 |
| Laurylsulfonic acid-Head | 96 | 93 | | |
| 2-hexyldecanoic acid-Tail | 199 | | 0.73 | 251 |
| 2-hexyldecanoic acid-Head | 52 | 93 | | |
| Adamantylacetic acid-Tail | 94 | | 1.55 | 145 |
| Adamantylacetic acid-Head | 52 | 93 | | |
| Cholic acid-Tail | 263 | | 0.55 | 315 |
| Cholic acid-Head | 52 | 93 | | |
| Dodecanoic acid-Tail | 135 | | 1.07 | 187 |
| Dodecanoic acid-Head | 52 | 93 | | |
| Hexadecanoic acid-Tail | 186 | | 0.78 | 238 |
| Hexadecanoic acid-Head | 52 | 93 | | |
| Icosanoic acid-Tail | 237 | | 0.61 | 289 |
| Icosanoic acid-Head | 52 | 93 | | |
| Lithocholic acid-Tail | 234 | | 0.62 | 286 |
| Lithocholic acid-Head | 52 | 93 | | |
| Octadecanoic acid-Tail | 212 | | 0.68 | 264 |
| Octadecanoic acid-Head | 52 | 93 | | |
| Octanoic acid-Tail | 85 | | 1.71 | 136 |
| Octanoic acid-Head | 52 | 93 | | |
| 1-hexadecyl succinic acid-Tail | 199 | | 0.98 | 302 |
| 1-hexadecyl succinic acid-Head | 103 | 93 | | |
| 3-decenoic acid-Tail | 110 | | 1.32 | 162 |
| 3-decenoic acid-Head | 52 | 93 | | |
| 3,7-Dihydroxy-5-cholan-24-oic acid-Tail | 256 | | 0.57 | 308 |
| 3,7-Dihydroxy-5-cholan-24-oic acid-Head | 52 | 93 | | |
| Cyclohexanepentanoic acid-Tail | 115 | | 1.26 | 166 |
| Cyclohexanepentanoic acid-Head | 52 | 93 | | |
| 2-hexadecylicosanoic acid-Tail | 454 | | 0.32 | 506 |
| 2-hexadecylicosanoic acid-Head | 52 | 93 | | |
| Decanoic acid-Tail | 110 | | 1.32 | 162 |
| Decanoic acid-Head | 52 | 93 | | |
| p-nonyloxybenzoic acid-Tail | 201 | | 0.72 | 253 |
| p-nonyloxybenzoic acid-Head | 52 | 93 | | |
| trans-4-pentylcyclohexanecarboxylic acid- | 140 | | 1.03 | 192 |
| Tail | | | | |
| trans-4-pentylcyclohexanecarboxylic acid- | 52 | 93 | | |
| Head | | | | |
| 2-methyloctadecanoic acid-Tail | 237 | | 0.61 | 289 |
| 2-methyloctadecanoic acid-Head | 52 | 93 | | |
| Arachidonic acid-Tail | 237 | | 0.61 | 289 |
| Arachidonic acid-Head | 52 | 93 | | |
| Elaidic acid-Tail | 212 | | 0.68 | 264 |
| Elaidic acid-Head | 52 | 93 | | |
| Linolenic acid-Tail | 212 | | 0.68 | 264 |
| Linolenic acid-Head | 52 | 93 | | |
| Linoleic acid-Tail | 212 | | 0.68 | 264 |
| Linoleic acid-Head | 52 | 93 | | |
| Oleic acid-Tail | 212 | | 0.68 | 263 |
| Oleic acid-Head | 52 | 93 | | |
| Palmitoleic acid-Tail | 186 | | 0.78 | 238 |
| Palmitoleic acid-Head | 52 | 93 | | |

In one embodiment, the negatively charged amphiphile is selected from the group consisting of:
a carboxylic acid;
a phosphonic acid;
a sulfate;
a sulfonate;
a hemiester of a dicarboxylic acid with diacylglycerol;
a hemiester of a dicarboxylic acid with cholesterol;
a phosphatidylserine, a phosphatidic acid or a phosphatidylglycerol;
or a mixture of any thereof.

In one embodiment, the negatively charged amphiphile is selected from the group consisting of:
a carboxylic acid selected from the group consisting of alkylcarboxylic acids having a total of 6 to 40 carbon atoms, optionally substituted by a hydroxyl group;
alkenylcarboxylic acids having a total of 6 to 40 carbon atoms; cycloalkylcarboxylic acids having a total of 6 to 40 carbon atoms; alkylcycloalkylcarboxylic acids having a total of 6 to 40 carbon atoms; and alkylarylcarboxylic acids having a total of 6 to 40 carbon atoms; or a mixture of any thereof;
an alkyl sulfate having 6 to 30 carbon atoms;
an alkyl sulfonate having 6 to 30 carbon atoms;
an alkylaryl sulfonate having a total of 12 to 40 carbon atoms;
an alkyl phosphonate having 6 to 30 carbon atoms;
a hemiester of a dicarboxylic acid having 2 to 10 carbon atoms with diacylglycerol;
a hemiester of a dicarboxylic acid having 2 to 10 carbon atoms with cholesterol;
a diacylphosphatidylserine, wherein the hydrocarbyl portion of each of the acyl moieties thereof is an alkenyl group having 6 to 40, preferably 14 to 22, carbon atoms and 1 to 6 carbon-carbon double bonds;
or a mixture of any thereof.

In one embodiment, the negatively charged amphiphile is selected from the group consisting of:
a carboxylic acid selected from the group consisting of, octanoic acid, decanoic acid, lauric acid, myristic acid, palmitic acid, stearic acid, arachidic acid, 2-butyloctanoic acid, 2-hexyldecanoic acid, 2-hydroxypalmitic acid, 2-methyloctadecanoic acid, hexadecenylsuccinic acid, neodecanoic acid, cyclohexanepentanoic acid, 1-adamantaneacetic acid, 4-pentylcyclohexanecarboxylic acid, cyclododecanecarboxylic acid, p-nonylbenzoic acid, 2-decenoic acid, 3-decenoic acid, palmitoleic acid, linolenic acid, linoleic acid, oleic acid, elaidic acid, arachidonic acid, lithocholic acid, chenodeoxycholic acid, deoxycholic acid, ursodeoxycholic acid, and cholic acid;
sodium dodecyl sulfate;
a sulfonate selected from the group consisting of sodium cetyl sulfonate and decyl benzenesulfonate;
a phosphonate selected from the group consisting of dodecyl phosphonate and octadecyl phosphonate;
cholesteryl hemisuccinate;
a diacylglycerol hemisuccinate selected from the group consisting of 1,2-dilauroylglyceryl hemisuccinate (DLGS), 1,2-dimyristoylglyceryl hemisuccinate (DMGS), 1,2-dipalmitoylglyceryl hemisuccinate (DPGS), 1-palmitoyl-2-stearoylglyceryl hemisuccinate (PSGS), 1,2-distearoylglyceryl hemisuccinate (DSGS), 1-stearoyl-2-myristoyl-glycerylhemisuccinate (SMGS), and 1-palmitoyl-2-oleoylglyceryl hemisuccinate (POGS);
or a mixture of any thereof.

In one embodiment, the negatively charged amphiphile is present in the composition in an amount of up to about 20 mol % of the total lipid present in the composition. In one embodiment, the negatively charged amphiphile is present in the composition in an amount of up to about 15 mol % of the total lipid present in the composition. In one embodiment, the negatively charged amphiphile is present in the composition in an amount of up to about 14 mol % of the total lipid present in the composition. In one embodiment, the negatively charged amphiphile is present in the composition in an amount of up to about 13 mol % of the total lipid present in the composition. In one embodiment, the negatively charged amphiphile is present in the composition in an amount of up to about 12 mol % of the total lipid present in the composition. In one embodiment, the negatively charged amphiphile is present in the composition in an amount of up to about 11 mol % of the total lipid present in the composition. In one embodiment, the negatively charged amphiphile is present in the composition in an amount of up to about 10 mol % of the total lipid present in the composition. In one embodiment, the negatively charged amphiphile is present in the composition in an amount of up to about 9 mol % of the total lipid present in the composition. In one embodiment, the negatively charged amphiphile is present in the composition in an amount of up to about 8 mol % of the total lipid present in the composition. In one embodiment, the negatively charged amphiphile is present in the composition in an amount of up to about 7 mol % of the total lipid present in the composition. In one embodiment, the negatively charged amphiphile is present in the composition in an amount of up to about 6 mol % of the total lipid present in the composition. In one embodiment, the negatively charged amphiphile is present in the composition in an amount of up to about 5 mol % of the total lipid present in the composition. It has surprisingly been found that the lipid nanoparticles remain colloidally stable and biocompatible even when only low amounts of anionic lipid are present in the lipid nanoparticle.

In one embodiment, the negatively charged amphiphile is present in the composition in an amount of at least about 0.1 mol % of the total lipid present in the composition. In one embodiment, the negatively charged amphiphile is present in the composition in an amount of at least about 0.2 mol % of the total lipid present in the composition. In one embodiment, the negatively charged amphiphile is present in the composition in an amount of at least about 0.5 mol % of the total lipid present in the composition. In one embodiment, the negatively charged amphiphile is present in the composition in an amount of at least about 1 mol % of the total lipid present in the composition. In one embodiment, the negatively charged amphiphile is present in the composition in an amount of at least about 2 mol % of the total lipid present in the composition. In one embodiment, the negatively charged amphiphile is present in the composition in an amount of at least about 3 mol % of the total lipid present in the composition. In one embodiment, the negatively charged amphiphile is present in the composition in an amount of at least about 4 mol % of the total lipid present in the composition. In one embodiment, the negatively charged amphiphile is present in the composition in an amount of at least about 5 mol % of the total lipid present in the composition. In one embodiment, the negatively charged amphiphile is present in the composition in an amount of at least about 6 mol % of the total lipid present in the composition. In one embodiment, the negatively charged amphiphile is present in the composition in an amount of at least about 7 mol % of the total lipid present in the composition.

In one embodiment, the negatively charged amphiphile is present in the composition in an amount of about 0.1 to about 20 mol % of the total lipid present in the composition. In one embodiment, the negatively charged amphiphile is present in the composition in an amount of about 0.2 to about 20 mol % of the total lipid present in the composition. In one embodiment, the negatively charged amphiphile is present in the composition in an amount of about 0.5 to about 20 mol % of the total lipid present in the composition. In one embodiment, the negatively charged amphiphile is present in the composition in an amount of about 1 to about 20 mol % of the total lipid present in the composition. In one embodiment, the negatively charged amphiphile is present in the composition in an amount of about 2 to about 15 mol % of the total lipid present in the composition. In one embodiment, the negatively charged amphiphile is present in the composition in an amount of about 4 to 12 about mol % of the total lipid present in the composition. In one embodiment, the negatively charged amphiphile is present in the composition in an amount of about 6 to about 10 mol % of the total lipid present in the composition. In one embodiment, the negatively charged amphiphile is present in the composition in an amount of about 7 to about 9 mol % of the total lipid present in the composition.

In one embodiment, the negatively charged amphiphile is present in the composition in an amount of about 0.1 to about 10 mol % of the total lipid present in the composition. In one embodiment, the negatively charged amphiphile is present in the composition in an amount of about 0.2 to about 7.5 mol % of the total lipid present in the composition. In one embodiment, the negatively charged amphiphile is present in the composition in an amount of about 0.5 to about 7 mol % of the total lipid present in the composition. In one embodiment, the negatively charged amphiphile is present in the composition in an amount of about 1 to about 6 mol % of the total lipid present in the composition. In one embodiment, the negatively charged amphiphile is present in the composition in an amount of about 2 to about 5 mol % of the total lipid present in the composition.

In one embodiment, the negatively charged amphiphile is a carboxylic acid, and the carboxylic acid is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is a carboxylic acid, and the carboxylic acid is present in an amount of at least about 1 mol %, such as at least about 2 mol %, such as at least about 3 mol %, such as at least about 4 mol %, such as at least about 5 mol %, such as at least about 6 mol %, such as at least about 7 mol % of the total lipid present in the composition.

In one embodiment, the negatively charged amphiphile is a carboxylic acid, and the carboxylic acid is present in the composition in an amount of about 1 to about 20 mol %, such as about 2 to about 15 mol %, such as about 4 to about 12 mol %, such as about 6 to about 10 mol %, such as about 7 to about 9 mol %, or such as about 1 to about 6 mol %, such as about 2 to about 5 mol %, of the total lipid present in the composition.

In one embodiment, the negatively charged amphiphile is a carboxylic acid having between 6 and 24 carbon atoms, such as hexanoic acid, octanoic acid, decanoic acid, lauric acid, myristic acid, palmitic acid or stearic acid, and the carboxylic acid is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is a carboxylic acid having between 6 and 24 carbon atoms, such as hexanoic acid, octanoic acid, decanoic acid, lauric acid, myristic acid, palmitic acid or stearic acid, and the carboxylic acid is present in an amount of at least about 1 mol %, such as at least about 2 mol %, such as at least about 3 mol %, such as at least about 4 mol %, such as at least about 5 mol %, such as at least about 6 mol %, such as at least about 7 mol % of the total lipid present in the composition.

In one embodiment, the negatively charged amphiphile is a carboxylic acid having between 6 and 24 carbon atoms, such as hexanoic acid, octanoic acid, decanoic acid, lauric acid, myristic acid, palmitic acid or stearic acid, and the carboxylic acid is present in the composition in an amount of about 1 to about 20 mol %, such as about 2 to about 15 mol %, such as about 4 to about 12 mol %, such as about 6 to about 10 mol %, such as about 7 to about 9 mol %, or such as about 1 to about 6 mol %, such as about 2 to about 5 mol %, of the total lipid present in the composition.

In one embodiment, the negatively charged amphiphile is a hemiester of a dicarboxylic acid with diacylglycerol, and the hemiester is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is a hemiester of a dicarboxylic acid with diacylglycerol, and the hemiester is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is a hemiester of a dicarboxylic acid with diacylglycerol, and the hemiester is present in the composition in an amount of about 0.1 to about 20 mol %, such as about 0.2 to about 20%, such as about 0.5 to about 20 mol %, such as about 1 to about 20 mol %, such as about 2 to about 15 mol %, such as about 4 to about 12 mol %, such as about 6 to about 10 mol %, such as about 7 to about 9 mol %, or about 0.2 to about 7.5 mol %, such as about 0.5 to about 7 mol %, or such as about 1 to about 6 mol %, such as about 2 to about 5 mol % of the total lipid present in the composition.

In one embodiment, the negatively charged amphiphile is 1,2-dimyristoylglyceryl hemisuccinate, and the 1,2-dimyristoylglyceryl hemisuccinate is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is 1,2-dipalmitoylglyceryl hemisuccinate, and the 1,2-dipalmitoylglyceryl hemisuccinate is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is 1-stearoyl, 2-myristoylglyceryl hemisuccinate, and the 1-stearoyl, 2-myistoylglyceryl hemisuccinate is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is 1,2-dimyristoylglyceryl hemisuccinate, and the 1,2-dimyristoylglyceryl hemisuccinate is present in the composition in an amount of about 0.1 to about 20 mol %, such as about 0.2 to about 20%, such as about 0.5 to about 20 mol %, such as about 1 to about 20 mol %, such as about 2 to about 15 mol %, such as about 4 to about 12 mol %, such as about 6 to about 10 mol %, such as about 7 to about 9 mol %, or about 0.2 to about 7.5 mol %, such as about 0.5 to about 7 mol %, such as about 1 to about 6 mol %, such as about 2 to 5 mol % of the total lipid present in the composition.

In one embodiment, the negatively charged amphiphile is 1,2-dipalmitoylglyceryl hemisuccinate, and the 1,2-dimyristoylglyceryl hemisuccinate is present in the composition in an amount of about 0.1 to about 20 mol %, such as about 0.2 to about 20%, such as about 0.5 to about 20 mol %, such as about 1 to about 20 mol %, such as about 2 to about 15 mol %, such as about 4 to about 12 mol %, such as about 6 to about 10 mol %, such as about 7 to about 9 mol %, or about 0.2 to about 7.5 mol %, such as about 0.5 to about 7 mol %, such as about 1 to about 6 mol %, such as about 2 to 5 mol %, of the total lipid present in the composition.

In one embodiment, the negatively charged amphiphile is 1-stearoyl, 2-myristoylglyceryl hemisuccinate, and the 1-stearoyl, 2-myristoylglyceryl hemisuccinate is present in the composition in an amount of about 0.1 to about 20 mol %, such as about 0.2 to about 20%, such as about 0.5 to about 20 mol %, such as about 1 to about 20 mol %, such as about 2 to about 15 mol %, such as about 4 to about 12 mol %, such as about 6 to about 10 mol %, such as about 7 to about 9 mol %, or about 0.2 to about 7.5 mol %, such as about 0.5 to about 7 mol %, such as about 1 to about 6 mol %, such as about 2 to 5 mol % of the total lipid present in the composition.

In one embodiment, the negatively charged amphiphile is a phosphatidylserine, and the phosphatidylserine is present in the composition in an amount of about 0.1 to about 20 mol %, such as about 0.2 to about 20%, such as about 0.5 to about 20 mol %, such as about 1 to about 20 mol %, such as about 2 to about 15 mol %, such as about 4 to about 12 mol %, such as about 6 to about 10 mol %, such as about 7 to about 9 mol %, or about 0.2 to about 7.5 mol %, such as about 0.5 to about 7 mol %, such as about 1 to about 6 mol %, such as about 2 to 5 mol % of the total lipid present in the composition.

In one embodiment, the negatively charged amphiphile is 1,2-dioleoylphosphatidyl-serine, and the 1,2-dioleoylphosphatidylserine is present in the composition in an amount of about 0.1 to about 20 mol %, such as about 0.2 to about 20%, such as about 0.5 to about 20 mol %, such as about 1 to about 20 mol %, such as about 2 to about 15 mol %, such as about 4 to about 12 mol %, such as about 6 to about 10 mol %, such as about 7 to about 9 mol %, or about 0.2 to about 7.5 mol %, such as about 0.5 to about 7 mol %, such as about 1 to about 6 mol %, such as about 2 to 5 mol % of the total lipid present in the composition.

In one embodiment, the negatively charged amphiphile is a hemiester of a dicarboxylic acid with cholesterol, and the hemiester is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is a hemiester of a dicarboxylic acid with cholesterol, and the hemiester is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is a hemiester of a dicarboxylic acid with cholesterol, and the hemiester is present in the composition in an amount of about 1 to about 20 mol %, such as about 2 to about 15 mol %, such as about 4 to about 12 mol %, such as about 6 to about 10 mol %, such as about 7 to about 9 mol %, or about 0.2 to about 7.5 mol %, such as about 0.5 to about 7 mol %, such as about 1 to about 6 mol %, such as about 2 to 5 mol %, of the total lipid present in the composition.

In one embodiment, the negatively charged amphiphile is cholesterol hemisuccinate, and the cholesterol hemisuccinate is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is cholesterol hemisuccinate, and the cholesterol hemisuccinate is present in an amount of up to about 20 mol %, such as up to about 15 mol %, such as up to about 14 mol %, such as up to about 13 mol %, such as up to about 12 mol %, such as up to about 11 mol %, such as up to about 10 mol %, such as up to about 9 mol %, such as up to about 8 mol %, such as up to about 7 mol %, such as up to about 6 mol %, such as up to about 5 mol %, of the total lipid present in the composition.

In one embodiment, the negatively charged amphiphile is cholesterol hemisuccinate, and the cholesterol hemisuccinate is present in the composition in an amount of about 1 to about 20 mol %, such as about 2 to about 15 mol %, such as about 4 to about 12 mol %, such as about 6 to about 10 mol %, such as about 7 to about 9 mol %, or about 0.2 to about 7.5 mol %, such as about 0.5 to about 7 mol %, such as about 1 to about 6 mol %, such as about 2 to 5 mol %, of the total lipid present in the composition.

### Neutral Lipid

The composition may also additionally comprise a neutral lipid. The neutral lipid is preferably a neutral phospholipid. In one embodiment, the phospholipid may be zwitterionic (i.e. it carries both a positive and a negative charge, so that it is neutral at a pH ranging around neutral).

In some embodiments, the phospholipid is selected from the group consisting of phosphatidylcholines, phosphatidylethanolamines, and sphingomyelins. The hydrocarbyl portion of the acyl moieties of phospholipids is as defined above, but is preferably an alkyl group (as defined above) having 6 to 40, preferably 8 to 24, carbon atoms or an alkenyl group (as defined above) having 6 to 40, preferably 14 to 22, carbon atoms and 1 to 6 carbon-carbon double bonds. The acyl moieties of the phospholipids may be the same or different. In one embodiment, the acyl moieties are present on the 1- and 2-positions of the phospholipid. In one embodiment, the acyl moieties are present on the 1- and 3-positions of the phospholipid. In one embodiment, the acyl moieties are saturated fatty acid moieties having 8 to 24 carbon atoms (including the acyl carbon), preferably selected from the group consisting of lignoceroyl, behenoyl, arachidoyl, stearoyl, palmitoyl, myristoyl, lauroyl, decanoyl and octanoyl moieties. In a specific embodiment, neutral phospholipids have a Tₘ of 30° C. or higher and are selected from di-stearoyl or di-palmitoyl or stearoyl-palmitoyl moieties. In one embodiment, the acyl moieties are unsaturated fatty acid moieties having 14 to 22 carbon atoms (including the acyl carbon), preferably selected from the group consisting of oleoyl, linoyl, and lineoyl moieties.

Examples of such phospholipids include 1,2-diacylphosphatidylcholines, such as 1,2-distearoylphosphatidylcholine (DSPC), 1,2-dioleoylphosphatidylcholine (DOPC), 1,2-dimyristoylphosphatidylcholine (DMPC), 1,2-dipentadecanoylphosphatidylcholine, 1,2-dilauroylphosphatidylcholine (DLPC), 1,2-dipalmitoylphosphatidylcholine (DPPC), 1,2-diarachidoylphosphatidylcholine (DAPC), 1,2-dibehenoylphosphatidylcholine (DBPC), 1,2-ditricosanoylphosphatidylcholine (DTPC), 1,2-dilignoceroylphosphatidylcholine (DLPC), 1-palmitoyl-2-oleoylphosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), and 1-hexadecyl-sn-10-glycero-3-phosphocholine (C16 Lyso PC); phosphatidylethanolamines, in particular 1,2-diacylphosphatidyl-ethanolamines, such as 1,2-dioleoylphosphatidylethanolamine (DOPE), 1,2-distearoylphosphatidylethanolamine (DSPE), 1,2-dipalmitoyl-phosphatidyl-ethanolamine (DPPE), 1,2-dimyristoylphosphatidylethanolamine (DMPE), 1,2-dilauroyl-phosphatidylethanolamine (DLPE), 1,2-diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), and 1-palmitoyl-2-oleoyl-sn-glycero-3-phosphoethanolamine (POPE), N-palmitoyl-D-erythro-sphingosylphosphorylcholine (SM), and further phosphatidyl-ethanolamine lipids with different hydrophobic chains.

In some embodiments, the neutral lipid is selected from the group consisting of DSPC, DOPC, DMPC, DPPC, POPC, DOPE, DPPG, POPE, DPPE, DMPE, DSPE, and SM.

In some embodiments, the neutral lipid is selected from the group consisting of DSPC, DPPC, DMPC, DOPC, POPC, and SM. In one embodiment, the neutral lipid is not DOPE. In some embodiments, the neutral lipid is DSPC or POPC.

Thus, in some embodiments, the lipid nanoparticle compositions described herein comprise a cationically ionizable lipid (as defined herein) and a phospholipid. In some embodiments, the lipid nanoparticle compositions described herein comprise a cationically ionizable lipid and a phospholipid selected from the group consisting of DSPC, DPPC, POPC, and DOPC. In some embodiments, the lipid nanoparticle compositions described herein comprise a cationically ionizable lipid (as defined herein) and DSPC or POPC.

In some embodiments, the neutral lipid is present in the lipid nanoparticle compositions in an amount of about 5 mol % to about 40 mol %, such as from about 5 mol % to about 25 mol %, from about 5 mol % to about 20 mol %, from about 5 mol % to about 15 mol %, or from about 5 mol % to about 10 mol %, of the total lipids present in the composition.

In some embodiments, the neutral lipid is selected from the group consisting of DSPC, DOPC, DMPC, DPPC, POPC, DOPE, POPE, DPPE, DMPE, DSPE, and SM, and the DSPC, DOPC, DMPC, DPPC, POPC, DOPE, POPE, DPPE, DMPE, DSPE, or SM is present in the lipid nanoparticle compositions in an amount of about 5 mol % to about 40 mol %, such as from about 5 mol % to about 25 mol %, from about 5 mol % to about 20 mol %, from about 5 mol % to about 15 mol %, or from about 5 mol % to about 10 mol %, of the total lipids present in the composition.

In some embodiments, the neutral lipid is selected from the group consisting of DSPC, DPPC, DMPC, DOPC, POPC, and SM, and the DSPC, DPPC, DMPC, DOPC, POPC, or SM is present in the lipid nanoparticle compositions in an amount of about 5 mol % to about 40 mol %, such as from about 5 mol % to about 25 mol %, from about 5 mol % to about 20 mol %, from about 5 mol % to about 15 mol %, or from about 5 mol % to about 10 mol %, of the total lipids present in the composition.

In some embodiments, the neutral lipid is DSPC or POPC, and the DSPC or POPC is present in the lipid nanoparticle compositions in an amount of about 5 mol % to about 40 mol %, such as from about 5 mol % to about 25 mol %, from about 5 mol % to about 20 mol %, from about 5 mol % to about 15 mol %, or from about 5 mol % to about 10 mol %, of the total lipids present in the composition.

### Steroid

The lipid nanoparticle compositions of the present invention also comprise a steroid. In one embodiment, the steroid comprises a sterol. In one embodiment, the steroid is cholesterol.

Thus, in some embodiments, the lipid nanoparticle compositions described herein comprise a cationically ionizable lipid (as defined herein) and cholesterol.

In some embodiments, the steroid is present in the lipid nanoparticle compositions described herein in a concentration ranging from about 10 mol % to about 65 mol %, such as from about 20 mol % to about 60 mol %, from about 30 mol % to about 50 mol %, or from about 25 mol % to about 35 mol % of the total lipids present in the compositions described herein.

In some embodiments, the steroid is cholesterol and the cholesterol is present in the lipid nanoparticle compositions described herein in a concentration ranging from about 10 mol % to about 65 mol %, such as from about 20 mol % to about 60 mol %, from about 30 mol % to about 50 mol %, or from about 25 mol % to about 35 mol % of the total lipids present in the compositions described herein.

In certain preferred embodiments, the lipid nanoparticle compositions described herein comprise a phospholipid and cholesterol, preferably in the concentrations given above. In some embodiments, the lipid nanoparticle compositions comprise a phospholipid selected from the group consisting of DSPC, DPPC, DSPE, and DPPE, and cholesterol, preferably in the concentrations given above. In some embodiments, the lipid nanoparticle compositions described herein comprise DSPC and cholesterol, preferably in the concentrations given above.

In some embodiments, the combined concentration of the neutral lipid (in particular, one or more phospholipids) and steroid (in particular, cholesterol) may comprise from about 0 mol % to about 70 mol %, such as from about 2 mol % to about 60 mol %, from about 5 mol % to about 55 mol %, from about 5 mol % to about 50 mol %, from of the total lipids present in the lipid nanoparticle compositions described herein.

### Stealth Lipids and Other Ingredients Preferably Absent

The lipid nanoparticle compositions described herein are substantially free (as defined above, either in its broadest aspect or a preferred aspect) of a polyethylene glycol-conjugated lipid (also referred to herein as a PEGylated lipid or PEG-lipid) having at least 5 consecutive ethylene glycol repeating units. The term “PEGylated lipid” refers to a molecule comprising both a lipid portion and a polyethylene glycol portion. PEGylated lipids are known in the art. In one embodiment, the lipid nanoparticle compositions described herein are substantially free (as defined above, either in its broadest aspect or a preferred aspect) of polyethylene glycol (PEG). The PEG may comprise at least 5 consecutive ethylene glycol repeating units. The PEG may be in any form, either lipid conjugated, or in the form of a surfactant. The PEG may be associated with or bound to the LNP. The PEG or PEG-lipid may comprise at least 5 consecutive ethylene glycol repeating units. The PEG or PEG-lipid may comprise 5-1000, 5-500, 5-100, 5-50, 8-1000, 8-500, 8-100, 8-50, 10-1000, 10-500, 10-100, or 10-50, ethylene glycol repeating units, which may be consecutive. The PEG or PEG-lipid may comprise 5-1000, optionally 8-100, ethylene glycol repeating units, which are preferably consecutive.

A “polymer” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties. If more than one type of repeat unit is present within the polymer, then the polymer is said to be a “copolymer.” The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In one embodiment, the lipid nanoparticle composition is substantially free (as defined above, either in its broadest aspect or a preferred aspect) of a stealth polymer. In this specification the term “stealth polymer” means a polymer (as defined above) (optionally conjugated to a lipid) having the following features: (a) polar (hydrophilic) functional groups; (b) hydrogen bond acceptor groups, (c) no hydrogen bond donor groups; and (d) no net charge. In some embodiments, a stealth polymer is designed to sterically stabilize a lipid particle by forming a protective hydrophilic layer that shields the hydrophobic lipid layer. In some embodiments, a stealth polymer can reduce its association with serum proteins and/or the resulting uptake by the reticuloendothelial system when such lipid particles are administered in vivo.

In one embodiment, the lipid nanoparticle composition is substantially free (as defined above, either in its broadest aspect of a preferred aspect) of a stealth polymer-conjugated lipid (also referred to herein simply as a “stealth lipid”).

Examples of such stealth polymers include poly(sarcosine) (pSar) (which may be conjugated to a lipid—to produce a polysarcosinylated lipid), poly(oxazoline) (POX); poly(oxazine) (POZ), poly(vinyl pyrrolidone) (PVP); poly(N-(2-hydroxypropyl)-methacrylamide) (pHPMA); poly(dehydroalanine) (pDha); poly(aminoethoxy ethoxy acetic acid) (pAEEA) and poly(2-methylaminoethoxy ethoxy acetic acid) (pmAEEA).

In one embodiment, the lipid nanoparticle composition is substantially free (as defined above, either in its broadest aspect of a preferred aspect) of a polysarcosine-conjugated lipid, also referred to herein as sarcosinylated lipid or pSar-lipid. The term “sarcosinylated lipid” refers to a molecule comprising both a lipid portion and a polysarcosine (poly(N-methylglycine) portion, the polysarcosine portion having the repeating unit shown below.

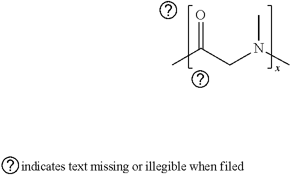

In one embodiment, the lipid nanoparticle composition is substantially free (as defined above, either in its broadest aspect of a preferred aspect) of a polyoxazoline (POX)-conjugated and/or a polyoxazine (POZ)-conjugated lipid and/or a POX/POZ-conjugated lipid, also referred to herein as a conjugate of a POX and/or POZ polymer and one or more hydrophobic chains or as oxazolinylated and/or oxazinylated lipid or POX and/or POZ-lipid. The term “oxazolinylated lipid” or “POX-lipid” refers to a molecule comprising both a lipid portion and a polyoxazoline portion, the polyoxazoline portion (pOx) having the repeating unit shown below. The term “oxazinylated lipid” or “POZ-lipid” refers to a molecule comprising both a lipid portion and a polyoxazine portion, the polyoxazine (pOz) portion having the repeating unit shown below. The term “oxazolinylated/oxazinylated lipid” or “POX/POZ-lipid” or “POXZ-lipid” refers to a molecule comprising both a lipid portion and a portion of a copolymer of polyoxazoline and polyoxazine, i.e. a polymer having both the pOx and pOz repeating units shown below.

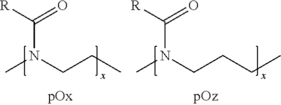

In one embodiment, the lipid nanoparticle composition is substantially free (as defined above, either in its broadest aspect of a preferred aspect) of a poly(vinyl pyrrolidone) (PVP). In one embodiment, the lipid nanoparticle composition is substantially free (as defined above, either in its broadest aspect of a preferred aspect) of a poly(vinyl pyrrolidone) (PVP) conjugated to a lipid. The term “poly(vinyl pyrrolidone)” or “PVP” means a polymer having a vinyl pyrrolidine repeating unit, i.e. the repeating unit shown below.

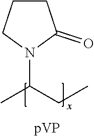

In one embodiment, the lipid nanoparticle composition is substantially free (as defined above, either in its broadest aspect of a preferred aspect) of poly(N-(2-hydroxypropyl) methacrylamide) (pHPMA). In one embodiment, the lipid nanoparticle composition is substantially free (as defined above, either in its broadest aspect of a preferred aspect) of poly(N-(2-hydroxypropyl) methacrylamide) (pHPMA) conjugated to a lipid. The term “poly(N-(2-hydroxypropyl)-methacrylamide” or “pHPMA” means a polymer having the repeating unit shown below.

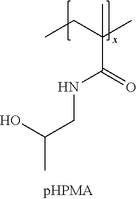

In one embodiment, the lipid nanoparticle composition is substantially free (as defined above, either in its broadest aspect of a preferred aspect) of poly(dehydroalanine) (pDha). In one embodiment, the lipid nanoparticle composition is substantially free (as defined above, either in its broadest aspect of a preferred aspect) of poly(dehydroalanine) (pDha) conjugated to a lipid. The term “pDha” means a polymer having the repeating unit shown below.

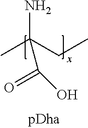

In one embodiment, the lipid nanoparticle composition is substantially free (as defined above, either in its broadest aspect of a preferred aspect) of amphiphilic oligoethylene glycol (OEG)-conjugated lipids. Examples of amphiphilic oligoethylene glycol (OEG)-conjugated lipids include poly(aminoethyl-ethylene glycol acetyl) (pAEEA) and/or poly(methylaminoethyl-ethylene glycol acetyl) (pmAEEA). The terms “pAEEA” and “pmAEAA” means a polymer having the repeating unit shown below:

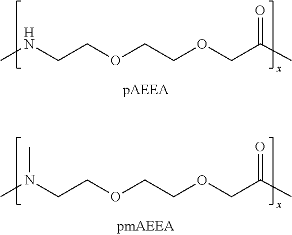

In one embodiment, the lipid nanoparticle composition is substantially free (as defined above, either in its broadest aspect of a preferred aspect) of phosphatidylserine. In one embodiment, the lipid nanoparticle composition is substantially free (as defined above, either in its broadest aspect of a preferred aspect) of surfactant. Surfactant may be understood to mean a non-ionic amphiphilic organic compound, such as a poly(ethylene glycol) (PEG) chain linked to a single hydrophobic chain, a polyoxyethylene sorbitan ester, D-α-tocopheryl polyethylene glycol-succinate (TPGS), a polyoxyethylene mono ester of a saturated C10 to C22 hydroxy fatty acid, a polyoxyethylene fatty acid ester, a polyoxyethylene alkyl ether, or a combination thereof. In one embodiment, the lipid nanoparticle composition is substantially free (as defined above, either in its broadest aspect of a preferred aspect) of PEG- or other stealth polymer-containing surfactants. In one embodiment, the lipid nanoparticle composition is substantially free (as defined above, either in its broadest aspect of a preferred aspect) of a surfactant selected from the group consisting of: polysorbates (TWEENS) such as polysorbate 20 (Tween 20), polysorbate 40, polysorbate 60, polysorbate 80, D-α-tocopherol polyethylene glycol succinate (TPGS), solutols, Myrjs, and Brijs. In one embodiment, the lipid nanoparticle composition is substantially free (as defined above, either in its broadest aspect of a preferred aspect) of Tween 20 (polysorbate 20), TPGS (tocopheryl polyethylene glycol succinate), Solutol (polyoxyethylene esters of 12-hydroxystearic acid), Tween 80 (polysorbate 80), and Myrj52 (polyoxyethylene (40) stearate). In one embodiment, the lipid nanoparticle composition is substantially free (as defined above, either in its broadest aspect of a preferred aspect) of a polyoxyethylene sorbitan ester, optionally a polysorbate, preferably polysorbate 20 (Tween 20).

In one embodiment, the lipid nanoparticle composition is substantially free (as defined above, either in its broadest aspect of a preferred aspect) of an inorganic polyphosphate. The inorganic polyphosphate can be any linear, cyclic, or branched inorganic polyphosphate. In some embodiments, the inorganic polyphosphate is a linear inorganic polyphosphate (such as a linear inorganic triphosphate). In some embodiments, the inorganic polyphosphate comprises the formula [PₓO₍₃ₓ₊₁₎]^{y-}, wherein x is an integer and is at least 3; and y is the anionic charge. For example, if x is 3, the inorganic polyphosphate is a linear inorganic triphosphate comprising the formula [P₃O₁₀]⁵⁻. Likewise, if x is 4, the inorganic polyphosphate is a linear or branched inorganic tetraphosphate comprising the formula [P₄O₁₃]⁶⁻. In some embodiments, the inorganic polyphosphate is selected from the group consisting of triphosphate, tetraphosphate, pentaphosphate, hexaphosphate, heptaphosphate, and mixtures thereof. In some embodiments, the inorganic polyphosphate is selected from the group consisting of triphosphate, tetraphosphate, pentaphosphate, and mixtures thereof. In some embodiments, the inorganic polyphosphate is triphosphate.

### Preferred Compositions

In one embodiment, the cationically ionisable lipid is selected from the group consisting of N,N-dimethyl-2,3-dioleyloxypropylamine (DODMA);
1,2-distearyloxy-N,N-dimethyl-3-aminopropane (DSDMA),
1,2-dilinoleyloxy-N,N-dimethylaminopropane (DLinDMA),
3-dimethylamino-2-(cholest-5-en-3-beta-oxybutan-4-oxy)-1-(cis,cis-9,12-octadecadienoxy) propane (CLinDMA),
2-[5′-(cholest-5-en-3-beta-oxy)-3′-oxapentoxy)-3-dimethyl-1-(cis,cis-9′,12′-octadecadienoxy) propane (CpLinDMA),
N,N-dimethyl-3,4-dioleyloxybenzylamine (DMOBA),
1,2-N,N′-dioleylcarbamyl-3-dimethylaminopropane (DOcarbDAP),
2,3-dilinoleoyloxy-N,N-dimethylpropylamine (DLinDAP),
1,2-N,N′-Dilinoleylcarbamyl-3-dimethylaminopropane (DLincarbDAP),
1,2-dilinoleoylcarbamyl-3-dimethylaminopropane (DLinCDAP),
2,2-dilinoleyl-4-dimethylaminomethyl-[1,3]-dioxolane (DLin-K-DMA),
2,2-dilinoleyl-4-dimethylaminoethyl-[1,3]-dioxolane (DLin-K5-XTC2-DMA),
2,2-dilinoleyl-4-(2-dimethylaminoethyl)-[1,3]-dioxolane (DLin-KC2-DMA),
heptatriaconta-6,9,28,31-tetraen-19-yl-4-(dimethylamino)butanoate (DLin-MC3-DMA),
2-({8-[(3)-cholest-5-en-3-yloxy]octyl}oxy)-N,N-dimethyl-3-[(9Z,12Z)-octadeca-9,12-dien-1-yloxy]propan-1-amine (octyl-CLinDMA),
di((Z)-non-2-en-1-yl) 8,8′-((((2 (dimethylamino)ethyl)thio) carbonyl) azanediyl)-dioctanoate (ATX),
N,N-dimethyl-2,3-bis(dodecyloxy) propan-1-amine (DLDMA),
N,N-dimethyl-2,3-bis(tetradecyloxy) propan-1-amine (DMDMA),
Di((Z)-non-2-en-1-yl)-9-((4-(dimethylaminobutanoyl)oxy) heptadecanedioate (L319),
N-dodecyl-3-((2-dodecylcarbamoyl-ethyl)-{2-[(2-dodecylcarbamoylethyl)-2-{(2-dodecylcarbamoyl-ethyl)-[2-(2-dodecylcarbamoyl-ethylamino)-ethyl]-amino}-ethylamino) propionamide (lipidoid 98N12-5),
1-[2-[bis(2-hydroxydodecyl)amino]ethyl-[2-[4-[2-[bis(2-hydroxydodecyl)-amino]ethyl]piperazin-1-yl]ethyl]amino]dodecan-2-ol (lipidoid C12-200),
(6Z,9Z,28Z,31Z)-heptatriaconta-6,9,28,31-tetraen-19-yl 4-(dimethylamino)butanoate;
bis-(2-butyloctyl) 10-(N-(3-(dimethylamino) propyl) nonanamido) nonadecanedioate (A9);
[(4-hydroxybutyl) azanediyl]di(hexane-6,1-diyl)bis(2-hexyldecanoate)) (ALC-315);
(heptadecan-9-yl 8-{(2-hydroxyethyl) [6-oxo-6-(undecyloxy) octyl]amino}octanoate) (L5);
heptadecan-9-yl 8-{(2-hydroxyethyl) [6-oxo-6-(undecyloxy) hexyl]amino}octanoate) (SM-102);
((2-((4-(dimethylamino)butanoyl)oxy)ethyl) azanediyl)bis(octane-8,1-diyl)bis(2-hexyldecanoate) (EA 405);
(((4-(dimethylamino)butanoyl)oxy) azanediyl)bis(octane-8,1-diyl)bis(2-hexyldecanoate) (HY405);
8-(((4-(dimethylamino)butanoyl)oxy)((9Z,12Z)-octadeca-9,12-dien-1-yl)amino) octyl 2-hexyldecanoate (HY501);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   and the neutral lipid is selected from the group consisting of DSPC, DOPC, DMPC, DPPC, POPC, DOPE, DPPG, POPE, DPPE, DMPE, DSPE, and SM.

In one embodiment, the cationically ionisable lipid is selected from the group consisting of 2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
N,N-dimethyl-2,3-dioleyloxypropylamine (DODMA);
di((Z)-non-2-en-1-yl)-9-((4-(dimethylaminobutanoyl)oxy) heptadecanedioate (L319);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
HY501 (as described further herein);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
di(heptadecan-9-yl) 3,3′-((2-(4-methylpiperazin-1-yl)ethyl) azanediyl)dipropionate (BHD-C2C2-PipZ); and
bis(2-hexyldecyl) 3,3′-((4-(4-methylpiperazin-1-yl)butyl) azanediyl)dipropionate (BHD-C2C4-PipZ);
di(nonadecan-9-yl) 3,3′-((4-(4-methylpiperazin-1-yl)butyl) azanediyl)dipropionate (DND-C2-C4-PipZ);
   and the neutral lipid is selected from the group consisting of DSPC, DPPC, DMPC, DOPC, POPC, and SM.

In one embodiment, the cationically ionisable lipid is selected from the group consisting of N,N-dimethyl-2,3-dioleyloxypropylamine (DODMA);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   and the neutral lipid is DSPC.

In one embodiment, the cationically ionisable lipid is selected from the group consisting of:
N,N-dimethyl-2,3-dioleyloxypropylamine (DODMA);
1,2-distearyloxy-N,N-dimethyl-3-aminopropane (DSDMA),
1,2-dilinoleyloxy-N,N-dimethylaminopropane (DLinDMA),
3-dimethylamino-2-(cholest-5-en-3-beta-oxybutan-4-oxy)-1-(cis,cis-9,12-octadecadienoxy) propane (CLinDMA),
2-[5′-(cholest-5-en-3-beta-oxy)-3′-oxapentoxy)-3-dimethyl-1-(cis,cis-9′,12′-octadecadienoxy) propane (CpLinDMA),
N,N-dimethyl-3,4-dioleyloxybenzylamine (DMOBA),
1,2-N,N′-dioleylcarbamyl-3-dimethylaminopropane (DOcarbDAP),
2,3-dilinoleoyloxy-N,N-dimethylpropylamine (DLinDAP),
1,2-N,N′-Dilinoleylcarbamyl-3-dimethylaminopropane (DLincarbDAP),
1,2-dilinoleoylcarbamyl-3-dimethylaminopropane (DLinCDAP),
2,2-dilinoleyl-4-dimethylaminomethyl-[1,3]-dioxolane (DLin-K-DMA),
2,2-dilinoleyl-4-dimethylaminoethyl-[1,3]-dioxolane (DLin-K5-XTC2-DMA),
2,2-dilinoleyl-4-(2-dimethylaminoethyl)-[1,3]-dioxolane (DLin-KC2-DMA),
heptatriaconta-6,9,28,31-tetraen-19-yl-4-(dimethylamino)butanoate (DLin-MC3-DMA),
2-({8-[(3B)-cholest-5-en-3-yloxy]octyl}oxy)-N,N-dimethyl-3-[(9Z,12Z)-octadeca-9,12-dien-1-yloxy]propan-1-amine (octyl-CLinDMA),
di((Z)-non-2-en-1-yl) 8,8′-((((2 (dimethylamino)ethyl)thio) carbonyl) azanediyl)-dioctanoate (ATX),
N,N-dimethyl-2,3-bis(dodecyloxy) propan-1-amine (DLDMA),
N,N-dimethyl-2,3-bis(tetradecyloxy) propan-1-amine (DMDMA),
Di((Z)-non-2-en-1-yl)-9-((4-(dimethylaminobutanoyl)oxy) heptadecanedioate (L319),
N-dodecyl-3-((2-dodecylcarbamoyl-ethyl)-{2-[(2-dodecylcarbamoylethyl)-2-{(2-dodecylcarbamoyl-ethyl)-[2-(2-dodecylcarbamoyl-ethylamino)-ethyl]-amino}-ethylamino) propionamide (lipidoid 98N12-5),
1-[2-[bis(2-hydroxydodecyl)amino]ethyl-[2-[4-[2-[bis(2-hydroxydodecyl)-amino]ethyl]piperazin-1-yl]ethyl]amino]dodecan-2-ol (lipidoid C12-200),
(6Z,9Z,28Z,31Z)-heptatriaconta-6,9,28,31-tetraen-19-yl 4-(dimethylamino)butanoate;
bis-(2-butyloctyl) 10-(N-(3-(dimethylamino) propyl) nonanamido) nonadecanedioate (A9);
[(4-hydroxybutyl) azanediyl]di(hexane-6,1-diyl)bis(2-hexyldecanoate)) (ALC-315);
(heptadecan-9-yl 8-{(2-hydroxyethyl) [6-oxo-6-(undecyloxy) octyl]amino}octanoate) (L5);
heptadecan-9-yl 8-{(2-hydroxyethyl) [6-oxo-6-(undecyloxy) hexyl]amino}octanoate) (SM-102);
((2-((4-(dimethylamino)butanoyl)oxy)ethyl) azanediyl)bis(octane-8,1-diyl)bis(2-hexyldecanoate) (EA 405);
(((4-(dimethylamino)butanoyl)oxy) azanediyl)bis(octane-8,1-diyl)bis(2-hexyldecanoate) (HY405);
8-(((4-(dimethylamino)butanoyl)oxy)((9Z,12Z)-octadeca-9,12-dien-1-yl)amino) octyl 2-hexyldecanoate (HY501);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   and the steroid is cholesterol.

In one embodiment, the cationically ionisable lipid is selected from the group consisting of: 2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
N,N-dimethyl-2,3-dioleyloxypropylamine (DODMA);
di((Z)-non-2-en-1-yl)-9-((4-(dimethylaminobutanoyl)oxy) heptadecanedioate (L319);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
HY501 (as described further herein);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
di(heptadecan-9-yl) 3,3′-((2-(4-methylpiperazin-1-yl)ethyl) azanediyl)dipropionate (BHD-C2C2-PipZ);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   and the steroid is cholesterol.

In one embodiment, the cationically ionisable lipid is selected from the group consisting of N,N-dimethyl-2,3-dioleyloxypropylamine (DODMA);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   and the steroid is cholesterol.

In one embodiment, the cationically ionisable lipid is selected from the group consisting of:
N,N-dimethyl-2,3-dioleyloxypropylamine (DODMA);
1,2-distearyloxy-N,N-dimethyl-3-aminopropane (DSDMA),
1,2-dilinoleyloxy-N,N-dimethylaminopropane (DLinDMA),
3-dimethylamino-2-(cholest-5-en-3-beta-oxybutan-4-oxy)-1-(cis,cis-9,12-octadecadienoxy) propane (CLinDMA),
2-[5′-(cholest-5-en-3-beta-oxy)-3′-oxapentoxy)-3-dimethyl-1-(cis,cis-9′,12′-octadecadienoxy) propane (CpLinDMA),
N,N-dimethyl-3,4-dioleyloxybenzylamine (DMOBA),
1,2-N,N′-dioleylcarbamyl-3-dimethylaminopropane (DOcarbDAP),
2,3-dilinoleoyloxy-N,N-dimethylpropylamine (DLinDAP),
1,2-N,N′-Dilinoleylcarbamyl-3-dimethylaminopropane (DLincarbDAP),
1,2-dilinoleoylcarbamyl-3-dimethylaminopropane (DLinCDAP),
2,2-dilinoleyl-4-dimethylaminomethyl-[1,3]-dioxolane (DLin-K-DMA),
2,2-dilinoleyl-4-dimethylaminoethyl-[1,3]-dioxolane (DLin-K5-XTC2-DMA),
2,2-dilinoleyl-4-(2-dimethylaminoethyl)-[1,3]-dioxolane (DLin-KC2-DMA),
heptatriaconta-6,9,28,31-tetraen-19-yl-4-(dimethylamino)butanoate (DLin-MC3-DMA),
2-({8-[(3)-cholest-5-en-3-yloxy]octyl}oxy)-N,N-dimethyl-3-[(9Z,12Z)-octadeca-9,12-dien-1-yloxy]propan-1-amine (octyl-CLinDMA),
di((Z)-non-2-en-1-yl) 8,8′-((((2 (dimethylamino)ethyl)thio) carbonyl) azanediyl)-dioctanoate (ATX),
N,N-dimethyl-2,3-bis(dodecyloxy) propan-1-amine (DLDMA),
N,N-dimethyl-2,3-bis(tetradecyloxy) propan-1-amine (DMDMA),
Di((Z)-non-2-en-1-yl)-9-((4-(dimethylaminobutanoyl)oxy) heptadecanedioate (L319),
N-dodecyl-3-((2-dodecylcarbamoyl-ethyl)-{2-[(2-dodecylcarbamoylethyl)-2-{(2-dodecylcarbamoyl-ethyl)-[2-(2-dodecylcarbamoyl-ethylamino)-ethyl]-amino}-ethylamino) propionamide (lipidoid 98N12-5),
1-[2-[bis(2-hydroxydodecyl)amino]ethyl-[2-[4-[2-[bis(2-hydroxydodecyl)-amino]ethyl]piperazin-1-yl]ethyl]amino]dodecan-2-ol (lipidoid C12-200),
(6Z,9Z,28Z,31Z)-heptatriaconta-6,9,28,31-tetraen-19-yl 4-(dimethylamino)butanoate;
bis-(2-butyloctyl) 10-(N-(3-(dimethylamino) propyl) nonanamido) nonadecanedioate (A9);
[(4-hydroxybutyl) azanediyl]di(hexane-6,1-diyl)bis(2-hexyldecanoate)) (ALC-315);
(heptadecan-9-yl 8-{(2-hydroxyethyl) [6-oxo-6-(undecyloxy) octyl]amino}octanoate) (L5);
heptadecan-9-yl 8-{(2-hydroxyethyl) [6-oxo-6-(undecyloxy) hexyl]amino}octanoate) (SM-102);
((2-((4-(dimethylamino)butanoyl)oxy)ethyl) azanediyl)bis(octane-8,1-diyl)bis(2-hexyldecanoate) (EA 405);
(((4-(dimethylamino)butanoyl)oxy) azanediyl)bis(octane-8,1-diyl)bis(2-hexyldecanoate) (HY405);
8-(((4-(dimethylamino)butanoyl)oxy)((9Z,12Z)-octadeca-9,12-dien-1-yl)amino) octyl 2-hexyldecanoate (HY501);
di(heptadecan-9-yl) 3,3′-((2-(4-methylpiperazin-1-yl)ethyl) azanediyl)dipropionate (BHD-C2C2-PipZ)
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ); and
   the anionic amphiphile is selected from the group consisting of a carboxylic acid; a phosphonic acid; a sulfate; a sulfonate; a hemiester of a dicarboxylic acid with diacylglycerol; a hemiester of a dicarboxylic acid with cholesterol; a phosphatidylserine, a phosphatidic acid or a phosphatidylglycerol.

In one embodiment, the cationically ionisable lipid is selected from the group consisting of 2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
N,N-dimethyl-2,3-dioleyloxypropylamine (DODMA);
di((Z)-non-2-en-1-yl)-9-((4-(dimethylaminobutanoyl)oxy) heptadecanedioate (L319);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
HY501 (as described further herein);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
di(heptadecan-9-yl) 3,3′-((2-(4-methylpiperazin-1-yl)ethyl) azanediyl)dipropionate (BHD-C2C2-PipZ);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ); and
   the anionic amphiphile is selected from the group consisting of:
   a carboxylic acid selected from the group consisting of alkylcarboxylic acids having a total of 6 to 40 carbon atoms, optionally substituted by a hydroxyl group;
   alkenylcarboxylic acids having a total of 6 to 40 carbon atoms; cycloalkylcarboxylic acids having a total of 6 to 40 carbon atoms; alkylcycloalkylcarboxylic acids having a total of 6 to 40 carbon atoms; and alkylarylcarboxylic acids having a total of 6 to 40 carbon atoms; or a mixture of any thereof;
   an alkyl sulfate having 6 to 30 carbon atoms;
   an alkyl sulfonate having 6 to 30 carbon atoms;
   an alkylaryl sulfonate having a total of 12 to 40 carbon atoms;
   an alkyl phosphonate having 6 to 30 carbon atoms;
   a hemiester of a dicarboxylic acid having 2 to 10 carbon atoms with diacylglycerol;
   a hemiester of a dicarboxylic acid having 2 to 10 carbon atoms with cholesterol;
   a diacylphosphatidylserine, wherein the hydrocarbyl portion of each of the acyl moieties thereof is an alkenyl group having 6 to 40, preferably 14 to 22, carbon atoms and 1 to 6 carbon-carbon double bonds;
   or a mixture of any thereof.

In one embodiment, the cationically ionisable lipid is selected from the group consisting of N,N-dimethyl-2,3-dioleyloxypropylamine (DODMA);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ); and
   the anionic amphiphile is selected from the group consisting of a carboxylic acid selected from the group consisting of, octanoic acid, decanoic acid, lauric acid, myristic acid, palmitic acid, stearic acid, arachidic acid, 2-butyloctanoic acid, 2-hexyldecanoic acid, 2-hydroxypalmitic acid, 2-methyloctadecanoic acid, hexadecenylsuccinic acid, neodecanoic acid, cyclohexanepentanoic acid, 1-adamantaneacetic acid, 4-pentylcyclohexanecarboxylic acid, cyclododecanecarboxylic acid, p-nonylbenzoic acid, 2-decenoic acid, 3-decenoic acid, palmitoleic acid, linolenic acid, linoleic acid, oleic acid, elaidic acid, arachidonic acid, lithocholic acid, chenodeoxycholic acid, deoxycholic acid, ursodeoxycholic acid, cholic acid;
   sodium dodecyl sulfate;
   a sulfonate selected from the group consisting of sodium cetyl sulfonate and decyl benzenesulfonate;
   a phosphonate selected from the group consisting of dodecyl phosphonate acid, and octadecyl phosphonate;
   cholesteryl hemisuccinate;
   a diacylglycerol hemisuccinate selected from the group consisting of 1,2-dilauroylglyceryl hemisuccinate (DLGS), 1,2-dimyristoylglyceryl hemisuccinate (DMGS), 1,2-dipalmitoylglyceryl hemisuccinate (DPGS), 1-palmitoyl-2-stearoylglyceryl hemisuccinate (PSGS), 1,2-distearoylglyceryl hemisuccinate (DSGS), 1-stearoyl, 2-myristoyl-glycerylhemisuccinate (SMGS), 1-palmitoyl-2-oleoylglyceryl hemisuccinate (POGS);
   or a mixture of any thereof.

In one embodiment, the neutral lipid is selected from the group consisting of DSPC, DOPC, DMPC, DPPC, POPC, DOPE, DPPG, POPE, DPPE, DMPE, DSPE, and SM, and the steroid is cholesterol.

In one embodiment, the neutral lipid is selected from the group consisting of DSPC, DPPC, DMPC, DOPC, POPC, and SM, and the steroid is cholesterol.

In one embodiment, the neutral lipid is DSPC, and the steroid is cholesterol.

In one embodiment, the neutral lipid is selected from the group consisting of DSPC, DOPC, DMPC, DPPC, POPC, DOPE, DPPG, POPE, DPPE, DMPE, DSPE, and SM, and the anionic amphiphile is selected from the group consisting of a carboxylic acid; a phosphonic acid; a sulfate; a sulfonate; a hemiester of a dicarboxylic acid with diacylglycerol; a hemiester of a dicarboxylic acid with cholesterol; a phosphatidylserine, a phosphatidic acid or a phosphatidylglycerol;

In one embodiment, the neutral lipid is selected from the group consisting of DSPC, DPPC, DMPC, DOPC, POPC, and SM, and the anionic amphiphile is selected from the group consisting of:
a carboxylic acid selected from the group consisting of alkylcarboxylic acids having a total of 6 to 40 carbon atoms, optionally substituted by a hydroxyl group;
alkenylcarboxylic acids having a total of 6 to 40 carbon atoms; cycloalkylcarboxylic acids having a total of 6 to 40 carbon atoms; alkylcycloalkylcarboxylic acids having a total of 6 to 40 carbon atoms; and alkylarylcarboxylic acids having a total of 6 to 40 carbon atoms; or a mixture of any thereof;
an alkyl sulfate having 6 to 30 carbon atoms;
an alkyl sulfonate having 6 to 30 carbon atoms;
an alkylaryl sulfonate having a total of 12 to 40 carbon atoms;
an alkyl phosphonate having 6 to 30 carbon atoms;
a hemiester of a dicarboxylic acid having 2 to 10 carbon atoms with diacylglycerol;
a hemiester of a dicarboxylic acid having 2 to 10 carbon atoms with cholesterol;
a diacylphosphatidylserine, wherein the hydrocarbyl portion of each of the acyl moieties thereof is an alkenyl group having 6 to 40, preferably 14 to 22, carbon atoms and 1 to 6 carbon-carbon double bonds;
or a mixture of any thereof.

In one embodiment, the neutral lipid is DSPC, and the anionic amphiphile is selected from the group consisting of:
a carboxylic acid selected from the group consisting of, octanoic acid, decanoic acid, lauric acid, myristic acid, palmitic acid, stearic acid, arachidic acid, 2-butyloctanoic acid, 2-hexyldecanoic acid, 2-hydroxypalmitic acid, 2-methyloctadecanoic acid, hexadecenylsuccinic acid, neodecanoic acid, cyclohexanepentanoic acid, 1-adamantaneacetic acid, 4-pentylcyclohexanecarboxylic acid, cyclododecane-carboxylic acid, p-nonylbenzoic acid, 2-decenoic acid, 3-decenoic acid, palmitoleic acid, linolenic acid, linoleic acid, oleic acid, elaidic acid, arachidonic acid, lithocholic acid, chenodeoxycholic acid, deoxycholic acid, ursodeoxycholic acid, cholic acid; sodium dodecyl sulfate;
a sulfonate selected from the group consisting of sodium cetyl sulfonate and decyl benzenesulfonate;
a phosphonate selected from the group consisting of dodecyl phosphonate acid, and octadecyl phosphonate;
cholesteryl hemisuccinate;
a diacylglycerol hemisuccinate selected from the group consisting of 1,2-dilauroylglyceryl hemisuccinate (DLGS), 1,2-dimyristoylglyceryl hemisuccinate (DMGS), 1,2-dipalmitoylglyceryl hemisuccinate (DPGS), 1-palmitoyl-2-stearoylglyceryl hemisuccinate (PSGS), 1,2-distearoylglyceryl hemisuccinate (DSGS), 1-stearoyl, 2-myristoyl-glycerylhemisuccinate (SMGS), 1-palmitoyl-2-oleoylglyceryl hemisuccinate (POGS);
or a mixture of any thereof.

In one embodiment, the steroid is cholesterol, and the anionic amphiphile is selected from the group consisting of a carboxylic acid; a phosphonic acid; a sulfate; a sulfonate; a hemiester of a dicarboxylic acid with diacylglycerol; a hemiester of a dicarboxylic acid with cholesterol; a phosphatidylserine, a phosphatidic acid or a phosphatidylglycerol.

In one embodiment, the steroid is cholesterol, and the anionic amphiphile is selected from the group consisting of:
a carboxylic acid selected from the group consisting of alkylcarboxylic acids having a total of 6 to 40 carbon atoms, optionally substituted by a hydroxyl group;
alkenylcarboxylic acids having a total of 6 to 40 carbon atoms; cycloalkylcarboxylic acids having a total of 6 to 40 carbon atoms; alkylcycloalkylcarboxylic acids having a total of 6 to 40 carbon atoms; and alkylarylcarboxylic acids having a total of 6 to 40 carbon atoms; or a mixture of any thereof;
an alkyl sulfate having 6 to 30 carbon atoms;
an alkyl sulfonate having 6 to 30 carbon atoms;
an alkylaryl sulfonate having a total of 12 to 40 carbon atoms;
an alkyl phosphonate having 6 to 30 carbon atoms;
a hemiester of a dicarboxylic acid having 2 to 10 carbon atoms with diacylglycerol;
a hemiester of a dicarboxylic acid having 2 to 10 carbon atoms with cholesterol;
a diacylphosphatidylserine, wherein the hydrocarbyl portion of each of the acyl moieties thereof is an alkenyl group having 6 to 40, preferably 14 to 22, carbon atoms and 1 to 6 carbon-carbon double bonds;
or a mixture of any thereof.

In one embodiment, the steroid is cholesterol, and the anionic amphiphile is selected from the group consisting of:
a carboxylic acid selected from the group consisting of, octanoic acid, decanoic acid, lauric acid, myristic acid, palmitic acid, stearic acid, arachidic acid, 2-butyloctanoic acid, 2-hexyldecanoic acid, 2-hydroxypalmitic acid, 2-methyloctadecanoic acid, hexadecenylsuccinic acid, neodecanoic acid, cyclohexanepentanoic acid, 1-adamantaneacetic acid, 4-pentylcyclohexanecarboxylic acid, cyclododecane-carboxylic acid, p-nonylbenzoic acid, 2-decenoic acid, 3-decenoic acid, palmitoleic acid, linolenic acid, linoleic acid, oleic acid, elaidic acid, arachidonic acid, lithocholic acid, chenodeoxycholic acid, deoxycholic acid, ursodeoxycholic acid, and cholic acid; sodium dodecyl sulfate;
a sulfonate selected from the group consisting of sodium cetyl sulfonate and decyl benzenesulfonate;
a phosphonate selected from the group consisting of dodecyl phosphonate acid, and octadecyl phosphonate;
cholesteryl hemisuccinate;
a diacylglycerol hemisuccinate selected from the group consisting of 1,2-dilauroylglyceryl hemisuccinate (DLGS), 1,2-dimyristoylglyceryl hemisuccinate (DMGS), 1,2-dipalmitoylglyceryl hemisuccinate (DPGS), 1-palmitoyl-2-stearoylglyceryl hemisuccinate (PSGS), 1,2-distearoylglyceryl hemisuccinate (DSGS), 1-stearoyl, 2-myristoyl-glycerylhemisuccinate (SMGS), and 1-palmitoyl-2-oleoylglyceryl hemisuccinate (POGS);
or a mixture of any thereof.

In one embodiment, the cationically ionisable lipid is selected from the group consisting of:
N,N-dimethyl-2,3-dioleyloxypropylamine (DODMA);
1,2-distearyloxy-N,N-dimethyl-3-aminopropane (DSDMA),
1,2-dilinoleyloxy-N,N-dimethylaminopropane (DLinDMA),
3-dimethylamino-2-(cholest-5-en-3-beta-oxybutan-4-oxy)-1-(cis,cis-9,12-octadecadienoxy) propane (CLinDMA),
2-[5′-(cholest-5-en-3-beta-oxy)-3′-oxapentoxy)-3-dimethyl-1-(cis,cis-9′,12′-octadecadienoxy) propane (CpLinDMA),
N,N-dimethyl-3,4-dioleyloxybenzylamine (DMOBA),
1,2-N,N′-dioleylcarbamyl-3-dimethylaminopropane (DOcarbDAP),
2,3-dilinoleoyloxy-N,N-dimethylpropylamine (DLinDAP),
1,2-N,N′-Dilinoleylcarbamyl-3-dimethylaminopropane (DLincarbDAP),
1,2-dilinoleoylcarbamyl-3-dimethylaminopropane (DLinCDAP),
2,2-dilinoleyl-4-dimethylaminomethyl-[1,3]-dioxolane (DLin-K-DMA),
2,2-dilinoleyl-4-dimethylaminoethyl-[1,3]-dioxolane (DLin-K5-XTC2-DMA),
2,2-dilinoleyl-4-(2-dimethylaminoethyl)-[1,3]-dioxolane (DLin-KC2-DMA),
heptatriaconta-6,9,28,31-tetraen-19-yl-4-(dimethylamino)butanoate (DLin-MC3-DMA),
2-({8-[(3B)-cholest-5-en-3-yloxy]octyl}oxy)-N,N-dimethyl-3-[(9Z,12Z)-octadeca-9,12-dien-1-yloxy]propan-1-amine (octyl-CLinDMA),
di((Z)-non-2-en-1-yl) 8,8′-((((2 (dimethylamino)ethyl)thio) carbonyl) azanediyl)-dioctanoate (ATX),
N,N-dimethyl-2,3-bis(dodecyloxy) propan-1-amine (DLDMA),
N,N-dimethyl-2,3-bis(tetradecyloxy) propan-1-amine (DMDMA),
Di((Z)-non-2-en-1-yl)-9-((4-(dimethylaminobutanoyl)oxy) heptadecanedioate (L319),
N-dodecyl-3-((2-dodecylcarbamoyl-ethyl)-{2-[(2-dodecylcarbamoylethyl)-2-{(2-dodecylcarbamoyl-ethyl)-[2-(2-dodecylcarbamoyl-ethylamino)-ethyl]-amino}-ethylamino) propionamide (lipidoid 98N12-5),
1-[2-[bis(2-hydroxydodecyl)amino]ethyl-[2-[4-[2-[bis(2-hydroxydodecyl)-amino]ethyl]piperazin-1-yl]ethyl]amino]dodecan-2-ol (lipidoid C12-200),
(6Z,9Z,28Z,31Z)-heptatriaconta-6,9,28,31-tetraen-19-yl 4-(dimethylamino)butanoate;
bis-(2-butyloctyl) 10-(N-(3-(dimethylamino) propyl) nonanamido) nonadecanedioate (A9);
[(4-hydroxybutyl) azanediyl]di(hexane-6,1-diyl)bis(2-hexyldecanoate)) (ALC-315);
(heptadecan-9-yl 8-{(2-hydroxyethyl) [6-oxo-6-(undecyloxy) octyl]amino}octanoate) (L5);
heptadecan-9-yl 8-{(2-hydroxyethyl) [6-oxo-6-(undecyloxy) hexyl]amino}octanoate) (SM-102);
((2-((4-(dimethylamino)butanoyl)oxy)ethyl) azanediyl)bis(octane-8,1-diyl)bis(2-hexyldecanoate) (EA 405);
(((4-(dimethylamino)butanoyl)oxy) azanediyl)bis(octane-8,1-diyl)bis(2-hexyldecanoate) (HY405);
8-(((4-(dimethylamino)butanoyl)oxy)((9Z,12Z)-octadeca-9,12-dien-1-yl)amino) octyl 2-hexyldecanoate (HY501);
di(heptadecan-9-yl) 3,3′-((2-(4-methylpiperazin-1-yl)ethyl) azanediyl)dipropionate (BHD-C2C2-PipZ)
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the neutral lipid is selected from the group consisting of DSPC, DOPC, DMPC, DPPC, POPC, DOPE, DPPG, POPE, DPPE, DMPE, DSPE, and SM, and the steroid is cholesterol.

In one embodiment, the cationically ionisable lipid is selected from the group consisting of 2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
N,N-dimethyl-2,3-dioleyloxypropylamine (DODMA);
di((Z)-non-2-en-1-yl)-9-((4-(dimethylaminobutanoyl)oxy) heptadecanedioate (L319);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
HY501 (as described further herein);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
di(heptadecan-9-yl) 3,3′-((2-(4-methylpiperazin-1-yl)ethyl) azanediyl)dipropionate (BHD-C2C2-PipZ);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the neutral lipid is selected from the group consisting of DSPC, DPPC, DMPC, DOPC, POPC, and SM, and
   the steroid is cholesterol.

In one embodiment, the cationically ionisable lipid is selected from the group consisting of:
N,N-dimethyl-2,3-dioleyloxypropylamine (DODMA);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate
(SM102);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(pyrrolidin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the neutral lipid is DSPC, and the steroid is cholesterol.

In one embodiment, the cationically ionisable lipid is selected from the group consisting of N,N-dimethyl-2,3-dioleyloxypropylamine (DODMA);

1,2-distearyloxy-N,N-dimethyl-3-aminopropane (DSDMA),
1,2-dilinoleyloxy-N,N-dimethylaminopropane (DLinDMA),
3-dimethylamino-2-(cholest-5-en-3-beta-oxybutan-4-oxy)-1-(cis,cis-9,12-octadecadienoxy) propane (CLinDMA),
2-[5′-(cholest-5-en-3-beta-oxy)-3′-oxapentoxy)-3-dimethyl-1-(cis,cis-9′,12′-octadecadienoxy) propane (CpLinDMA),
N,N-dimethyl-3,4-dioleyloxybenzylamine (DMOBA),
1,2-N,N′-dioleylcarbamyl-3-dimethylaminopropane (DOcarbDAP),
2,3-dilinoleoyloxy-N,N-dimethylpropylamine (DLinDAP),
1,2-N,N′-Dilinoleylcarbamyl-3-dimethylaminopropane (DLincarbDAP),
1,2-dilinoleoylcarbamyl-3-dimethylaminopropane (DLinCDAP),
2,2-dilinoleyl-4-dimethylaminomethyl-[1,3]-dioxolane (DLin-K-DMA),
2,2-dilinoleyl-4-dimethylaminoethyl-[1,3]-dioxolane (DLin-K5-XTC2-DMA),
2,2-dilinoleyl-4-(2-dimethylaminoethyl)-[1,3]-dioxolane (DLin-KC2-DMA),
heptatriaconta-6,9,28,31-tetraen-19-yl-4-(dimethylamino)butanoate (DLin-MC3-DMA),
2-({8-[(3B)-cholest-5-en-3-yloxy]octyl}oxy)-N,N-dimethyl-3-[(9Z,12Z)-octadeca-9,12-dien-1-yloxy]propan-1-amine (octyl-CLinDMA),
di((Z)-non-2-en-1-yl) 8,8′-((((2 (dimethylamino)ethyl)thio) carbonyl) azanediyl)-dioctanoate (ATX),
N,N-dimethyl-2,3-bis(dodecyloxy) propan-1-amine (DLDMA),
N,N-dimethyl-2,3-bis(tetradecyloxy) propan-1-amine (DMDMA),
Di((Z)-non-2-en-1-yl)-9-((4-(dimethylaminobutanoyl)oxy) heptadecanedioate (L319),
N-dodecyl-3-((2-dodecylcarbamoyl-ethyl)-{2-[(2-dodecylcarbamoylethyl)-2-{(2-dodecylcarbamoyl-ethyl)-[2-(2-dodecylcarbamoyl-ethylamino)-ethyl]-amino}-ethylamino) propionamide (lipidoid 98N12-5),
1-[2-[bis(2-hydroxydodecyl)amino]ethyl-[2-[4-[2-[bis(2-hydroxydodecyl)-amino]ethyl]piperazin-1-yl]ethyl]amino]dodecan-2-ol (lipidoid C12-200),
(6Z,9Z,28Z,31Z)-heptatriaconta-6,9,28,31-tetraen-19-yl 4-(dimethylamino)butanoate;
bis-(2-butyloctyl) 10-(N-(3-(dimethylamino) propyl) nonanamido) nonadecanedioate (A9);
[(4-hydroxybutyl) azanediyl]di(hexane-6,1-diyl)bis(2-hexyldecanoate)) (ALC-315);
(heptadecan-9-yl 8-{(2-hydroxyethyl) [6-oxo-6-(undecyloxy) octyl]amino}octanoate) (L5);
heptadecan-9-yl 8-{(2-hydroxyethyl) [6-oxo-6-(undecyloxy) hexyl]amino}octanoate) (SM-102);
((2-((4-(dimethylamino)butanoyl)oxy)ethyl) azanediyl)bis(octane-8,1-diyl)bis(2-hexyldecanoate) (EA 405);
(((4-(dimethylamino)butanoyl)oxy) azanediyl)bis(octane-8,1-diyl)bis(2-hexyldecanoate) (HY405);
8-(((4-(dimethylamino)butanoyl)oxy)((9Z,12Z)-octadeca-9,12-dien-1-yl)amino) octyl 2-hexyldecanoate (HY501);
di(heptadecan-9-yl) 3,3′-((2-(4-methylpiperazin-1-yl)ethyl) azanediyl)dipropionate (BHD-C2C2-PipZ)
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the neutral lipid is selected from the group consisting of DSPC, DOPC, DMPC, DPPC, POPC, DOPE, DPPG, POPE, DPPE, DMPE, DSPE, and SM, and the anionic amphiphile is selected from the group consisting of a carboxylic acid; a phosphonic acid; a sulfate; a sulfonate; a hemiester of a dicarboxylic acid with diacylglycerol; a hemiester of a dicarboxylic acid with cholesterol; a phosphatidylserine, a phosphatidic acid or a phosphatidylglycerol.

In one embodiment, the cationically ionisable lipid is selected from the group consisting of 2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
N,N-dimethyl-2,3-dioleyloxypropylamine (DODMA);
di((Z)-non-2-en-1-yl)-9-((4-(dimethylaminobutanoyl)oxy) heptadecanedioate (L319);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
HY501 (as described further herein);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
di(heptadecan-9-yl) 3,3′-((2-(4-methylpiperazin-1-yl)ethyl) azanediyl)dipropionate (BHD-C2C2-PipZ);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the neutral lipid is selected from the group consisting of DSPC, DPPC, DMPC, DOPC, POPC, and SM, and
   the anionic amphiphile is selected from the group consisting of:
   a carboxylic acid selected from the group consisting of alkylcarboxylic acids having a total of 6 to 40 carbon atoms, optionally substituted by a hydroxyl group;
   alkenylcarboxylic acids having a total of 6 to 40 carbon atoms; cycloalkylcarboxylic acids having a total of 6 to 40 carbon atoms; alkylcycloalkylcarboxylic acids having a total of 6 to 40 carbon atoms; and alkylarylcarboxylic acids having a total of 6 to 40 carbon atoms; or a mixture of any thereof;
   an alkyl sulfate having 6 to 30 carbon atoms;
   an alkyl sulfonate having 6 to 30 carbon atoms;
   an alkylaryl sulfonate having a total of 12 to 40 carbon atoms;
   an alkyl phosphonate having 6 to 30 carbon atoms;
   a hemiester of a dicarboxylic acid having 2 to 10 carbon atoms with diacylglycerol;
   a hemiester of a dicarboxylic acid having 2 to 10 carbon atoms with cholesterol;
   a diacylphosphatidylserine, wherein the hydrocarbyl portion of each of the acyl moieties thereof is an alkenyl group having 6 to 40, preferably 14 to 22, carbon atoms and 1 to 6 carbon-carbon double bonds;
   or a mixture of any thereof.

In one embodiment, the cationically ionisable lipid is selected from the group consisting of:
N,N-dimethyl-2,3-dioleyloxypropylamine (DODMA);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate
(SM102);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the neutral lipid is DSPC, and
   the anionic amphiphile is selected from the group consisting of: a carboxylic acid selected from the group consisting of octanoic acid, decanoic acid, lauric acid, myristic acid, palmitic acid, stearic acid, arachidic acid, 2-butyloctanoic acid, 2-hexyldecanoic acid, 2-hydroxypalmitic acid, 2-methyloctadecanoic acid, hexadecenylsuccinic acid, neodecanoic acid, cyclohexanepentanoic acid, 1-adamantaneacetic acid, 4-pentylcyclohexanecarboxylic acid, cyclododecanecarboxylic acid, p-nonylbenzoic acid, 2-decenoic acid, 3-decenoic acid, palmitoleic acid, linolenic acid, linoleic acid, oleic acid, elaidic acid, arachidonic acid, lithocholic acid, chenodeoxycholic acid, deoxycholic acid, ursodeoxycholic acid, and cholic acid;
   sodium dodecyl sulfate;
   a sulfonate selected from the group consisting of sodium cetyl sulfonate and decyl benzenesulfonate;
   a phosphonate selected from the group consisting of dodecyl phosphonate and octadecyl phosphonate;
   cholesteryl hemisuccinate;
   a diacylglycerol hemisuccinate selected from the group consisting of 1,2-dilauroylglyceryl hemisuccinate (DLGS), 1,2-dimyristoylglyceryl hemisuccinate (DMGS), 1,2-dipalmitoylglyceryl hemisuccinate (DPGS), 1-palmitoyl-2-stearoylglyceryl hemisuccinate (PSGS), 1,2-distearoylglyceryl hemisuccinate (DSGS), 1-stearoyl, 2-myristoyl-glycerylhemisuccinate (SMGS), and 1-palmitoyl-2-oleoylglyceryl hemisuccinate (POGS);
   or a mixture of any thereof.

In one embodiment, the cationically ionisable lipid is selected from the group consisting of:
N,N-dimethyl-2,3-dioleyloxypropylamine (DODMA);
1,2-distearyloxy-N,N-dimethyl-3-aminopropane (DSDMA),
1,2-dilinoleyloxy-N,N-dimethylaminopropane (DLinDMA),
3-dimethylamino-2-(cholest-5-en-3-beta-oxybutan-4-oxy)-1-(cis,cis-9,12-octadecadienoxy) propane (CLinDMA),
2-[5′-(cholest-5-en-3-beta-oxy)-3′-oxapentoxy)-3-dimethyl-1-(cis,cis-9′,12′-octadecadienoxy) propane (CpLinDMA),
N,N-dimethyl-3,4-dioleyloxybenzylamine (DMOBA),
1,2-N,N′-dioleylcarbamyl-3-dimethylaminopropane (DOcarbDAP),
2,3-dilinoleoyloxy-N,N-dimethylpropylamine (DLinDAP),
1,2-N,N′-Dilinoleylcarbamyl-3-dimethylaminopropane (DLincarbDAP),
1,2-dilinoleoylcarbamyl-3-dimethylaminopropane (DLinCDAP),
2,2-dilinoleyl-4-dimethylaminomethyl-[1,3]-dioxolane (DLin-K-DMA),
2,2-dilinoleyl-4-dimethylaminoethyl-[1,3]-dioxolane (DLin-K5-XTC2-DMA),
2,2-dilinoleyl-4-(2-dimethylaminoethyl)-[1,3]-dioxolane (DLin-KC2-DMA),
heptatriaconta-6,9,28,31-tetraen-19-yl-4-(dimethylamino)butanoate (DLin-MC3-DMA),
2-({8-[(3B)-cholest-5-en-3-yloxy]octyl}oxy)-N,N-dimethyl-3-[(9Z,12Z)-octadeca-9,12-dien-1-yloxy]propan-1-amine (octyl-CLinDMA),
di((Z)-non-2-en-1-yl) 8,8′-((((2 (dimethylamino)ethyl)thio) carbonyl) azanediyl)-dioctanoate (ATX),
N,N-dimethyl-2,3-bis(dodecyloxy) propan-1-amine (DLDMA),
N,N-dimethyl-2,3-bis(tetradecyloxy) propan-1-amine (DMDMA),
Di((Z)-non-2-en-1-yl)-9-((4-(dimethylaminobutanoyl)oxy) heptadecanedioate (L319),
N-dodecyl-3-((2-dodecylcarbamoyl-ethyl)-{2-[(2-dodecylcarbamoylethyl)-2-{(2-dodecylcarbamoyl-ethyl)-[2-(2-dodecylcarbamoyl-ethylamino)-ethyl]-amino}-ethylamino) propionamide (lipidoid 98N12-5),
1-[2-[bis(2-hydroxydodecyl)amino]ethyl-[2-[4-[2-[bis(2-hydroxydodecyl)-amino]ethyl]piperazin-1-yl]ethyl]amino]dodecan-2-ol (lipidoid C12-200),
(6Z,9Z,28Z,31Z)-heptatriaconta-6,9,28,31-tetraen-19-yl 4-(dimethylamino)butanoate;
bis-(2-butyloctyl) 10-(N-(3-(dimethylamino) propyl) nonanamido) nonadecanedioate (A9);
[(4-hydroxybutyl) azanediyl]di(hexane-6,1-diyl)bis(2-hexyldecanoate)) (ALC-315);
(heptadecan-9-yl 8-{(2-hydroxyethyl) [6-oxo-6-(undecyloxy) octyl]amino}octanoate) (L5);
heptadecan-9-yl 8-{(2-hydroxyethyl) [6-oxo-6-(undecyloxy) hexyl]amino}octanoate) (SM-102);
((2-((4-(dimethylamino)butanoyl)oxy)ethyl) azanediyl)bis(octane-8,1-diyl)bis(2-hexyldecanoate) (EA 405);
(((4-(dimethylamino)butanoyl)oxy) azanediyl)bis(octane-8,1-diyl)bis(2-hexyldecanoate) (HY405);
8-(((4-(dimethylamino)butanoyl)oxy)((9Z,12Z)-octadeca-9,12-dien-1-yl)amino) octyl 2-hexyldecanoate (HY501);
di(heptadecan-9-yl) 3,3′-((2-(4-methylpiperazin-1-yl)ethyl) azanediyl)dipropionate (BHD-C2C2-PipZ)
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the steroid is cholesterol, and
   the anionic amphiphile is selected from the group consisting of a carboxylic acid; a phosphonic acid; a sulfate; a sulfonate; a hemiester of a dicarboxylic acid with diacylglycerol; a hemiester of a dicarboxylic acid with cholesterol; a phosphatidylserine, a phosphatidic acid or a phosphatidylglycerol.

In one embodiment, the cationically ionisable lipid is selected from the group consisting of:
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
N,N-dimethyl-2,3-dioleyloxypropylamine (DODMA);
di((Z)-non-2-en-1-yl)-9-((4-(dimethylaminobutanoyl)oxy) heptadecanedioate (L319);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
HY501;
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
di(heptadecan-9-yl) 3,3′-((2-(4-methylpiperazin-1-yl)ethyl) azanediyl)dipropionate (BHD-C2C2-PipZ);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the steroid is cholesterol, and
   the anionic amphiphile is selected from the group consisting of:
   a carboxylic acid selected from the group consisting of alkylcarboxylic acids having a total of 6 to 40 carbon atoms, optionally substituted by a hydroxyl group;
   alkenylcarboxylic acids having a total of 6 to 40 carbon atoms; cycloalkylcarboxylic acids having a total of 6 to 40 carbon atoms; alkylcycloalkylcarboxylic acids having a total of 6 to 40 carbon atoms; alkylarylcarboxylic acids having a total of 6 to 40 carbon atoms; or a mixture of any thereof;
   an alkyl sulfate having 6 to 30 carbon atoms;
   an alkyl sulfonate having 6 to 30 carbon atoms;
   an alkylaryl sulfonate having a total of 12 to 40 carbon atoms;
   an alkyl phosphonate having 6 to 30 carbon atoms;
   a hemiester of a dicarboxylic acid having 2 to 10 carbon atoms with diacylglycerol;
   a hemiester of a dicarboxylic acid having 2 to 10 carbon atoms with cholesterol;
   a diacylphosphatidylserine, wherein the hydrocarbyl portion of each of the acyl moieties thereof is an alkenyl group having 6 to 40, preferably 14 to 22, carbon atoms and 1 to 6 carbon-carbon double bonds;
   or a mixture of any thereof.

In one embodiment, the cationically ionisable lipid is selected from the group consisting of:
N,N-dimethyl-2,3-dioleyloxypropylamine (DODMA);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the steroid is cholesterol, and the anionic amphiphile is selected from the group consisting of:
   a carboxylic acid selected from the group consisting of octanoic acid, decanoic acid, lauric acid, myristic acid, palmitic acid, stearic acid, arachidic acid, 2-butyloctanoic acid, 2-hexyldecanoic acid, 2-hydroxypalmitic acid, 2-methyloctadecanoic acid, hexadecenylsuccinic acid, neodecanoic acid, cyclohexanepentanoic acid, 1-adamantaneacetic acid, 4-pentylcyclohexanecarboxylic acid, cyclododecanecarboxylic acid, p-nonylbenzoic acid, 2-decenoic acid, 3-decenoic acid, palmitoleic acid, linolenic acid, linoleic acid, oleic acid, elaidic acid, arachidonic acid, lithocholic acid, chenodeoxycholic acid, deoxycholic acid, ursodeoxycholic acid, and cholic acid;
   sodium dodecyl sulfate;
   a sulfonate selected from the group consisting of sodium cetyl sulfonate and decyl benzenesulfonate;
   a phosphonate selected from the group consisting of dodecyl phosphonate and octadecyl phosphonate;
   cholesteryl hemisuccinate;
   a diacylglycerol hemisuccinate selected from the group consisting of 1,2-dilauroylglyceryl hemisuccinate (DLGS), 1,2-dimyristoylglyceryl hemisuccinate (DMGS), 1,2-dipalmitoylglyceryl hemisuccinate (DPGS), 1-palmitoyl-2-stearoylglyceryl hemisuccinate (PSGS), 1,2-distearoylglyceryl hemisuccinate (DSGS), 1-stearoyl, 2-myristoyl-glycerylhemisuccinate (SMGS), and 1-palmitoyl-2-oleoylglyceryl hemisuccinate (POGS);
   or a mixture of any thereof.

In one embodiment, the neutral lipid is selected from the group consisting of DSPC, DOPC, DMPC, DPPC, POPC, DOPE, DPPG, POPE, DPPE, DMPE, DSPE, and SM, the steroid is cholesterol, and
the anionic amphiphile is selected from the group consisting of a carboxylic acid; a phosphonic acid; a sulfate; a sulfonate; a hemiester of a dicarboxylic acid with diacylglycerol; a hemiester of a dicarboxylic acid with cholesterol; a phosphatidylserine, a phosphatidic acid or a phosphatidylglycerol.

In one embodiment, the neutral lipid is selected from the group consisting of DSPC, DPPC, DMPC, DOPC, POPC, and SM;
the steroid is cholesterol; and
the anionic amphiphile is selected from the group consisting of: a carboxylic acid selected from the group consisting of alkylcarboxylic acids having a total of 6 to 40 carbon atoms, optionally substituted by a hydroxyl group; alkenylcarboxylic acids having a total of 6 to 40 carbon atoms; cycloalkylcarboxylic acids having a total of 6 to 40 carbon atoms; alkylcycloalkylcarboxylic acids having a total of 6 to 40 carbon atoms; alkylarylcarboxylic acids having a total of 6 to 40 carbon atoms; or a mixture of any thereof;
an alkyl sulfate having 6 to 30 carbon atoms;
an alkyl sulfonate having 6 to 30 carbon atoms;
an alkylaryl sulfonate having a total of 12 to 40 carbon atoms;
an alkyl phosphonate having 6 to 30 carbon atoms;
a hemiester of a dicarboxylic acid having 2 to 10 carbon atoms with diacylglycerol;
a hemiester of a dicarboxylic acid having 2 to 10 carbon atoms with cholesterol;
a diacylphosphatidylserine, wherein the hydrocarbyl portion of each of the acyl moieties thereof is an alkenyl group having 6 to 40, preferably 14 to 22, carbon atoms and 1 to 6 carbon-carbon double bonds;
or a mixture of any thereof.

In one embodiment, the neutral lipid is DSPC, the steroid is cholesterol, and the anionic amphiphile is selected from the group consisting of a carboxylic acid selected from the group consisting of, octanoic acid, decanoic acid, lauric acid, myristic acid, palmitic acid, stearic acid, arachidic acid, 2-butyloctanoic acid, 2-hexyldecanoic acid, 2-hydroxypalmitic acid, 2-methyloctadecanoic acid, hexadecenylsuccinic acid, neodecanoic acid, cyclohexanepentanoic acid, 1-adamantaneacetic acid, 4-pentylcyclohexanecarboxylic acid, cyclododecanecarboxylic acid, p-nonylbenzoic acid, 2-decenoic acid, 3-decenoic acid, palmitoleic acid, linolenic acid, linoleic acid, oleic acid, elaidic acid, arachidonic acid, lithocholic acid, chenodeoxycholic acid, deoxycholic acid, ursodeoxycholic acid, and cholic acid;
sodium dodecyl sulfate;
a sulfonate selected from the group consisting of sodium cetyl sulfonate and decyl benzenesulfonate;
a phosphonate selected from the group consisting of dodecyl phosphonate and octadecyl phosphonate;
cholesteryl hemisuccinate;
a diacylglycerol hemisuccinate selected from the group consisting of 1,2-dilauroylglyceryl hemisuccinate (DLGS), 1,2-dimyristoylglyceryl hemisuccinate (DMGS), 1,2-dipalmitoylglyceryl hemisuccinate (DPGS), 1-palmitoyl-2-stearoylglyceryl hemisuccinate (PSGS), 1,2-distearoylglyceryl hemisuccinate (DSGS), 1-stearoyl, 2-myristoyl-glycerylhemisuccinate (SMGS), and 1-palmitoyl-2-oleoylglyceryl hemisuccinate (POGS);
or a mixture of any thereof.

In one embodiment, the cationically ionisable lipid is selected from the group consisting of N,N-dimethyl-2,3-dioleyloxypropylamine (DODMA);
1,2-distearyloxy-N,N-dimethyl-3-aminopropane (DSDMA),
1,2-dilinoleyloxy-N,N-dimethylaminopropane (DLinDMA),
3-dimethylamino-2-(cholest-5-en-3-beta-oxybutan-4-oxy)-1-(cis,cis-9,12-octadecadienoxy) propane (CLinDMA),
2-[5′-(cholest-5-en-3-beta-oxy)-3′-oxapentoxy)-3-dimethyl-1-(cis,cis-9′,12′-octadecadienoxy) propane (CpLinDMA),
N,N-dimethyl-3,4-dioleyloxybenzylamine (DMOBA),
1,2-N,N′-dioleylcarbamyl-3-dimethylaminopropane (DOcarbDAP),
2,3-dilinoleoyloxy-N,N-dimethylpropylamine (DLinDAP),
1,2-N,N′-Dilinoleylcarbamyl-3-dimethylaminopropane (DLincarbDAP),
1,2-dilinoleoylcarbamyl-3-dimethylaminopropane (DLinCDAP),
2,2-dilinoleyl-4-dimethylaminomethyl-[1,3]-dioxolane (DLin-K-DMA),
2,2-dilinoleyl-4-dimethylaminoethyl-[1,3]-dioxolane (DLin-K5-XTC2-DMA),
2,2-dilinoleyl-4-(2-dimethylaminoethyl)-[1,3]-dioxolane (DLin-KC2-DMA),
heptatriaconta-6,9,28,31-tetraen-19-yl-4-(dimethylamino)butanoate (DLin-MC3-DMA),
2-({8-[(3B)-cholest-5-en-3-yloxy]octyl}oxy)-N,N-dimethyl-3-[(9Z,12Z)-octadeca-9,12-dien-1-yloxy]propan-1-amine (octyl-CLinDMA),
di((Z)-non-2-en-1-yl) 8,8′-((((2 (dimethylamino)ethyl)thio) carbonyl) azanediyl)-dioctanoate (ATX),
N,N-dimethyl-2,3-bis(dodecyloxy) propan-1-amine (DLDMA),
N,N-dimethyl-2,3-bis(tetradecyloxy) propan-1-amine (DMDMA),
Di((Z)-non-2-en-1-yl)-9-((4-(dimethylaminobutanoyl)oxy) heptadecanedioate (L319),
N-dodecyl-3-((2-dodecylcarbamoyl-ethyl)-{2-[(2-dodecylcarbamoylethyl)-2-{(2-dodecylcarbamoyl-ethyl)-[2-(2-dodecylcarbamoyl-ethylamino)-ethyl]-amino}-ethylamino) propionamide (lipidoid 98N12-5),
1-[2-[bis(2-hydroxydodecyl)amino]ethyl-[2-[4-[2-[bis(2-hydroxydodecyl)-amino]ethyl]piperazin-1-yl]ethyl]amino]dodecan-2-ol (lipidoid C12-200),
(6Z,9Z,28Z,31Z)-heptatriaconta-6,9,28,31-tetraen-19-yl 4-(dimethylamino)butanoate;
bis-(2-butyloctyl) 10-(N-(3-(dimethylamino) propyl) nonanamido) nonadecanedioate (A9);
[(4-hydroxybutyl) azanediyl]di(hexane-6,1-diyl)bis(2-hexyldecanoate)) (ALC-315);
(heptadecan-9-yl 8-{(2-hydroxyethyl) [6-oxo-6-(undecyloxy) octyl]amino}octanoate) (L5);
heptadecan-9-yl 8-{(2-hydroxyethyl) [6-oxo-6-(undecyloxy) hexyl]amino}octanoate) (SM-102);
((2-((4-(dimethylamino)butanoyl)oxy)ethyl) azanediyl)bis(octane-8,1-diyl)bis(2-hexyldecanoate) (EA 405);
(((4-(dimethylamino)butanoyl)oxy) azanediyl)bis(octane-8,1-diyl)bis(2-hexyldecanoate) (HY405);
8-(((4-(dimethylamino)butanoyl)oxy)((9Z,12Z)-octadeca-9,12-dien-1-yl)amino) octyl 2-hexyldecanoate (HY501);
di(heptadecan-9-yl) 3,3′-((2-(4-methylpiperazin-1-yl)ethyl) azanediyl)dipropionate (BHD-C2C2-PipZ)
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr); and
bis(2-hexyldecyl) 3,3′-((4-(4-methylpiperazin-1-yl)butyl) azanediyl)dipropionate (BHD-C2C4-PipZ);
di(nonadecan-9-yl) 3,3′-((4-(4-methylpiperazin-1-yl)butyl) azanediyl)dipropionate (DND-C2-C4-PipZ);
   the neutral lipid is selected from the group consisting of DSPC, DOPC, DMPC, DPPC, POPC, DOPE, DPPG, POPE, DPPE, DMPE, DSPE, and SM;
   the steroid is cholesterol; and
   the anionic amphiphile is selected from the group consisting of a carboxylic acid; a phosphonic acid; a sulfate; a sulfonate; a hemiester of a dicarboxylic acid with diacylglycerol; a hemiester of a dicarboxylic acid with cholesterol; a phosphatidylserine, a phosphatidic acid or a phosphatidylglycerol.

In one embodiment, the cationically ionisable lipid is selected from the group consisting of:
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
N,N-dimethyl-2,3-dioleyloxypropylamine (DODMA);
di((Z)-non-2-en-1-yl)-9-((4-(dimethylaminobutanoyl)oxy) heptadecanedioate (L319);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
HY501 (as described further herein);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
di(heptadecan-9-yl) 3,3′-((2-(4-methylpiperazin-1-yl)ethyl) azanediyl)dipropionate (BHD-C2C2-PipZ);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the neutral lipid is selected from the group consisting of DSPC, DPPC, DMPC, DOPC, POPC, and SM;
   the steroid is cholesterol; and
   the anionic amphiphile is selected from the group consisting of:
   a carboxylic acid selected from the group consisting of alkylcarboxylic acids having a total of 6 to 40 carbon atoms, optionally substituted by a hydroxyl group; alkenylcarboxylic acids having a total of 6 to 40 carbon atoms; cycloalkylcarboxylic acids having a total of 6 to 40 carbon atoms; alkylcycloalkylcarboxylic acids having a total of 6 to 40 carbon atoms; alkylarylcarboxylic acids having a total of 6 to 40 carbon atoms; or a mixture of any thereof;
   an alkyl sulfate having 6 to 30 carbon atoms;
   an alkyl sulfonate having 6 to 30 carbon atoms;
   an alkylaryl sulfonate having a total of 12 to 40 carbon atoms;
   an alkyl phosphonate having 6 to 30 carbon atoms;
   a hemiester of a dicarboxylic acid having 2 to 10 carbon atoms with diacylglycerol;
   a hemiester of a dicarboxylic acid having 2 to 10 carbon atoms with cholesterol;
   a diacylphosphatidylserine, wherein the hydrocarbyl portion of each of the acyl moieties thereof is an alkenyl group having 6 to 40, preferably 14 to 22, carbon atoms and 1 to 6 carbon-carbon double bonds;
   or a mixture of any thereof.

In one embodiment, the cationically ionisable lipid is selected from the group consisting of:
N,N-dimethyl-2,3-dioleyloxypropylamine (DODMA);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ); and
di(nonadecan-9-yl) 3,3′-((4-(4-methylpiperazin-1-yl)butyl) azanediyl)dipropionate (DND-C2-C4-PipZ);
   the neutral lipid is DSPC;
   the steroid is cholesterol; and
   the anionic amphiphile is selected from the group consisting of:
   a carboxylic acid selected from the group consisting of octanoic acid, decanoic acid, lauric acid, myristic acid, palmitic acid, stearic acid, arachidic acid, 2-butyloctanoic acid, 2-hexyldecanoic acid, 2-hydroxypalmitic acid, 2-methyloctadecanoic acid, hexadecenylsuccinic acid, neodecanoic acid, cyclohexanepentanoic acid, 1-adamantaneacetic acid, 4-pentylcyclohexanecarboxylic acid, cyclododecane-carboxylic acid, p-nonylbenzoic acid, 2-decenoic acid, 3-decenoic acid, palmitoleic acid, linolenic acid, linoleic acid, oleic acid, elaidic acid, arachidonic acid, lithocholic acid, chenodeoxycholic acid, deoxycholic acid, ursodeoxycholic acid, and cholic acid; sodium dodecyl sulfate;
   a sulfonate selected from the group consisting of sodium cetyl sulfonate and decyl benzenesulfonate;
   a phosphonate selected from the group consisting of dodecyl phosphonate and octadecyl phosphonate;
   cholesteryl hemisuccinate;
   a diacylglycerol hemisuccinate selected from the group consisting of 1,2-dilauroylglyceryl hemisuccinate (DLGS), 1,2-dimyristoylglyceryl hemisuccinate (DMGS), 1,2-dipalmitoylglyceryl hemisuccinate (DPGS), 1-palmitoyl-2-stearoylglyceryl hemisuccinate (PSGS), 1,2-distearoylglyceryl hemisuccinate (DSGS), 1-stearoyl, 2-myristoyl-glycerylhemisuccinate (SMGS), and 1-palmitoyl-2-oleoylglyceryl hemisuccinate (POGS);
   or a mixture of any thereof.

### Pharmaceutical Compositions

The lipid nanoparticle compositions described herein are useful as or for preparing pharmaceutical compositions or medicaments for therapeutic or prophylactic treatments.

The lipid nanoparticle compositions described herein may be administered in the form of any suitable pharmaceutical composition.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. In some embodiments, the therapeutically effective agent is or comprises the active ingredient, as described herein. In the context of the present disclosure, the pharmaceutical composition preferably comprises RNA (such as mRNA) as described herein. In some embodiments, the therapeutically effective agent is or comprises an RNA (preferably mRNA), as described in the present disclosure, which comprises a nucleic acid sequence (e.g., an ORF) encoding one or more polypeptides, e.g., a peptide or protein, preferably a pharmaceutically active peptide or protein.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-γ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys, as well as lipophilic components, such as saponins, trehalose-6,6-dibehenate (TDB), monophosphoryl lipid-A (MPL), monomycoloyl glycerol (MMG), or glucopyranosyl lipid adjuvant (GLA).

The pharmaceutical compositions of the present disclosure may be in in a frozen form or in a “ready-to-use form” (i.e., in a form, in particular a liquid form, which can be immediately administered to a subject, e.g., without any processing such as thawing, reconstituting or diluting). Thus, prior to administration of a storable form of a pharmaceutical composition, this storable form has to be processed or transferred into a ready-to-use or administrable form. E.g., a frozen pharmaceutical composition has to be thawed. Ready to use injectables can be presented in containers such as vials, ampoules or syringes wherein the container may contain one or more doses.

In one embodiment, the pharmaceutical composition is lyophilized. In one embodiment, the pharmaceutical composition is spray dried. These techniques are well known to those skilled in the art.

In some embodiments, the pharmaceutical composition is in frozen form and can be stored at a temperature of about −90° C. or higher, such as about −90° C. to about −10° C. For example, the frozen pharmaceutical compositions described herein can be stored at a temperature ranging from about −90° C. to about −10° C., such as from about −905° C. to about −40° C. or from about −40° C. to about −25° C., or from about −25° C. to about −10° C., or a temperature of about −20° C.

In some embodiments of the pharmaceutical compositions in frozen form, the pharmaceutical composition can be stored for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks. For example, the frozen pharmaceutical composition can be stored for at least 4 weeks, preferably at least 1 month, more preferably at least 2 months, more preferably at least 3 months, more preferably at least 6 months at −20° C.

In some embodiments of the pharmaceutical compositions in frozen form, the nucleic acid (such as RNA) integrity after thawing the frozen pharmaceutical composition is at least 90%, at least 95%, at least 97%, at least 98%, or substantially 100%, e.g., after thawing the frozen composition which has been stored (for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks) at −20° C.

In some embodiments, the initial nucleic acid integrity (such as the initial RNA integrity) of the pharmaceutical composition (i.e., after its preparation but before freezing) is at least 50% and the nucleic acid (such as RNA) integrity of the composition after thawing the frozen composition is at least 90%, preferably at least 95%, more preferably at least 97%, more preferably at least 98%, more preferably substantially 100%, of the initial nucleic acid integrity (such as the initial RNA integrity).

In some embodiments of the pharmaceutical compositions in frozen form, the size (Z_{average}) and/or size distribution and/or PDI of the particles after thawing the frozen pharmaceutical composition is essentially equal to the size (Z_{average}) and/or size distribution and/or PDI of the particles before freezing. For example, if a ready-to-use pharmaceutical composition is prepared from a frozen pharmaceutical composition as described herein, it is preferred that the size (Z_{average}) and/or size distribution and/or PDI of the particles contained in the ready-to-use pharmaceutical composition is essentially equal to the size (Z_{average}) and/or size distribution and/or PDI of the particles contained in the frozen pharmaceutical composition before freezing (such as contained in the formulation prepared in step (I) of the method of the second aspect).

In some embodiments, the size of the nucleic acid (such as RNA) particles and the nucleic acid (such as RNA) integrity of the pharmaceutical composition after one freeze/thaw cycle, preferably after two freeze/thaw cycles, more preferably after three freeze/thaw cycles, more preferably after four freeze/thaw cycles, more preferably after five freeze/thaw cycles or more, are essentially equal to the size of the nucleic acid (such as RNA) particles and the nucleic acid (such as RNA) integrity of the initial pharmaceutical composition (i.e., before the pharmaceutical composition has been frozen for the first time).

In some embodiments, the pharmaceutical composition is in liquid form and can be stored at a temperature ranging from about 0° C. to about 20° C. For example, the liquid pharmaceutical compositions described herein (such as the liquid compositions prepared by the methods of the second, fourth or seventh aspect, or the liquid compositions of the first, fifth, eighth, ninth, or tenth aspect) can be stored at a temperature ranging from about 1° C. to about 15° C., such as from about 2° C. to about 10° C., or from about 2° C. to about 8° C., or at a temperature of about 5° C.

In some embodiments of the pharmaceutical compositions in liquid form, the pharmaceutical composition can be stored for at least 1 week, such as at least 2 weeks, at least 3 weeks, at least 4 weeks, at least 1 month, at least 2 months, at least 3 months, at least 6 months, at least 12 months, or at least 24 months, preferably at least 4 weeks. For example, the liquid pharmaceutical composition can be stored for at least 4 weeks, preferably at least 1 month, more preferably at least 2 months, more preferably at least 3 months, more preferably at least 6 months at 5° C.

In some embodiments of the pharmaceutical composition in liquid form, the nucleic acid (such as RNA) integrity of the liquid composition, when stored, e.g., at 0° C. or higher for at least one week, is such that the desired effect, e.g., to induce an immune response, can be achieved. For example, the nucleic acid (such as RNA) integrity of the liquid composition, when stored, e.g., at 0° C. or higher for at least one week (such as for at least 2 weeks, at least three weeks, at least four weeks, at least one month, at least two months, at least three months, at least 4 months, or at least 6 months), may be at least 90%, compared to the nucleic acid (such as RNA) integrity of the initial composition, i.e., the nucleic acid (such as RNA) integrity before the composition has been stored. In some embodiments, the nucleic acid (such as RNA) integrity of the composition after storage for at least four weeks (e.g., for at least three months), preferably at a temperature of 0° C. or higher, such as about 2° C. to about 8° C., is at least 90%, compared to the nucleic acid (such as RNA) integrity before storage.

In some embodiments, the initial nucleic acid integrity (such as the initial RNA integrity) of the pharmaceutical composition (i.e., after its preparation but before storage) is at least 50% and the nucleic acid (such as RNA) integrity of the pharmaceutical composition after storage for at least one week (such as for at least 2 weeks, at least three weeks, at least four weeks, at least one month, at least two months, or at least 3 months), preferably at a temperature of 0° C. or higher, such as about 2° C. to about 8° C., is at least 90% of the initial nucleic acid integrity (such as the initial RNA integrity).

In some embodiments of the pharmaceutical composition in liquid form, the size (Z_{average}) (and/or size distribution and/or polydispersity index (PDI)) of the particles of the pharmaceutical composition, when stored, e.g., at 0° C. or higher for at least one week, is such that the desired effect, e.g., to induce an immune response, can be achieved. For example, the size (Z_{average}) (and/or size distribution and/or polydispersity index (PDI)) of the particles of the pharmaceutical composition, when stored, e.g., at 0° C. or higher for at least one week, is essentially equal to the size (Z_{average}) (and/or size distribution and/or PDI) of the particles of the initial pharmaceutical composition, i.e., before storage. In some embodiments, the size (Z_{average}) of the particles after storage of the pharmaceutical composition, e.g., at 0° C. or higher for at least one week is between about 50 nm and about 500 nm, preferably between about 40 nm and about 200 nm, more preferably between about 40 nm and about 120 nm. In some embodiments, the PDI of the particles after storage of the pharmaceutical composition, e.g., at 0° C. or higher for at least one week is less than 0.3, preferably less than 0.2, more preferably less than 0.1. In some embodiments, the size (Z_{average}) of the particles after storage of the pharmaceutical composition, e.g., at 0° C. or higher for at least one week is between about 50 nm and about 500 nm, preferably between about 40 nm and about 200 nm, more preferably between about 40 nm and about 120 nm, and the size (Z_{average}) (and/or size distribution and/or PDI) of the particles after storage of the pharmaceutical composition, e.g., at 0° C. or higher for at least one week is essentially equal to the size (Z_{average}) (and/or size distribution and/or PDI) of the particles before storage. In some embodiments, the size (Z_{average}) of the particles after storage of the pharmaceutical composition, e.g., at 0° C. or higher for at least one week is between about 50 nm and about 500 nm, preferably between about 40 nm and about 200 nm, more preferably between about 40 nm and about 120 nm, and the PDI of the particles after storage of the pharmaceutical composition, e.g., at 0° C. or higher for at least one week is less than 0.3 (preferably less than 0.2, more preferably less than 0.1).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the particles or pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the particles or pharmaceutical compositions described herein may depend on various of such parameters. In the case that a reaction in a patient is insufficient with an initial dose, higher doses (or effectively higher doses achieved by a different, more localized route of administration) may be used.

In particular embodiments, a pharmaceutical composition of the present disclosure (e.g., an immunogenic composition, i.e., a pharmaceutical composition which can be used for inducing an immune response) is formulated as a single-dose in a container, e.g., a vial. In some embodiments, the immunogenic composition is formulated as a multi-dose formulation in a vial. In some embodiments, the multi-dose formulation includes at least 2 doses per vial. In some embodiments, the multi-dose formulation includes a total of 2-20 doses per vial, such as, for example, 2, 3, 4, 5, 6, 7, 8, 9, 10, 11, or 12 doses per vial. In some embodiments, each dose in the vial is equal in volume. In some embodiments, a first dose is a different volume than a subsequent dose.

A “stable” multi-dose formulation preferably exhibits no unacceptable levels of microbial growth, and substantially no or no breakdown or degradation of the active biological molecule component(s). As used herein, a “stable” immunogenic composition includes a formulation that remains capable of eliciting a desired immunologic response when administered to a subject.

The pharmaceutical compositions of the present disclosure may contain buffers (in particular, derived from the nucleic acid (such as RNA) compositions with which the pharmaceutical compositions have been prepared), preservatives, and optionally other therapeutic agents. In one embodiment, the pharmaceutical compositions of the present disclosure, in particular the ready-to-use pharmaceutical compositions, comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants

“Pharmaceutically acceptable salts” comprise, for example, acid addition salts which may, for example, be formed by using a pharmaceutically acceptable acid such as hydrochloric acid, acetic acid, lactic acid, 2-(N-morpholino) ethanesulfonic acid (MES), 3-(N-morpholino) propanesulfonic acid (MOPS), 2-[4-(2-hydroxyethyl) piperazin-1-yl]ethanesulfonic acid (HEPES) or benzoic acid. Furthermore, suitable pharmaceutically acceptable salts may include alkali metal salts (e.g., sodium or potassium salts); alkaline earth metal salts (e.g., calcium or magnesium salts); ammonium (NH₄⁺); and salts formed with suitable organic ligands (e.g., quaternary ammonium and amine cations). Illustrative examples of pharmaceutically acceptable salts can be found in the prior art; see, for example, S. M. Berge et al., “Pharmaceutical Salts”, J. Pharm. Sci., 66, pp. 1-19 (1977)). Salts which are not pharmaceutically acceptable may be used for preparing pharmaceutically acceptable salts and are included in the present disclosure.

The term “diluent” relates a diluting and/or thinning agent. Moreover, the term “diluent” includes any one or more of fluid, liquid or solid suspension and/or mixing media. Examples of suitable diluents include ethanol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, the compositions described herein, such as the pharmaceutical compositions or ready-to-use pharmaceutical compositions described herein, may be administered intravenously, intraarterially, subcutaneously, intradermally, dermally, intranodally, intramuscularly or intratumorally. In certain embodiments, the (pharmaceutical) composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the (pharmaceutical) compositions, in particular the ready-to-use pharmaceutical compositions, are formulated for systemic administration. In another preferred embodiment, the systemic administration is by intravenous administration. In another preferred embodiment, the (pharmaceutical) compositions, in particular the ready-to-use pharmaceutical compositions, are formulated for intramuscular administration.

### Medical Uses and Methods of Treatment

The lipid nanoparticle compositions described herein may be used in the therapeutic or prophylactic treatment of various diseases, in particular diseases in which provision of a peptide or protein to a subject results in a therapeutic or prophylactic effect. For example, provision of an antigen or epitope which is derived from a virus may be useful in the treatment or prevention of a viral disease caused by said virus. Provision of a tumor antigen or epitope may be useful in the treatment of a cancer disease wherein cancer cells express said tumor antigen. Provision of a functional protein or enzyme may be useful in the treatment of genetic disorder characterized by a dysfunctional protein, for example in lysosomal storage diseases (e.g. Mucopolysaccharidoses) or factor deficiencies. Provision of a cytokine or a cytokine-fusion may be useful to modulate tumor microenvironment.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories.

The term “infectious disease” refers to any disease which can be transmitted from individual to individual or from organism to organism, and is caused by a microbial agent. Infectious diseases are known in the art and include, for example, a viral disease, a bacterial disease, or a parasitic disease, which diseases are caused by a virus, a bacterium, and a parasite, respectively. In this regard, the infectious disease can be, for example, sexually transmitted diseases (e.g., chlamydia, gonorrhea, or syphilis), SARS, coronavirus diseases (e.g., COVID-19), acquired immune deficiency syndrome (AIDS), measles, chicken pox, cytomegalovirus infections, herpes simplex virus (e.g., HSV-1, HSV-2), hepatitis (such as hepatitis B or C), influenza (flu, such as human flu, swine flu, dog flu, horse flu, and avian flu), HPV infection, shingles, rabies, common cold, gastroenteritis, rubella, mumps, anthrax, cholera, diphtheria, foodborne illnesses, leprosy, meningitis, peptic ulcer disease, pneumonia, sepsis, septic shock, tetanus, tuberculosis, typhoid fever, urinary tract infection, Lyme disease, Rocky Mountain spotted fever, chlamydia, pertussis, tetanus, meningitis, scarlet fever, malaria, trypanosomiasis, Chagas disease, leishmaniasis, trichomoniasis, dientamoebiasis, giardiasis, amebic dysentery, coccidiosis, toxoplasmosis, sarcocystosis, rhinosporidiosis, and balantidiasis.

In some embodiments, the lipid nanoparticle compositions described herein may be used in the therapeutic or prophylactic treatment of an infectious disease.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In some embodiments of the disclosure, the aim is to provide protection against an infectious disease by vaccination.

In some embodiments of the disclosure, the aim is to provide secreted therapeutic proteins, such as antibodies, bispecific antibodies, cytokines, cytokine fusion proteins, enzymes, to a subject, in particular a subject in need thereof.

In some embodiments of the disclosure, the aim is to provide a protein replacement therapy, such as production of erythropoietin, Factor VII, Von Willebrand factor, β-galactosidase, Alpha-N-acetylglucosaminidase, to a subject, in particular a subject in need thereof.

In some embodiments of the disclosure, the aim is to modulate/reprogram immune cells in the blood.

In some embodiments of the disclosure (in particular those relating to inhibitory RNA), the aim is to reduce or inhibit the expression of a peptide or polypeptide (such as the transcription and/or translation of a target mRNA). In some embodiments, the target mRNA comprises an ORF encoding a pharmaceutically active peptide or polypeptide, in particular a pharmaceutically active peptide or polypeptide whose expression (in particular increased expression, e.g., compared to the expression in a healthy subject) is associated with a disease. In some embodiments, the target mRNA comprises an ORF encoding a pharmaceutically active peptide or polypeptide whose expression (in particular increased expression, e.g., compared to the expression in a healthy subject) is associated with cancer.

In some embodiments, the nucleic acid (such as RNA) compositions described herein which contain nucleic acid (such as RNA) encoding a SARS-COV-2 S protein, an immunogenic variant thereof, or an immunogenic fragment of the SARS-COV-2 S protein or the immunogenic variant thereof (in the following simply “SARS-COV-2 S nucleic acid compositions” which explicitly include SARS-COV-2 S RNA compositions) following administration to a subject induce an antibody response, in particular a neutralizing antibody response, in the subject that targets a panel of different S protein variants such as SARS-COV-2 S protein variants, in particular naturally occurring S protein variants. In some embodiments, the panel of different S protein variants comprises at least 5, at least 10, at least 15, or even more S protein variants. In some embodiments, such S protein variants comprise variants having amino acid modifications in the RBD domain and/or variants having amino acid modifications outside the RBD domain. In some embodiments, the SARS-COV-2 S nucleic acid compositions described herein following administration to a subject induce an immune response (cellular and/or antibody response, in particular a neutralizing antibody response) in the subject that targets VOC-202012/01.

In some embodiments, the SARS-COV-2 S nucleic acid compositions described herein following administration to a subject induce an immune response (cellular and/or antibody response, in particular a neutralizing antibody response) in the subject that targets 501.V2.

In some embodiments, the SARS-COV-2 S nucleic acid compositions described herein following administration to a subject induce an immune response (cellular and/or antibody response, in particular a neutralizing antibody response) in the subject that targets “Cluster 5”.

In some embodiments, the SARS-COV-2 S nucleic acid compositions described herein following administration to a subject induce an immune response (cellular and/or antibody response, in particular a neutralizing antibody response) in the subject that targets “B.1.1.28”.

In some embodiments, the SARS-COV-2 S nucleic acid compositions described herein following administration to a subject induce an immune response (cellular and/or antibody response, in particular a neutralizing antibody response) in the subject that targets “B.1.1.248”.

In some embodiments, the SARS-COV-2 S nucleic acid compositions described herein following administration to a subject induce an immune response (cellular and/or antibody response, in particular a neutralizing antibody response) in the subject that targets the Omicron (B.1.1.529) variant.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

The terms “immunization” or “vaccination” describe the process of administering an antigen to an individual with the purpose of inducing an immune response, for example, for therapeutic or prophylactic reasons.

### EXAMPLES

### Example 1

In the composition of this Example, a fraction of the neutral lipids in the lipid nanoparticle (LNP) composition were replaced with anionic amphiphiles. The objectives were to demonstrate colloidal stability of PEGylated LNPs having anionic amphiphiles (as a reference); demonstrate colloidal stability of LNPs having anionic amphiphiles in the absence of PEG; and study the influence of the replacement on the in vitro expression, optionally upon incubation with serum.

### Study Design

To estimate the influence of introducing the anionic amphiphiles, experiments followed a linear gradient exchange of the neutral lipid. The initial focus is in the regions surrounding a formulation rich in DSPC (DSPC+: 46.7% cationically ionizable lipid, 20.8% DSPC, 32.5 cholesterol). DSPC was then replaced with different levels (from 0 to 100%) of one anionic amphiphile.

### Generation of LNP

Stock solution concentrations were prepared with the following concentrations: mRNA: 0.22 mg/mL in deionized water, cationically ionizable lipid: 50 mM (acidified with 75 mM acetic acid), cholesterol: 50 mM, neutral lipids: 33.33 mM, anionic lipids: 33.33 mM, PEG-lipid: 8 mM, wherein ethanol was used as solvent for all lipids.

Drop mixing process: Cationically ionizable lipid was added to the wellplate, followed by cholesterol, neutral lipid, anionic lipid, and PEG (used for the reference formulations) in that order. Enough ethanol was then added to complete a final volume of 33 μL ethanol. The mRNA solution was added to a final concentration of 0.055 mg/mL, followed by buffer (35 mM Tris:Hac @ pH=5.5). Both the mRNA and buffer solutions completed the final volume of 200 μL. Aliquots were taken and further diluted stepwise using buffer (35 mM Tris:Hac @ pH=7.5). Particle size was monitored at this point using a Wyatt Dynapro III Dynamic Light Scattering Instrument.

For in vitro expression, 10 μL samples were transferred to a new wellplate, and 10 μL of human serum were posteriorly added. The mixture was incubated at room temperature for 30 minutes before transfection. A second 10 μL sample was transferred into another wellplate, and 10 μL of DI water were added. Cells were then transfected with a total mRNA content of 50 ng for both the serum-naive and serum-containing nanoparticles. The final ethanol volume in cell culture was 0.125%.

In vitro expression was measured in four cell lines: HEK-293, HEPG2, THP-1 and Jurkat. All in vitro expression data is presented compared to a benchmark. The expression of small aggregates (<40 nm) and large particles (>250 nm in diameter) were not considered, as large particles have a different internalization mechanism.

TABLE 8
Selected Lipid Compositions of PEGylated LNPs

| | | | | | Formulation |
|---|---|---|---|---|---|
| | | | Structural | Anionic | composition |
| | Lipid | Sterol | lipid | lipid | (mol %) |
| 1 | Cationically | Cholesterol | DSPC | Stearic | 46:32:13:7:2 |
| | ionizable lipid | | | acid | |
| 2 | Cationically | Cholesterol | DSPC | CHEMS | 46:32:13:7:2 |
| | ionizable lipid | | | | |
| 3 | Cationically | Cholesterol | DSPC | DMGS | 46:32:13:7:2 |
| | ionizable lipid | | | | |

Composition is in the format cationically ionizable lipid:cholesterol:DSPC:Anionic lipid:PEG lipid.

TABLE 9
Selected Lipid Compositions of Non-PEGylated LNPs

| | | | | | Formulation |
|---|---|---|---|---|---|
| | | | Structural | Anionic | composition |
| | Lipid | Sterol | lipid | lipid | (mol %) |
| 4 | Cationically | Cholesterol | DSPC | Stearic | 47:32:13:8 |
| | ionizable lipid | | | acid | |
| 5 | Cationically | Cholesterol | DSPC | CHEMS | 47:32:13:8 |
| | ionizable lipid | | | | |
| 6 | Cationically | Cholesterol | DSPC | DMGS | 47:32:13:8 |
| | ionizable lipid | | | | |

For hit selection, the particles with the largest expression (without serum incubation) were selected, as long as they complied with the following filters: (1) no evidence of serum suppression, and (2) maximum particle size of 160 nm. Preference was given to formulations in stable neighbourhoods (no drastic changes in expression nor size). For practicality, the hits were harmonised into a single formulation point (30% neutral lipid replaced).

### Results

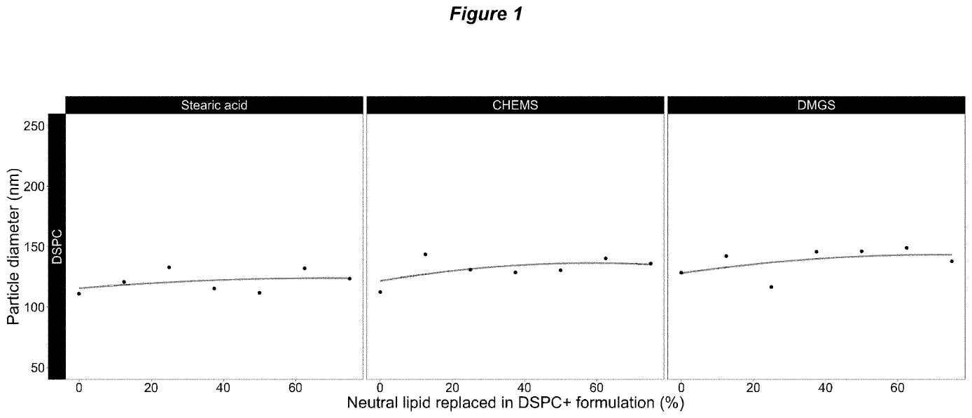

The results are shown in FIGS. 1 to 4.

FIG. 1 illustrates the particle size of the lipid nanoparticle upon partial replacement of the neutral lipid DSPC by anionic amphiphiles in the presence of PEG-lipid. Particles between 100 and 150 nm are obtained for any combination of DSPC and anionic amphiphile tested.

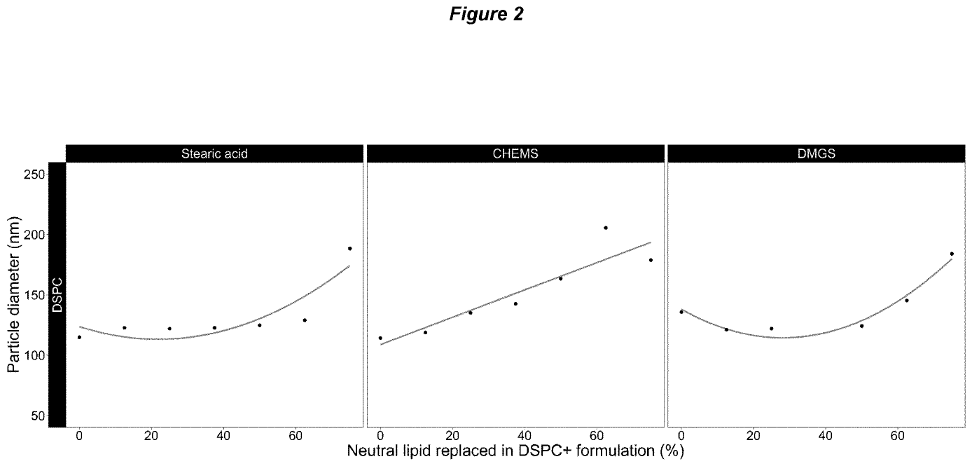

FIG. 2 illustrates the particle size of the lipid nanoparticle upon partial replacement of the neutral lipid DSPC by anionic amphiphiles in the absence of PEG-lipid. Particles between 100 and 150 nm are obtained for most of the combinations of DSPC and anionic amphiphiles even in the absence of PEG-lipid.

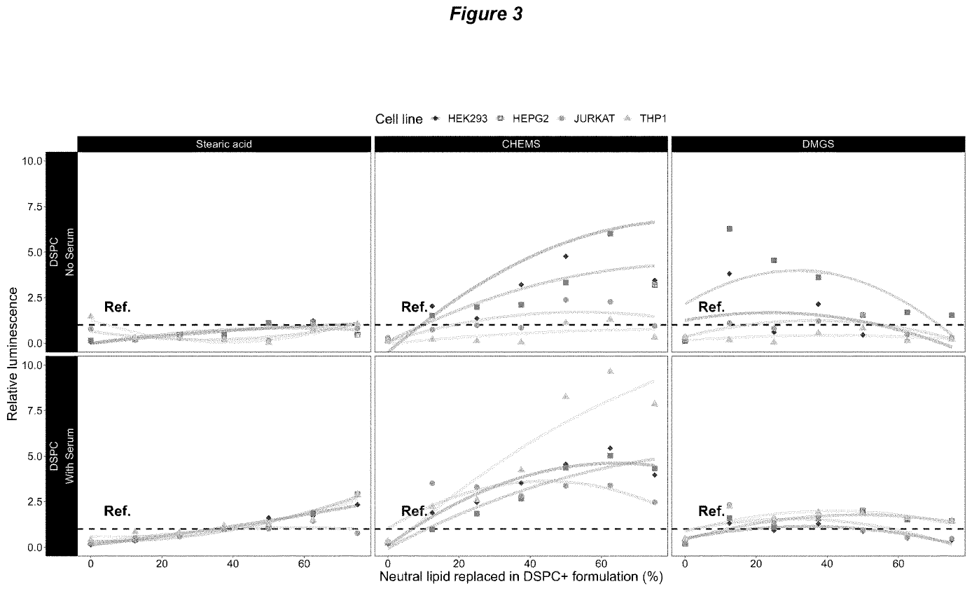

FIG. 3 shows the in vitro expression for PEGylated LNPs with upon partial replacement of the neutral lipid DSPC by anionic amphiphiles in the presence or absence of serum. Expression levels were normalized to an internal control formulation (dotted line). Expression levels equal or higher than reference were observed for most of the combinations of DSPC and anionic amphiphiles tested. Expression levels are typically higher for combinations of DSPC and anionic amphiphiles compared to DSPC alone.

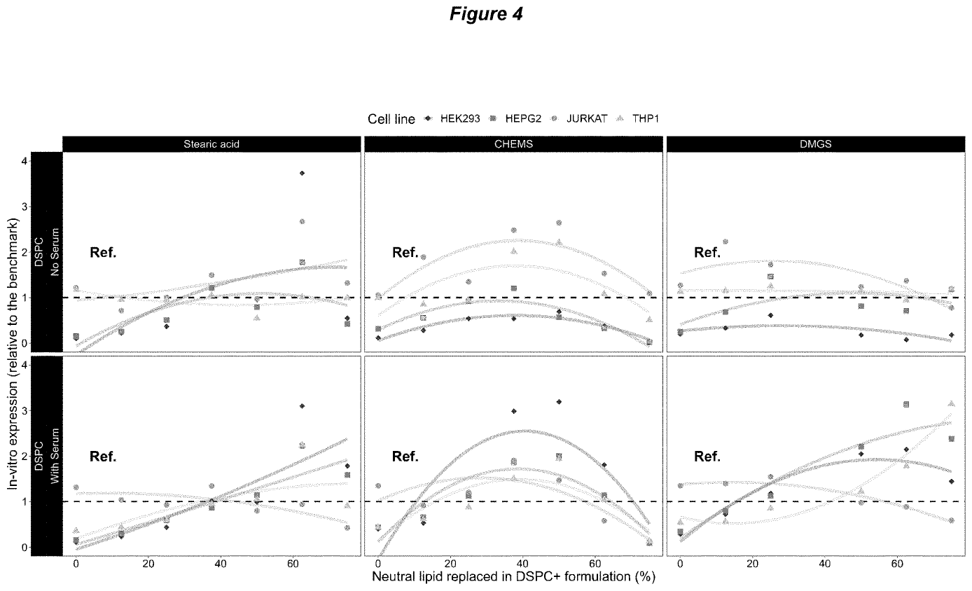

FIG. 4 shows the in vitro expression for LNPs not comprising PEG-lipid with upon partial replacement of the neutral lipid DSPC by anionic amphiphiles in the presence or absence of serum. Expression levels were normalized to an internal control formulation (dotted line). Expression levels equal or higher than reference were observed for most of the combinations of DSPC and anionic amphiphiles tested. Expression levels are typically higher for combinations of DSPC and anionic amphiphiles compared to DSPC alone and often reach an optimum for combinations wherein 20 to 60% of DSPC are replaced by anionic amphiphiles.

### Example 2

In this Example, neutral lipids in the lipid nanoparticle (LNP) composition were replaced partially or in full with anionic amphiphiles. A wide range of neutral lipids was used to determine suitable neutral lipids LNP having anionic amphiphiles. In addition, a wide selection of anionic amphiphiles was used to investigate the criticality of the respective charged and lipophilic moieties. A particle size of <=200 nm and in vitro expression levels no less than 10^{-1.5} were used as acceptance criteria.

### Study Design

To estimate the influence of introducing the anionic amphiphiles, experiments followed a linear gradient exchange of the neutral lipid. The general lipid composition is 47% cationically ionizable lipid, 21% neutral lipid and/or anionic lipid and 32% cholesterol. Substitution of neutral lipid by the anionic lipid is expressed as % substitution. As an example, a lipid composition presented as 50% exchange has a total lipid composition of 47% cationically ionizable lipid, 10.5% neutral lipid, 10.5% anionic amphiphile and 32% cholesterol.

LNP were generated, particles size determined and in vitro expression was monitored as described in example 1. The anionic amphiphiles were selected from the compounds in Table 10 below:

TABLE 10

| Charged | Lipophilic | Lipophilic | | |
|---|---|---|---|---|
| Moiety | Moiety | Moiety Name | Compound | Compound Name |
| Carboxyl | C01 | Saturated | A01 | acetic acid |
| | | | A02 | octanoic acid |
| | | | A03 | decanoic acid |
| | | | A04 | lauric acid |
| | | | A05 | myristic acid |
| | | | A06 | palmitic acid |
| | | | A07 | stearic acid |
| | | | A08 | arachidic acid |
| | C02 | Branched | A09 | 2-butyloctanoic acid |
| | | | A10 | 2-hexyldecanoic acid |
| | | | A11 | 2-hydroxypalmitic acid |
| | | | A12 | 2-methyloctadecanoic acid |
| | | | A13 | hexadecenylsuccinic acid |
| | | | A14 | 9-PAHSA |
| | | | A15 | DMGS |
| | C03 | Complex | A16 | neodecanoic aicd |
| | | | A17 | cyclohexanepentanoic acid |
| | | | A18 | 1-adamantaneacetic acid |
| | | | A19 | 4-pentylcyclohexanecarboxylic acid |
| | | | A20 | cyclododecanecarboxylic acid |
| | | | A21 | p-nonylbenzoic acid |
| | | | A22 | DACA |
| | C04 | Unsaturated | A23 | 2-decenoic acid |
| | | | A24 | 3-decenoic acid |
| | | | A25 | palmitoleic acid |
| | | | A26 | linolenic acid |
| | | | A27 | linoleic acid |
| | | | A28 | oleic acid |
| | | | A29 | elaidic acid |
| | | | A30 | arachidonic acid |
| | C05 | sterol | A31 | lithocholic acid |
| | | | A32 | chenodeoxycholic acid |
| | | | A33 | deoxycholic acid |
| | | | A34 | ursodeoxycholic acid |
| | | | A35 | cholic acid |
| | | | A36 | CHEMS |
| Phosphonic | C01 | Saturated | A37 | dodecyl phosphonic acid |
| | | | A38 | octadecyl phosphonic acid |
| Sulfonic | C01 | Saturated | A39 | sodium dodecyl sulfate |
| | C03 | complex | A40 | sodium cetyl sulfonate |
| | | | A41 | decyl benzenesulfonic acid |

### Results

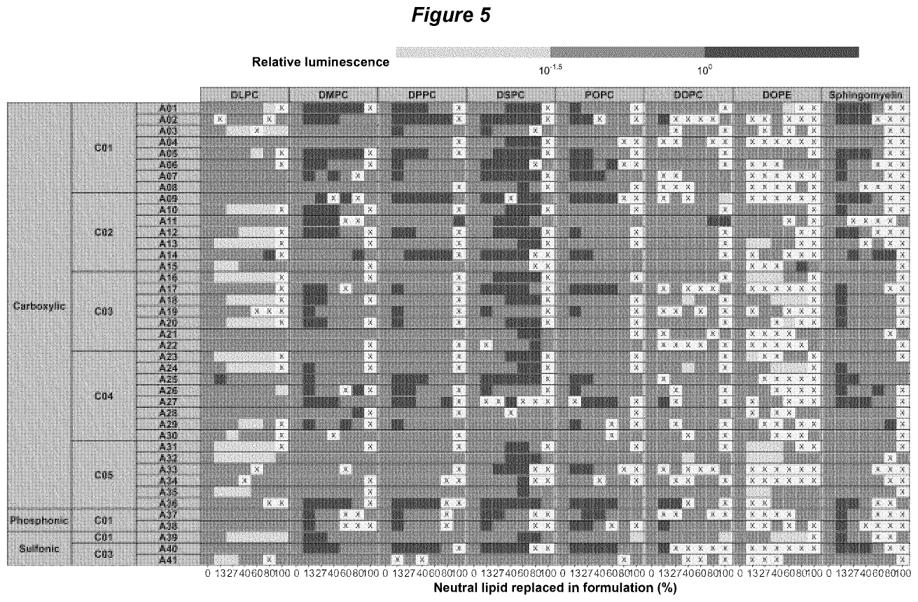

The results are shown in FIGS. 5 to 7.

FIG. 5 shows in vitro expression for LNP not comprising PEG-lipid upon replacement of neutral lipid by anionic amphiphiles in a full combinatorial design. LNP were made from cationically ionizable lipid, cholesterol, a neutral lipid selected from DLPC, DMPC, DPPC, DSPC, POPC, DOPC, DOPE or sphingomyelin as indicated at the top. Neutral lipids were partially or fully replaced by the anionic amphiphiles as listed in Table 10 above.

LNP were made as described above and particle size was monitored. LNP larger than 200 nm are denoted with “x” and excluded from further analysis. Levels of in vitro expression were obtained without prior incubation with serum on HEK293 cells. Signals were normalized to reference=1 and categorized to “low activity” in light grey having an expression less than 10^{-1.5} of the reference, “high activity” in grey having an expression between 10^{-1.5} and 10⁺⁰ of the reference and “superior activity” in dark grey having an expression higher than reference.

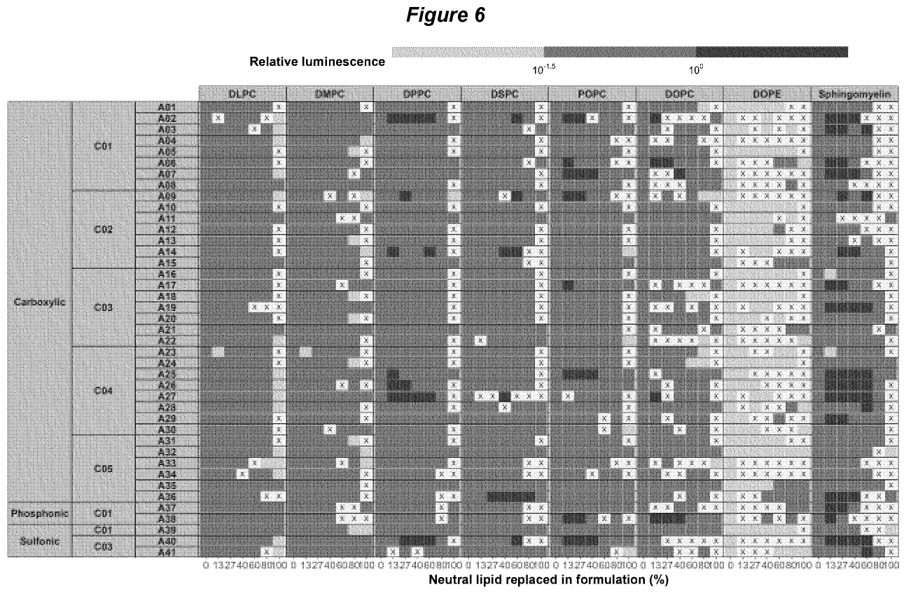

FIG. 6 shows in vitro expression for LNP not comprising PEG-lipid upon replacement of neutral lipid by anionic amphiphiles in a full combinatorial design. LNP were made from a cationically ionizable lipid, cholesterol, a neutral lipid selected from DLPC, DMPC, DPPC, DSPC, POPC, DOPC, DOPE or sphingomyelin as indicated at the top. Neutral lipids were partially or fully replaced by the anionic amphiphiles as listed in Table 10 above.

LNP were made as described in example 2 and particle size was monitored. LNP larger than 200 nm are denoted with “x” and excluded from further analysis. Levels of in vitro expression were obtained after prior incubation with serum on HepG2 cells. Signals were normalized to reference=1 and categorized to “low activity” in light grey having an expression less than 10^{-1.5} of the reference, “high activity” in grey having an expression between 10^{-1.5} and 10⁺⁰ of the reference and “superior activity” in dark grey having an expression higher than reference.

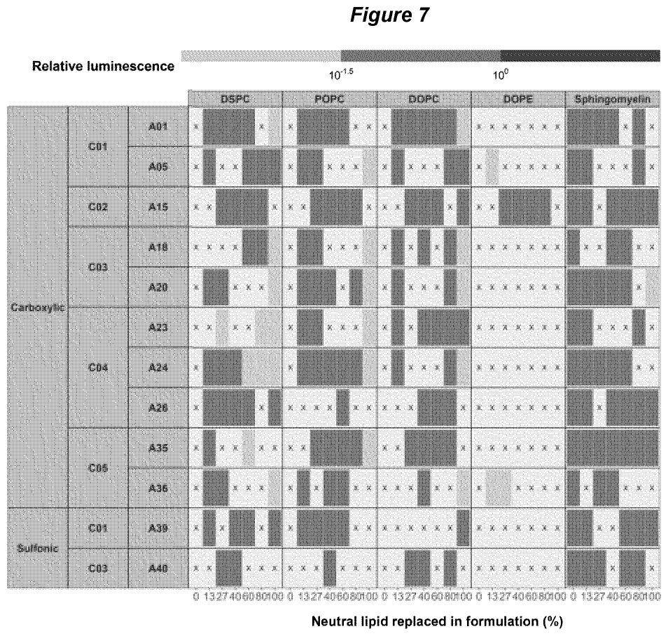

FIG. 7 shows in vitro expression for LNP not comprising PEG-lipid upon replacement of neutral lipid by anionic amphiphiles in a full combinatorial design. LNP were made from an ionizable lipid (described in WO2018/087753), cholesterol, a neutral lipid selected from DSPC, POPC, DOPC, DOPE or sphingomyelin as indicated at the top. Neutral lipids were partially or fully replaced by the anionic amphiphiles as listed in Table 10 above.

LNP were made as described above and particle size was monitored. LNP larger than 200 nm are denoted with “x” and excluded from further analysis. Levels of in vitro expression were obtained after prior incubation with serum on HepG2 cells. Signals were normalized to reference=1 and categorized to “low activity” in light grey having an expression less than 10^{-1.5} of the reference, “high activity” in grey having an expression between 10^{-1.5} and 10+0 of the reference and “superior activity” in dark grey having an expression higher than reference.

The data in FIG. 5 demonstrate that LNP can be made in the absence of PEG-lipid. Materials having a particle size<200 nm can be obtained for materials comprising either cationically ionizable lipid and cholesterol plus various neutral lipids, or cationically ionizable lipid in combination with anionic amphiphiles, for certain anionic amphiphiles alone, such as compounds A03, A06, A11, A 21, A22, A29, A29, A30, A32, A35, A36, A39 or A41.

When tested on HEK293 cells without prior incubation in human serum, LNP having superior efficacy are frequently found. Exemplary working combinations have neutral lipids-selected from DMPC, DPPC, DSPC (saturated lipid tails) or POPC or sphingomyelin (unsaturated lipid tails)—with anionic amphiphiles—including carboxylic, phosphonic or sulfonic moieties and having saturated, non-saturated, aliphatic, branched, cyclic or aromatic side chains. The combination of neutral lipid with anionic amphiphiles is typically superior to use of components selected only form one of the groups.

The data in FIG. 6 analyze the same materials upon incubation with serum and in vitro expression was monitored on HepG2 cells. LNP having superior efficacy are obtained when combining neutral lipids-selected from DPPC, DSPC (saturated lipid tails) or POPC or sphingomyelin (unsaturated lipid tails)—with various anionic amphiphiles regardless if selected from carboxylic, phosphonic or sulfonic moieties. The combination of neutral lipid with anionic amphiphiles is typically superior to use of components selected only form one of the groups.

The data in FIG. 7 demonstrate that LNP can be made in the absence of PEG-lipid using the cationically ionizable lipid. Materials having a particle size<200 nm can be obtained for materials comprising either the cationically ionizable lipid and cholesterol plus various neutral lipids, or cationically ionizable lipid and cholesterol in combination with anionic amphiphiles, or for certain anionic amphiphiles alone, such as compounds A15, A23, A24, A26, A35 or A39. LNP having superior efficacy are obtained when combining neutral lipids-selected from DPPC, DSPC (saturated lipid tails) or POPC or sphingomyelin (unsaturated lipid tails)—with various anionic amphiphiles regardless if selected from carboxylic, phosphonic or sulfonic moieties. The combination of neutral lipid with anionic amphiphiles is typically superior to use of components selected only from one of the groups.

### Example 3

Anionic LNPs were prepared using an aqueous-ethanol mixing protocol in a volume part ratio of 3:1:2 (Acidified mRNA:organic phase:quench buffer). For that, mRNA having a concentration of 0.4 mg/mL was provided in 50 mM sodium acetate pH5.5. The organic phase comprising 47.4 mM of total lipids (PEG-free modality: 47 mol % cationically ionizable lipid, 13 mol % DSPC, 32 mol % cholesterol and 8 mol % anionic amphiphile OR modality comprising PEG-lipid: 46 mol % cationically ionizable lipid, 13 mol % DSPC, 32 mol % cholesterol and 7 mol % anionic amphiphile and 2% PEG-lipid) was provided in ethanol. Quench buffer was water for injection. A raw colloid was produced by continuous mixing of the acidified mRNA with the organic phase, immediately followed by quenching. Directly after production, 2 volumes parts of 50 mM HEPES pH 7.7 were added to the raw colloid. This fast pH shift is a critical process step. A slow pH transition resulted in the formation of large particles. The neutralized colloid was dialyzed against HEPES 50 mM pH 7.7 followed by up-concentration using a cross-flow membrane (MikroKros 100K MPES 0.5 mm). Eventually, HEPES buffer solution and sucrose were added to arrive at a final product having 0.05 mg/mL of RNA, 50 mM HEPES, 300 mM sucrose, pH7.5, said product was filtered through 0.2 μm membrane and filled in glass vials.

The product specifications are listed in Table 11 below.

TABLE 11

| | | | | RNA | | RNA- | Titer | Zeta- |
|---|---|---|---|---|---|---|---|---|
| Anionic | PEGylated | Size | Poly- | content | Encapsu- | integrity | [part./mg | potential |
| amphiphile | lipid | [nm] | dispersity | [mg/ml] | lation [%] | [%] | RNA] | [mV] |
| Stearic acid | Yes | 59 | 0.145 | 0.047 | 62 | 84 | 3.00E+14 | −10.6 |
| | No | 87 | 0.061 | 0.049 | 93 | 84 | 1.23E+14 | −27.2 |
| CHEMS | Yes | 66 | 0.086 | 0.047 | 84 | 82 | 2.01E+14 | −9.3 |
| | No | 112 | 0.043 | 0.048 | 97 | 83 | 5.50E+13 | −32.0 |
| DMGS | Yes | 57 | 0.126 | 0.049 | 64 | 82 | 1.66E+14 | −10.8 |
| | No | 81 | 0.089 | 0.050 | 95 | 80 | 1.11E+14 | −27.5 |

The results of the manufacturing show LNP comprising anionic amphiphiles can be manufactured and processed to a final drug product. LNP not comprising PEGylated lipid were of better homogeneity as judged by the lower polydispersity in pairwise comparisons. LNP not comprising PEGylated lipid have a higher percentage of encapsulated RNA. Eventually, LNP not comprising PEGylated lipid are negatively charged as demonstrated by a zeta potential lower than −20 mV, whereas a zeta potential between −10 and +10 mV is considered essentially neutral.

The materials obtained are stable colloids as demonstrated in Table 12 below.

TABLE 12

| | | Size [nm] | | | polydispersity | | |
|---|---|---|---|---|---|---|---|
| | | | | 4 | | | 4 |
| Anionic | PEGylated | | 4 weeks | weeks −70° | | 4 weeks | weeks −70° |
| amphiphile | lipid | T = 0 | 5° C. | C. | T = 0 | 5° C. | C. |
| Stearic | Yes | 57 | 62 | 82 | 0.11 | 0.24 | 0.36 |
| acid | No | 76 | 72 | 83 | 0.04 | 0.09 | 0.13 |
| CHEMS | Yes | 59 | 68 | 67 | 0.19 | 0.27 | 0.18 |
| | No | 109 | 100 | 105 | 0.05 | 0.05 | 0.04 |
| DMGS | Yes | 55 | 67 | 62 | 0.11 | 0.27 | 0.25 |
| | No | 78 | 73 | 77 | 0.06 | 0.08 | 0.12 |

The results for colloidal stability show LNP not comprising PEGylated lipid are stable colloids, essentially not changing their particle size or polydispersity over time when kept liquid. LNP not comprising PEGylated lipid can also be frozen and thawed.

The material obtained also maintain intact and functional as demonstrated in Table 13 below. The in vitro expression was characterized using mRNA expressing luciferase and HEK293 cells upon pre-incubation with human serum.

TABLE 13

| | | | | | In vitro expression [Mio | | |
|---|---|---|---|---|---|---|---|
| | | RNA integrity | | | light units] | | |
| | | | 4 | 4 | | 4 | 4 |
| | PEGylated | | weeks | weeks −70° | | weeks | weeks −70° |
| | lipid | T = 0 | 5° C. | C. | T = 0 | 5° C. | C. |
| Anionic | No | 83 | 74 | 77 | 3.0 | 3.5 | 5.3 |
| amphiphile | No | 82 | 75 | 76 | 5.8 | 6.5 | 9.1 |
| | No | 83 | 74 | 74 | 1.4 | 0.7 | 1.0 |

The results for RNA integrity and functionality show LNP not comprising PEGylated lipid are suitable to maintain the integrity and functionality of the encapsulated mRNA over time and upon freeze/thaw.

### Example 4

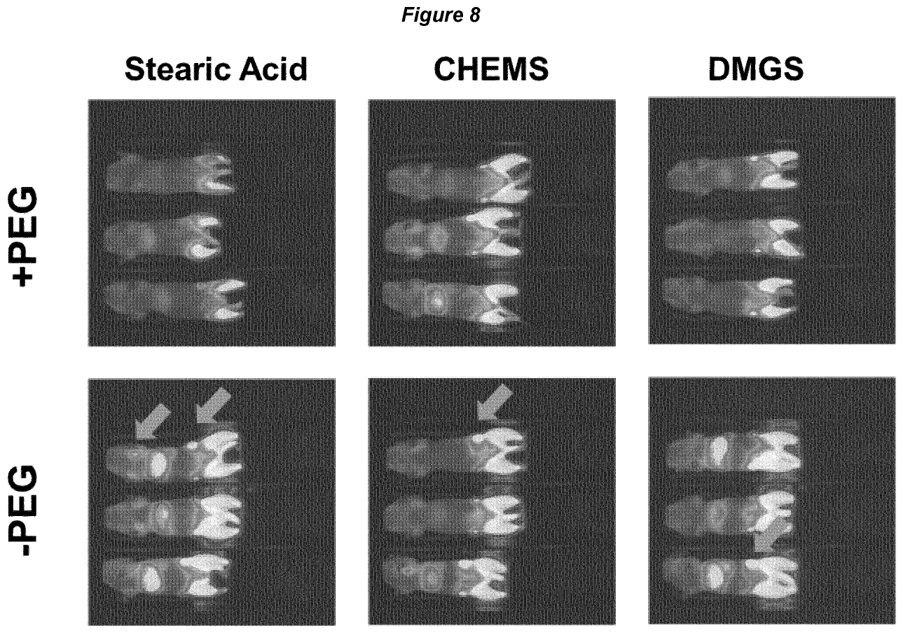

Materials obtained in example 3 were tested in vivo. Mice (n=3) were injected intramuscularly using a dose of lug RNA per injection site. The distribution of the expression signal is shown in FIG. 8. Expression of luciferase was observed at all injection sites for all LNP tested. LNP not comprising PEG-lipid feature a stronger signal at the injection site. In addition, a higher expression of luciferase is observed in liver and lymph nodes (arrows) in pairwise comparisons between PEGylated and non-PEGylated LNP.

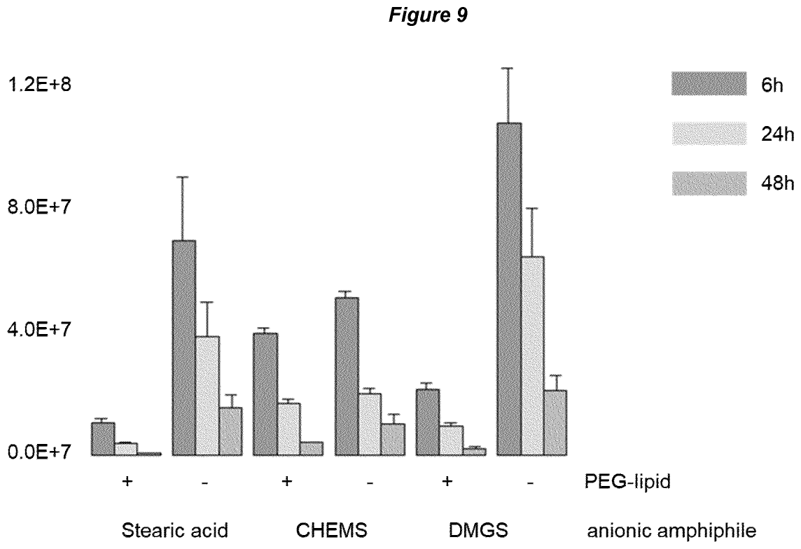

Expression of luciferase was monitored for 48 hours and quantified, results are shown in FIG. 9. LNP not comprising PEG-lipid show a higher expression of luciferase when compared to their PEGylated counterparts. The difference was most striking for LNP comprising stearic acid or DMGS as amphiphile. Expression of luciferase was comparable amongst LNP comprising anionic amphiphile but not PEG-lipid.

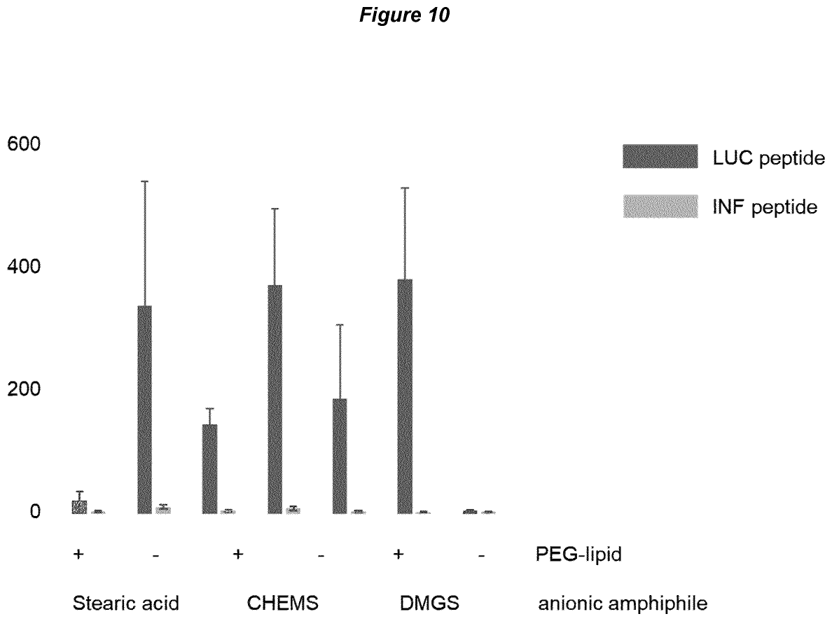

Eventually, the ability of LNP comprising anionic amphiphiles for induction of an immune response was analyzed using an ELISPOT assay. For that, LNP were manufactured as described in example 3 in the presence or absence of PEG-lipid and administered into the musculus gastrocnemius of mice. Upon sacrifice of the animals, splenocytes were isolated and challenged with peptides specific to luciferase or influenza (control peptide). As demonstrated in FIG. 10, LNP comprising anionic amphiphiles but not PEG-lipid induce a strong and specific T-cell immunity upon intramuscular injection.

### Example 5

LNPs were prepared using an aqueous-ethanol mixing protocol in a volume part ratio of 3:1:2 (Acidified mRNA:organic phase:quench buffer). For that, mRNA having a concentration of 0.4 mg/mL was provided in 50 mM sodium acetate pH 5.5. The organic phase comprising 47.4 mM of total lipids (47.0 mol BHD-C2C2-PipZ, 13.0 mol % DSPC, 32.0 mol % cholesterol and 8.0 mol % DMGS) was provided in ethanol. Quench buffer was 50 mM Tris pH 7.7. A raw colloid was produced by continuous mixing of the acidified mRNA with the organic phase, immediately followed by quenching.

Flow rates ranged for the 0.15 mm ID mixer from 5-30 ml/min, for the 0.3 mm ID mixer from 10-240 ml/min and for the 0.5 mm ID mixer from 10-480 ml/min.

PDI and the average particle diameter size were determined by dynamic light scattering using a Zetasizer available from Malvern. Results are shown in Table 14.

Reynolds numbers in the range of from about 1000 to about 10000 generally yielded particularly advantageous PDI and/or particle sizes. Thus, this range is a particularly good candidate for choosing the Reynolds number for the flow of the liquid composition after the liquid with the RNA and the liquid with the lipids have been mixed as such a flow seems to promote the formation of uniform, e.g. PDI<0.1 or PDI<0.15, and/or small, e.g. size<100 nm, particles. In some cases, high quality particles were also achieved with Reynolds numbers greater than 10000, e.g. up to about 14000.

TABLE 14
Overview on particle attributes of the raw colloid after quenching for the BHD-C2C2-PipZ formulation
stabilized with DMGS as anionic lipid, produced with different mixer outlet diameters along Re#.

| Reynolds Numbers | | | | PDI | | | | Size | | | |
|---|---|---|---|---|---|---|---|---|---|---|---|
| | T- | T- | T- | | T- | T- | T- | | T- | T- | T- |
| | Mixer | Mixer | Mixer | | Mixer | Mixer | Mixer | | Mixer | Mixer | Mixer |
| | 0.15 | 0.30 | 0.50 | | 0.15 | 0.30 | 0.50 | | 0.15 | 0.30 | 0.50 |
| Flowrate | mm | mm | mm | Flowrate | mm | mm | mm | Flowrate | mm | mm | mm |
| [ml/min] | i.d. | i.d. | i.d. | [ml/min] | i.d. | i.d. | i.d. | [ml/min] | i.d. | i.d. | i.d. |
| 5 | 691 | | | 5 | 0.104 | | | 5 | 137 | | |
| 7 | 967 | | | 7 | 0.129 | | | 7 | 155 | | |
| 10 | 1382 | 691 | 415 | 10 | 0.090 | 0.057 | 0.134 | 10 | 85 | 94 | 201 |
| 12 | 1658 | | | 12 | 0.073 | | | 12 | 75 | | |
| 15 | 2073 | | | 15 | 0.075 | | | 15 | 80 | | |
| 20 | 2764 | 1383 | 830 | 20 | 0.094 | 0.066 | 0.014 | 20 | 78 | 84 | 103 |
| 40 | | 2765 | 1660 | 40 | | 0.028 | 0.031 | 40 | | 81 | 91 |
| 60 | | 4148 | 2490 | 60 | | 0.033 | 0.013 | 60 | | 78 | 86 |
| 80 | | 5530 | 3320 | 80 | | 0.059 | | 80 | | 77 | |
| 100 | | 6913 | 4150 | 100 | | 0.036 | 0.036 | 100 | | 77 | 83 |
| 150 | | 10364 | 6225 | 150 | | 0.196 | 0.010 | 150 | | 87 | 81 |
| 200 | | 13818 | 8300 | 200 | | 0.183 | 0.011 | 200 | | 93 | 79 |
| 240 | | 16582 | 9960 | 240 | | 0.175 | 0.067 | 240 | | 90 | 77 |
| 480 | | | 19898 | 480 | | | 0.100 | 480 | | | 87 |

### Example 6

In the compositions of this Example, various ionizable lipids were used to generate anionic LNP not comprising any stealth moiety. Libraries of lipid compositions were prepared comprising 7.8 mol % dimyristoylglycerolhemisuccinate (DMGS), between 2.9 and 17.6 mol % of distearoylphosphatidylcholine (DSPC), between 27.5 and 56.9 mol % of the ionizable lipid and the remainder being cholesterol. The objectives were (i) to demonstrate colloidal stability of LNPs having anionic amphiphiles in the absence of PEG for various, structurally different ionizable lipids and (ii) to study the influence of the replacement on the in vitro expression.

LNP were generated, particle size determined, and in vitro expression was monitored as described in Example 1.

The ionizable lipids are provided in Table 15 below:

TABLE 15

| | | # of |
|---|---|---|
| Name | Formula | nitrogens |
| BODD-C2C2-PipZ | 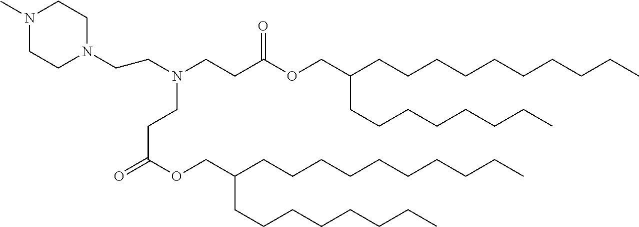 [SMILES: CCCCCCCCCCC(CCCCCCCC)COC(=O)CCN(CCC(=O)OCC(CCCCCCCC)CCCCCCCCCC)CCN1CCN(C)CC1] | 3 |
| | BODD-C2C2-PipZ | |
| BODD-C2C2-Pyr | 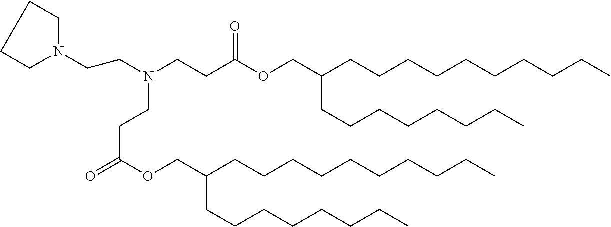 [SMILES: CCCCCCCCCCC(CCCCCCCC)COC(=O)CCN(CCC(=O)OCC(CCCCCCCC)CCCCCCCCCC)CCN1CCCC1] | 2 |
| | BODD-C2C2-Pyr | |
| BODD-C2C2- 1MePyr | 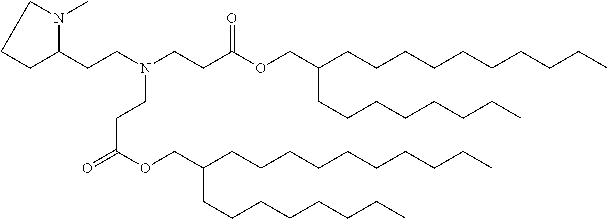 [SMILES: CCCCCCCCCCC(CCCCCCCC)COC(=O)CCN(CCC(=O)OCC(CCCCCCCC)CCCCCCCCCC)CCC1CCCN1C] | 2 |
| | BODD-C2C2-1Me-Pyr | |
| BODD-C2C2- 1Me-3PipD | 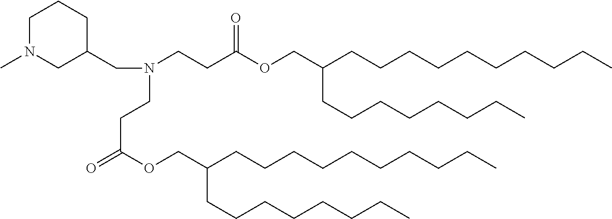 [SMILES: CCCCCCCCCCC(CCCCCCCC)COC(=O)CCN(CCC(=O)OCC(CCCCCCCC)CCCCCCCCCC)CC1CCCN(C)C1] | 2 |
| | BODD-C2C2-3PipD | |
| BODD-C2C2- DMA | 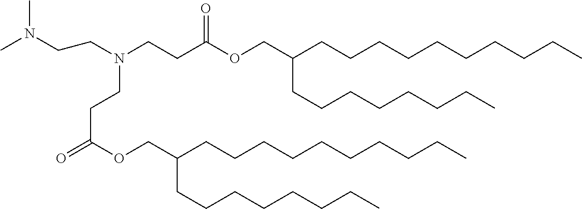 [SMILES: CCCCCCCCCCC(CCCCCCCC)COC(=O)CCN(CCC(=O)OCC(CCCCCCCC)CCCCCCCCCC)CCN(C)C] | 2 |
| | BODD-C2C2-DMA | |
| BODD-C2C4- PipZ | 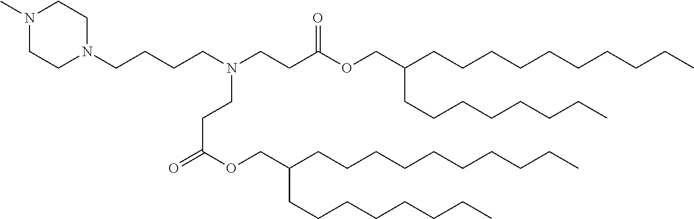 [SMILES: CCCCCCCCCCC(CCCCCCCC)COC(=O)CCN(CCCCN1CCN(C)CC1)CCC(=O)OCC(CCCCCCCC)CCCCCCCCCC] | 3 |
| | BODD-C2C4-PipZ | |
| BODD-C2C4-Pyr | 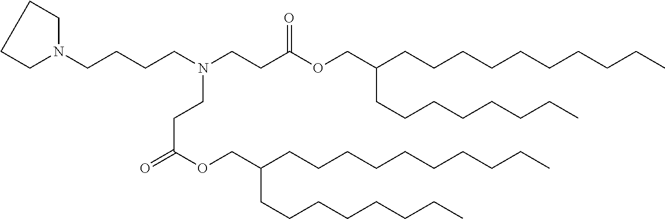 [SMILES: CCCCCCCCCCC(CCCCCCCC)COC(=O)CCN(CCCCN1CCCC1)CCC(=O)OCC(CCCCCCCC)CCCCCCCCCC] | 2 |
| | BODD-C2C4-Pyr | |
| BHD-C2C2-PipZ | 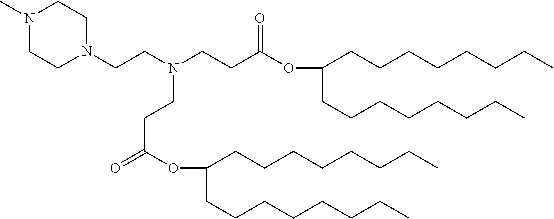 [SMILES: CCCCCCCCC(CCCCCCCC)OC(=O)CCN(CCC(=O)OC(CCCCCCCC)CCCCCCCC)CCN1CCN(C)CC1] | 3 |
| | BHD-C2C2-PipZ | |
| BHD-C2C4-PipZ | 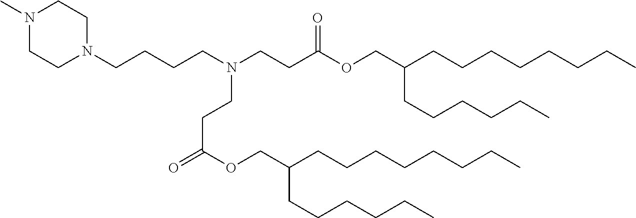 [SMILES: CCCCCCCCC(CCCCCC)COC(=O)CCN(CCCCN1CCN(C)CC1)CCC(=O)OCC(CCCCCC)CCCCCCCC] | 3 |
| DND-C2C4-PipZ | 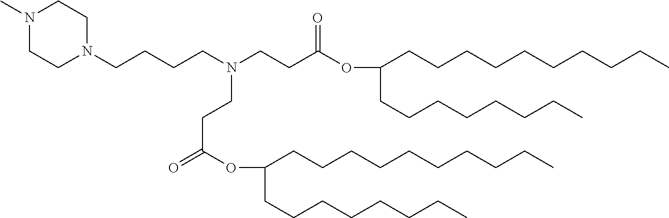 [SMILES: CCCCCCCCCCC(CCCCCCCC)OC(=O)CCN(CCCCN1CCN(C)CC1)CCC(=O)OC(CCCCCCCC)CCCCCCCCCC] | 3 |

### Results

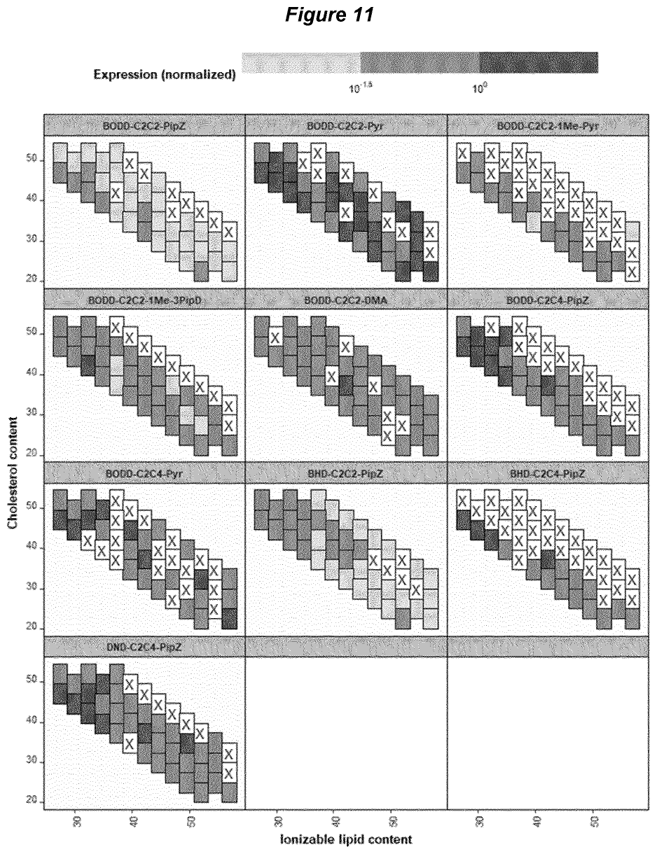

The results are shown in FIG. 11, which shows in vitro expression for LNP not comprising a stealth lipid. LNP were made from cationically ionizable lipid selected from Table 15, cholesterol, DSPC and DMGS.

LNP were made as described above and particle size was monitored. LNP larger than 250 nm are denoted with “x” and excluded from further analysis. Levels of in vitro expression were obtained without prior incubation with serum on HEPG2 cells. Signals were normalized to reference=1 and categorized to “low activity” in light grey having an expression less than 10^{-1.5} of the reference, “high activity” in grey having an expression between 10^{-1.5} and 10⁺⁰ of the reference and “superior activity” in dark grey having an expression higher than reference.

The results in FIG. 11 demonstrate that the invention can be practiced using a wide variety of ionizable lipids. The tested ionizable lipids contained one, two or three nitrogens. The distance between the central nitrogen and nitrogens in the head group was different. The tested ionizable lipids had a variety of different structures in their apolar tails, including: primary (BHD) or secondary (BODD) alcohols, branched and symmetric or branched and asymmetric. The results therefore demonstrate that stable LNP having good in vitro activity can be made using a broad range of different cationically ionizable lipids.

### Example 7

In the compositions of this Example, various ionizable lipids were used to generate anionic LNP not comprising any stealth moiety and anionic amphiphiles selected from the group of diacylglycerolhemisuccinates were used. Libraries of lipid compositions were prepared comprising 8 mol % diacylglycerolhemisuccinate, 13 mol % of distearoylphosphatidylcholine (DSPC), between 28 and 58 mol % of the ionizable lipid and the remainder being cholesterol. The objectives were (i) to demonstrate colloidal stability of LNPs having anionic amphiphiles in the absence of PEG for various ionizable lipids and diacylglycerolhemisuccinates and (ii) to study the influence of the replacement on the in vitro expression.

LNP were generated, particle size determined, and in vitro expression was monitored as described in Example 1.

The ionizable lipids are provided in Table 15 of Example 6. The diacylglycerols are provided in Table 16 below:

TABLE 16

| Name | Formula |
|---|---|
| DLGS | 1,2 dilauroylglyceryl hemisuccinate |
| DMGS | 1,2 dimyristoylglyceryl hemisuccinate |
| DPGS | 1,2 dipalmitoylglyceryl hemisuccinate |
| DSGS | 1,2 distearoylglyceryl hemisuccinate |
| SMGS | 1-stearoyl-2-myristoyl-sn-glycero-3-succinate |

### Results

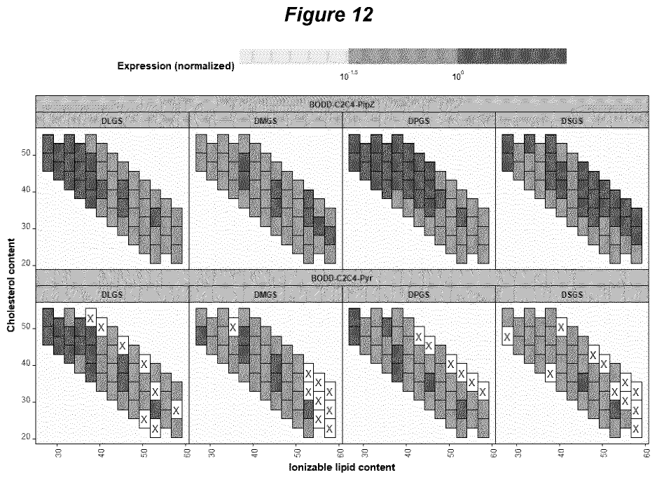

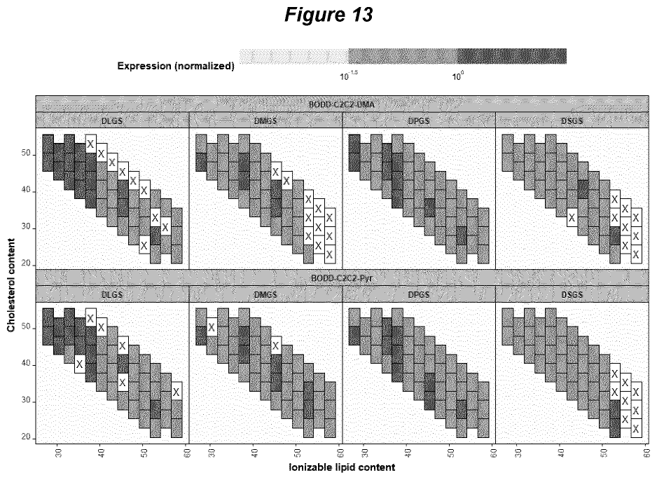

The results are shown in FIGS. 12 and 13, which shows in vitro expression for LNP not comprising a stealth lipid. LNP were made from cationically ionizable lipid selected from Table 14, cholesterol, DSPC and diacylglycerol selected from Table 16.

LNP were made as described above and particle size was monitored. LNP larger than 250 nm are denoted with “x” and excluded from further analysis. Levels of in vitro expression were obtained without prior incubation with serum on HEPG2 cells. Signals were normalized to reference=1 and categorized to “low activity” in light grey having an expression less than 10^{-1.5} of the reference, “high activity” in grey having an expression between 10^{-1.5} and 10⁺⁰ of the reference and “superior activity” in dark grey having an expression higher than reference.

The results in FIG. 12 demonstrate that the invention can be practiced using combinations of BODD-C2C4-PipZ and BODD-C2C4-Pyr with any of the diacylglycerols. The results of FIG. 13 demonstrate that the invention can be practiced using combinations of BODD-C2C2-DMA and BODD-C2C2-Pyr with any of the diacylglycerols. The results therefore demonstrate that stable LNP having good in vitro activity can be made with a broad range of different anionic amphiphiles.

### Example 8

In the compositions of this Example, various ionizable lipids were used to generate anionic LNP not comprising any stealth moiety and anionic amphiphiles selected from the group of diacylglycerolhemisuccinates were used. Libraries of lipid compositions were generated wherein the molar percentage of the anionic amphiphile was between 0.5 and 2.5 mol %, the molar percentage of DSPC was between 2.5 and 25.0 mol %, the molar percentage of the ionizable lipid between 25 and 55% and the remainder being cholesterol.

Ionizable lipids were selected from the group of BHD-C2C2-PipZ and MC-3. Anionic amphiphiles were selected from the group of 1,2 dimyristoylglyceryl-hemisuccinate (DMGS), 1,2 dipalmitoylglycerylhemisuccinate (DPGS), and 1-stearoyl-2-myristoyl-sn-glycero-3-succinate (SMGS).

The objectives were (i) to demonstrate colloidal stability of LNPs having low amounts of anionic amphiphiles in the absence of PEG for various ionizable lipids and diacylglycerolhemisuccinates and (ii) to study the influence of the replacement on the in vitro expression.

LNP were generated, particle size determined, and in vitro expression was monitored as described in Example 1.

### Results

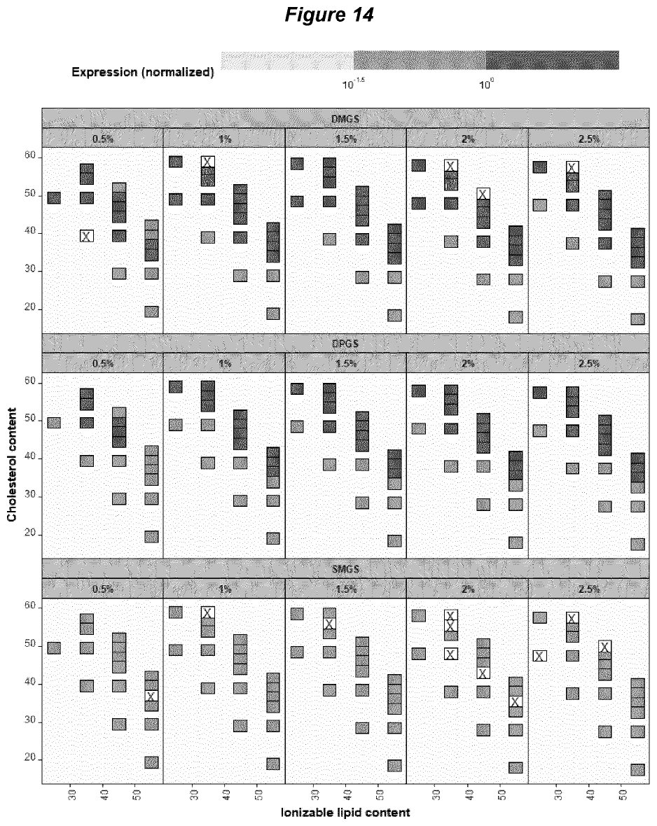

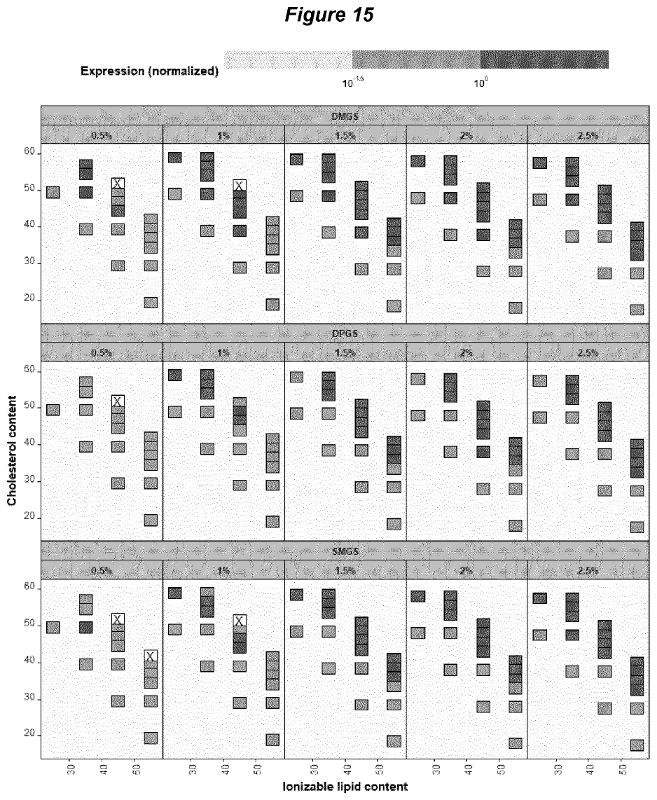

The results are shown in FIGS. 14 and 15, which shows in vitro expression for LNP not comprising a stealth lipid.

LNP were made as described above and particle size was monitored. LNP larger than 250 nm are denoted with “x” and excluded from further analysis. Levels of in vitro expression were obtained without prior incubation with serum on HEPG2 cells. Signals were normalized to reference=1 and categorized to “low activity” in light grey having an expression less than 10^{-1.5} of the reference, “high activity” in grey having an expression between 10^{-1.5} and 10⁺⁰ of the reference and “superior activity” in dark grey having an expression higher than reference.

The results in FIG. 14 demonstrate that the invention can be practiced using low amounts of DMGS, DPGS, DSGS, or SMGS when the ionizable lipid is BHD-C2C2-PipZ. The results in FIG. 15 demonstrate that the invention can be practiced using low amounts of DMGS, DPGS, DSGS, or SMGS when the ionizable lipid is MC-3. The results therefore demonstrate that stable LNP having good in vitro activity can be made even with low amounts of anionic amphiphile, and that this is true across a broad range of cationically ionizable lipids and for different anionic amphiphiles.

### Example 9

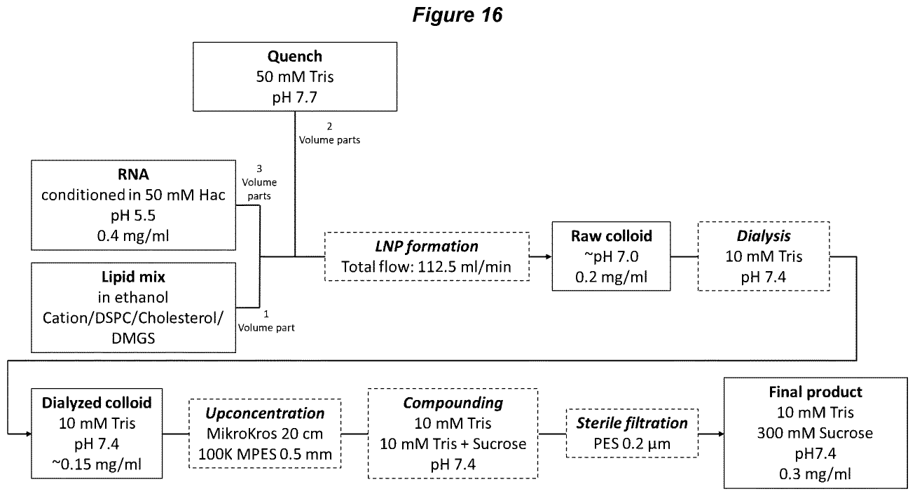

mRNA having a concentration of 0.4 mg/ml was provided in 50 mM sodium acetate pH 5.5. The organic phase comprising 47.4 mM of total lipids (47.0 mol % ionizable lipid, 13.0 mol % DSPC and 32.0 mol % cholesterol and 8.0 mol % DMGS) was provided in ethanol. Quench buffer was 50 mM Tris*HCl pH 7.7. A raw colloid was produced by continuous mixing 3 volumes of the acidified mRNA with 1 volume of the organic phase in a first mixing T, followed by inline addition of 2 volumes of quench buffer in second mixing T at a total flow rate of 112.5 ml/min. The neutralized raw colloid so obtained was dialyzed against Tris*HCl 10 mM pH 7.4 followed by up-concentration using a crossflow membrane (MikroKros 20 cm 100K MPES 0.5 mm (C02-E100-05)). Eventually, Tris*HCL 10 mM pH 7.4 buffer and Tris*HCl 10 mM+1.2 M sucrose pH 7.4 were added to arrive at a final product having 0.3 mg/ml of RNA in Tris 10 mM*HCl pH 7.4, 300 mM sucrose. See also the process flow diagram in FIG. 16. The product characteristics are shown in Table 17.

TABLE 17

| | BODD-C2C2-1Me- | 4DODL-CyHex, | BHD-C2C2-PipZ, | BODD-C2C2- |
|---|---|---|---|---|
| | Pyr, Chol, DSPC, | Chol, DSPC, | Chol, DSPC, | DMA, Chol, DSPC, |
| Formulation | DMGS | DMGS | DMGS | DMGS |
| [%] | (47.5:40.7:10:8%) | (47.5:40.7:10:8%) | (47:32:13:8%) | (47:32:13:8%) |
| Size [nm] | 86 | 94 | 82 | 83 |
| PDI | 0.07 | 0.04 | 0.07 | 0.06 |
| EE [%] | 99 | 97 | 98 | >99 |
| RNA conc. | 0.293 | 0.297 | 0.280 | 0.276 |
| [mg/mL] | | | | |
| Zeta potential | −35.4 | −30.4 | −54.8 | −47.3 |
| [mV] | | | | |
| IVE % BM | 245 | 139 | 416 | 219 |
| HepG2 − S | | | | |
| IVE % BM | 271 | 332 | 591 | 327 |
| HepG2 + S | | | | |

LNP comprising an anionic moiety but no stealth lipid can be manufactured when the pH of the primary colloid obtained after mixing of the acidified RNA and the organic phase is adjusted instantaneously. This is in contrast to the state of the art where water is used to reduce the content of ethanol at this mixing step, and the so-called quenching and a transition of the pH in subsequent processing steps is typically achieved in minutes or hours. In the present Example, the pH transition was immediate within the time of mixing. Since the process was carried out in a continuous mode the mixing time was below one second. Quenching and adjustment of pH was combined into a single step, as compared to other known processes which use two distinct steps.

The materials obtained have a low polydispersity substantially below PDI of 0.1 thus representing a homogeneous dispersed phase. The encapsulation of RNA is essentially complete. The activity of the materials using in vitro expression (IVE) is high and exceeds that of the benchmark. The activity is obtained both in the presence and absence of human serum in a pre-incubation period before cell testing. The zeta potential of the dispersed phase is clearly negative indicating exposure of the anionic amphiphile at the surface of the lipid nanoparticle.

### Example 10

mRNA encoding an antibody having a concentration of 0.4 mg/ml was provided in 50 mM sodium acetate pH 5.5. The organic phase comprising 47.4 mM of total lipids (47.0 mol % ionizable lipid, 13.0 mol % DSPC and 32.0 mol % cholesterol and 8.0 mol % DMGS) was provided in ethanol. Quench buffer was 50 mM Tris*HCl pH 7.7. A raw colloid was produced by continuous mixing 3 volumes of the acidified mRNA with 1 volume of the organic phase in a first mixing T, followed by inline addition of 2 volumes of quench buffer in second mixing T at a total flow rate of 112.5 ml/min. The neutralized raw colloid so obtained was purified and concentrated using tangential flow filtration against Tris*HCl 10 mM pH 7.4 using a crossflow membrane (MidiKros 41.5 cm 100K, 235 cm², mPES 0.5 mm (D04-E100-05-S)). Eventually, Tris*HCl 10 mM pH 7.4 buffer and Tris*HCl 10 mM+1.2 M sucrose pH 7.4 were added to arrive at a final product having 1.0 mg/ml of RNA in Tris 10 mM*HCl pH 7.4, 300 mM sucrose. The product characteristics are shown in Table 18.

TABLE 18

| Product characteristic | Value |
|---|---|
| Particle size (nm) | 74 |
| Polydispersity | 0.092 |
| SVP > 10 μm (N particles/mL) | 8 |
| SVP > 25 μm (N particles/mL) | 3 |
| RNA content (mg/mL) | 1.017 |
| Encapsulation efficiency (%) | 90 |
| Heavy chain RNA integrity (%)¹ | 30.3 |
| Light chain RNA integrity (%)² | 55.0 |
| pH | 7.42 |
| Osmolality (mOsmol/kg) | 356 |
| Zeta potential (mV) | −32 |

¹the RNA integrity of the heavy chain is expressed as % of intact RNA relative to the total RNA signal, the theoretical limit is 60% based on the relative amounts of RNA in the heavy and light chain and the initial integrity before LNP production.

The material obtained is stable when exposed to multiple cycles of freeze-thaw (Table 19a), as well as up to 6 months at −80° C. (Table 19b) or +5° C. (Table 19c).

TABLE 19a

| FT cycles | 0 | 1 | 3 | 5 |
|---|---|---|---|---|
| Particle size (nm) | 74 | 82 | 84 | 85 |
| Polydispersity | 0.092 | 0.143 | 0.152 | 0.143 |

TABLE 19b
Frozen stability at −80° C. up to 6 months

| | T0 | T1 | T2 |
|---|---|---|---|
| | Time (months) | | |
| | 0 | 3 | 6 |
| Particle size (nm) | 74 | 77.3 | 76.5 |
| Polydispersity | 0.092 | 0.086 | 0.09 |
| HC RNA integrity (%) | 30.3 | 29.70 | 30.50 |
| LC RNA integrity (%) | 55.0 | 48.73 | 57.38 |

TABLE 19c
Liquid stability at 4° C. up to 6 months

| | T0 | T1 | T2 | T3 | T4 |
|---|---|---|---|---|---|
| | Time (months) | | | | |
| | 0 | 1 | 2 | 3 | 6 |
| Particle size (nm) | 74 | — | 101.7 | 77.4 | 82.5 |
| Polydispersity | 0.092 | — | 0.125 | 0.08 | 0.233 |
| SVP > 10 μm | 8 | — | — | 93 | 43 |
| (N particles/mL) | | | | | |
| SVP > 25 μm | 3 | — | — | 3 | 11 |
| (N particles/mL) | | | | | |
| RNA accessibility | 90 | — | — | — | 91.9 |
| HC RNA integrity (%) | 30.3 | 30.10 | 31.75 | 30.15 | 30.48 |
| LC RNA integrity (%) | 55.0 | 56.35 | 53.90 | 54.30 | 53.60 |

The material obtained has a particle size below 100 nm and a polydispersity below 0.1 at the time of release. The material is stable upon repeated freeze/thaw and stable for at least 6 months when frozen. The material is also essentially stable in a liquid state for at 6 months. The dispersed phase has a negative zeta potential in line with a presence of anionic amphiphiles at the surface of the lipid nanoparticle.

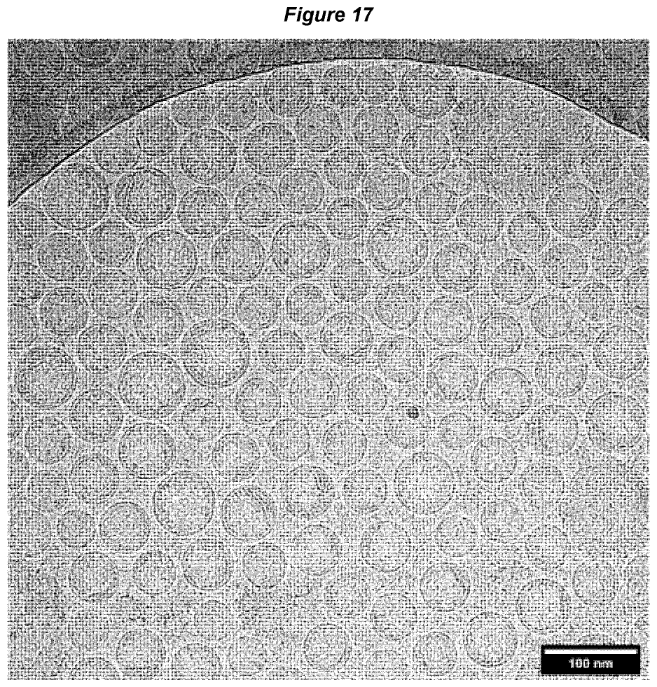

The structure of the dispersed phase was investigated using cryoTEM and a representative image is shown in FIG. 17. It can be observed that particles are homogeneous in size and structure. Semi-quantitative analysis of >150 particles indicates that ~94% of particles feature an electron dense core surrounded by a layer of higher electron density at an average size of 54 nm±9 nm. A small population of particles (~6%) was found with a similar electron dense core but displaying minor phase separation of a bilayer with an aqueous content and a size of 64 nm±10 nm. Without wishing to be bound to any theory such layers at the surface of the electron dense particles may represent a lipid layer comprising DSPC and the anionic amphiphile.

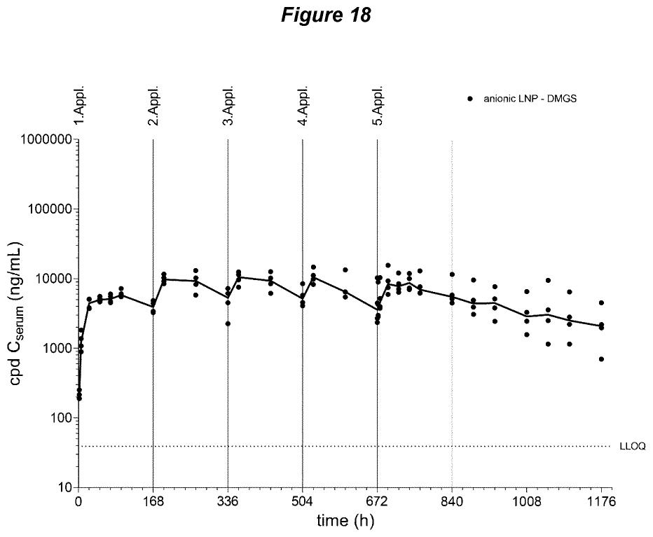

The material can repeatedly be administered into animals. A dose of 10 μg (based on mRNA) was administered into the tail vein of Wistar rats q1w, n=4. Expression of the antibody was monitored in plasma for five repeated injections. The peak and trough concentrations were found to be similar over the entire period of treatment, as shown in FIG. 18.

Without wishing to be bound by any theory, the absence of a PEG lipid moiety eliminates the formation of anti-PEG antibodies upon repeated dosing which in turn may interfere with the pharmacological action. Hence the LNP comprising anionic amphiphiles and not having PEG lipids or other stealth lipid moieties have a design advantage over LNP known in the art which converts into a functional advantage of such materials.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in chemistry, biochemistry, molecular biology, biotechnology or related fields are intended to be within the scope of the following claims.

1. A composition comprising:
(a) an active ingredient; and
(b) a lipid mixture comprising:
(i) a cationically ionisable lipid capable of forming a lipid nanoparticle;
(ii) a steroid; and
(iii) a negatively charged amphiphile having a hydrophilic portion and a lipophilic portion;
wherein the composition is a lipid nanoparticle composition and is substantially free of a polyethylene glycol-conjugated lipid, wherein the polyethylene glycol (PEG) moiety of the PEG-conjugated lipid has at least 5 consecutive ethylene glycol repeating units.

2. The composition of claim 1, wherein the lipophilic portion of the negatively charged amphiphile has from 6 to 40 carbon atoms, preferably 6 to 20 carbon atoms.

3. The composition of claim 1, wherein the negatively charged amphiphile is a carboxylic acid.

4. The composition of claim 3, wherein the negatively charged amphiphile is selected from the group consisting of hexanoic acid, octanoic acid, decanoic acid, lauric acid, myristic acid, palmitic acid, stearic acid, eicosanoic acid, tricosanic acid, 2-hydroxytetradecanoic acid, 2-methyloctadecanoic acid, 2-bromo-hexadecanoic acid, 2-propylpentanoic acid, 2-butyloctanoic acid, 2-hexyldecanoic acid, 9-hydroxy-stearic-acid, trans-2-decenoic acid, (9Z)-9-hexadecenoic acid, linolic acid, linolenic acid, oleic acid, elaidic acid, arachidonic acid, cyclododecanoic acid, adamantylacetic acid, dicyclohexylacetic acid, trans-4-pentylcyclohexane-carboxylic acid, 4-(decyloxy)benzoic acid, 4-octylbenzoic acid, cholic acid, and lithocholic acid, or a mixture of any thereof.

5. The composition of claim 1, wherein the negatively charged amphiphile is a hemiester of a dicarboxylic acid with diacylglycerol.

6. The composition of claim 1, wherein the negatively charged amphiphile is selected from the group consisting of 1,2-dimyristoylglyceryl hemisuccinate, 1,2-dioleoylglyceryl hemisuccinate, and 1-stearoyl-2-myristoyl-glyceryl hemisuccinate, or a mixture of any thereof.

7. The composition of claim 1, wherein the negatively charged amphiphile is a hemiester of a dicarboxylic acid with cholesterol.

8. The composition of claim 7, wherein the negatively charged amphiphile is selected from the group consisting of cholesterol hemisuccinate, cholesterol hemimalonate and cholesterol hemiadipate, or a mixture of any thereof.

9. The composition of claim 1, wherein the negatively charged amphiphile is an organic sulfate or sulfonate.

10. The composition of claim 9, wherein the negatively charged amphiphile is selected from the group consisting of sodium lauryl sulfate, sodium hexadecane sulfonate and sodium dodecylbenzene sulfonate, or a mixture of any thereof.

11. The composition of claim 1, wherein the negatively charged amphiphile is an organic phosphonate.

12. The composition of claim 11, wherein the negatively charged amphiphile is selected from the group consisting of octadecylphosphonic acid and dodecylphosphonic acid, or a mixture thereof.

13. The composition of claim 1, wherein the negatively charged amphiphile is an anionic phospholipid.

14. The composition of claim 13, wherein the negatively charged amphiphile is selected from the group consisting of a phosphatidylserine, a phosphatidylglycerol and a phosphatidic acid.

15. The composition of claim 14, wherein the negatively charged amphiphile is a phosphatidylserine.

16. The composition of claim 15, wherein the negatively charged amphiphile is 1,2-dioleoylphosphatidylserine.

17. The composition of any preceding claim, wherein the negatively charged amphiphile is present in the composition in an amount of up to 20 mol % of the total lipid present in the composition.

18. The composition of any preceding claim, wherein the cationically ionisable lipid is selected from the group consisting of:
[(4-hydroxybutyl) azanediyl]di(hexane-6,1-diyl)bis(2-hexyldecanoate)) (ALC-315);
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
N,N-dimethyl-2,3-dioleyloxypropylamine (DODMA);
di((Z)-non-2-en-1-yl)-9-((4-(dimethylaminobutanoyl)oxy) heptadecanedioate (L319);
bis-(2-butyloctyl) 10-(N-(3-(dimethylamino) propyl) nonanamido)-nonadecanedioate (A9);
(heptadecan-9-yl 8-{(2-hydroxyethyl) [6-oxo-6-(undecyloxy) octyl]amino}-octanoate) (L5);
heptadecan-9-yl 8-{(2-hydroxyethyl) [6-oxo-6-(undecyloxy) hexyl]amino}-octanoate) (SM-102);
O—[N-{(9Z,12Z)-octadeca-9,12-dien-1-yl)}-N-{7-pentadecylcarbonyloxyoctyl}-amino]4-(dimethylamino)butanoate (HY501);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1Me-Pyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ);
di(nonadecan-9-yl) 3,3′-((4-(4-methylpiperazin-1-yl)butyl) azanediyl)dipropionate (DND-C2-C4-PipZ);
or a mixture of any thereof.

19. The composition of claim 18, wherein the cationically ionisable lipid is selected from the group consisting of:
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
N,N-dimethyl-2,3-dioleyloxypropylamine (DODMA);
di((Z)-non-2-en-1-yl)-9-((4-(dimethylaminobutanoyl)oxy) heptadecanedioate (L319);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
O—[N-{(9Z,12Z)-octadeca-9,12-dien-1-yl)}-N-{7-pentadecylcarbonyloxyoctyl}-amino]4-(dimethylamino)butanoate (HY501);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ);
di(nonadecan-9-yl) 3,3′-((4-(4-methylpiperazin-1-yl)butyl) azanediyl)dipropionate (DND-C2-C4-PipZ);
or a mixture of any thereof.

20. The composition of claim 19, wherein the cationically ionisable lipid is selected from the group consisting of:
N,N-dimethyl-2,3-dioleyloxypropylamine (DODMA);
[(4-hydroxybutyl) azanediyl]di(hexane-6,1-diyl)bis(2-hexyldecanoate)) (ALC-315);
9-heptadecanyl 8-{(2-hydroxyethyl) [6-oxo-6-(undecyloxy) hexyl]amino}octanoate (SM102);
di(heptadecan-9-yl) 3,3′-((2-(4-methylpiperazin-1-yl)ethyl) azanediyl)dipropionate (BHD-C2C2-PipZ);
bis(2-octyldodecyl) 3,3′-((2-(1-methylpyrrolidin-2-yl)ethyl) azanediyl)dipropionate (BODD-C2C2-1MePyr);
bis(2-octyldodecyl) 3,3′-((2-(pyrrolidin-1-yl)ethyl) azanediyl)dipropionate (BODD-C2C2-Pyr);
bis(2-octyldodecyl) 3,3′-(((1-methylpiperidin-3-yl)methyl) azanediyl)dipropionate (BODD-C2C2-1Me-3PipD);
bis(2-octyldodecyl) 3,3′-((2-(dimethylamino)ethyl) azanediyl)dipropionate (BODD-C2C2-DMA);
bis(2-octyldodecyl) 3,3′-((4-(4-methylpiperazin-1-yl)butyl) azanediyl)dipropionate (BODD-C2C4-PipZ);
bis(2-octyldodecyl) 3,3′-((4-(pyrrolidin-1-yl)butyl) azanediyl)dipropionate (BODD-C2C4-Pyr);
bis(2-hexyldecyl) 3,3′-((4-(4-methylpiperazin-1-yl)butyl) azanediyl)dipropionate (BHD-C2C4-PipZ);
di(nonadecan-9-yl) 3,3′-((4-(4-methylpiperazin-1-yl)butyl) azanediyl)dipropionate (DND-C2-C4-PipZ);
or a mixture of any thereof.

21. The composition of any preceding claim, wherein the cationically ionisable lipid is present in the composition in an amount of 20 to 80 mol % of the total lipid present in the composition.

22. The composition of any preceding claim, wherein the lipid mixture comprises one or more additional lipids.

23. The composition of claim 22, wherein the one or more additional lipids comprise a neutral or zwitterionic lipid.

24. The composition of claim 23, wherein the one or more additional lipids comprise a neutral or zwitterionic phospholipid.

25. The composition of claim 24, wherein the neutral or zwitterionic phospholipid is selected from the group consisting of:
distearoylphosphatidylcholine (DSPC);
dioleoylphosphatidylcholine (DOPC);
dimyristoylphosphatidylcholine (DMPC);
dipalmitoylphosphatidylcholine (DPPC);
palmitoyloleoyl-phosphatidylcholine (POPC);
N-palmitoyl-D-erythro-sphingosylphosphorylcholine (SM);
or a mixture of any thereof.

26. The composition of claim 25, wherein the neutral or zwitterionic phospholipid Is distearoylphosphatidylcholine (DSPC).

27. The composition of any one of claims 24 to 26, wherein the phospholipid is present in the composition in an amount of 5 to 30 mol % of the total lipid present in the composition.

28. The composition of any preceding claim, wherein the one or more additional lipids comprise a steroid.

29. The composition of claim 28, wherein the steroid is cholesterol.

30. The composition of claim 28 or 29, wherein the steroid is present in the composition in an amount of 10 to 60 mol % of the total lipid present in the composition.

31. The composition of any preceding claim, wherein the active ingredient is a nucleic acid.

32. The composition of claim 31, wherein the nucleic acid is RNA.

33. The composition of claim 31, wherein the nucleic acid is DNA.

34. The composition of claim 32, wherein the nucleic acid is mRNA.

35. The composition of any preceding claim, which is substantially free of a stealth lipid.

36. A pharmaceutical composition comprising the composition of any preceding claim, together with a pharmaceutically acceptable carrier.

37. The composition of any one of claims 1-35, or the pharmaceutical composition of claim 36, for use in medicine.

38. The composition of any one of claims 1-35, or the pharmaceutical composition of claim 36, for use in a prophylactic and/or therapeutic treatment of a disease involving an antigen.

39. The composition of any one of claims 1-35, or the pharmaceutical composition of claim 36, for use in treating cancer.

40. The composition of any one of claims 1-35, or the pharmaceutical composition of claim 36, for use in inducing an immune response.

41. The composition of any one of claims 1-35, or the pharmaceutical composition of claim 36, additionally including another therapeutic agent.

42. A method of preparing the composition according to any one of claims 1-35, wherein the method comprises:
(a) providing the active ingredient in an aqueous phase;
(b) providing an organic phase comprising the lipid mixture; and
(c) mixing the aqueous phase provided under (a) with the organic phase provided under (b), to form the composition.

43. A method of preparing the composition according to any one of claims 1-35, wherein the method comprises:
(a) providing the active ingredient in an aqueous phase;
(b) providing an organic phase comprising the lipid mixture;
(c) mixing the aqueous phase provided under (a) with the organic phase provided under (b), to form an intermediate composition; and
(d) immediately after step (c), adjusting the pH of the intermediate composition;
to form the composition.
